SEQUENCE LISTING (27 sequences)

SEQ ID NO: 1 - protein, 106 aa, Homo sapiens
MAVTALAARTWLGVWGVRTMQARGFGSDQSENVDRGAGSIREAGGAFGKREQAEEERYFRAQSREQLAALKKHHEEEIVHHKKEIERLQKEIERHKQKIKMLKHDD

SEQ ID NO: 2 - protein, 71 aa, Homo sapiens
MAVTALAARTWLGVWGVRTMQARGFGSDQSENVDRGAGSIREAGGAFGKREQAEEERYFRHYRLCFEISLG

SEQ ID NO: 3 - protein, 60 aa, Homo sapiens
MAVTALAARTWLGVWGVRTMQARGFGSDQSENVDRGAGSIREAGGAFGKREQAEEERYFR

SEQ ID NO: 4 - DNA, 21 bp, Artificial
  shRNA target sequence
ccatgaagaagaaatcgttca

SEQ ID NO: 5 - DNA, 60 bp, Artificial
  shRNA hairpin sequence
gccggccatgaagaagaaatcgttcactcgagtgaacgatttcttcttcatggttttttg

SEQ ID NO: 6 - DNA, 21 bp, Artificial
  shRNA target sequence
caccatgaagaagaaatcgtt

SEQ ID NO: 7 - DNA, 60 bp, Artificial
  shRNA hairpin sequence
gccggcaccatgaagaagaaatcgttctcgagaacgatttcttcttcatggtgttttttg

SEQ ID NO: 8 - DNA, 21 bp, Artificial
  shRNA target sequence
gagcgtctgcagaaagaaatt

SEQ ID NO: 9 - DNA, 60 bp, Artificial
  shRNA hairpin sequence
cccgggagcgtctgcagaaagaaattctcgagaatttctttctgcagacgctcttttttg

SEQ ID NO: 10 - DNA, 18 bp, Artificial
  primer
cttcggctcggatcagtc

SEQ ID NO: 11 - DNA, 22 bp, Artificial
  primer
ctgccagttgttctctactctg

SEQ ID NO: 12 - DNA, 16 bp, Artificial
  primer
ccctggcacccagcac

SEQ ID NO: 13 - DNA, 19 bp, Artificial
  primer
gccgatccacacggagtac

SEQ ID NO: 14 - DNA, 20 bp, Artificial
  primer
ccctaaaacccgccacatct

SEQ ID NO: 15 - DNA, 22 bp, Artificial
  primer
gagcgatggtgagagctaaggt

SEQ ID NO: 16 - DNA, 21 bp, Artificial
  primer
cgcattcctcatcccagtatg

SEQ ID NO: 17 - DNA, 23 bp, Artificial
  primer
aaaggacttggtgcagagttcag

SEQ ID NO: 18 - DNA, 27 bp, Artificial
  primer
attaccggtatggcagtgacggcgttg

SEQ ID NO: 19 - DNA, 43 bp, Artificial
  primer
attgaattcttaatcatcatgttttagcattttgatcttctgc

SEQ ID NO: 20 - DNA, 32 bp, Artificial
  primer
atggatccgccaccatggcagtgacggcgttg

SEQ ID NO: 21 - DNA, 50 bp, Artificial
  primer
atgcggccgcttaatcatcatgttttagcattttgatcttctgcttatgg

SEQ ID NO: 22 - DNA, 35 bp, Artificial
  primer
gagagagcaggctgaagcggaacgatatttccgag

SEQ ID NO: 23 - DNA, 35 bp, Artificial
  primer
ctcggaaatatcgttccgcttcagcctgctctctc

SEQ ID NO: 24 - DNA, 20 bp, Artificial
  primer
cggaaaaacagcagggaaat

SEQ ID NO: 25 - DNA, 20 bp, Artificial
  primer
ggcattggactggggtttac

SEQ ID NO: 26 - DNA, 20 bp, Artificial
  primer
attagggccgcaagaaaact

SEQ ID NO: 27 - DNA, 20 bp, Artificial
  primer
ctgtagcggctgatgttgaa

(19) United States
(12) Patent Application Publication
(10) Pub. No.: US 2015/0065556 A1
(43) Pub. Date: Mar. 5, 2015

(54) THERAPEUTIC TARGETS FOR MITOCHONDRIAL DISORDERS

(71) Applicant: Whitehead Institute for Biomedical Research, Cambridge, MA (US)

(72) Inventors: Kivanc Birsoy, Cambridge, MA (US); Walter Chen, Cambridge, MA (US); David M. Sabatini, Cambridge, MA (US)

(21) Appl. No.: 14/452,298

(22) Filed: Aug. 5, 2014

Related U.S. Application Data
(60) Provisional application No. 61/952,646, filed on Mar. 13, 2014.
(60) Provisional application No. 61/862,315, filed on Aug. 5, 2013.

(51) Int. Cl. C12N 15/113; C12N 5/071; G01N 33/68; C12Q 1/68

(52) U.S. Cl. CPC C12N 15/113; C12Q 1/6881; C12N 5/067; G01N 33/6893; C12Q 2600/136; C12Q 2600/158; C12N 2501/998; G01N 2500/04; C12N 2310/141; C12N 2310/14; C12N 2310/11; C12N 2310/531
USPC 514/44 A; 435/6.12; 436/501; 435/375

(57) ABSTRACT

In some aspects, compositions and methods for identifying therapeutic targets for treatment of mitochondrial disorders are provided. In some aspects compositions and methods for identifying therapeutic agents for treatment of mitochondrial disorders. In some aspects, the disclosure identifies ATPIF1 as a therapeutic target for mitochondrial disorders.

### CROSS-REFERENCE TO RELATED APPLICATIONS

This application claims the benefit of U.S. Provisional Application Nos. 61/862,315, filed Aug. 5, 2013, and 61/952,646, filed Mar. 13, 2014. The entire teachings of the above applications are incorporated herein by reference.

### BACKGROUND

Mitochondria are membrane-enclosed organelles composed of four compartments: the outer membrane, the inner membrane, the intermembrane space, and the matrix (the region inside the inner membrane). Mitochondria are found in almost all eukaryotic cells and perform a variety of different functions such as pyruvate oxidation, the tricarboxylic acid (TCA) cycle, and the generation of adenosine triphosphate (ATP) by oxidative phosphorylation. Defects in mitochondrial function are associated with a variety of human disorders. Although understanding of the molecular mechanisms underlying a number of these diseases has improved in recent years, current therapies are limited. There is a need in the art for new drug targets and new approaches to identifying agents useful for treating mitochondrial disorders.

### SUMMARY OF THE INVENTION

In some aspects, the invention relates to identification of targets for discovery of drugs for treatment of mitochondrial disorders.

In some aspects, the invention provides a method of identifying a gene that affects mitochondrial phenotype, the method comprising: (a) providing a plurality of mutagenized near-haploid mammalian cells; (b) isolating a cell that exhibits a mitochondrial phenotype of interest; and (c) identifying a gene that is mutated in the cell, thereby identifying a gene that affects mitochondrial phenotype. In some embodiments mutagenized near-haploid mammalian cells are near-haploid cells, e.g., near-haploid human cells, e.g., KBM7 cells. In some embodiments mutagenized near-haploid mammalian cells are mutagenized by insertional mutagenesis, e.g., using a gene trap vector. In some embodiments, identifying a gene mutated in the cell comprises identifying a gene containing at least a portion of the gene trap vector. In some embodiments the mutagenized near-haploid mammalian cells comprise abnormal or dysfunctional mitochondria, e.g., the cells were obtained by mutagenizing near-haploid mammalian cells that comprise abnormal or dysfunctional mitochondria. In some embodiments the method comprises (i) providing a plurality of near-haploid mammalian cells; and (ii) mutagenizing the cells. In some embodiments the method comprises isolating multiple cells that exhibit the mitochondrial phenotype of interest, and step (c) comprises identifying a gene, mutation of which is correlated with the phenotype of interest. In some embodiments step (b) comprises isolating multiple cells that exhibit the mitochondrial phenotype of interest, and step (c) comprises identifying a gene whose mutation frequency in cells that exhibit the phenotype of interest is significantly greater than a reference frequency. In some embodiments the reference frequency is approximately equal to (i) the mutation frequency of the gene in cells in the plurality of step (a); (ii) the mutation frequency of the gene in cells in the plurality of step (a) that do not exhibit the mitochondrial phenotype of interest; or (iii) an estimated average mutation frequency of the gene in unselected cells. In some embodiments the method comprises contacting the plurality of mutagenized near-haploid mammalian cells with a test agent or exposing the mutagenized near-haploid mammalian cells to a test condition prior to step (b). In some embodiments the method comprises contacting the plurality of mutagenized near-haploid mammalian cells with a test agent or exposing the mutagenized near-haploid mammalian cells to a test condition prior to step (b), and the phenotype of interest comprises a response or altered response to the test agent or test condition. In some embodiments the method comprises contacting the plurality of mutagenized near-haploid mammalian cells with a mitochondrial poison, and the mitochondrial phenotype of interest comprises resistance to the mitochondrial poison. In some embodiments, a method of identifying a gene that affects mitochondrial phenotype further comprises confirming that the gene affects the mitochondrial phenotype of interest by (i) inhibiting the gene in a control cell and observing the effect on the mitochondrial phenotype of interest; or (ii) at least partly restoring the function of the gene in a cell in which the gene is mutated and observing the effect on the mitochondrial phenotype of interest. In some embodiments, in a method of identifying a gene that affects mitochondrial phenotype, the mitochondrial phenotype of interest comprises mitochondrial number, mitochondrial size, mitochondrial morphology, mitochondrial DNA content, mitochondrial DNA replication, mitochondrial biogenesis, respiratory activity, ATP synthesis, matrix pH, mitochondrial membrane potential, or an alteration in any of the foregoing. In some embodiments, in a method of identifying a gene that affects mitochondrial phenotype, the mitochondrial phenotype of interest is associated with or indicative of a mitochondrial function, and the method comprises: (a) providing a plurality of mutagenized near-haploid mammalian cells; (b) isolating a cell that exhibits an alteration in mitochondrial function as compared with a control cell; and (c) identifying a gene that is mutated in the cell, thereby identifying a gene that affects mitochondrial function. In some embodiments, in a method of identifying a gene that affects mitochondrial phenotype the mitochondrial phenotype of interest is associated with or indicative of a mitochondrial function which is (a) respiration; (b) oxidative phosphorylation (OXPHOS); (c) OXPHOS-independent respiration; (d) regulation of mitochondrial membrane potential; (e) regulation of mitochondrial membrane potential; (f) ATP synthesis; or (g) regulation of apoptosis.

In some aspects, the invention provides a method identifying a gene whose modulation has potential to confer protection against mitochondrial dysfunction, the method comprising: (a) contacting a mammalian cell with a mitochondrial poison, wherein the cell has increased or decreased functional expression of a gene as compared to control cells; (b) determining whether the mammalian cell has altered sensitivity to the mitochondrial poison as compared to the control cells; and (c) identifying the gene as one whose modulation has potential to confer protection against mitochondrial dysfunction if the mammalian cell has altered sensitivity to the mitochondrial poison as compared to the control cells. In some embodiments the mammalian cell has decreased functional expression of a gene as compared to control cells. In some embodiments the mammalian cell has decreased functional expression of a gene as compared to control cells, and wherein step (c) comprises identifying the gene as one whose inhibition has potential to confer protection against mitochondrial dysfunction if the cell has increased resistance to the mitochondrial poison as compared to the control cells. In some embodiments the mammalian cell has increased functional expression of a gene as compared to control cells. In some embodiments the mammalian cell has increased functional expression of a gene as compared to control cells, and wherein step (c) comprises identifying the gene as one whose expression or activation has potential to confer protection against mitochondrial dysfunction if the cell has increased resistance to the mitochondrial poison as compared to the control cells. In some embodiments step (a) comprises contacting the mammalian cell with a mitochondrial poison at a concentration and for a time sufficient to kill at least 95% of control cells; step (b) comprises determining that the mammalian cell survived; and step (c) comprises identifying the gene as one whose modulation has potential to confer protection against mitochondrial dysfunction. In some embodiments the method comprises: (a) contacting a plurality of mammalian cells with the mitochondrial poison at a concentration and for a time sufficient to kill at least 95% of control cells, wherein members of the population have increased or decreased functional expression of different genes; (b) isolating surviving cells; and (c) identifying a gene that has increased or decreased functional expression in at least some of the surviving cells as compared to control cells. In some embodiments the plurality of mammalian cells comprises at least 1,000 distinct members, each having increased or decreased functional expression of a different gene. In some embodiments the plurality of mammalian cells is transfected with an shRNA, siRNA, or open reading frame (ORF) library prior to step (a), wherein the library comprises shRNAs, siRNAs, or ORFs that correspond in sequence to multiple distinct genes. In some embodiments the plurality of mammalian cells is transfected with an shRNA, siRNA, or ORF library prior to step (a), and wherein step (c) comprises determining the identity of an shRNA, siRNA, or ORF sequence present in cells isolated in step (b), thereby identifying a gene that has increased or decreased functional expression in at least some of the surviving cells as compared to control cells. In some embodiments a method of identifying a gene whose modulation has potential to confer protection against mitochondrial dysfunction further comprises confirming that modulation of the gene confers protection against mitochondrial dysfunction. In some embodiments a method of identifying a gene whose modulation has potential to confer protection against mitochondrial dysfunction further comprises performing an assay or screening a library to identify a modulator of the gene. In some embodiments any of the methods further comprises contacting a cell with a modulator of the gene. In some embodiments a method of identifying a gene whose modulation has potential to confer protection against mitochondrial dysfunction further comprises (a) contacting a mammalian cell with a modulator of the gene; and (b) performing an assay to assess at least one phenotype or function of the cell's mitochondria. In some embodiments a method of identifying a gene whose modulation has potential to confer protection against mitochondrial dysfunction further comprises (a) contacting a mammalian cell with a modulator of the gene; and (b) performing an assay to assess at least one phenotype or function of the cell's mitochondria. In some embodiments a method of identifying a gene whose modulation has potential to confer protection against mitochondrial dysfunction further comprises (a) contacting a mammalian cell that has deficient mitochondrial function with a modulator of the gene; and (b) performing an assay to assess at least one phenotype or function of the cell's mitochondria. In some embodiments a method of identifying a gene whose modulation has potential to confer protection against mitochondrial dysfunction further comprises (a) contacting a mammalian cell that has mitochondrial dysfunction with a modulator of the gene; (b) performing an assay of at least one phenotype or function of the cell's mitochondria; and (c) identifying the modulator as a candidate therapeutic agent for treatment of a mitochondrial disorder if the cell exhibits protection against mitochondrial dysfunction. In some embodiments a method of identifying a gene whose modulation has potential to confer protection against mitochondrial dysfunction further comprises (a) contacting a mammalian cell with a modulator of the gene; (b) performing an assay of at least one phenotype or function of the cell's mitochondria; and (c) identifying the modulator as a candidate therapeutic agent for treatment of a mitochondrial disorder if the cell exhibits improved mitochondrial phenotype or function. In some embodiments a method of identifying a gene whose modulation has potential to confer protection against mitochondrial dysfunction further comprises (a) contacting a mammalian cell that has deficient mitochondrial function with a modulator of the gene; (b) performing an assay of at least one phenotype or function of the cell's mitochondria; and (c) identifying the modulator as a candidate therapeutic agent for treatment of a mitochondrial disorder if the cell exhibits improved mitochondrial phenotype or function. In some embodiments a method of identifying a gene whose modulation has potential to confer protection against mitochondrial dysfunction further comprises administering a modulator of the gene to a subject. In some embodiments a method of identifying a gene whose modulation has potential to confer protection against mitochondrial dysfunction further comprises administering a modulator of the gene to a subject suffering from a mitochondrial disorder. In some embodiments a method of identifying a gene whose modulation has potential to confer protection against mitochondrial dysfunction further comprises administering a modulator of the gene to a subject suffering from a mitochondrial disorder and assessing the effect of the modulator on the subject.

In some aspects, the invention provides a method of identifying a gene whose modulation has potential to confer protection against mitochondrial dysfunction, the method comprising: (a) providing a plurality of mutagenized mammalian cells; (b) contacting the plurality of mutagenized mammalian cells with a mitochondrial poison; (c) isolating a cell that exhibits altered sensitivity to the mitochondrial poison as compared to control cells; and (d) identifying a gene that is mutagenized in the cell, thereby identifying a gene whose modulation has potential to confer protection against mitochondrial dysfunction. In some embodiments the mutagenized mammalian cells are near-haploid. In some embodiments the mutagenized mammalian cells are human cells, e.g., KBM7 cells. In some embodiments the cells are insertionally mutagenized, e.g., by a gene trap vector. In some embodiments step (c) comprises isolating a cell that exhibits increased resistance to the mitochondrial poison as compared to control cells. In some embodiments step (c) comprises isolating a cell that exhibits increased resistance to the mitochondrial poison as compared to control cells, and step (d) comprises identifying the gene as one whose inhibition has potential to confer protection against mitochondrial dysfunction. In some embodiments step (c) comprises isolating a cell that exhibits increased sensitivity to the mitochondrial poison as compared to control cells, and step (d) comprises identifying the gene as one whose expression or activation has potential to confer protection against mitochondrial dysfunction. In some embodiments step (a) comprises contacting the plurality of mutagenized mammalian cells with a mitochondrial poison at a concentration and for a time sufficient to kill at least 95% of control cells; step (c) comprises isolating surviving cells; and step (d) comprises identifying a gene that is mutated in at least some of the surviving cells, thereby identifying a gene whose modulation has potential to confer protection against mitochondrial dysfunction. In some embodiments the method comprises: (b) contacting the plurality of mutagenized mammalian cells with the mitochondrial poison at a concentration and for a time sufficient to kill at least 95% of control cells, wherein members of the population have increased or decreased functional expression of different genes; (c) isolating cells that survive; and (d) identifying a gene whose mutation frequency in surviving cells is significantly greater than a reference frequency. In some embodiments the reference frequency is approximately equal to (i) the mutation frequency of the gene in the cells of step (a); or (ii) an estimated average mutation frequency of the gene in unselected cells. In some embodiments a method of identifying a gene whose modulation has potential to confer protection against mitochondrial dysfunction further comprises confirming that modulation of the gene confers protection against mitochondrial dysfunction. In some embodiments a method of identifying a gene whose modulation has potential to confer protection against mitochondrial dysfunction further comprises performing an assay or screening a library to identify a modulator of the gene. In some embodiments a method of identifying a gene whose modulation has potential to confer protection against mitochondrial dysfunction further comprises contacting a cell with a modulator of the gene. In some embodiments a method of identifying a gene whose modulation has potential to confer protection against mitochondrial dysfunction further comprises (a) contacting a mammalian cell with a modulator of the gene; and (b) performing an assay to assess at least one phenotype or function of the cell's mitochondria. In some embodiments a method of identifying a gene whose modulation has potential to confer protection against mitochondrial dysfunction further comprises: (a) contacting a mammalian cell that has deficient mitochondrial function with a modulator of the gene; and (b) performing an assay to assess at least one phenotype or function of the cell's mitochondria. In some embodiments a method of identifying a gene whose modulation has potential to confer protection against mitochondrial dysfunction further comprises (a) contacting a mammalian cell that has mitochondrial dysfunction with a modulator of the gene; (b) performing an assay of at least one phenotype or function of the cell's mitochondria; and (c) identifying the modulator as a candidate therapeutic agent for treatment of a mitochondrial disorder if the cell exhibits protection against mitochondrial dysfunction. In some embodiments a method of identifying a gene whose modulation has potential to confer protection against mitochondrial dysfunction further comprises (a) contacting a mammalian cell with a modulator of the gene; (b) performing an assay of at least one phenotype or function of the cell's mitochondria; and (c) identifying the modulator as a candidate therapeutic agent for treatment of a mitochondrial disorder if the cell exhibits improved mitochondrial phenotype or function. In some embodiments a method of identifying a gene whose modulation has potential to confer protection against mitochondrial dysfunction further comprises (a) contacting a mammalian cell that has deficient mitochondrial function with a modulator of the gene; (b) performing an assay of at least one phenotype or function of the cell's mitochondria; and (c) identifying the modulator as a candidate therapeutic agent for treatment of a mitochondrial disorder if the cell exhibits improved mitochondrial phenotype or function. In some embodiments a method of identifying a gene whose modulation has potential to confer protection against mitochondrial dysfunction further comprises administering a modulator of the gene to a subject. In some embodiments a method of identifying a gene whose modulation has potential to confer protection against mitochondrial dysfunction further comprises administering a modulator of the gene to a subject suffering from a mitochondrial disorder. In some embodiments a method of identifying a gene whose modulation has potential to confer protection against mitochondrial dysfunction further comprises administering a modulator of the gene to a subject suffering from a mitochondrial disorder and assessing the effect of the modulator on the subject.

In some aspects, the invention provides a method of identifying a gene that affects sensitivity of a cell to a mitochondrial poison, the method comprising: (a) contacting a mammalian cell with a mitochondrial poison, wherein the cell has increased or decreased functional expression of a gene as compared to control cells; (b) determining whether the mammalian cell has altered sensitivity to the mitochondrial poison as compared to the control cells; and (c) identifying the gene as one that affects sensitivity of a cell to a mitochondrial poison if the mammalian cell has altered sensitivity to the mitochondrial poison as compared to the control cells. In some embodiments the mammalian cell has decreased functional expression of a gene as compared to control cells. In some embodiments the mammalian cell has decreased functional expression of a gene as compared to control cells, and step (c) comprises identifying the gene as one whose inhibition has potential to confer protection against mitochondrial dysfunction if the cell has increased resistance to the mitochondrial poison as compared to the control cells. In some embodiments the mammalian cell has increased functional expression of a gene as compared to control cells. In some embodiments the mammalian cell has increased functional expression of a gene as compared to control cells, and step (c) comprises identifying the gene as one whose expression or activation confers protection against mitochondrial dysfunction if the cell has increased resistance to the mitochondrial poison as compared to the control cells. In some embodiments step (a) comprises contacting the mammalian cell with a mitochondrial poison at a concentration and for a time sufficient to kill at least 95% of control cells; step (b) comprises determining that the mammalian cell survived; and step (c) comprises identifying the gene as one whose modulation has potential to confer protection against mitochondrial dysfunction. In some embodiments the method comprises: (a) contacting a plurality of mammalian cells with the mitochondrial poison at a concentration and for a time sufficient to kill at least 95% of control cells, wherein members of the population have increased or decreased functional expression of different genes; (b) isolating surviving cells; and (c) identifying a gene that has increased or decreased functional expression in at least some of the surviving cells as compared to control cells. In some embodiments the plurality of mammalian cells comprises at least 1,000 distinct members, each having increased or decreased functional expression of a different gene. In some embodiments the plurality of mammalian cells is transfected with an shRNA, siRNA, or ORF library prior to step (a), wherein the library comprises shRNAs, siRNAs, or cDNAs that correspond in sequence to multiple distinct genes. In some embodiments the plurality of mammalian cells is transfected with an shRNA, siRNA, or ORF library prior to step (a), and wherein step (c) comprises determining the identity of an shRNA, siRNA, or ORF sequence present in cells isolated in step (b), thereby identifying a gene that has increased or decreased functional expression in at least some of the surviving cells as compared to control cells. In some embodiments a method of identifying a gene that affects sensitivity of a cell to a mitochondrial poison further comprises confirming that modulation of the gene confers protection against mitochondrial dysfunction. In some embodiments a method of identifying a gene that affects sensitivity of a cell to a mitochondrial poison further comprises performing an assay or screening a library to identify a modulator of the gene. In some embodiments a method of identifying a gene that affects sensitivity of a cell to a mitochondrial poison further comprises contacting a cell with a modulator of the gene. In some embodiments a method of identifying a gene that affects sensitivity of a cell to a mitochondrial poison further comprises (a) contacting a mammalian cell with a modulator of the gene; and (b) performing an assay to assess at least one phenotype or function of the cell's mitochondria. In some embodiments a method of identifying a gene that affects sensitivity of a cell to a mitochondrial poison further comprises (a) contacting a mammalian cell that has deficient mitochondrial function with a modulator of the gene; and (b) performing an assay to assess at least one phenotype or function of the cell's mitochondria. In some embodiments a method of identifying a gene that affects sensitivity of a cell to a mitochondrial poison further comprises (a) contacting a mammalian cell that has mitochondrial dysfunction with a modulator of the gene; (b) performing an assay of at least one phenotype or function of the cell's mitochondria; and (c) identifying the modulator as a candidate therapeutic agent for treatment of a mitochondrial disorder if the cell exhibits protection against mitochondrial dysfunction. In some embodiments a method of identifying a gene that affects sensitivity of a cell to a mitochondrial poison further comprises (a) contacting a mammalian cell with a modulator of the gene; (b) performing an assay of at least one phenotype or function of the cell's mitochondria; and (c) identifying the modulator as a candidate therapeutic agent for treatment of a mitochondrial disorder if the cell exhibits improved mitochondrial phenotype or function. In some embodiments a method of identifying a gene that affects sensitivity of a cell to a mitochondrial poison further comprises (a) contacting a mammalian cell that has deficient mitochondrial function with a modulator of the gene; (b) performing an assay of at least one phenotype or function of the cell's mitochondria; and (c) identifying the modulator as a candidate therapeutic agent for treatment of a mitochondrial disorder if the cell exhibits improved mitochondrial phenotype or function. In some embodiments a method of identifying a gene that affects sensitivity of a cell to a mitochondrial poison further comprises administering a modulator of the gene to a subject. In some embodiments a method of identifying a gene that affects sensitivity of a cell to a mitochondrial poison further comprises administering a modulator of the gene to a subject suffering from a mitochondrial disorder. In some embodiments a method of identifying a gene that affects sensitivity of a cell to a mitochondrial poison further comprises administering a modulator of the gene to a subject suffering from a mitochondrial disorder and assessing the effect of the modulator on the subject.

In some aspects, the invention provides a method of identifying a gene that affects sensitivity of a cell to a mitochondrial poison, the method comprising: (a) providing a plurality of mutagenized mammalian cells; (b) contacting the plurality of mutagenized mammalian cells with a mitochondrial poison; (c) isolating a cell that exhibits altered sensitivity to the mitochondrial poison; and (d) identifying a gene that is mutagenized in the cell, thereby identifying a gene that affects sensitivity of a cell to the mitochondrial poison. In some embodiments the mutagenized mammalian cells are near-haploid cells. In some embodiments the mutagenized mammalian cells are human cells, e.g., KBM7 cells. In some embodiments the cells are insertionally mutagenized, e.g., by a gene trap vector. In some embodiments step (c) comprises isolating a cell that exhibits increased resistance to the mitochondrial poison as compared to control cells. In some embodiments step (c) comprises isolating a cell that exhibits increased resistance to the mitochondrial poison as compared to control cells, and step (d) comprises identifying the gene as one whose inhibition has potential to confer protection against mitochondrial dysfunction. In some embodiments step (c) comprises isolating a cell that exhibits increased sensitivity to the mitochondrial poison as compared to control cells, and step (d) comprises identifying the gene as one expression or activation has potential to confer protection against mitochondrial dysfunction. In some embodiments step (a) comprises contacting the plurality of mutagenized mammalian cells with a mitochondrial poison at a concentration and for a time sufficient to kill at least 95% of control cells; step (c) comprises isolating surviving cells; and step (d) comprises identifying a gene that is mutated in at least some of the surviving cells, thereby identifying a gene whose modulation has potential to confer protection against mitochondrial dysfunction. In some embodiments method comprises: (b) contacting the plurality of mutagenized mammalian cells with the mitochondrial poison at a concentration and for a time sufficient to kill at least 95% of control cells, wherein members of the population have increased or decreased functional expression of different genes; (c) isolating cells that survive; and (d) identifying a gene whose mutation frequency in surviving cells is significantly greater than a reference frequency. In some embodiments the reference frequency is approximately equal to (i) the mutation frequency of the gene in the cells of step (a); or (ii) an estimated average mutation frequency of the gene in unselected cells. In some embodiments a method of identifying a gene that affects sensitivity of a cell to a mitochondrial poison further comprises confirming that modulation of the gene confers protection against mitochondrial dysfunction. In some embodiments a method of identifying a gene that affects sensitivity of a cell to a mitochondrial poison further comprises performing an assay or screening a library to identify a modulator of the gene. In some embodiments a method of identifying a gene that affects sensitivity of a cell to a mitochondrial poison further comprises contacting a cell with a modulator of the gene. In some embodiments a method of identifying a gene that affects sensitivity of a cell to a mitochondrial poison further comprises (a) contacting a mammalian cell with a modulator of the gene; and (b) performing an assay to assess at least one phenotype or function of the cell's mitochondria. In some embodiments a method of identifying a gene that affects sensitivity of a cell to a mitochondrial poison further comprises (a) contacting a mammalian cell that has deficient mitochondrial function with a modulator of the gene; and (b) performing an assay to assess at least one phenotype or function of the cell's mitochondria. In some embodiments a method of identifying a gene that affects sensitivity of a cell to a mitochondrial poison further comprises (a) contacting a mammalian cell that has mitochondrial dysfunction with a modulator of the gene; (b) performing an assay of at least one phenotype or function of the cell's mitochondria; and (c) identifying the modulator as a candidate therapeutic agent for treatment of a mitochondrial disorder if the cell exhibits protection against mitochondrial dysfunction. In some embodiments a method of identifying a gene that affects sensitivity of a cell to a mitochondrial poison further comprises (a) contacting a mammalian cell with a modulator of the gene; (b) performing an assay of at least one phenotype or function of the cell's mitochondria; and (c) identifying the modulator as a candidate therapeutic agent for treatment of a mitochondrial disorder if the cell exhibits improved mitochondrial phenotype or function. In some embodiments a method of identifying a gene that affects sensitivity of a cell to a mitochondrial poison further comprises (a) contacting a mammalian cell that has deficient mitochondrial function with a modulator of the gene; (b) performing an assay of at least one phenotype or function of the cell's mitochondria; and (c) identifying the modulator as a candidate therapeutic agent for treatment of a mitochondrial disorder if the cell exhibits improved mitochondrial phenotype or function. In some embodiments a method of identifying a gene that affects sensitivity of a cell to a mitochondrial poison further comprises administering a modulator of the gene to a subject. In some embodiments a method of identifying a gene that affects sensitivity of a cell to a mitochondrial poison further comprises administering a modulator of the gene to a subject suffering from a mitochondrial disorder. In some embodiments a method of identifying a gene that affects sensitivity of a cell to a mitochondrial poison further comprises administering a modulator of the gene to a subject suffering from a mitochondrial disorder and assessing the effect of the modulator on the subject.

In some aspects, the invention provides a method of identifying a candidate target for drug development for mitochondrial disorders, the method comprising: (a) contacting a mammalian cell with a mitochondrial poison, wherein the cell has increased or decreased functional expression of a gene as compared to a control cell; (b) determining whether the mammalian cell has altered sensitivity to the mitochondrial poison as compared to the control cell; and (c) identifying the gene as a candidate target for drug development for mitochondrial disorders if the mammalian cell has altered sensitivity to the mitochondrial poison as compared to the control cell. In some embodiments the mammalian cell has decreased functional expression of a gene as compared to control cells. In some embodiments the mammalian cell has decreased functional expression of a gene as compared to control cells, and step (c) comprises identifying the gene as one whose inhibition has potential to confer protection against mitochondrial dysfunction if the cell has increased resistance to the mitochondrial poison as compared to the control cells. In some embodiments the mammalian cell has increased functional expression of a gene as compared to control cells. In some embodiments the mammalian cell has increased functional expression of a gene as compared to control cells, and step (c) comprises identifying the gene as one whose expression or activation has potential to confer protection against mitochondrial dysfunction if the cell has increased resistance to the mitochondrial poison as compared to the control cells. In some embodiments step (a) comprises contacting the mammalian cell with a mitochondrial poison at a concentration and for a time sufficient to kill at least 95% of control cells; step (b) comprises determining that the mammalian cell survived; and step (c) comprises identifying the gene as one whose modulation has potential to confer protection against mitochondrial dysfunction. In some embodiments the method comprises: (a) contacting a plurality of mammalian cells with the mitochondrial poison at a concentration and for a time sufficient to kill at least 95% of control cells, wherein members of the population have increased or decreased functional expression of different genes; (b) isolating surviving cells; and (c) identifying a gene that has increased or decreased functional expression in at least some of the surviving cells as compared to control cells. In some embodiments the plurality of mammalian cells comprises at least 1,000 distinct members, each having increased or decreased functional expression of a different gene. In some embodiments the plurality of mammalian cells is transfected with an shRNA, siRNA, or ORF library prior to step (a), wherein the library comprises shRNAs, siRNAs, or ORFs that correspond in sequence to multiple distinct genes. In some embodiments the plurality of mammalian cells is transfected with an shRNA, siRNA, or ORF library prior to step (a), and wherein step (c) comprises determining the identity of an shRNA, siRNA, or ORF sequence present in cells isolated in step (b), thereby identifying a gene that has increased or decreased functional expression in at least some of the surviving cells as compared to control cells. In some embodiments a method of identifying a candidate target for drug development for mitochondrial disorders further comprises confirming that modulation of the gene confers protection against mitochondrial dysfunction. In some embodiments a method of identifying a candidate target for drug development for mitochondrial disorders further comprises performing an assay or screening a library to identify a modulator of the gene. In some embodiments a method of identifying a candidate target for drug development for mitochondrial disorders further comprises contacting a cell with a modulator of the gene. In some embodiments a method of identifying a candidate target for drug development for mitochondrial disorders further comprises (a) contacting a mammalian cell with a modulator of the gene; and (b) performing an assay to assess at least one phenotype or function of the cell's mitochondria. In some embodiments a method of identifying a candidate target for drug development for mitochondrial disorders further comprises (a) contacting a mammalian cell that has deficient mitochondrial function with a modulator of the gene; and (b) performing an assay to assess at least one phenotype or function of the cell's mitochondria. In some embodiments a method of identifying a candidate target for drug development for mitochondrial disorders further comprises (a) contacting a mammalian cell that has mitochondrial dysfunction with a modulator of the gene; (b) performing an assay of at least one phenotype or function of the cell's mitochondria; and (c) identifying the modulator as a candidate therapeutic agent for treatment of a mitochondrial disorder if the cell exhibits protection against mitochondrial dysfunction. In some embodiments a method of identifying a candidate target for drug development for mitochondrial disorders further comprises (a) contacting a mammalian cell with a modulator of the gene; (b) performing an assay of at least one phenotype or function of the cell's mitochondria; and (c) identifying the modulator as a candidate therapeutic agent for treatment of a mitochondrial disorder if the cell exhibits improved mitochondrial phenotype or function. In some embodiments a method of identifying a candidate target for drug development for mitochondrial disorders further comprises (a) contacting a mammalian cell that has deficient mitochondrial function with a modulator of the gene; (b) performing an assay of at least one phenotype or function of the cell's mitochondria; and (c) identifying the modulator as a candidate therapeutic agent for treatment of a mitochondrial disorder if the cell exhibits improved mitochondrial phenotype or function. In some embodiments a method of identifying a candidate target for drug development for mitochondrial disorders further comprises administering a modulator of the gene to a subject. In some embodiments a method of identifying a candidate target for drug development for mitochondrial disorders further comprises administering a modulator of the gene to a subject suffering from a mitochondrial disorder. In some embodiments a method of identifying a candidate target for drug development for mitochondrial disorders further comprises administering a modulator of the gene to a subject suffering from a mitochondrial disorder and assessing the effect of the modulator on the subject.

In some aspects, the invention provides method of identifying a candidate target for drug development for mitochondrial disorders, comprising: (a) providing a plurality of mutagenized mammalian cells; (b) contacting the plurality of mutagenized mammalian cells with a mitochondrial poison; (c) isolating a cell that has altered sensitivity to a mitochondrial poison as compared to a control cell; and (d) identifying a gene that is mutated in the cell, thereby identifying a gene that is a candidate target for drug development for mitochondrial disorders. In some embodiments the mutagenized mammalian cells are near-haploid. In some embodiments the mutagenized mammalian cells are human cells, e.g., KBM7 cells. In some embodiments the cells are insertionally mutagenized, e.g., by a gene trap vector. In some embodiments step (c) comprises isolating a cell that exhibits increased resistance to the mitochondrial poison as compared to control cells. In some embodiments step (c) comprises isolating a cell that exhibits increased resistance to the mitochondrial poison as compared to control cells, and step (d) comprises identifying the gene as one whose inhibition has potential to confer protection against mitochondrial dysfunction. In some embodiments step (c) comprises isolating a cell that exhibits increased sensitivity to the mitochondrial poison as compared to control cells, and wherein step (d) comprises identifying the gene as one expression or activation has potential to confer protection against mitochondrial dysfunction. In some embodiments step (b) comprises contacting the plurality of mutagenized mammalian cells with a mitochondrial poison at a concentration and for a time sufficient to kill at least 95% of control cells; step (c) comprises isolating surviving cells; and step (d) comprises identifying a gene that is mutated in at least some of the surviving cells, thereby identifying a gene whose modulation has potential to confer protection against mitochondrial dysfunction. In some embodiments the method comprises: (b) contacting the plurality of mutagenized mammalian cells with the mitochondrial poison at a concentration and for a time sufficient to kill at least 95% of control cells, wherein members of the population have increased or decreased functional expression of different genes; (c) isolating cells that survive; and (d) identifying a gene whose mutation frequency in surviving cells is significantly greater than a reference frequency. In some embodiments the reference frequency is approximately equal to (i) the mutation frequency of the gene in the cells of step (a); or (ii) an estimated average mutation frequency of the gene in unselected cells. In some embodiments a method of identifying a candidate target for drug development for mitochondrial disorders further comprises confirming that modulation of the gene confers protection against mitochondrial dysfunction. In some embodiments a method of identifying a candidate target for drug development for mitochondrial disorders further comprises performing an assay or screening a library to identify a modulator of the gene. In some embodiments any method of identifying a candidate target for drug development for mitochondrial disorders further comprises contacting a cell with a modulator of the gene. In some embodiments any method of identifying a candidate target for drug development for mitochondrial disorders further comprises: (e) contacting a mammalian cell with a modulator of the gene; and (f) performing an assay to assess at least one phenotype or function of the cell's mitochondria. In some embodiments any method of identifying a candidate target for drug development for mitochondrial disorders further comprises (e) contacting a mammalian cell that has deficient mitochondrial function with a modulator of the gene; and (f) performing an assay to assess at least one phenotype or function of the cell's mitochondria. In some embodiments any method of identifying a candidate target for drug development for mitochondrial disorders further comprises (e) contacting a mammalian cell that has mitochondrial dysfunction with a modulator of the gene; (f) performing an assay of at least one phenotype or function of the cell's mitochondria; and (g) identifying the modulator as a candidate therapeutic agent for treatment of a mitochondrial disorder if the cell exhibits protection against mitochondrial dysfunction. In some embodiments any method of identifying a candidate target for drug development for mitochondrial disorders further comprises (e) contacting a mammalian cell with a modulator of the gene; (f) performing an assay of at least one phenotype or function of the cell's mitochondria; and (g) identifying the modulator as a candidate therapeutic agent for treatment of a mitochondrial disorder if the cell exhibits improved mitochondrial phenotype or function.) In some embodiments any method of identifying a candidate target for drug development for mitochondrial disorders further comprises (e) contacting a mammalian cell that has deficient mitochondrial function with a modulator of the gene; (f) performing an assay of at least one phenotype or function of the cell's mitochondria; and (g) identifying the modulator as a candidate therapeutic agent for treatment of a mitochondrial disorder if the cell exhibits improved mitochondrial phenotype or function. In some embodiments a method of identifying a candidate target for drug development for mitochondrial disorders further comprises administering a modulator of the gene to a subject. In some embodiments a method of identifying a candidate target for drug development for mitochondrial disorders further comprises administering a modulator of the gene to a subject suffering from a mitochondrial disorder. In some embodiments a method of identifying a candidate target for drug development for mitochondrial disorders further comprises administering a modulator of the gene to a subject suffering from a mitochondrial disorder and assessing the effect of the modulator on the subject.

In some embodiments of any method or composition relating at least in part to a mitochondrial poison, the mitochondrial poison comprises a Complex I inhibitor.

In some embodiments of any method or composition relating at least in part to a mitochondrial poison, the mitochondrial poison comprises a Complex II inhibitor.

In some embodiments of any method or composition relating at least in part to a mitochondrial poison, the mitochondrial poison comprises a Complex III inhibitor.

In some embodiments of any method or composition relating at least in part to a mitochondrial poison, the mitochondrial poison comprises a Complex IV inhibitor.

In some embodiments of any method or composition relating at least in part to a mitochondrial poison, the mitochondrial poison comprises a Complex V inhibitor.

In some embodiments of any method or composition relating at least in part to a mitochondrial poison, the mitochondrial poison comprises an uncoupling agent.

In some embodiments of any method or composition relating at least in part to a mitochondrial poison, the mitochondrial poison comprises an OXPHOS inhibitor.

In some embodiments of any method or composition relating at least in part to a mitochondrial poison, the mitochondrial poison is a small molecule.

In some aspects, the invention provides a method of identifying a candidate drug for a mitochondrial disorder, the method comprising: (a) identifying a candidate target for drug development for mitochondrial disorders according to any of the afore-mentioned methods; and (b) identifying a modulator of the candidate target, thereby identifying a candidate drug for a mitochondrial disorder. In some embodiments step (b) comprises performing an assay or screening a library to identify a modulator of the candidate target, thereby identifying a candidate drug for a mitochondrial disorder. In some embodiments the method further comprises testing the effect of the modulator on mitochondrial function. In some embodiments the method further comprises testing the effect of the modulator on cells that have mitochondrial dysfunction. In some embodiments the method further comprises testing the modulator in a model of a mitochondrial disorder. In some embodiments the method further comprises generating an analog of the modulator.

In some embodiments a method of validating a candidate drug for mitochondrial disorders is provided, the method comprising: (a) providing a candidate drug for mitochondrial disorders, wherein the candidate drug was identified according to any of the preceding methods; (b) contacting the candidate drug with mitochondria; and (c) determining whether the candidate drug improves at least one phenotype or function of the mitochondria wherein if the candidate drug improves at least one phenotype or function of mitochondria, the candidate drug is validated as a candidate drug for mitochondrial disorders. In some embodiments the mitochondria comprise dysfunctional mitochondria. In some embodiments the method comprises contacting the candidate drug with cultured mammalian cells comprising dysfunctional mitochondria. In some embodiments the method comprises administering the candidate drug to a subject with a mitochondrial disorder.

In some aspects, the invention provides a composition comprising: (a) a plurality of mutagenized near-haploid mammalian cells; and (b) a mitochondrial poison. In some embodiments the cells are insertionally mutagenized, e.g., by a gene trap vector. In some embodiments the cells are human cells, e.g., KBM7 cells. In some embodiments the mitochondrial poison is present at a concentration sufficient to inhibit survival or proliferation of control cells by at least 95% in 2 weeks.

In some aspects, the invention provides a near-haploid mammalian cell line, wherein cells of the cell line have a mutation that confers altered sensitivity to a mitochondrial poison. In some embodiments, cells of the cell line have reduced sensitivity to a mitochondrial poison. In some embodiments cells of the cell line have a mutation in the gene that encodes ATPIF1. In some embodiments the cells are human cells, e.g., KBM7 cells. In some embodiments cells of the cell line have a mutation in a gene that encodes a mitochondrial protein. In some embodiments the gene is a nuclear gene. In some embodiments the mutation is associated with a mitochondrial disorder. In some embodiments the mutation is an insertion. In some embodiments the mutation is an insertion of at least a portion of a gene trap vector.

In some aspects, the invention provides a method of identifying a candidate drug for a mitochondrial disorder, the method comprising identifying an ATPIF1 modulator. In some embodiments the method comprises identifying an ATPIF1 inhibitor. In some embodiments the method comprises (a) contacting a test agent with a polypeptide comprising an ATPIF1 polypeptide; (b) determining whether the test agent binds to ATPIF1; and (c) identifying the test agent as a candidate drug for a mitochondrial disorder if the test agent binds to the ATPIF1 polypeptide. In some embodiments the method comprises identifying or designing a compound that inhibits binding of an ATPIF1 polypeptide to the F1-F0 ATPase. In some embodiments the method comprises identifying or designing a compound that inhibits dimerization of an ATPIF1 polypeptide. In some embodiments the method comprises (a) contacting a test agent with a cell; (b) determining whether the test agent inhibits expression or activity of ATPIF1 in the cell; and (c) identifying the test agent as a candidate drug for a mitochondrial disorder if the test agent inhibits expression or activity of ATPIF1 in the cell. In some embodiments the method comprises (a) contacting a test agent with a cell, wherein the cell comprises a reporter construct comprising at least a portion of the regulatory region of a mammalian ATPIF1 gene operably linked to a sequence encoding a reporter molecule; and (b) determining whether the test agent inhibits expression of the reporter construct by the cell, wherein if the test agent inhibits expression of the reporter construct the test agent is identified as a candidate drug for a mitochondrial disorder. In some embodiments the method comprises steps of: (a) providing a composition comprising a polypeptide comprising an ATPIF1 polypeptide and a test agent; (b) determining whether the test agent inhibits at least one activity of ATPIF1 exhibited by the polypeptide, wherein if the test agent inhibits at least one activity of ATPIF1, the test agent is identified as a candidate drug for a mitochondrial disorder. In some embodiments step (b) comprises determining whether the test agent inhibits binding of the polypeptide to at least one ATPIF1 interacting protein. In some embodiments step (b) comprises determining whether the test agent inhibits dimerization of the polypeptide. In some embodiments step (b) comprises determining whether the test agent inhibits binding of the polypeptide to the F1-F0 ATPase or an F1 subunit thereof. In some embodiments any of the methods comprising identifying an ATPIF1 modulator further comprises synthesizing an analog of the ATPIF1 modulator. In some embodiments any of the methods comprising identifying an ATPIF1 modulator further comprises synthesizing an analog of the ATPIF1 modulator, wherein the analog exhibits at least one altered property relative to the ATPIF1 modulator. In some embodiments any of the methods comprising identifying an ATPIF1 modulator further comprises synthesizing an analog of the ATPIF1 modulator; and testing the ability of the analog to modulate expression or activity of ATPIF1.

In some aspects, the invention provides a reporter construct comprising at least a portion of the regulatory region of a mammalian ATPIF1 gene operably linked to a sequence encoding a reporter molecule. In some aspects, a cell comprising the reporter construct is disclosed. In some aspects, a composition comprising the reporter construct or the cell and (b) an agent to be tested for ability to modulate expression of the reporter molecule is disclosed.

In some aspects, the invention provides a composition comprising a polypeptide comprising an ATPIF1 polypeptide; and an agent to be tested for ability to modulate mammalian ATPIF1. In some embodiments the ATPIF1 polypeptide is isolated. In some embodiments the ATPIF1 polypeptide is in a cell. In some embodiments at least some of the polypeptide molecules are detectably labeled or attached to a support. In some embodiments the agent is a small molecule. In some embodiments at least some of the polypeptide molecules are in solution and at least some of the polypeptide molecules attached to a support. In some embodiments the composition comprises at least one compound capable of generating a detectable signal based on interaction of the polypeptide with a second polypeptide. In some embodiments the composition comprises comprising a mammalian F1-F0 ATPase or F1 subunit thereof. In some embodiments, an article comprising at least distinct 10 compositions, the compositions comprising different test agents, is disclosed. In some embodiments the article is a multiwell plate, and the compositions are in wells of the multiwell plate.

In some aspects, the invention provides a method of modulating a biological activity in a mammalian cell, the method comprising contacting the cell with an ATPIF1 modulator. In some embodiments the ATPIF1 modulator is an ATPIF1 inhibitor. In some embodiments the biological activity is a mitochondrial function. In some embodiments the biological activity is (a) respiration; (b) oxidative phosphorylation; (c) OXPHOS-independent respiration (d) electron transport by the mitochondrial electron transport chain; (d) ATP synthesis; (e) regulation of mitochondrial membrane potential; (f) regulation of mitochondrial membrane permeability; or (g) regulation of cell death. In some embodiments the mammalian cell has impaired mitochondrial function. In some embodiments the mammalian cell has a defect in oxidative phosphorylation. In some embodiments the mammalian cell is a human cell. In some embodiments the mammalian cell originates from a subject suffering from a mitochondrial disorder. In some embodiments the mammalian cell originates from a subject suffering from a mitochondrial disorder characterized by loss or degeneration of cells having mitochondrial dysfunction. In some embodiments the mammalian cell originates from a subject suffering from a mitochondrial disorder characterized by apoptosis of cells having mitochondrial dysfunction. In some embodiments the mammalian cell originates from a subject suffering from a mitochondrial disorder characterized by liver dysfunction. In some embodiments the mammalian cell originates from a subject suffering from a mitochondrial disorder, wherein the mitochondria; disorder is a neurodegenerative disorder In some embodiments the mammalian cell originates from a subject suffering from a mitochondrial disorder, wherein the mitochondrial disorder is Parkinson's disease, an optic atrophy, or GRACILE syndrome. In some embodiments the mammalian cell is a human cell. In some embodiments the mammalian cell is a hepatocyte. In some embodiments the mammalian cell is a neuron. In some embodiments the mammalian cell has been exposed to a toxic agent. In some embodiments the mammalian cell has been exposed to a mitochondrial poison. In some embodiments the mammalian cell is in a subject, e.g., a human subject. In some embodiments the mammalian cell is in a subject suffering from a mitochondrial disorder, e.g., a human subject.

In some aspects the invention provides a method of inhibiting death or degeneration of a mammalian cell, the method comprising contacting the cell with an APTIF1 inhibitor. In some embodiments the mammalian cell has mitochondrial dysfunction. In some embodiments the mammalian cell has a defect in oxidative phosphorylation. In some embodiments the mammalian cell originates from a subject suffering from a mitochondrial disorder. In some embodiments the mammalian cell originates from a subject suffering from a mitochondrial disorder characterized by loss or degeneration of cells having mitochondrial dysfunction. In some embodiments the mammalian cell originates from a subject suffering from a mitochondrial disorder characterized by apoptosis of cells having mitochondrial dysfunction. In some embodiments the mammalian cell originates from a subject suffering from a mitochondrial disorder characterized by liver dysfunction. In some embodiments the mammalian cell originates from a subject suffering from a mitochondrial disorder, wherein the mitochondrial disorder is a neurodegenerative disorder. In some embodiments the mammalian cell originates from a subject suffering from a mitochondrial disorder, wherein the mitochondrial disorder is Parkinson's disease, an optic atrophy, or GRACILE syndrome. In some embodiments the mammalian cell is a human cell. In some embodiments the mammalian cell is a hepatocyte. In some embodiments the mammalian cell is a neuron. In some embodiments the mammalian cell has been exposed to a toxic agent. In some embodiments the mammalian cell has been exposed to a mitochondrial poison. In some embodiments the mammalian cell is in a subject. In some embodiments the mammalian cell is in a subject suffering from a mitochondrial disorder.

In some aspects, the invention provides a method of treating a mammalian subject in need of treatment for a mitochondrial disorder, the method comprising administering an ATPIF1 inhibitor to the subject. In some embodiments the mitochondrial disorder is characterized by liver dysfunction. In some embodiments the mitochondrial disorder is a neurodegenerative disorder. In some embodiments the mitochondrial disorder is Parkinson's disease, an optic atrophy, or GRACILE syndrome. In some embodiments the mitochondrial disorder is caused at least in part from exposure to a toxic agent. In some embodiments the toxic agent is a neurotoxin, i.e., the agent is toxic towards neurons. In some embodiments the mitochondrial disorder is caused at least in part by exposure to a mitochondrial poison. In some embodiments the subject serves as a model for a human mitochondrial disorder. In some embodiments the subject is human. In some embodiments the ATPIF1 inhibitor inhibits expression of ATPIF1. In some embodiments the ATPIF1 inhibitor inhibits interaction of ATPIF1 with an ATPIF1-interacting protein. In some embodiments the inhibitor inhibits dimerization of ATPIF1.

In some aspects, the invention provides a pharmaceutical composition comprising an ATPIF1 inhibitor. In some embodiments the ATPIF1 inhibitor binds to ATPIF1. In some embodiments the ATPIF1 inhibitor inhibits expression of ATPIF1. In some embodiments the ATPIF1 inhibitor inhibits interaction of ATPIF1 with an ATPIF1-interacting protein. In some embodiments the ATPIF1 inhibitor inhibits dimerization of ATPIF1. In some embodiments the ATPIF1 inhibitor inhibits binding of ATPIF1 to the F1-F0 ATPase or the F1 subunit thereof. In some embodiments the ATPIF1 inhibitor comprises a small molecule, nucleic acid, peptide, or peptidomimetic. In some embodiments the ATPIF1 inhibitor comprises an RNAi agent or antisense agent. In some embodiments the ATPIF1 inhibitor comprises a nucleic acid construct comprising a sequence that encodes a polynucleotide or polypeptide that inhibits ATPIF1 expression or activity when expressed in a mammalian cell, wherein the sequence is operably linked to a promoter capable of directing transcription of the sequence in a mammalian cell. In some embodiments the ATPIF1 inhibitor comprises a gene therapy vector comprising a nucleic acid construct comprising a sequence that encodes a polynucleotide or polypeptide that inhibits ATPIF1 expression or activity when expressed in a mammalian cell, wherein the sequence is operably linked to a promoter capable of directing transcription of the sequence in mammalian cell. In some embodiments method of treating mammalian subject in need of treatment for a mitochondrial disorder is provided, the method comprising administering one or more of the compositions to the subject.

In some aspects, the invention provides a vector comprising a nucleic acid construct comprising a sequence that encodes a polynucleotide or polypeptide that inhibits ATPIF1 expression or activity when expressed in a mammalian cell, wherein the sequence is operably linked to a promoter capable of directing transcription of the sequence in a mammalian cell. In some embodiments the vector comprises a viral vector. In some embodiments the vector comprises a viral vector capable of transducing human hepatocytes or neurons. In some embodiments the vector comprises a replication-defective viral vector. In some embodiments a method of treating a mammalian subject in need of treatment for a mitochondrial disorder is provided, the method comprising administering one or more of the vectors to the subject.

The practice of certain aspects of the present invention may employ conventional techniques of molecular biology, cell culture, recombinant nucleic acid (e.g., DNA) technology, immunology, transgenic biology, microbiology, nucleic acid and polypeptide synthesis, detection, manipulation, and quantification, and RNA interference that are within the ordinary skill of the art. See, e.g., Ausubel, F., et al., (eds.), Current Protocols in Molecular Biology, Current Protocols in Immunology, Current Protocols in Protein Science, and Current Protocols in Cell Biology, all John Wiley & Sons, N.Y., edition as of December 2008; Sambrook, Russell, and Sambrook, Molecular Cloning: A Laboratory Manual, ^{3rd} ed., Cold Spring Harbor Laboratory Press, Cold Spring Harbor, 2001; Harlow, E. and Lane, D., Antibodies—A Laboratory Manual, Cold Spring Harbor Laboratory Press, Cold Spring Harbor, 1988. Information regarding various mitochondrial disorders and diagnosis and certain treatments of such disorders is found in Longo, D., et al. (eds,), Harrison's Principles of Internal Medicine, 18th Edition; McGraw-Hill Professional, 2011. Information regarding various therapeutic agents and human diseases is found in Brunton, L., et al. (eds.) Goodman and Gilman's The Pharmacological Basis of Therapeutics, 12^{th} Ed., McGraw Hill, 2010 and/or Katzung, B. (ed.) Basic and Clinical Pharmacology, McGraw-Hill/Appleton & Lange; 11th edition (July 2009). All patents, patent applications, books, journal articles, documents, databases, websites, articles, publications, references, etc., cited herein are incorporated by reference in their entirety. In the event of a conflict or inconsistency with the specification, the specification shall control. Applicants reserve the right to amend the specification based, e.g., on any of the incorporated material and/or to correct obvious errors. None of the content of the incorporated material shall limit the invention.

All patents, patent applications, and other publications (e.g., scientific articles, books, websites, and databases) mentioned herein are incorporated by reference in their entirety. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1E. Haploid genetic screen identifies genes, loss of function of which confers resistance to the mitochondrial poison antimycin A. Mutagenized haploid cells were contacted with antimycin A for 2 weeks. Antimycin-resistant cells were selected, pooled and genomic DNA was isolated. Gene trap insertion sites were identified using an inverse-PCR protocol followed by massively parallel sequencing. Sequences were mapped to the human genome and enrichment for mutations in genes was calculated by comparing a gene's mutation frequency in resistant cells to that in the unselected control data set. (A) Plot showing enrichment of particular genes in the antimycin A resistance screen, calculated by comparing how often a gene was mutated in the screen compared to how often the gene carries an insertion in the control dataset. For each gene a p-value (corrected for false discovery rate) was calculated using the one-sided Fisher exact test run in the R software environment. The Y axis represents the inverse logarithm of p values, calculated by Fisher Exact Test. The X-axis represents the insertion sites ordered by their genomic position. The diameter of the bubbles denotes the number of insertions for each gene. Mutations are highly enriched in the genes encoding ATPIF1, WT1, and TP53. The structure of antimycin is also depicted. (B) Schematic diagram of ATPIF1 locus showing location of insertion sites. (C) Upper panel: Western blot showing absence of ATPIF1 protein in two antimycin-resistant clonal cell lines (clone 13 and clone 18) and presence in non-mutagenized KBM7 cells (wild type). Lower panel: Representative light microscopy images of cells of clone 18 (left) and non-mutagenized KBM7 cells (wild type) in the presence of antimycin. (D) Plots showing fold-change in proliferation (quantified as cell number) of clone 18 as compared with “wild type” KBM cells in the absence (left) or presence (right) of antimycin A. Wild type and clone 18 cells proliferate robustly under standard culture conditions. Loss of ATPIF1 expression has relatively little effect on proliferation under standard culture conditions (left). In the presence of antimycin A (right), wild type cells cease proliferating, whereas clone 18 cells continue proliferating. Thus, loss of ATPIF1 expression allows these cells to survive and proliferate in the presence of antimycin A at a concentration (121 μm) toxic to wild type KBM7 cells. (E) Left panel: Western blot showing that cDNA encoding ATPIF1 restores ATPIF1 expression in clone 13 cells to approximately the level present in wild type KBM7 cells. Raptor protein level was assessed as a loading control. Right panel: Plot showing that restoration of ATPIF1 expression in clone 18 cells restores sensitivity to antimycin A (121 μm).

FIG. 2. Plot showing that loss of ATPIF1 in KBM7 cells confers decreased sensitivity (increased resistance) to the mitochondrial poisons FCCP, piercidin, and TTFA. Blue bars show wild type KBM7 cells. Red bars show ATPIF1 mutant KBM7 cells. Relative cell numbers are shown.

FIGS. 3A-3C. (A) Plot showing cellular ATP levels in wild type KBM7 cells in the absence (aqua circles) or presence (pink squares) of antimycin A (121 μm) and in ATPIF1 null KBM cells in the absence (blue triangles) or presence (light blue inverted triangles) of antimycin A (121 μm) (B) Plot showing that mitochondrial number as assessed using Mitotracker Green dye, is not significantly different in wild type and ATPIF1 null KBM7 cells (C) Upper panel: Western blot showing that short hairpin RNA effectively inhibit expression of ATPIF1 in wild type KBM7 cells. S6 level was assessed as a loading control. Lower panel: Plot showing that shRNA-mediated inhibition of KBM7 expression confers decreased sensitivity to antimycin A across a range of concentrations.

FIG. 4. Plot showing that loss of mitochondrial membrane potential resulting from exposure to Antimycin A is rescued (in part) by loss of ATPIF1. In contrast, loss of membrane potential resulting from exposure to oligomycin is not rescued by loss of APTIF1.

FIG. 5. Schematic outline of gene-trap vector integration in an endogenous gene.

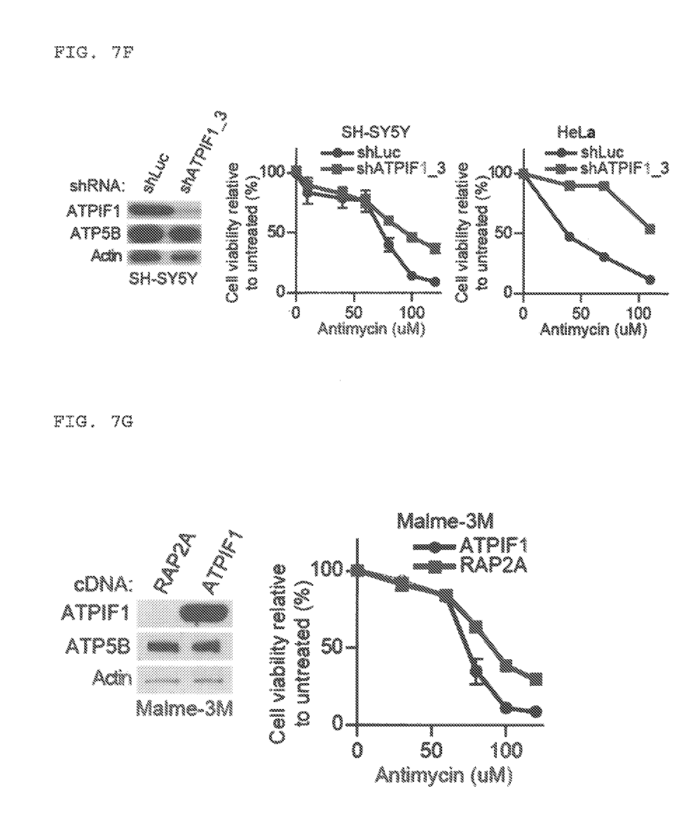

FIG. 6. Human ATPIF1 polypeptide sequences.

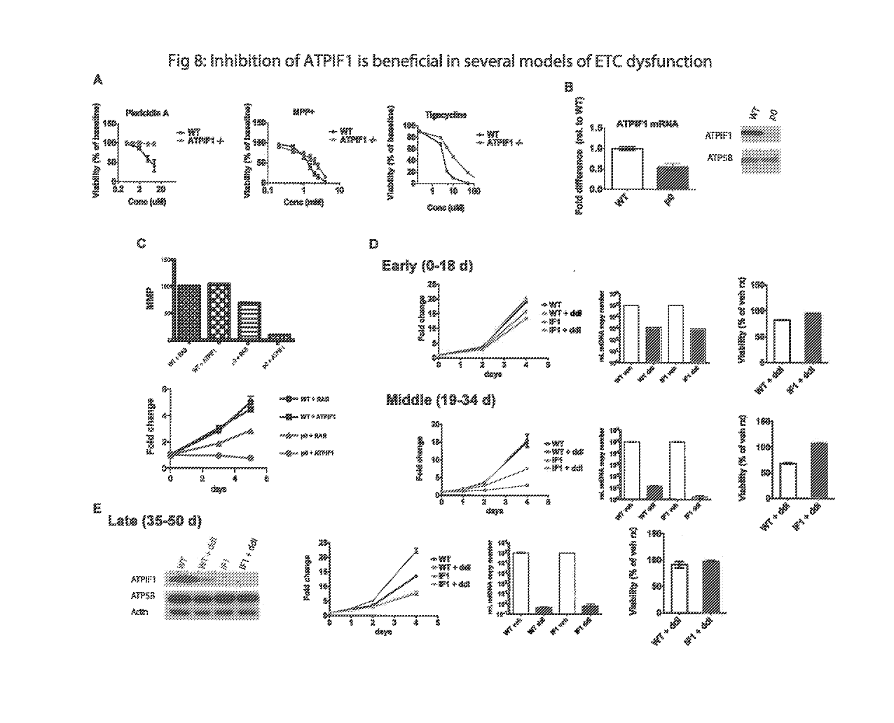

FIGS. 7A-7G. ATPIF1 loss protects against ETC dysfunction by rescue of MMP via the ATP synthase. (A) Mitochondrial mass of WT and ATPIF1 KO KBM7 cells as determined by Mitotracker Green staining and FACS. (B) MMP and ATP in WT and ATPIF1 KO KBM7 cells in response to different mitochondrial toxins as determined by TMRM staining with FACS and CellTiterGlo assays, respectively. (C) Metabolite profiling of WT and ATPIF1 KO KBM7 cells after 1 hour of antimycin treatment. (D) Viability of WT and ATPIF1 KO KBM7 cells in response to different mitochondrial toxins (Anti=antimycin, Oligo=oligomycin) as assessed by 7-AAD staining and FACS. (E) MMP and viability of cells with mtDNA derived from a normal (WT) human subject and a patient with a deficiency in COXI. All experiments: n=3 and error bars are SEM. ATPIF1 is abbreviated IF1 in this figure. (F) Immunoblots for indicated proteins in SH-SY5Y cells expressing a control shRNA against Luciferase (shLuc) or an shRNA against ATPIF1 (shATPIF1_{—}3) (left). Viability of SH-SY5Y (middle) and HeLa (right) cells treated with antimycin for 4 days. Error bars are ±s.e.m. (n=3). (G) Immunoblots for indicated proteins in Malme-3M cells overexpressing control RAP2A or ATPIF1 (left). Viability of Malme-3M cells treated with antimycin for 4 days (right). Error bars are ±s.e.m. (n=3).

FIGS. 8A-8G. Inhibition of ATPIF1 is beneficial in several models of electron transport chain dysfunction. (A) Viability of WT vs IF1 KO KBM7 cells in response to different inhibitors of the ETC as assessed by 7AAD. Piericidin A and MPP+ are both complex I inhibitors, whereas tigecycline is an inhibitor of mitochondrial translation. (B) Q-RT-PCR and immunoblot analysis of ATPIF1 mRNA and protein levels in WT and ρ0 (mtDNA-depleted) cells, respectively. (C) MMP and proliferation of WT vs ρ⁰ cells overexpressing a control protein (RAB) or ATPIF1. (D) & (E) Proliferation, mtDNA copy number, and viability of WT and ATPIF1 KO KBM7 cells during short, intermediate, and long-term treatments with ddI, a drug that depletes mtDNA. mtDNA copy number was determined using Q-PCR. ATPIF1 is abbreviated IF1 in this figure. (F) Relative ATPIF1 mRNA levels (top) and immunoblots for indicated proteins (bottom) in HeLa WT and ρ⁰ cells. Error bars are ±s.e.m. (n=3). **P<0.01. (G) Immunoblots (left) and relative proliferation (right) of HeLa WT and ρ⁰ cells transduced with control vector, ATPIF1 (WI), or ATPIF1 (E55A) constructs. Error bars are ±s.e.m. (n=3).

FIGS. 9A-9H. (A) Mitochondrial mass of WT and ATPIF1_KO KBM7 cells as determined by MitoTracker Green FM staining. (B) mtDNA copy number of WI and ATPIF1_KO KBM7 cells. (C) Representative EM micrographs of WT and ATPIF1_KO KBM7 cells. Scale bars, 200 nm. (D) Quantitative EM analysis of mitochondria in WT and ATPIF1_KO KBM7 cells. Shown are data for WT KBM7 cells expressing control shLuc or shATPIF1_{—}3, and uninfected WT and ATPIF1_KO KBM7 cells with and without treatment with 125 uM antimycin for 3 hours. Error bars are ±s.e.m. (n=10). (E) TMRM staining of WT and ATPIF1_KO KBM7 cells as determined by FACS. (F) Cellular ATP of WT and ATPIF1_KO KBM7 cells. (G) Viability of untreated WT and ATPIF1_KO KBM7 cells as determined by 7-AAD staining. (H) Oxygen consumption rate (OCR) of WT and ATPIF1_KO KBM7 cells. Error bars are ±s.e.m. (n=3), unless otherwise indicated.

FIGS. 10A-10B. Generation of ATPIF1^{-/-} mice (A) Schematic of gene-trap knockout design. The presence of a splice acceptor (SA) site downstream of exon 2 leads to the generation of transcripts lacking exon 3. Mice homozygous for the gene-trap allele are constitutive knockouts but can be converted to conditional knockouts through the activity of FLP recombinase. Conditional knockouts can then be manipulated with Cre recombinase. Black bars represent exons 1, 2, 3 of ATPIF1. FRT=FLP recombinase sites, loxP=Cre recombinase sites, lacZ=lacZ cassette, neo=neon cassette, SA=splice acceptor site, pA=polyadenylation signal. (B) Genotyping PCR of WT, ATPIF1^{-/-}, and ATPIF1^{+/-} mice. WT=native ATPIF1 allele, lacZ=lacZ cassette, KO=gene-trap ATPIF1 allele.

FIGS. 11A-11E. Inhibition of ATPIF1 ameliorates the effects of complex III blockade in primary hepatocytes. (A) Immunoblots for indicated proteins of primary hepatocytes derived from WT and ATPIF1^{-/-} mice. (B) Cellular ATP of WT and ATPIF1^{-/-} primary hepatocytes treated with antimycin (0.625 μM) for 1.5 hours. Error bars are ±s.e.m. (n=3). ***P<0.001. (C) ΔΨm of WT and ATPIF1^{-/-} primary hepatocytes treated with antimycin (10 μM) for 1.5 hours. Error bars are ±s.e.m. (n=3). **P<0.01. (D) Viability of WT and ATPIF1^{-/-} primary hepatocytes treated with antimycin (1.25 μM) for 2 days. Error bars are s.e.m. (n=3). **P<0.01. (E) Schematic diagramming the behavior of cells with FTC dysfunction under conditions where ATPIF1 is active or inhibited. Inhibition of ATPIF1 is depicted by absence of the protein but represents any strategy to block ATPIF1 activity on the F1-F0 ATP synthase.

FIG. 12. Loss of ATPIF1 does not alter mitochondrial mass in primary hepatocytes. Mitochondrial mass of WT and ATPIF1^{-/-} primary hepatocytes as determined by MitoTracker Green FM staining. Error bars are ±s.e.m. (n=3).

### DETAILED DESCRIPTION OF CERTAIN EMBODIMENTS

### I. Glossary

Descriptions and information relating to certain terms used in the present disclosure are collected here for convenience.

“Agent” is used herein to refer to any substance, compound (e.g., molecule), supramolecular complex, material, or combination or mixture thereof. A compound may be any agent that can be represented by a chemical formula, chemical structure, or sequence. Example of agents, include, e.g., small molecules, polypeptides, nucleic acids (e.g., RNAi agents, antisense oligonucleotide, aptamers), lipids, polysaccharides, etc. In general, agents may be obtained using any suitable method known in the art. The ordinary skilled artisan will select an appropriate method based, e.g., on the nature of the agent. An agent may be at least partly purified. In some embodiments an agent may be provided as part of a composition, which may contain, e.g., a counter-ion, aqueous or non-aqueous diluent or carrier, buffer, preservative, or other ingredient, in addition to the agent, in various embodiments. In some embodiments an agent may be provided as a salt, ester, hydrate, or solvate. In some embodiments an agent is cell-permeable, e.g., within the range of typical agents that are taken up by cells and act intracellularly, e.g., within mammalian cells, to produce a biological effect. Certain compounds may exist in particular geometric or stereoisomeric forms. Such compounds, including cis- and trans-isomers, E- and Z-isomers, R- and S-enantiomers, diastereomers, (D)-isomers, (L)-isomers, (−)- and (+)-isomers, racemic mixtures thereof, and other mixtures thereof are encompassed by this disclosure in various embodiments unless otherwise indicated. Certain compounds may exist in a variety or protonation states, may have a variety of configurations, may exist as solvates (e.g., with water (i.e. hydrates) or common solvents) and/or may have different crystalline forms (e.g., polymorphs) or different tautomeric forms. Embodiments exhibiting such alternative protonation states, configurations, solvates, and forms are encompassed by the present disclosure where applicable.

An “analog” of a first agent refers to a second agent that is structurally and/or functionally similar to the first agent. A “structural analog” of a first agent is an analog that is structurally similar to the first agent. A structural analog of an agent may have substantially similar physical, chemical, biological, and/or pharmacological propert(ies) as the agent or may differ in at least one physical, chemical, biological, or pharmacological property. In some embodiments at least one such property may be altered in a manner that renders the analog more suitable for a purpose of interest. In some embodiments a structural analog of an agent differs from the agent in that at least one atom, functional group, or substructure of the agent is replaced by a different atom, functional group, or substructure in the analog. In some embodiments, a structural analog of an agent differs from the agent in that at least one hydrogen or substituent present in the agent is replaced by a different moiety (e.g., a different substituent) in the analog. In some embodiments an analog may comprise a moiety that reacts with a target to form a covalent bond.

The term “antibody” refers to an immunoglobulin, whether natural or wholly or partially synthetically produced. An antibody may be a member of any immunoglobulin class, including any of the mammalian, e.g., human, classes: IgG, IgM, IgA, IgD, and IgE, or subclasses thereof, and may be an antibody fragment, in various embodiments. An antibody may originate from any of a variety of vertebrate (e.g., mammalian or avian) organisms, e.g., mouse, rat, rabbit, hamster, goat, chicken, human, camelid, shark, etc., or may be encoded at least in part by immunoglobulin gene sequences derived from any of the foregoing organisms. In some embodiments an antibody is a nanobody. As used herein, the term “antibody fragment” refers to any of various portions of an antibody that contain less than a complete antibody structure (e.g., less than the complete structure of a conventional antibody composed of two heavy and two light chains). In general, an antibody fragment retains at least a significant portion of the complete antibody's specific binding ability. Examples of antibody fragments include, but are not limited to, Fab, Fab′, and F(ab′)2 fragments. The term “antibody” encompasses single chain variable (scFv), Fv, dsFv, diabody, minibody, Fd fragments, single domain antibodies (e.g., antibodies comprising a single variable domain, e.g., a heavy chain variable domain, e.g., VH or VHH domain), and nanobodies. Standard methods of antibody identification and production known in the art can be used to produce an antibody that binds to a target molecule or complex of interest. In some embodiments an antibody is a monoclonal antibody. Monoclonal antibodies can be identified and/or produced using, e.g., hybridoma technology or recombinant nucleic acid technology in various embodiments. In some embodiments an antibody or portion thereof (e.g., an antigen-binding portion thereof) is selected from a library and/or using a display technique, e.g., a phage or yeast or ribosome display technique. In some embodiments, an antibody is a chimeric, humanized, or fully human antibody. In some embodiments an antibody is a polyclonal antibody. In some embodiments an antibody comprises at least two distinct antigen-binding sites that bind to distinct epitopes. In some embodiments an antibody has a label attached (e.g., covalently attached) thereto (e.g., the label may comprise a radioisotope, fluorescent agent, enzyme, hapten). In some embodiments a single chain antibody (scFv) may be created by joining the antigen-binding variable regions of heavy chain (VH) and light chain (VL) with a linking domain. A linking domain may comprise a peptide of, e.g., about 10 to about 25 amino acids. In some embodiments an antibody is a single polypeptide chain that can be expressed intracellularly in functional form. The polypeptide may comprise a targeting signal directing it to a particular intracellular location.

The term “aptamer” refers to an oligonucleotide that binds specifically and with high affinity to a target of interest, e.g., a polypeptide. An aptamer may be identified through a selection process using, e.g., systematic evolution of ligands by exponential enrichment (SELEX) or various directed evolution techniques. See, e.g., Turek, C. and Gold, L., Science 249: 505-10, 1990; Brody E N and Gold L J, Biotechnol. J, 74(1):5-13, 2000; L. Cerchia and V. de Franciscis, Trends Biotechnol., 28: 517-525, 2010; Keefe, A. Nat. Rev. Drug Discov. 9: 537-550, 2010. An aptamer is typically single-stranded (although it may form regions of double-stranded secondary structure through intramolecular complementarity).

The term “assay” encompasses any procedure or process of sequence of procedures or processes that may be used to identify or assess something. As used herein, “assess”, “assessing”, and similar terms encompass characterizing, detecting, determining, measuring, evaluating, estimating, analyzing, testing, etc. In various embodiments the thing being identified or assessed may be e.g., a gene, gene product, reactant or product of a reaction, a pathway, an agent, a composition, a cell, a cell line, a subject, a reagent for use in a composition or method, etc. In some embodiments an assay may be qualitative or may be at least in part quantitative, e.g., it may provide a measurement, which may be expressed numerically. A measurement may be relative or absolute in various embodiments. In some embodiments an assay provides a measurement of a magnitude, concentration, level, amount, intensity, degree of modulation (e.g., reduction or enhancement), activity, or a change in any of the foregoing, etc. In various embodiments the thing being or to identified or assessed may be, e.g., a gene, gene product, reactant or product of a reaction, a pathway, an agent, a composition, a cell, a cell line, a subject, a reagent for use in a composition or method, etc., or may be a sequence, structure, or other information or representation that may be manipulated, analyzed, processed, or displayed using a computer.

“Cellular marker” refers to a molecule (e.g., a protein, RNA, DNA, lipid, carbohydrate), complex, or portion thereof, the level of which in or on a cell (e.g., at least partly exposed at the cell surface) characterizes, indicates, or identifies one or more cell type(s), cell lineage(s), or tissue type(s) or characterizes, indicates, or identifies a particular state (e.g., a diseased or physiological state such as apoptotic or non-apoptotic, a differentiation state, a stem cell state). A level may be reported in a variety of different ways, e.g., high/low; +/−; numerically, etc. The presence, absence, or level of certain cellular marker(s) may indicate a particular physiological or diseased state of a patient, organ, tissue, or cell. It will be understood that multiple cellular markers may be assessed to, e.g., identify or isolate a cell type of interest, diagnose a disease, etc. In some embodiments between 2 and 10 cellular markers may be assessed. A cellular marker present on or at the surface of cells may be referred to as a “cell surface marker” (CSM). It will be understood that a CSM may be only partially exposed at the cell surface. In some embodiments a CSM or portion thereof is accessible to a specific binding agent present in the environment in which such cell is located, so that the binding agent may be used to, e.g., identify, label, isolate, or target the cell. In some embodiments a CSM is a protein at least part of which is located outside the plasma membrane of a cell. Examples of CSMs include CD molecules, receptors with an extracellular domain, channels, and cell adhesion molecules. In some embodiments, a receptor is a growth factor receptor, hormone receptor, integrin receptor, folate receptor, or transferrin receptor. A cellular marker may be cell type specific. A cell type specific marker is generally expressed or present at a higher level in or on (at the surface of) a particular cell type or cell types than in or on many or most other cell types (e.g., other cell types in the body or in an artificial environment). In some cases a cell type specific marker is present at detectable levels only in or on a particular cell type of interest. However, useful cell type specific markers may not be and often are not absolutely specific for the cell type of interest. A cellular marker, e.g., a cell type specific marker, may be present at levels at least 1.5-fold, at least 2-fold or at least 3-fold greater in or on the surface of a particular cell type than in a reference population of cells which may consist, for example, of a mixture containing cells from multiple (e.g., 5-10; 10-20, or more) of different tissues or organs in approximately equal amounts. In some embodiments a cellular marker, e.g., a cell type specific marker, may be present at levels at least 4-5 fold, between 5-10 fold, or more than 10-fold greater than its average expression in a reference population. In general, the level of a cellular marker may be determined using standard techniques such as Northern blotting, in situ hybridization, RT-PCR, sequencing, immunological methods such as immunoblotting, immunohistochemistry, fluorescence detection following staining with fluorescently labeled antibodies (e.g., flow cytometry, fluorescence microscopy), similar methods using non-antibody ligands that specifically bind to the marker, oligonucleotide or cDNA microarray or membrane array, protein microarray analysis, mass spectrometry. A CSM, e.g., a cell type specific CSM, may be used to detect or isolate cells or as a target in order to deliver an agent to cells. For example, the agent may be linked to a moiety that binds to a CSM. Suitable binding moieties include, e.g., antibodies or ligands, e.g., small molecules, aptamers, or polypeptides. Methods known in the art can be used to separate cells that express a cellular marker, e.g., a CSM, from cells that do not, if desired. In some embodiments a specific binding agent can be used to physically separate cells that express a CSM from cells that do not. In some embodiments, flow cytometry is used to quantify cells that express a cellular marker, e.g., a CSM, or to separate cells that express a cellular marker, e.g., a CSM, from cells that do not. For example, in some embodiments cells are contacted with a fluorescently labeled antibody that binds to the CSM. Fluorescence activated cell sorting (FACS) is then used to separate cells based on fluorescence.

“Computer-aided” as used herein encompasses methods in which a computer system is used to gather, process, manipulate, display, visualize, receive, transmit, store, or otherwise handle information (e.g., data results, structures, sequences, etc.). A method may comprise causing the processor of a computer to execute instructions to gather, process, manipulate, display, receive, transmit, or store data or other information. The instructions may be embodied in a computer program product comprising a computer-readable medium. In some embodiments a method comprises transmitting or receiving data or other information over a communication network. A communication network may, for example, comprise one or more intranets or the Internet.

“Disorder” refers to any disease or deviation from the normal structure or function of any tissue, part, organ or system of the body (or any combination thereof). A disorder frequently results in characteristic symptoms and signs, such as biological, chemical and physical changes, and may be associated with a variety of other factors including, but not limited to, demographic, environmental, occupational, genetic, and/or medical historical factors. Certain characteristic signs, symptoms, and/or related factors can be assessed through a variety of methods to yield information that may be useful in diagnosis or treatment selection. The term “disorder” may be used interchangeably with “disease”. Certain disorders are sometimes termed “syndrome” in the art and may be so referred to herein.

An “effective amount” or “effective dose” of an agent (or composition containing such agent) refers to the amount sufficient to achieve a desired biological and/or pharmacological effect, e.g., when delivered to a cell or organism according to a selected administration form, route, and/or schedule. As will be appreciated by those of ordinary skill in this art, the absolute amount of a particular agent or composition that is effective may vary depending on such factors as the desired biological or pharmacological endpoint, the agent to be delivered, the target tissue, etc. Those of ordinary skill in the art will further understand that an “effective amount” may be contacted with cells or administered to a subject in a single dose, or through use of multiple doses, in various embodiments. In some embodiments an effective amount is an amount sufficient to reduce the severity of at least one symptom or other manifestation of a mitochondrial disorder. In some embodiments an effective amount results in a statistically significant improvement in a clinically relevant parameter or score associated with a mitochondrial disorder. In some embodiments a reduction in severity or an improvement is statistically significant. In some embodiments a reduction in severity or an improvement is sufficiently great as to be considered clinically meaningful by one of ordinary skill in medicine (e.g., a physician).

The term “expression” encompasses the processes by which polynucleic acids (e.g., DNA) are transcribed to produce RNA, and (where applicable) RNA transcripts are processed and translated into polypeptides.

The term “gene product” (also referred to herein as “gene expression product” or “expression product”) encompasses products resulting from expression of a gene, such as RNA transcribed from a gene and polypeptides arising from translation of such RNA. It will be appreciated that certain gene products may undergo processing or modification, e.g., in a cell. For example, RNA transcripts may be spliced, polyadenylated, etc., prior to mRNA translation, and/or polypeptides may undergo co-translational or post-translational processing such as removal of secretion signal sequences, removal of organelle targeting sequences, or modifications such as phosphorylation, fatty acylation, etc. The term “gene product” encompasses such processed or modified forms. Genomic, mRNA, polypeptide sequences from a variety of species, including human, are known in the art and are available in publicly accessible databases such as those available at the National Center for Biotechnology Information (www.ncbi.nih.gov) or Universal Protein Resource (www.uniprot.org). Databases include, e.g., GenBank, RefSeq, Gene, UniProtKB/SwissProt, UniProtKB/Trembl, and the like. In general, sequences, e.g., mRNA and polypeptide sequences, in the NCBI Reference Sequence database may be used as gene product sequences for a gene of interest. It will be appreciated that multiple alleles of a gene may exist among individuals of the same species. For example, differences in one or more nucleotides (e.g., up to about 1%, 2%, 3-5% of the nucleotides) of the nucleic acids encoding a particular protein may exist among individuals of a given species. Due to the degeneracy of the genetic code, such variations often do not alter the encoded amino acid sequence, although DNA polymorphisms that lead to changes in the sequence of the encoded proteins can exist. Examples of polymorphic variants can be found in, e.g., the Single Nucleotide Polymorphism Database (dbSNP), available at the NCBI website at www.ncbi.nlm.nih.gov/projects/SNP/. (Sherry S T, et al. (2001). “dbSNP: the NCBI database of genetic variation”. Nucleic Acids Res. 29 (I): 308-311; Kitts A, and Sherry S, (2009). The single nucleotide polymorphism database (dbSNP) of nucleotide sequence variation in The NCBI Handbook [Internet]. McEntyre J, Ostell J, editors. Bethesda (MD): National Center for Biotechnology Information (US); 2002 (www.ncbi.nlm.mih.gov/bookshelf/br.fcgi?book→handbook&patt=ch5). Multiple isoforms of certain proteins may exist, e.g., as a result of alternative RNA splicing or editing. In general, where aspects of this disclosure pertain to a gene or gene product, embodiments pertaining to allelic variants or isoforms are encompassed unless indicated otherwise. Certain embodiments may be directed to particular sequence(s), e.g., particular allele(s) or isoform(s).

“Identity” or “percent identity” is a measure of the extent to which the sequence of two or more nucleic acids or polypeptides is the same. The percent identity between a sequence of interest A and a second sequence B may be computed by aligning the sequences, allowing the introduction of gaps to maximize identity, determining the number of residues (nucleotides or amino acids) that are opposite an identical residue, dividing by the minimum of TG_{A} and TG_{B} (here TG_{A} and TG_{B} are the sum of the number of residues and internal gap positions in sequences A and B in the alignment), and multiplying by 100. When computing the number of identical residues needed to achieve a particular percent identity, fractions are to be rounded to the nearest whole number. Sequences can be aligned with the use of a variety of) computer programs known in the art. For example, computer programs such as BLAST2, BLASTN, BLASTP, Gapped BLAST, etc., may be used to generate alignments and/or to obtain a percent identity. The algorithm of Karlin and Altschul (Karlin and Altschul, Proc. Natl. Acad. Sci. USA 87:22264-2268, 1990) modified as in Karlin and Altschul, Proc. Natl. Acad. Sci. USA 90:5873-5877, 1993 is incorporated into the NBLAST and XBLAST programs of Altschul et al. (Altschul, et al., J. Mol. Biol. 215:403-410, 1990). In some embodiments, to obtain gapped alignments for comparison purposes, Gapped BLAST is utilized as described in Altschul et al. (Altschul, et al. Nucleic Acids Res. 25: 3389-3402, 1997). When utilizing BLAST and Gapped BLAST programs, the default parameters of the respective programs may be used. See the Web site having URL www.ncbi.nlm.nih.gov and/or McGinnis, S, and Madden, T L, W20-W25 Nucleic Acids Research, 2004, Vol. 32, Web server issue. Other suitable programs include CLUSTALW (Thompson J D, Higgins D G, Gibson T J, Nuc Ac Res, 22:4673-4680, 1994) and GAP (GCG Version 9.1; which implements the Needleman & Wunsch, 1970 algorithm (Needleman S B, Wunsch C D, J Mol Biol, 48:443-453, 1970.) Percent identity may be evaluated over a window of evaluation. In some embodiments a window of evaluation may have a length of at least 10%, 15%, 20%, 25%, 30%, 35%, 40%, 45%, 50%, 55%, 60%, 65%, 70%, 75%, 80%, 85%, 90%, 95%, 96%, 97%, 98%, 99%, or more, e.g., 100%, of the length of the shortest of the sequences being compared. In some embodiments a window of evaluation is at least 100; 200; 300; 400; 500; 600; 700; 800; 900; 1,000; 1,200; 1,500; 2,000; 2,500; 3,000; 3,500; 4,000; 4,500; or 5,000 amino acids. In some embodiments no more than 20%, 10%, 5%, or 1% of positions in either sequence or in both sequences over a window of evaluation are occupied by a gap. In some embodiments no more than 20%, 10%, 5%, or 1% of positions in either sequence or in both sequences are occupied by a gap.

“Inhibit” may be used interchangeably with terms such as “suppress”, “decrease”, “reduce” and like terms, as appropriate in the context. It will be understood that the extent of inhibition may vary. For example, inhibition may refer to a reduction of the relevant level by at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99%. In some embodiments inhibition refers to a decrease of 100%, e.g., to background levels or undetectable levels. In some embodiments inhibition is statistically significant.

“Isolated” means 1) separated from at least some of the components with which it is usually associated in nature; 2) prepared or purified by a process that involves the hand of man; and/or 3) not occurring in nature, e.g., present in an artificial environment. In some embodiments an isolated cell is a cell that has been separated from at least some other cells in a cell population or that remains after at least some cells in a cell population have been removed or been eliminated (e.g., killed).

“Mitochondrial disorder” refers to any disorder in which abnormal mitochondrial function, structure, and/or number plays a role. The term encompasses any disorder caused at least in part by a defect in amount (e.g., reduced expression), structure, or activity of one or more mitochondrial proteins, protein complexes, or substructures and/or a defect in mitochondrial number. The terms “abnormal mitochondrial function” and “mitochondrial dysfunction” are used interchangeably herein. In some embodiments “mitochondrial dysfunction” or “abnormal mitochondrial function” refers to a deficiency or lack of one or more mitochondrial functions, e.g., relative to a normal level, e.g., a level found in cells or tissues of or obtained from a normal, healthy subject. In some embodiments abnormal mitochondrial function refers to an abnormally low level of at least one mitochondrial activity in one or more mammalian cell types or tissues, where such activity is beneficial to a mammalian cell, tissue, or organism comprising the cell or tissue. In some embodiments abnormal mitochondrial function refers to an abnormally high level of at least one mitochondrial activity in one or more mammalian cell types or tissues, where such activity is detrimental to a mammalian cell, tissue, or organism comprising the cell or tissue. In some embodiments abnormal mitochondrial number refers to an abnormally low number of mitochondria in one or more mammalian cell types or tissues, e.g., relative to a normal level, e.g., a level found in cells or tissues of or obtained from a normal, healthy subject. A disorder may be classified according to a tissue, organ, or body system that is frequently or typically significantly affected in individuals suffering from the disorder and/or on which the effect of the disorder results in significant morbidity or mortality. It will be appreciated that many mitochondrial disorders frequently or typically affect multiple organs, body systems, or processes. In some embodiments a mitochondrial disorder is a multi-system or multi-organ disorder characterized by significant manifestations affecting at least two different tissues, organs, or body systems. It will be understood that many disorders can reasonably be classified under multiple categories, and the classification of a disorder into one or more categories herein should not be taken to imply that it the disorders is not also a member of one or more other categories, whether or not listed herein.

“Mitochondrial poison” refers to an agent that inhibits at least one mitochondrial function, reduces the average number of mitochondria per cell, and/or causes an abnormality in mitochondrial structure. In some embodiments a mitochondrial poison binds to a mitochondrial protein, mitochondrial complex, or cofactor (e.g., heme or a component thereof such as iron). In some embodiments a mitochondrial poison is a small molecule. In some embodiments a mitochondrial poison causes depletion of mitochondrial DNA.

“Mitochondrial protein” refers to any protein that is found within mitochondria, e.g., in one or more mammalian cell types. In some embodiments a mitochondrial protein is encoded by nuclear DNA. In some embodiments a mitochondrial protein is encoded by mitochondrial DNA.

“Modulate”, “modulating”, “modulation” and like terms refer to causing or facilitating a qualitative or quantitative change, alteration, or modification of a target, e.g., activating (stimulating, upregulating) or inhibiting (suppressing, downregulating) a target. In some aspects, “modulating” comprises increasing (enhancing) or decreasing (reducing) the amount or activity of a target. In various embodiments a “target” may be a gene, gene product, molecule, complex, biological process, biological pathway, biological activity, biological process, chemical reaction, or a component of any of these. A “modulator” is an agent that modulates, and may be, e.g., an activator or an inhibitor.

“Mutagenize” encompasses application of any means of causing a change (“mutation”) in the sequence of a nucleic acid. A mutation may comprise an insertion, substitution (e.g., a point mutation), or deletion. In some embodiments the nucleic acid is in a cell, e.g., a mutation is a change in the DNA sequence of a cell's genome. In some embodiments a mutation results in altered sequence of a gene product. The gene product may be said to have a mutation. For example, an altered amino acid sequence arising from a mutation in a gene encoding a protein may be referred to as a mutation. In some embodiments a nucleic acid is an isolated nucleic acid, which isolated nucleic acid may be subsequently introduced into a cell or subject. In some embodiments cells are mutagenized by insertional mutagenesis, e.g., using a gene trap vector. In some embodiments cells are mutagenized using a chemical mutagen such as an alkylating agent (e.g., ethyl methanesulfonate or N-ethyl-N-nitrosourea) or using ultraviolet radiation. It will be understood that in many instances less than 100% of the cells in a population of cells subjected to mutagenesis will acquire a change in DNA sequence. A mutagenesis procedure may be selected to achieve a desired percent of cells that harbor one or more mutations. In some embodiments at least 5%, 10%, 20%, 30%, 40%, 50%, 60%, 70%, 80%, 90%, or more cells in a population of mutagenized cells have at least one mutation. In some embodiments the average number of mutations per cell is between about 0.1 and about 10, e.g., between about 0.2 and about 5.0, e.g., between about 0.5 and about 1.0. A cell comprising a genome harboring at least one mutation may be referred to as a “mutant cell”.

“Nucleic acid” is used interchangeably with “polynucleotide” and encompasses polymers of nucleotides. “Oligonucleotide” refers to a relatively short nucleic acid, e.g., typically between about 4 and about 100 nucleotides (nt) long, e.g., between 8-60 nt or between 10-40 nt long. Nucleotides include, e.g., ribonucleotides or deoxyribonucleotides. In some embodiments a nucleic acid comprises or consists of DNA or RNA. In some embodiments a nucleic acid comprises or includes only standard nucleobases (often referred to as “bases”). The standard bases are cytosine, guanine, adenine (which are found in DNA and RNA), thymine (which is found in DNA) and uracil (which is found in RNA), abbreviated as C, G, A, T, and U, respectively. In some embodiments a nucleic acid may comprise one or more non-standard nucleobases, which may be naturally occurring or non-naturally occurring (i.e., artificial; not found in nature) in various embodiments. In some embodiments a nucleic acid may comprise chemically or biologically modified bases (e.g., alkylated (e.g., methylated) bases), modified sugars (e.g., 2′-O-alkyribose (e.g., 2′-O methylribose), 2′-fluororibose, arabinose, or hexose), modified phosphate groups (e.g., phosphorothioates or 5′-N-phosphoramidite linkages). In some embodiments a nucleic acid comprises subunits (residues), e.g., nucleotides, that are linked by phosphodiester bonds. In some embodiments, at least some subunits of a nucleic acid are linked by a non-phosphodiester bond or other backbone structure. In some embodiments, a nucleic acid comprises a locked nucleic acid, morpholino, or peptide nucleic acid. A nucleic acid may be linear or circular in various embodiments. A nucleic acid may be single-stranded, double-stranded, or partially double-stranded in various embodiments. An at least partially double-stranded nucleic acid may be blunt-ended or may have one or more overhangs, e.g., 5′ and/or 3′ overhang(s). Nucleic acid modifications (e.g., base, sugar, and/or backbone modifications), non-standard nucleotides or nucleosides, etc., such as those known in the art as being useful in the context of RNA interference (RNAi), aptamer, or antisense-based molecules for research or therapeutic purposes may be incorporated in various embodiments. Such modifications may, for example, increase stability (e.g., by reducing sensitivity to cleavage by nucleases), decrease clearance in vivo, increase cell uptake, or confer other properties that improve the potency, efficacy, specificity, or otherwise render the nucleic acid more suitable for an intended use. Various non-limiting examples of nucleic acid modifications are described in, e.g., Deleavey G F, et al., Chemical modification of siRNA. Curr. Protoc. Nucleic Acid Chem. 2009; 39:16.3.1-16.3.22; Crooke, S T (ed.) Antisense drug technology: principles, strategies, and applications, Boca Raton: CRC Press, 2008; Kurreck, J. (ed.) Therapeutic oligonucleotides, RSC biomolecular sciences. Cambridge: Royal Society of Chemistry, 2008; U.S. Pat. Nos. 4,469,863; 5,536,821; 5,541,306; 5,637,683; 5,637,684; 5,700,922; 5,717,083; 5,719,262; 5,739,308; 5,773,601; 5,886,165; 5,929,226; 5,977,296; 6,140,482; 6,455,308 and/or in PCT application publications WO 00/56746 and WO 01/14398. Different modifications may be used in the two strands of a double-stranded nucleic acid. A nucleic acid may be modified uniformly or on only a portion thereof and/or may contain multiple different modifications.

A “polypeptide” refers to a polymer of amino acids linked by peptide bonds. A protein is a molecule comprising one or more polypeptides. A peptide is a relatively short polypeptide, typically between about 2 and 100 amino acids (aa) in length, e.g., between 4 and 60 aa; between 8 and 40 aa; between 10 and 30 aa. The terms “protein”, “polypeptide”, and “peptide” may be used interchangeably. In general, a polypeptide may contain only standard amino acids or may comprise one or more non-standard amino acids (which may be naturally occurring or non-naturally occurring amino acids) and/or amino acid analogs in various embodiments. A “standard amino acid” is any of the 20 L-amino acids that are commonly utilized in the synthesis of proteins by mammals and are encoded by the genetic code. A “non-standard amino acid” is an amino acid that is not commonly utilized in the synthesis of proteins by mammals. Non-standard amino acids include naturally occurring amino acids (other than the 20 standard amino acids) and non-naturally occurring amino acids. In some embodiments, a non-standard, naturally occurring amino acid is found in mammals. For example, ornithine, citrulline, and homocysteine are naturally occurring non-standard amino acids that have important roles in mammalian metabolism. Examples of non-standard amino acids include, e.g., singly or multiply halogenated (e.g., fluorinated) amino acids, D-amino acids, homo-amino acids, N-alkyl amino acids (other than proline), dehydroamino acids, aromatic amino acids (other than histidine, phenylalanine, tyrosine and tryptophan), and α,α disubstituted amino acids. An amino acid, e.g., one or more of the amino acids in a polypeptide, may be modified, for example, by addition, e.g., covalent linkage, of a moiety such as an alkyl group, an alkanoyl group, a carbohydrate group, a phosphate group, a lipid, a polysaccharide, a halogen, a linker for conjugation, a protecting group, etc. Modifications may occur anywhere in a polypeptide, e.g., the peptide backbone, the amino acid side-chains and the amino or carboxyl termini. A given polypeptide may contain many types of modifications. Polypeptides may be branched or they may be cyclic, with or without branching. Polypeptides may be conjugated with, encapsulated by, or embedded within a polymer or polymeric matrix, dendrimer, nanoparticle, microparticle, liposome, or the like. Modification may occur prior to or after an amino acid is incorporated into a polypeptide in various embodiments. Polypeptides may, for example, be purified from natural sources, produced in vitro or in vivo in suitable expression systems using recombinant DNA technology (e.g., by recombinant host cells or in transgenic animals or plants), synthesized through chemical means such as conventional solid phase peptide synthesis, and/or methods involving chemical ligation of synthesized peptides (see, e.g., Kent, S., J Pept Sci., 9(9):574-93, 2003 or U.S. Pub. No. 20040115774), or any combination of the foregoing.

As used herein, the term “purified” refers to agents that have been separated from most of the components with which they are associated in nature or when originally generated or with which they were associated prior to purification. In general, such purification involves action of the hand of man. Purified agents may be partially purified, substantially purified, or pure. Such agents may be, for example, at least 50%, 60%, 70%, 75%, 80%, 85%, 90%, 95%, 96%, 97%, 98%, 99%, or more than 99% pure. In some embodiments, a nucleic acid, polypeptide, or small molecule is purified such that it constitutes at least 75%, 80%, 85%, 90%, 95%, 96%, 97%, 98%, 99%, or more, of the total nucleic acid, polypeptide, or small molecule material, respectively, present in a preparation. In some embodiments, an organic substance, e.g., a nucleic acid, polypeptide, or small molecule, is purified such that it constitutes at least 75%, 80%, 85%, 90%, 95%, 96%, 97%, 98%, 99%, or more, of the total organic material present in a preparation. Purity may be based on, e.g., dry weight, size of peaks on a chromatography tracing (GC, HPLC, etc.), molecular abundance, electrophoretic methods, intensity of bands on a gel, spectroscopic data (e.g., NMR), elemental analysis, high throughput sequencing, mass spectrometry, or any art-accepted quantification method. In some embodiments, water, buffer substances, ions, and/or small molecules (e.g., synthetic precursors such as nucleotides or amino acids), can optionally be present in a purified preparation. A purified agent may be prepared by separating it from other substances (e.g., other cellular materials), or by producing it in such a manner to achieve a desired degree of purity. In some embodiments “partially purified” with respect to a molecule produced by a cell means that a molecule produced by a cell is no longer present within the cell, e.g., the cell has been lysed and, optionally, at least some of the cellular material (e.g., cell wall, cell membrane(s), cell organelle(s)) has been removed and/or the molecule has been separated or segregated from at least some molecules of the same type (protein, RNA, DNA, etc.) that were present in the lysate.

The term “reporter” or “reporter molecule” often refers to an RNA or protein that, when expressed by a cell, can be used to distinguish or separate the cell from otherwise similar cells that do not express the RNA or protein or can be used to distinguish or separate the cells from other cells that express the RNA or protein at different levels or in which the RNA or protein has a lower or higher activity. The term “reporter gene” refers to a nucleic acid that encodes a reporter. In some embodiments a reporter gene comprises DNA that is transcribed to mRNA that is translated by the cell to produce a protein. The protein has a property that allows the cell to be distinguished or separated from cells that do not produce the protein. In some embodiments DNA encoding a reporter molecule is operably linked to expression control signals, e.g., a promoter.

In some embodiments, a reporter comprises a selectable marker. As used herein, the term “selectable marker” refers to a reporter that, when expressed by a cell, confers on the cell a proliferation or survival advantage under at least some conditions (“selective conditions”), relative to otherwise similar cells not expressing the reporter. Selectable markers that confer a proliferation or survival advantage and methods of selecting cells based on expression of such markers are known in the art. Examples of selectable markers include proteins that confer resistance to various drugs (“drug resistance markers”). Selective conditions for drug resistance markers typically comprise culturing cells in media that contains the relevant drug in concentrations sufficient to significantly reduce cell viability and/or proliferation. One of skill in the art will be aware of appropriate concentrations. Optimum concentrations for any particular cell type or cell line can be readily determined. Examples of drug resistance markers include enzymes conferring resistance to various aminoglycoside antibiotics such as G418 and neomycin (e.g., an aminoglycoside 3′-phosphotransferase, 3′ APH II, also known as neomycin phosphotransferase II (nptII or “neo”)), Zeocin™ or bleomycin (e.g., the protein encoded by the ble gene from Streptoalloteichus hindustanus), hygromycin (e.g., hygromycin resistance gene, hph, from Streptomyces hygroscopicus or from a plasmid isolated from Escherichia coli or Klebsiella pneumoniae, which codes for a kinase (hygromycin phosphotransferase, HPT) that inactivates Hygromycin B through phosphorylation), puromycin (e.g., the Streptomyces alboniger puromycin-N-acetyl-transferase (pac) gene), or blasticidin (e.g., an acetyl transferase encoded by the bls gene from Streptoverticillum sp. JCM 4673, or a deaminase encoded by a gene such as bsr, from Bacillus cereus or the BSD resistance gene from Aspergillus terreus). Other drug resistance markers are dihydrofolate reductase (DHFR), adenosine deaminase (ADA), thymidine kin (TK), and hypoxanthine-guanine phosphoribosyltransferase (HPRT). Proteins such as P-glycoprotein and other multidrug resistance proteins act as pumps through which various cytotoxic compounds, e.g., chemotherapeutic agents such as vinblastine and anthracyclines, are expelled from cells. (See Ambudkar S V, et al., Oncogene, 22(47):7468-85, 2003) could also be used as selectable markers. In some embodiments a drug resistance marker other than neo, such as a puromycin-N-acetyl-transferase, is used. In some embodiments a drug resistance marker is not a mitochondrial poison.

Proteins that function in biosynthetic pathways and confer prototrophy with respect to particular compounds required for cell viability or proliferation (“nutritional markers”) may be used as selectable markers. Selective conditions for nutritional markers often comprise culturing cells in media that lacks a sufficient concentration of the relevant compound to support cell viability and/or proliferation. In general, under nonselective conditions the required compound is present in the environment or is produced by an alternative pathway in the cell. Under selective conditions, functioning of the biosynthetic pathway is needed since the cell must produce the compound. HPRT and TK are examples. Cells lacking HPRT or TK expression (e.g., lacking a functional copy of the HPRT gene or TK gene, respectively) can grow in standard culture medium but die in HAT medium. In cells lacking HPRT or TK expression, HPRT or TK, respectively, can be used as a selectable marker whose presence may be selected for in HAT medium.

Culturing a population of cells under selective conditions, wherein some of the cells express a selectable marker that confers a proliferation or survival advantage and other cells do not express the selectable marker, will, in general, eventually result in a population enriched for cells that express the selectable marker. In many embodiments, most or all cells that do not express the selectable marker will be eliminated from the population after a sufficient time. The time required to eliminate a given percentage of cells not expressing the selectable marker will depend on the marker, the conditions, and the cells, and can be readily determined by the skilled artisan. It will be understood that “selective conditions” can refer to a single set of conditions or to multiple sets of conditions, which may be applied in sequence. It will also be understood that cells need not be maintained continuously under the selective conditions.

In some embodiments a reporter is or comprises a readily detectable molecule, e.g., a protein that can be readily detected such as a fluorescent or luminescent protein or an enzyme that acts on a substrate to produce a colored, fluorescent, or luminescent substance or are capable of absorbing light of a particular wavelength. In some embodiments a readily detectable molecule produces a signal or a change in a signal based on light or an interaction with light (an “optically detectable signal”), which signal can be detected e.g., visually or using suitable instrumentation. Fluorescent markers include green fluorescent protein (GFP), blue, sapphire, yellow, red, orange, and cyan fluorescent proteins and fluorescent variants such as enhanced GFP (eGFP), mCherry, etc. Enzymes useful as reporters in certain embodiments include, e.g., beta-galactosidase, horseradish peroxidase, alkaline phosphatase, and luciferase (e.g., firefly, Renilla, or Gaussia luciferase). In some embodiments, e.g., in the case of an enzyme that acts on a substrate, cells may be contacted with a cell-permeable substrate. Cells expressing the enzyme can be distinguished from cells that do not. In some embodiments selection is based at least in part on lack of expression of a protein.

In some embodiments, a reporter is a protein that is ordinarily secreted by a cell. In some embodiments a nucleic acid sequence encoding a secretion signal sequence may be removed from the coding sequence in constructing a reporter gene, so that the protein is not secreted when used as a reporter. In some embodiments the reporter protein is secreted. A secreted protein may be detected in culture medium. Cells in a vessel (e.g., a well) in which the secreted reporter protein is detected may be subcloned one or more times to obtain a clonal cell line.

In some embodiments, a reporter is encoded by a sequence that is codon-optimized for expression in a cell from an organism of interest.

It will be understood that a reporter can be used for a variety of purposes other than identifying or selecting cells based on expression or activity of the reporter. For example, expression or activity of a reporter can “report on”, e.g., provide information relating to, a cell process such as transcription, translation, degradation, signal transduction, protein translocation, enzyme activity, metabolism, protein-protein interaction, or any of a variety of other processes or phenotypes of interest. Such information may relate to particular genes, RNAs, proteins, or signaling pathways. The information may be qualitative or, in some embodiments, quantitative.

The term “RNA interference” (RNAi) encompasses processes in which a molecular complex known as an RNA-induced silencing complex (RISC) silences or “knocks down” gene expression in a sequence-specific manner in, e.g., eukaryotic cells, e.g., vertebrate cells, or in an appropriate in vitro system. RISC may incorporate a short nucleic acid strand (e.g., about 16-about 30 nucleotides (nt) in length) that pairs with and directs or “guides” sequence-specific degradation or translational repression of RNA (e.g., mRNA) to which the strand has complementarity. The short nucleic acid strand may be referred to as a “guide strand” or “antisense strand”. An RNA strand to which the guide strand has complementarity may be referred to as a “target RNA”. A guide strand may initially become associated with RISC components (in a complex sometimes termed the RISC loading complex) as part of a short double-stranded RNA (dsRNA), e.g., a short interfering RNA (siRNA). The other strand of the short dsRNA may be referred to as a “passenger strand” or “sense strand”. The complementarity of the structure formed by hybridization of a target RNA and the guide strand may be such that the strand can (i) guide cleavage of the target RNA in the RNA-induced silencing complex (RISC) and/or (ii) cause translational repression of the target RNA. Reduction of expression due to RNAi may be essentially complete (e.g., the amount of a gene product is reduced to background levels) or may be less than complete in various embodiments. For example, mRNA and/or protein level may be reduced by 50%, 60%, 70%, 75%, 80%, 85%, 90%, 95%, or more, in various embodiments. As known in the art, the complementarity between the guide strand and a target RNA need not be perfect (100%) but need only be sufficient to result in inhibition of gene expression. For example, in some embodiments 1, 2, 3, 4, 5, or more nucleotides of a guide strand may not be matched to a target RNA. “Not matched” or “unmatched” refers to a nucleotide that is mismatched (not complementary to the nucleotide located opposite it in a duplex, i.e., wherein Watson-Crick base pairing does not take place) or forms at least part of a bulge. Examples of mismatches include, without limitation, an A opposite a G or A, a C opposite an A or C, a U opposite a C or U, a G opposite a G. A bulge refers to a sequence of one or more nucleotides in a strand within a generally duplex region that are not located opposite to nucleotide(s) in the other strand. “Partly complementary” refers to less than perfect complementarity. In some embodiments a guide strand has at least about 80%, 85%, or 90%, e.g., least about 91%, 92%, 93%, 94%, 95%, 96%, 97%, 98%, 99%, or 100% sequence complementarity to a target RNA over a continuous stretch of at least about 15 nt, e.g., between 15 nt and 30 nt, between 17 nt and 29 nt, between 18 nt and 25 nt, between 19 nt and 23 nt, of the target RNA. In some embodiments at least the seed region of a guide strand (the nucleotides in positions 2-7 or 2-8 of the guide strand) is perfectly complementary to a target RNA. In some embodiments, a guide strand and a target RNA sequence may form a duplex that contains no more than 1, 2, 3, or 4 mismatched or bulging nucleotides over a continuous stretch of at least 10 nt, e.g., between 10-30 nt. In some embodiments a guide strand and a target RNA sequence may form a duplex that contains no more than 1, 2, 3, 4, 5, or 6 mismatched or bulging nucleotides over a continuous stretch of at least 12 nt, e.g., between 10-30 nt. In some embodiments, a guide strand and a target RNA sequence may form a duplex that contains no more than 1, 2, 3, 4, 5, 6, 7, or 8 mismatched or bulging nts over a continuous stretch of at least 15 nt, e.g., between 10-30 nt. In some embodiments, a guide strand and a target RNA sequence may form a duplex that contains no mismatched or bulging nucleotides over a continuous stretch of at least 10 nt, e.g., between 10-30 nt. In some embodiments, between 10-30 nt is 10, 11, 12, 13, 14, 15, 16, 17, 18, 19, 20, 21, 22, 23, 24, 25, 26, 27, 28, 29, or 30 nt.

As used herein, the term “RNAi agent” encompasses nucleic acids that can be used to achieve RNAi in eukaryotic cells. Short interfering RNA (siRNA), short hairpin RNA (shRNA), and microRNA (miRNA) are examples of RNAi agents. siRNAs typically comprise two separate nucleic acid strands that are hybridized to each other to form a structure that contains a double stranded (duplex) portion at least 15 nt in length, e.g., about 15-about 30 nt long, e.g., between 17-27 nt long, e.g., between 18-25 nt long, e.g., between 19-23 nt long, e.g., 15, 16, 17, 18, 19, 20, 21, 22, 23, 24, 25, 26, 27, 28, 29, or 30 nucleotides. In some embodiments the strands of an siRNA are perfectly complementary to each other within the duplex portion. In some embodiments the duplex portion may contain one or more unmatched nucleotides, e.g., one or more mismatched (non-complementary) nucleotide pairs or bulged nucleotides. In some embodiments either or both strands of an siRNA may contain up to about 1, 2, 3, or 4 unmatched nucleotides within the duplex portion. In some embodiments a strand may have a length of between 15-35 nt, e.g., between 17-29 nt, e.g., 19-25 nt, e.g., 21-23 nt. Strands may be equal in length or may have different lengths in various embodiments. In some embodiments strands may differ by between 1-10 nt in length. A strand may have a 5′ phosphate group and/or a 3′ hydroxyl (—OH) group. Either or both strands of an siRNA may comprise a 3′ overhang of, e.g., about 1-10 nt (e.g., 1-5 nt, e.g., 2 nt). Overhangs may be the same length or different in lengths in various embodiments. In some embodiments an overhang may comprise or consist of deoxyribonucleotides, ribonucleotides, or modified nucleotides or modified ribonucleotides such as 2′-O-methylated nucleotides, or 2′-O-methyl-uridine. An overhang may be perfectly complementary, partly complementary, or not complementary to a target RNA in a hybrid formed by the guide strand and the target RNA in various embodiments.

shRNAs are nucleic acid molecules that comprise a stem-loop structure and a length typically between about 40-150 nt, e.g., about 50-100 nt, e.g., 60-80 nt. A “stem-loop structure” (also referred to as a “hairpin” structure) refers to a nucleic acid having a secondary structure that includes a region of nucleotides which are known or predicted to form a double strand (stem portion; duplex) that is linked on one side by a region of (usually) predominantly single-stranded nucleotides (loop portion). Such structures are well known in the art and the term is used consistently with its meaning in the art. A guide strand sequence may be positioned in either arm of the stem, i.e., 5′ with respect to the loop or 3′ with respect to the loop in various embodiments. As is known in the art, the stem structure does not require exact base-pairing (perfect complementarity). Thus, the stem may include one or more unmatched residues or the base-pairing may be exact, i.e., it may not include any mismatches or bulges. In some embodiments the stem is between 15-30 nt, e.g., between 17-29 nt, e.g., 19-25 nt. In some embodiments the stem is between15-19 nt. In some embodiments the stem is between19-30 nt. The primary sequence and number of nucleotides within the loop may vary. Examples of loop sequences include, e.g., UGGU; ACUCGAGA; UUCAAGAGA. In some embodiments a loop sequence found in a naturally occurring miRNA precursor molecule (e.g., a pre-miRNA) may be used. In some embodiments a loop sequence may be absent (in which case the termini of the duplex portion may be directly linked). In some embodiments a loop sequence may be at least partly self-complementary. In some embodiments the loop is between 1 and 20 nt in length, e.g., 1-15 nt, e.g., 4-9 nt. The shRNA structure may comprise a 5′ or 3′ overhang. As known in the art, an shRNA may undergo intracellular processing, e.g., by the ribonuclease (RNase) III family enzyme known as Dicer, to remove the loop and generate an siRNA.

Mature endogenous miRNAs are short (typically 18-24 nt, e.g., about 22 nt), single-stranded RNAs that are generated by intracellular processing from larger, endogenously encoded precursor RNA molecules termed miRNA precursors (see, e.g., Bartel, D., Cell. 116(2):281-97 (2004); Bartel D P. Cell. 136(2):215-33 (2009); Winter, J., et al., Nature Cell Biology 11: 228-234 (2009). Artificial miRNA may be designed to take advantage of the endogenous RNAi pathway in order to silence a target RNA of interest.

An RNAi agent that contains a strand sufficiently complementary to an RNA of interest so as to result in reduced expression of the RNA of interest (e.g., as a result of degradation or repression of translation of the RNA) in a cell or in an in vitro system capable of mediating RNAi and/or that comprises a sequence that is at least 80%, 90%, 95%, or more (e.g., 100%) complementary to a sequence comprising at least 10, 12, 15, 17, or 19 consecutive nucleotides of an RNA of interest may be referred to as being “targeted to” the RNA of interest. An RNAi agent targeted to an RNA transcript may also considered to be targeted to a gene from which the transcript is transcribed.

In some embodiments an RNAi agent is a vector (e.g., an expression vector) suitable for causing intracellular expression of one or more transcripts that give rise to a siRNA, shRNA, or miRNA in the cell. Such a vector may be referred to as an “RNAi vector”. An RNAi vector may comprise a template that, when transcribed, yields transcripts that may form a siRNA (e.g., as two separate strands that hybridize to each other), shRNA, or miRNA precursor (e.g., pri-miRNA or pre-mRNA).

An RNAi agent may be produced in any of variety of ways in various embodiments. For example, nucleic acid strands may be chemically synthesized (e.g., using standard nucleic acid synthesis techniques) or may be produced in cells or using an in vitro transcription system. Strands may be allowed to hybridize (anneal) in an appropriate liquid composition (sometimes termed an “annealing buffer”). An RNAi vector may be produced using standard recombinant nucleic acid techniques.

A “sample” may be any biological specimen. In some embodiments a sample comprises a body fluid such as blood, cerebrospinal fluid, (CSF), sputum, lymph, mucus, saliva, a glandular secretion, or urine. In some embodiments a sample comprises cells, tissue, or cellular material (e.g., material derived from cells, such as a cell lysate or fraction thereof). A sample may be obtained from (i.e., originates from, was initially removed from) a subject. Methods of obtaining samples are known in the art and include, e.g., tissue biopsy, such as excisional biopsy, incisional biopsy, biopsy, or core biopsy; fine needle aspiration biopsy; brushings; lavage; or collecting body fluids that may contain cells, such as blood, sputum, lymph, mucus, saliva, or urine. In some embodiments a sample contains at least some intact cells at the time it is removed from a subject. In some embodiments a sample retains at least some of the microarchitecture of a tissue from which it was removed. A sample may be subjected to one or more processing steps after having been obtained from a subject and/or may be split into one or more portions. For example, in some embodiments a sample comprises plasma or serum obtained from a blood sample that has been processed to obtain such plasma or serum. The term “sample” encompasses processed samples, portions of samples, etc., and such samples are considered to have been obtained from the subject from whom the initial sample was removed. In some embodiments a sample may be obtained from an individual who has been diagnosed with or is suspected of having a mitochondrial disorder. A sample, e.g., a sample used in a method or composition disclosed herein, may have been procured directly from a subject, or indirectly, e.g., by receiving the sample from one or more persons who procured the sample directly from the subject, e.g., by performing a biopsy, surgery, or other procedure on the subject.

A “small molecule” as used herein, is an organic molecule that is less than about 2 kilodaltons (kDa) in mass. In some embodiments, the small molecule is less than about 1.5 kDa, or less than about 1 kDa. In some embodiments, the small molecule is less than about 800 daltons (Da), 600 Da, 500 Da, 400 Da, 300 Da, 200 Da, or 100 Da. Often, a small molecule has a mass of at least 50 Da. In some embodiments, a small molecule is non-polymeric. In some embodiments, a small molecule is not an amino acid. In some embodiments, a small molecule is not a nucleotide. In some embodiments, a small molecule is not a saccharide. In some embodiments, a small molecule contains multiple carbon-carbon bonds and can comprise one or more heteroatoms and/or one or more functional groups important for structural interaction with proteins (e.g., hydrogen bonding), e.g., an amine, carbonyl, hydroxyl, or carboxyl group, and in some embodiments at least two functional groups. Small molecules often comprise one or more cyclic carbon or heterocyclic structures and/or aromatic or polyaromatic structures, optionally substituted with one or more of the above functional groups.

“Specific binding” generally refers to a physical association between a target molecule (e.g., a polypeptide) or complex and a binding agent such as an antibody, aptamer or ligand. The association is typically dependent upon the presence of a particular structural feature of the target such as an antigenic determinant, epitope, binding pocket or cleft, recognized by the binding agent. For example, if an antibody is specific for epitope A, the presence of a polypeptide containing epitope A or the presence of free unlabeled A in a reaction containing both free labeled A and the binding agent that binds thereto, will typically reduce the amount of labeled A that binds to the binding agent. It is to be understood that specificity need not be absolute but generally refers to the context in which the binding occurs. For example, it is well known in the art that antibodies may in some instances cross-react with other epitopes in addition to those present in the target. Such cross-reactivity may be acceptable depending upon the application for which the antibody is to be used. One of ordinary skill in the art will be able to select binding agents, e.g., antibodies, aptamers, or ligands, having a sufficient degree of specificity to perform appropriately in any given application (e.g., for detection of a target molecule). It is also to be understood that specificity may be evaluated in the context of additional factors such as the affinity of the binding agent for the target versus the affinity of the binding agent for other targets, e.g., competitors. If a binding agent exhibits a high affinity for a target molecule that it is desired to detect and low affinity for nontarget molecules, the binding agent will likely be an acceptable reagent. Once the specificity of a binding agent is established in one or more contexts, it may be employed in other contexts, e.g., similar contexts such as similar assays or assay conditions, without necessarily re-evaluating its specificity. In some embodiments specificity of a binding agent can be tested by performing an appropriate assay on a sample expected to lack the target (e.g., a sample from cells in which the gene encoding the target has been disabled or effectively inhibited) and showing that the assay does not result in a signal significantly different to background. In some embodiments, a first entity (e.g., molecule, complex) is said to “specifically bind” to a second entity if it binds to the second entity with substantially greater affinity than to most or all other entities present in the environment where such binding takes place and/or if the two entities bind with an equilibrium dissociation constant, K_{d}, of 10⁻⁴ or less, e.g., 10⁻⁵ M or less, e.g., 10⁻⁶ M or less, 10⁻⁷ M or less, 10⁻⁸ M or less, or 10⁻⁹ M or less. K_{d} can be measured using any suitable method known in the art, e.g., surface plasmon resonance-based methods, isothermal titration calorimetry, spectroscopy-based methods, etc. “Specific binding agent” refers to an entity that specifically binds to another entity, e.g., a molecule or molecular complex, which may be referred to as a “target”. “Specific binding pair” refers to two entities (e.g., molecules or molecular complexes) that specifically bind to one another. Examples are biotin-avidin, antibody-antigen, complementary nucleic acids, receptor-ligand, etc.

A “subject” may be any vertebrate organism in various embodiments. A subject may be individual to whom an agent is administered, e.g., for experimental, diagnostic, and/or therapeutic purposes or from whom a sample is obtained or on whom a procedure is performed. In some embodiments a subject is a mammal, e.g. a human, non-human primate, rodent (e.g., mouse, rat, rabbit), ungulate (e.g., ovine, bovine, equine, caprine species), canine, or feline. In some embodiments, a human subject is between newborn and 6 months old. In some embodiments, a human subject is between 6 and 24 months old. In some embodiments, a human subject is between 2 and 6, 6 and 12, or 12 and 18 years old. In some embodiments a human subject is between 18 and 30, 30 and 50, 50 and 80, or greater than 80 years old. In some embodiments, a subject is an adult. For purposes hereof a human at least 18 years of age is considered an adult. In some embodiments a subject is an embryo. In some embodiments a subject is a fetus. In certain embodiments an agent is administered to a pregnant female in order to treat or cause a biological effect on an embryo or fetus in utero.

“Treat”, “treating” and similar terms refer to providing medical and/or surgical management of a subject. Treatment may include, but is not limited to, administering an agent or composition (e.g., a pharmaceutical composition) to a subject. Treatment is typically undertaken in an effort to alter the course of a disease (which term is used to indicate any disease, disorder, or undesirable condition warranting therapy) in a manner beneficial to the subject. The effect of treatment may include reversing, alleviating, reducing severity of, delaying the onset of, curing, inhibiting the progression of, and/or reducing the likelihood of occurrence or recurrence of the disease or one or more symptoms or manifestations of the disease. A therapeutic agent may be administered to a subject who has a disease or is at increased risk of developing a disease relative to a member of the general population. In some embodiments a therapeutic agent may be administered to a subject who has had a disease but no longer shows evidence of the disease. The agent may be administered e.g., to reduce the likelihood of recurrence of evident disease. A therapeutic agent may be administered prophylactically, i.e., before development of any symptom or manifestation of a disease. “Prophylactic treatment” refers to providing medical and/or surgical management to a subject who has not developed a disease or does not show evidence of a disease in order, e.g., to reduce the likelihood that the disease will occur or to reduce the severity of the disease should it occur. The subject may have been identified as being at risk of developing the disease (e.g., at increased risk relative to the general population or as having a risk factor that increases the likelihood of developing the disease.

A “variant” of a particular polypeptide or polynucleotide has one or more alterations (e.g., additions, substitutions, and/or deletions) with respect to the polypeptide or polynucleotide, which may be referred to as the “original polypeptide” or “original polynucleotide”, respectively. An addition may be an insertion or may be at either terminus. A variant may be shorter or longer than the original polypeptide or polynucleotide. The term “variant” encompasses “fragments”. A “fragment” is a continuous portion of a polypeptide or polynucleotide that is shorter than the original polypeptide. In some embodiments a variant comprises or consists of a fragment. In some embodiments a fragment or variant is at least 20%, 30%, 40%, 50%, 60%, 70%, 80%, 90%, 95%, 96%, 97%, 98%, 99%, or more as long as the original polypeptide or polynucleotide. A fragment may be an N-terminal, C-terminal, or internal fragment. In some embodiments a variant polypeptide comprises or consists of at least one domain of an original polypeptide. In some embodiments a variant polynucleotide hybridizes to an original polynucleotide under stringent conditions, e.g., high stringency conditions, for sequences of the length of the original polypeptide. In some embodiments a variant polypeptide or polynucleotide comprises or consists of a polypeptide or polynucleotide that is at least 50%, 60%, 70%, 80%, 90%, 95%, 96%, 97%, 98%, 99%, or more identical in sequence to the original polypeptide or polynucleotide over at least 20%, 30%, 40%, 50%, 60%, 70%, 80%, 90%, 95%, 96%, 97%, 98%, 99%, or 100% of the original polypeptide or polynucleotide. In some embodiments a variant polypeptide comprises or consists of a polypeptide that is at least 50%, 60%, 70%, 80%, 90%, 95%, 96%, 97%, 98%, 99%, or more identical in sequence to the original polypeptide over at least 20%, 30%, 40%, 50%, 60%, 70%, 80%, 90%, 95%, 96%, 97%, 98%, 99%, or 100% of the original polypeptide, with the proviso that, for purposes of computing percent identity, a conservative amino acid substitution is considered identical to the amino acid it replaces. In some embodiments variant polypeptide comprises or consists of a polypeptide that is at least 50%, 60%, 70%, 80%, 90%, 95%, 96%, 97%, 98%, 99%, or more identical to the original polypeptide over at least 20%, 30%, 40%, 50%, 60%, 70%, 80%, 90%, 95%, 96%, 97%, 98%, 99%, or 100% of the original polypeptide, with the proviso that any one or more amino acid substitutions (up to the total number of such substitutions) may be restricted to conservative substitutions. In some embodiments a percent identity is measured over at least 100; 200; 300; 400; 500; 600; 700; 800; 900; 1,000; 1,200; 1,500; 2,000; 2,500; 3,000; 3,500; 4,000; 4,500; or 5,000 amino acids. In some embodiments the sequence of a variant polypeptide comprises or consists of a sequence that has N amino acid differences with respect to an original sequence, wherein N is any integer between 1 and 10 or between 1 and 20 or any integer up to 1%, 2%, 5%, or 10% of the number of amino acids in the original polypeptide, where an “amino acid difference” refers to a substitution, insertion, or deletion of an amino acid. In some embodiments a difference is a conservative substitution. Conservative substitutions may be made, e.g., on the basis of similarity in side chain size, polarity, charge, solubility, hydrophobicity, hydrophilicity and/or the amphipathic nature of the residues involved. In some embodiments, conservative substitutions may be made according to Table A, wherein amino acids in the same block in the second column and in the same line in the third column may be substituted for one another other in a conservative substitution. Certain conservative substitutions are substituting an amino acid in one row of the third column corresponding to a block in the second column with an amino acid from another row of the third column within the same block in the second column.

TABLE A

| | | |
|---|---|---|
| Aliphatic | Non-polar | G A P |
| | | I L V |
| | Polar—uncharged | C S T M |
| | | N Q |
| | Polar—charged | D E |
| | | K R |
| Aromatic | | H F W Y |

In some embodiments, proline (P) is considered to be in an individual group. In some embodiments, cysteine (C) is considered to be in an individual group. In some embodiments, proline (P) and cysteine (C) are each considered to be in an individual group. Within a particular group, certain substitutions may be of particular interest in certain embodiments, e.g., replacements of leucine by isoleucine (or vice versa), serine by threonine (or vice versa), or alanine by glycine (or vice versa).

In some embodiments a variant is a functional variant, i.e., the variant at least in part retains at least one activity of the original polypeptide or polynucleotide. In some embodiments a variant at least in part retains more than one or substantially all known biologically significant activities of the original polypeptide or polynucleotide. An activity may be, e.g., a catalytic activity, binding activity, ability to perform or participate in a biological function or process, etc. In some embodiments an activity of a variant may be at least 10%, 20%, 30%, 40%, 50%, 60%, 70%, 80%, 90%, 95%, or more, of the activity of the original polypeptide or polynucleotide, up to approximately 100%, approximately 125%, or approximately 150% of the activity of the original polypeptide or polynucleotide, in various embodiments. In some embodiments a variant, e.g., a functional variant, comprises or consists of a polypeptide at least 95%, 96%, 97%, 98%, 99%. 99.5% or 100% identical to an original polypeptide or polynucleotide over at least 70%, 75%, 80%, 85%, 90%, 95%, 96%, 97%, 98%, or 99% or 100% of the original polypeptide or polynucleotide. In some embodiments an alteration, e.g., a substitution or deletion, e.g., in a functional variant, does not alter or delete an amino acid or nucleotide that is known or predicted to be important for an activity, e.g., a known or predicted catalytic residue or residue involved in binding a substrate or cofactor. In some embodiments nucleotide(s), amino acid(s), or region(s) exhibiting lower degrees of conservation across species as compared with other amino acids or regions may be selected for alteration. Variants may be tested in one or more suitable assays to assess activity.

A “vector” may be any of a number of nucleic acid molecules or viruses or portions thereof that are ca-pable of mediating entry of, e.g., transferring, transporting, etc., a nucleic acid of interest between different genetic environments or into a cell. The nucleic acid of interest may be linked to, e.g., inserted into, the vector using, e.g., restriction and ligation. Vectors include, for example, DNA or RNA plasmids, cosmids, naturally occurring or modified viral genomes or portions thereof, nucleic acids that can be packaged into viral capsids, mini-chromosomes, artificial chromosomes, etc. Plasmid vectors typically include an origin of replication (e.g., for replication in prokaryotic cells). A plasmid may include part or all of a viral genome (e.g., a viral promoter, enhancer, processing or packaging signals, and/or sequences sufficient to give rise to a nucleic acid that can be integrated into the host cell genome and/or to give rise to infectious virus). Viruses or portions thereof that can be used to introduce nucleic acids into cells may be referred to as viral vectors. Viral vectors include, e.g., adenoviruses, adeno-associated viruses, retroviruses (e.g., lentiviruses), vaccinia virus and other poxviruses, herpesviruses (e.g., herpes simplex virus), and others. Viral vectors may or may not contain sufficient viral genetic information for production of infectious virus when introduced into host cells, i.e., viral vectors may be replication-competent or replication-defective. In some embodiments, e.g., where sufficient information for production of infectious virus is lacking, it may be supplied by a host cell or by another vector introduced into the cell, e.g., if production of virus is desired. In some embodiments such information is not supplied, e.g., if production of virus is not desired. A nucleic acid to be transferred may be incorporated into a naturally occurring or modified viral genome or a portion thereof or may be present within a viral capsid as a separate nucleic acid molecule. A vector may contain one or more nucleic acids encoding a marker suitable for identifying and/or selecting cells that have taken up the vector. Markers include, for example, various proteins that increase or decrease either resistance or sensitivity to antibiotics or other agents (e.g., a protein that confers resistance to an antibiotic such as puromycin, hygromycin or blasticidin), enzymes whose activities are detectable by assays known in the art (e.g., β-galactosidase or alkaline phosphatase), and proteins or RNAs that detectably affect the phenotype of cells that express them (e.g., fluorescent proteins). Vectors often include one or more appropriately positioned sites for restriction enzymes, which may be used to facilitate insertion into the vector of a nucleic acid, e.g., a nucleic acid to be expressed. An expression vector is a vector into which a desired nucleic acid has been inserted or may be inserted such that it is operably linked to regulatory elements (also termed “regulatory sequences”, “expression control elements” or “expression control sequences”) and may be expressed as an RNA transcript (e.g., an mRNA that can be translated into protein or a noncoding RNA such as an shRNA or miRNA precursor). Expression vectors include regulatory sequence(s), e.g., expression control sequences, sufficient to direct transcription of an operably linked nucleic acid under at least some conditions; other elements required or helpful for expression may be supplied by, e.g., the host cell or by an in vitro expression system. Such regulatory sequences typically include a promoter and may include enhancer sequences or upstream activator sequences. In some embodiments a vector may include sequences that encode a 5′ untranslated region and/or a 3′ untranslated region, which may comprise a cleavage and/or polyadenylation signal, and/or a vector may include a terminator. For example, a vector comprising an RNA pol III promoter may comprise an RNA pol III terminator sequence such as at least four-six consecutive T residues. In general, regulatory elements may be contained in a vector prior to insertion of a nucleic acid whose expression is desired or may be contained in an inserted nucleic acid or may be inserted into a vector following insertion of a nucleic acid whose expression is desired. As used herein, a nucleic acid and regulatory element(s) are said to be “operably linked” when they are covalently linked so as to place the expression or transcription of the nucleic acid under the influence or control of the regulatory element(s). For example, a promoter region would be operably linked to a nucleic acid if the promoter region were capable of effecting transcription of that nucleic acid. One of ordinary skill in the art will be aware that the precise nature of the regulatory sequences useful for gene expression may vary between species or cell types, but may in general include, as appropriate, sequences involved with the initiation of transcription, RNA processing, or initiation of translation. The choice and design of an appropriate vector and regulatory element(s) is within the ability and discretion of one of ordinary skill in the art. For example, one of skill in the art will select an appropriate promoter (or other expression control sequences) for expression in a desired species (e.g., a mammalian species) or cell type. A vector may contain a promoter capable of directing expression in mammalian cells, such as a suitable viral promoter, e.g., from a cytomegalovirus (CMV), retrovirus, simian virus (e.g., SV40), papilloma virus, herpes virus or other virus that infects mammalian cells, or a mammalian promoter from, e.g., a gene such as EF1alpha, ubiquitin (e.g., ubiquitin B or C), globin, actin, phosphoglycerate kinase (PGK), etc., or a composite promoter such as a CAG promoter (combination of the CMV early enhancer element and chicken beta-actin promoter). In some embodiments a human promoter may be used. In some embodiments, a promoter that ordinarily directs transcription by a eukaryotic RNA polymerase I (a “pol I promoter”), e.g., (a promoter for transcription of ribosomal RNA (other than 5S rRNA) or a functional variant thereof) may be used. In some embodiments, a promoter that ordinarily directs transcription by a eukaryotic RNA polymerase II (a “pol II promoter”) or a functional variant thereof is used. In some embodiments, a promoter that ordinarily directs transcription by a eukaryotic RNA polymerase III (a “pol III promoter”), e.g., a promoter for transcription of U6, H1, 7SK or tRNA or a functional variant thereof is used. One of ordinary skill in the art will select an appropriate promoter for directing transcription of a sequence of interest. Examples of expression vectors that may be used in mammalian cells include, e.g., the pcDNA vector series, pSV2 vector series, pCMV vector series, pRSV vector series, pEF1 vector series, Gateway® vectors, etc. Examples of virus vectors that may be used in mammalian cells include, e.g., adenoviruses, adeno-associated viruses, poxviruses such as vaccinia viruses and attenuated poxviruses, retroviruses (e.g., lentiviruses), Semliki Forest virus, Sindbis virus, etc. In some embodiments, regulatable (e.g., inducible or repressible) expression control element(s), e.g., a regulatable promoter, is/are used so that expression can be regulated, e.g., turned on or increased or turned off or decreased. For example, the tetracycline-regulatable gene expression system (Gossen & Bujard, Proc. Natl. Acad. Sci. 89:5547-5551, 1992) or variants thereof (see, e.g., Allen, N, et al. (2000) Mouse Genetics and Transgenics: 259-263; Urlinger, S, et al. (2000). Proc. Natl. Acad. Sci. U.S.A. 97 (14): 7963-8; Zhou, X., et al (2006). Gene Ther. 13 (19): 1382-1390 for examples) can be employed to provide inducible or repressible expression. Other inducible/repressible systems may be used in various embodiments. For example, expression control elements that can be regulated by small molecules such as artificial or naturally occurring hormone receptor ligands (e.g., steroid receptor ligands such as naturally occurring or synthetic estrogen receptor or glucocorticoid receptor ligands), tetracycline or analogs thereof, metal-regulated systems (e.g., metallothionein promoter) may be used in certain embodiments. In some embodiments, tissue-specific or cell type specific regulatory element(s) may be used, e.g., in order to direct expression in one or more selected tissues or cell types. A tissue-specific or cell type specific regulatory element generally directs expression at a higher level in one or more tissues or cell cell types than in many or most other tissues or cell types (e.g., other cell types in the body or in an artificial environment). In some cases a cell type specific regulatory element directs detectable levels of expression only in a particular cell type of interest. However, useful cell type regulatory elements may not be and often are not absolutely specific for a particular cell type. In some embodiments a cell type specific regulatory element may direct expression of an operably linked nucleic acid at a level at least 2-, 5-, 10, 25, 50, or 100-fold greater in a particular cell type than the level at which it would direct expression of the same nucleic acid in a reference population of cells. One of ordinary skill in the art will be aware of tissue and cell type specific regulatory elements and will be able to select an appropriate element to achieve a useful level of expression in one or more selected tissues or cell types in which expression is desired while avoiding substantial levels of expression that might otherwise occur in tissues or cell types in which expression is not desired. In some embodiments a vector may comprise a polynucleotide sequence that encodes a polypeptide, wherein the polynucleotide sequence is positioned in frame with a nucleic acid inserted into the vector so that an N- or C-terminal fusion is created. In some embodiments the polypeptide encoded by the polynucleotide sequence may be a targeting peptide. A targeting peptide may comprise a signal sequence (which directs secretion of a protein) or a sequence that directs the expressed protein to a specific organelle or location in the cell such as the nucleus or mitochondria. In some embodiments the polypeptide comprises a tag. A tag may be useful to facilitate detection and/or purification of a protein that contains it. Examples of tags include polyhistidine-tag (e.g., 6X-His tag), glutathione-5-transferase, maltose binding protein, NUS tag, SNUT tag, Strep tag, epitope tags such as V5, HA, Myc, or FLAG. In some embodiments a protease cleavage site is located in the region between the protein encoded by the inserted nucleic acid and the polypeptide, allowing the polypeptide to be removed by exposure to the protease.

### II. Overview

In some aspects, the invention provides methods of identifying a gene that affects mitochondrial phenotype or function. In some embodiments, such genes are targets for modulation in order to modulate mitochondrial phenotype or function. In some embodiments, such genes are targets for development of candidate agents to affect mitochondrial phenotype or function for, e.g., research or therapeutic purposes. In some embodiments, a gene identified using a method described herein has a role in performing or regulating at least one mitochondrial function. In some embodiments, a gene identified using a method described herein is a candidate gene for involvement in a mitochondrial disorder. In some embodiments, a gene identified as described herein is a potential drug target for treatment of a mitochondrial disorder, e.g., the gene is a candidate target for development of therapeutic agents to treat a mitochondrial disorder.

Among other things, the present disclosure encompasses the recognition that a screening platform utilizing near-haploid mammalian cells affords a powerful approach to identifying mammalian genes that affect mitochondrial phenotype or function. In some embodiments near-haploid mammalian cells are used to identify nuclear genes whose inhibition affects one or more mitochondrial phenotypes or functions. In some embodiments a mitochondrial phenotype is characteristic of a mitochondrial disorder. In some embodiments a mitochondrial function is one that, when abnormal, can result in a mitochondrial disorder.

The disclosure further provides the insight that mitochondrial poisons can be used to model mitochondrial disorders affecting mammalian subjects. In some embodiments, mammalian cells are contacted with a mitochondrial poison and used to identify genes whose modulation, e.g., inhibition, results in altered sensitivity to, e.g., resistance to, the mitochondrial poison. In some embodiments, modulation, e.g., inhibition, of such a gene protects a cell or subject against mitochondrial dysfunction associated with a mitochondrial disorder. In some embodiments modulation of such a gene improves mitochondrial function. In some embodiments, modulation, e.g., inhibition, of such a gene inhibits mitochondrial dysfunction associated with a mitochondrial disorder, e.g., improves mitochondrial function or counteracts or reduces the effect of mitochondrial dysfunction on a cell. In some embodiments, modulation of such a gene protects a cell against mitochondrial dysfunction that would otherwise kill the cell. In some embodiments, modulation of such a gene is of use to treat a subject in need of treatment for a mitochondrial disorder. Identification of genes whose modulation results in altered sensitivity (e.g., resistance) to a mitochondrial poison can be performed in a variety of ways, as discussed further below. In some embodiments of particular interest, a screening platform utilizing near-haploid mammalian cells is used.

It will be understood that where a gene is referred to as a “target” or “candidate target” herein, the gene products of the gene are also considered targets/candidate targets. A physical target to be modulated will typically be a gene product, e.g., an RNA or protein. Reference to a gene herein (e.g., as a candidate target) constitutes reference to the gene products of that gene, and vice versa.

### III. Haploid Genetic Screens to Identify Genes Affecting Mitochondrial Phenotype or Function

In some aspects the disclosure relates to genetic screens that use near-haploid mammalian cells for identifying genes that affect a mitochondrial phenotype. In some embodiments, a plurality of near-haploid mammalian cells is mutagenized. The cells are maintained in culture and assessed for presence of a mitochondrial phenotype of interest. Cells that exhibit the phenotype or are identified and/or isolated. In some embodiments one or more genes that are mutated in the cells are identified. Identified genes are candidate genes for affecting the mitochondrial phenotype. In some embodiments, a method of identifying a gene that affects mitochondrial phenotype comprises: (a) providing a plurality of mutagenized near-haploid mammalian cells; (b) isolating a cell that exhibits a mitochondrial phenotype of interest; and (c) identifying a gene that is mutated in the cell, thereby identifying a gene that affects mitochondrial phenotype. In some embodiments the role of an identified gene in) affecting the mitochondrial phenotype is confirmed. Confirmation may be obtained by, e.g., re-introducing a functional copy of the gene into a mutant cell and detecting an alteration in the mitochondrial phenotype, e.g., a restoration of a phenotype exhibited by unmutagenized cells or by inactivating the gene in an unmutagenized cell not exhibiting the phenotype and detecting the occurrence phenotype in such cells.

“Mitochondrial phenotype” encompasses any detectable structural or functional characteristic of mitochondria that can be detected in a cell or cell population. Mitochondria contain their own DNA (mtDNA) and machinery for synthesizing RNA and proteins. Human mtDNA has only 37 genes, of which most code for transfer RNAs. The great majority of gene products in mammalian mitochondria are encoded by nuclear DNA. Nuclear genes play a role in almost every mitochondrial phenotype and function. Most mitochondrial activities are carried out at least in part by proteins transported into the organelle from the cytoplasm. In some embodiments, a method described herein comprises identifying a nuclear gene that affects mitochondrial phenotype.

Near-haploid genetic screens can, in various embodiments, be used to identify genes or loci that affect any mitochondrial phenotype that can be detected in mutant cells, e.g., mutant cells generated using insertional mutagenesis (discussed further below). In some embodiments a mitochondrial phenotype is associated with a mitochondrial disorder. In some embodiments, a gene that affects a mitochondrial phenotype is a candidate target for drug development for mitochondrial disorders.

In almost all mammals, including humans, most somatic cells are normally diploid, i.e., their nucleus contains two homologous copies of each chromosome (other than the two sex chromosomes, which can be homologous or non-homologous depending on the sex and species). For example, human cells normally contain 23 pairs of chromosomes, i.e., 46 chromosomes in total. The members of a homologous pair are non-identical chromosomes that both contain the same genes at the same loci but possibly have different alleles of those genes. As used herein, a “near-haploid” mammalian cell refers to a mammalian cell in which no more than 5 chromosomes are present in two or more copies. In some embodiments a near-haploid mammalian cell has no more than 1, 2, 3, or 4 chromosomes present in two or more copies, A “fully haploid” cell contains no more than one copy of each chromosome. As used herein, the terms “near-haploid” and “haploid” are used interchangeably and encompass fully haploid cells, which contain no more than one copy of each chromosome, and cells that have two or more copies of 1, 2, 3, 4, or 5 chromosomes. Thus where the present disclosure refers to a “near-haploid” cell or cells, embodiments in which the cell or cells are fully haploid or not fully haploid are encompassed. In some embodiments a near-haploid mammalian cell is diploid for one chromosome, i.e., two copies of the chromosome are present. For example, in some embodiments, a near-haploid cell line has two copies of chromosome 8. One of ordinary skill in the art will appreciate that some cells harbor chromosomal translocations or fusions, in which portions of two chromosomes are exchanged or a portion of one chromosome is fused to another chromosome. Translocations or fusions can be recognized by a number of techniques, e.g., by detecting alterations in banding pattern or by fluorescence in situ hybridization. For purposes herein, if at least half of the genetic information present on a normal chromosome, as assessed using FISH or by examining banding pattern, remains present within a cell, the chromosome is considered to be present.

In some embodiments a near-haploid mammalian cell is a human cell. In some embodiments a near-haploid mammalian cell is a non-human mammalian cell, e.g., a non-human primate cell or a rodent cell, e.g., a mouse, rat, or rabbit cell. In some embodiments a near-haploid mammalian cell is a hematopoietic lineage cell, e.g., a lymphoid or myeloid cell. In some embodiments a near-haploid mammalian cell is a tumor cell, e.g., a descendant of a cell that was originally obtained from a tumor. The tumor may be benign or malignant. In some embodiments a tumor is a carcinoma, sarcoma, or hematologic malignancy, e.g., a leukemia (such as chronic or acute myelogenous leukemia, chronic or acute lymphocytic leukemia) or a lymphoma or a myeloma.

In some embodiments a near-haploid mammalian cell line is isolated, e.g., subcloned, from a population of cells comprising at least some near-haploid cells. For example, subclones can be generated from individual cells and screened, e.g., using flow cytometry, to identify one or more subclones that have a near-haploid karyotype. In some embodiments a near-haploid cell line remains karyotypically stable for many weeks in culture (e.g., at least 8, 10, 12, 16, 20, 24, or more weeks). In some embodiments, near-haploid subclones can be repeatedly isolated from a near-haploid cell line, thus permitting continuous maintenance of near-haploid cells in culture. In some embodiments a karyotype (e.g., the number and appearance of chromosomes) of a cell or cell line may be assessed using various techniques known in the art such as “banding” with different stains (e.g., G-banding), spectral karyotyping (a molecular cytogenetic technique used to simultaneously visualize all the pairs of chromosomes in an organism in different colors in which fluorescently labeled probes for each chromosome used to label chromosome-specific DNA with different fluorophores). In some embodiments cells may be tested by any of a variety of methods known in the art such as DNA fingerprinting (e.g., short tandem repeat (STR) analysis) or single nucleotide polymorphism (SNP) analysis (which may be performed using, e.g., SNP arrays (e.g., SNP chips) or sequencing), e.g., in order to determine or confirm whether they are derived from a single individual or a particular cell line.

In some embodiments a near-haploid mammalian cell is a chronic myelogenous leukemia (CML) cell, e.g., a KBM7 cell. A subclone of the CML cell line KBM7 was described to carry a near-haploid chromosome set (Kotecki, M., et al. (1999). Isolation and characterization of a near-haploid human cell line. Exp Cell Res 252, 273-280). A subclone of this line was confirmed to be haploid for all chromosomes except chromosome 8 and to contain a Philadelphia chromosome (t(9;22)) (see PCT/US2010/041628 and ref. 5). The term “KBM7 cell line” encompasses near-haploid cell lines isolated from the original KBM7 cell line and subclones therefrom. As will be appreciated, KBM7 subclones can be further subcloned to give rise to additional KBM7 subclones. Similarly, other near-haploid cell lines can be further subcloned.

In some embodiments a near-haploid mammalian cell is a leiomyosarcoma cell (Dal Sin, P., et al., J Pathol., 185(1):112-5, 1988). In some embodiments a near-haploid mammalian cell is a malignant fibrous histiocytomas (MFH) cell (Aspberg F, et al., Cancer Genet Cytogenet. 1995; 79(2):119-22.). In some embodiments a near-haploid mammalian cell is a breast cancer cell (Flagiello D, Cancer Genet Cytogenet. 1998; 102(1):54-8). In some embodiments a near-haploid mammalian cell is a mesothelioma cell or a malignant peripheral nerve sheath tumor cell. Sukov W R, et al., Cancer Genet Cytogenet. 2010; 202(2):123-8 describes certain near-haploid cells of use in certain embodiments.

In some embodiments a near-haploid mammalian cell is an embryonic stem (ES) cell, e.g., a rodent ES cell or a primate ES cell, e.g., a human ES cell. In some embodiments haploid ES cells, e.g., fully haploid ES cells, may be generated by activation of unfertilized oocytes, isolation of blastocysts, derivation of ES cells, expansion of ESC lines, and analysis of DNA content to identify cells having haploid DNA content. Further enrichment may be obtained using cell sorting, e.g., following staining with a dye that stains DNA, such as Hoechst or DAPI. Certain haploid mouse ES cells are described Leeb, M. and Wutz, A., Nature. 2011; 479(7371):131-4.

In some embodiments a near-haploid mammalian cell is at least in part reprogrammed, using one or more reprogramming agents used in the art for generating induced pluripotent stem cells (iPS cells). Reprogramming of near-haploid mammalian cells is described in WO/2011/006145. In some embodiments reprogramming comprises expressing in a cell one or more transcription factors, e.g., one or more of the transcription factors Oct4, Sox2, Klf4, and c-Myc or one or more of the transcription factors Oct4, Nanog, Sox2, and Lin28 (see, e.g., Meissner, A., et al, Nat. Biotechnol., 25(10):1 177-81 (2007); Yu, J., et al, Science, 318(5858): 1917-20 (2007); and Nakagawa, M., et al., Nat. Biotechnol., 26(1): 101-6 (2008). In some embodiments one or more small molecules are used for reprogramming. A variety of small molecules are known in the art that can replace one or more transcription factors for purposes of reprogramming. In some embodiments reprogramming of a non-adherent near-haploid mammalian cell results in an adherent cell (see PCT/US2010/041628, which describes reprogramming non-adherent KBM7 cells to produce adherent cells).

In some embodiments a cell, e.g., a near-haploid mammalian cell, is genetically modified. For example, in some embodiments a cell is genetically modified to comprise a gene that encodes a reporter or sensor. In some embodiments the reporter or sensor is of use to identify a cell that has a mitochondrial phenotype of interest or to quantify a mitochondrial phenotype or to assess the effect of a compound on a mitochondrial phenotype. In some embodiments a gene encoding the reporter or sensor is stably integrated into the genome. In some embodiments the cell is genetically modified prior to being mutagenized. In some embodiments, a reporter or sensor comprises a mitochondrial targeting sequence (MTS) so that the reporter or sensor localizes to mitochondria (at least if the mitochondrial import system is functioning normally). In some embodiments a MTS can comprise any sequence that directs mitochondrial localization of a naturally occurring mitochondrial protein, or a functional variant thereof. (MTSs are discussed further below.) Detection of a reporter may be used to assess, e.g., mitochondrial number or morphology. A sensor may, for example, report on pH, analytes such as ions (e.g., Ca²) or small molecules (e.g., ATP), redox status, etc. A variety of genetically encoded sensors are known (see, e.g., Palmer, A E, et al., Trends Biotechnol. 2011; 29(3):144-52, and references therein). In some embodiments a first reporter molecule is used for purposes of identifying a gene that affects a particular phenotype of interest and a second, different reporter molecule is used for purposes of identifying cells that have a mutagenic nucleic acid construct inserted into their genome.

Mammalian cells typically contain between one and several thousand mitochondria. The average number of mitochondria per cell varies among different tissue types. In some embodiments a mitochondrial phenotype comprises the number, average size, size distribution, or shape of a cell's mitochondria or of a mitochondrial substructure. In some embodiments a mitochondrial phenotype may be described in terms of a deviation from a control phenotype. In some embodiments a mitochondrial phenotype comprises an alteration in at least one mitochondrial function or property as compared with suitable control cells. For example, in some embodiments a mitochondrial phenotype comprises an alteration in number, average size, size distribution, or shape of a cell's mitochondria or of a mitochondrial substructure, as compared with suitable control cells. In some embodiments, a mitochondrial phenotype comprises a loss or reduction of a property or function found in mitochondria of control cells. In some embodiments suitable control cells are nonmutagenized cells of the same cell line. In some embodiments control cells are maintained under the same or substantially the same conditions (other than exposure to the mutagenizing agent) as the cells used in the screen.

Mitochondria play important roles in metabolism of amino acids, fatty acids, and steroids, and calcium signaling. In some embodiments a mitochondrial phenotype comprises an alteration in amino acid metabolism, fatty acid or steroid metabolism, or calcium signaling.

In some embodiments a mitochondrial phenotype comprises transport, or altered transport, of one or more substances that is ordinarily transported into or out of mitochondria or across the outer or inner mitochondrial membrane, e.g., an ion (such as Ca²⁺) or a small molecule such as a metabolite or substrate for the TCA, to name but a few.

In some embodiments a mitochondrial phenotype comprises a concentration, or altered concentration, of one or more substances that is ordinarily present in mitochondria or in a particular mitochondrial compartment, e.g., an ion or small molecule such as a metabolite or substrate for the TCA, to name but a few.

In some embodiments a mitochondrial phenotype comprises release, or altered release, of one or more substances that is released from mitochondria during apoptosis, e.g., cytochrome c.

In some embodiments a mitochondrial phenotype comprises expression or altered expression or localization, as compared with control cells, of one or more mitochondrial proteins or protein complexes.

In some embodiments a mitochondrial phenotype comprises production of, or altered production of, a substance that is, under at least some conditions, produced in mitochondria. For example, mitochondria produce most of the ATP used by mammalian cells and have roles in synthesizing a variety of other substances such as heme and certain steroids. In some embodiments the substance is harmful to mammalian cells under at least some conditions. For example, reactive oxygen species (ROS) produced in mitochondria can cause cellular damage, and excessive amounts of ROS are believed to play a role in a number of mitochondrial disorders. In some embodiments a mitochondrial phenotype comprises catabolism of, or altered catabolism of, a molecule or complex that is, under at least some conditions, catabolized at least in part in mitochondria.

Mitochondria are responsible for producing most of the ATP used by eukaryotic cells as a source of chemical energy. Fuels such as carbohydrates and fats are transported across the inner mitochondrial membrane into the matrix, broken down, and further metabolized in the tricarboxylic acid (TCA) cycle, during which NAD+ and FAD are reduced to NADH and FADH2. Synthesis of ATP occurs via a two stage process. High energy electrons from FADH2 and NADH (from the TCA cycle or glycolysis) are shuttled through a series of protein complexes in the inner mitochondrial membrane to molecular oxygen. The loss of electrons from NADH and FADH2 regenerates the NAD+ and FADH needed for the process to continue. During the electron transport process, protons are pumped out of the mitochondrial matrix to the intermembrane space, resulting in an electrochemical gradient that includes contributions from both a membrane potential (Δψₘ) and a pH difference. The energy released when protons flow back into the matrix across the inner membrane is used by the protein complex termed ATP synthase to synthesize ATP from ADP and inorganic phosphate (Pᵢ). The electrochemical proton gradient drives a variety of other processes in addition to ATP synthesis, such as transport of charged small molecules.

The overall process of electron transport and ATP synthesis is referred to as “oxidative phosphorylation” (OXPHOS), and the components responsible for performing these processes are referred to as the “OXPHOS system”. The components involved in OXPHOS (“OXPHOS components”) include 5 multi-subunit protein complexes (referred to as complexes I, II, III, IV, and V), a small molecule (ubiquinone, also called coenzyme Q), and the protein cytochrome c (Cyt c). The set of proteins and small molecules involved in electron transport is referred to as the “electron transport chain” (ETC) or “respiratory chain”. Protons are pumped across the inner mitochondrial membrane (i.e., from the matrix to the intermembrane space) by complexes I, III, and IV. Ubiquinone, and cytochrome c function as electron carriers. Electrons from the oxidation of succinate to fumarate are channeled through this complex to ubiquinone. Complex V is ATP synthase (EC 3.6.3.14), which is composed of a head portion, called the F1 ATP synthase (or F1), and a transmembrane proton carrier, called F0. Both F1 and F0 are composed of multiple subunits. ATP synthase can function in reverse mode in which it hydrolyzes ATP. The energy of ATP hydrolysis can be used to pump protons across the inner mitochondrial membrane into the matrix. ATP synthase is also referred to as F0-F1 ATP synthase or F0-F1 ATPase. These terms are used interchangeably herein.

In some embodiments a mitochondrial phenotype relates to the OXPHOS sysem. In some embodiments a mitochondrial phenotype comprises an alteration in at least one function of the oxidative phosphorylation system, e.g., electron transport and/or mitochondrial adenosine triphosphate (ATP) production. In some embodiments a mitochondrial phenotype comprises an alteration in amount or activity of at least one component of the OXPHOS system, e.g., complex I, II, III, IV, or V or any of their component proteins.

In some embodiments a mitochondrial phenotype comprises pH, or an alteration in pH, of the matrix or intermembrane space.

In some embodiments, a mitochondrial phenotype comprises an alteration in mitochondrial fission, fusion, membrane remodeling, or motility.

Mammalian mitochondria typically harbor multiple mtDNA molecules, which are replicated and normally partitioned to daughter cells when a cell divides. In some embodiments a mitochondrial phenotype comprises a defect in mtDNA replication or mtDNA repair, an alteration in mtDNA copy number, or a defect in partitioning mtDNA molecules during mitochondrial fission.

In some embodiments a mitochondrial phenotype is inner mitochondrial membrane potential (Δψₘ)) or inner mitochondrial membrane permeability. Unless otherwise indicated the term “mitochondrial membrane” and “inner mitochondrial membrane” are used interchangeably herein.

In some embodiments a mitochondrial phenotype is oxygen consumption or respiratory capacity.

In some embodiments a mitochondrial phenotype comprises a response to an agent or condition. In some embodiments a mitochondrial phenotype comprises an alteration in a response to an agent or condition, as compared with the response of control cells.

In some embodiments a mitochondrial phenotype comprises opening or altered permeability or activity of a mitochondrial channel or transporter. In some embodiments a mitochondrial channel is the mitochondrial permeability transition pore (mPTP). mPTP opening can lead to matrix swelling, subsequent rupture of the outer membrane, and nonspecific release of proteins from the intermembrane space into the cytosol and is commonly associated with cell death.

In some embodiments a screen is performed using near-haploid mammalian cells that have been mutagenized by insertional mutagenesis. In some embodiments insertional mutagenesis is accomplished by introducing a gene trap vector into near-haploid mammalian cells. The term “gene trap vector” refers to a vector that comprises a nucleic acid construct capable of inserting into and potentially inactivating an endogenous cellular gene, e.g., a gene in the nucleus of a mammalian cell. Typically, insertion of the nucleic acid construct into the gene both disrupts the gene and facilitates its identification and/or isolation. A cell having such an insertion is considered a “mutant cell”. The inserted nucleic acid serves as a “molecular tag” that can be used to isolate or otherwise identify endogenous genomic DNA located nearby, as discussed further below. In some embodiments the nucleic acid construct comprises DNA that encodes a reporter molecule (“reporter”) that, when expressed, allows identification of a cell that contains the construct inserted into its genome. Such DNA may be referred to as a “reporter gene”. In some embodiments the reporter molecule facilitates detection and/or isolation of a cell that contains the construct. In some embodiments the construct lacks a genetic element, such as a promoter or a polyadenylation (polyA) sequence, which element is normally required for expression or that significantly increases expression, so that effective expression of the reporter following introduction of the vector into a cell occurs only if the construct inserts into an endogenous gene. Examples of reporters are discussed herein. For example, in some embodiments a readily detectable protein, such as a fluorescent protein or enzyme, may be used. In some embodiments a selectable marker is used. In some embodiments activity of a reporter is used to identify a cell having a gene trap construct insertion in an endogenous gene.

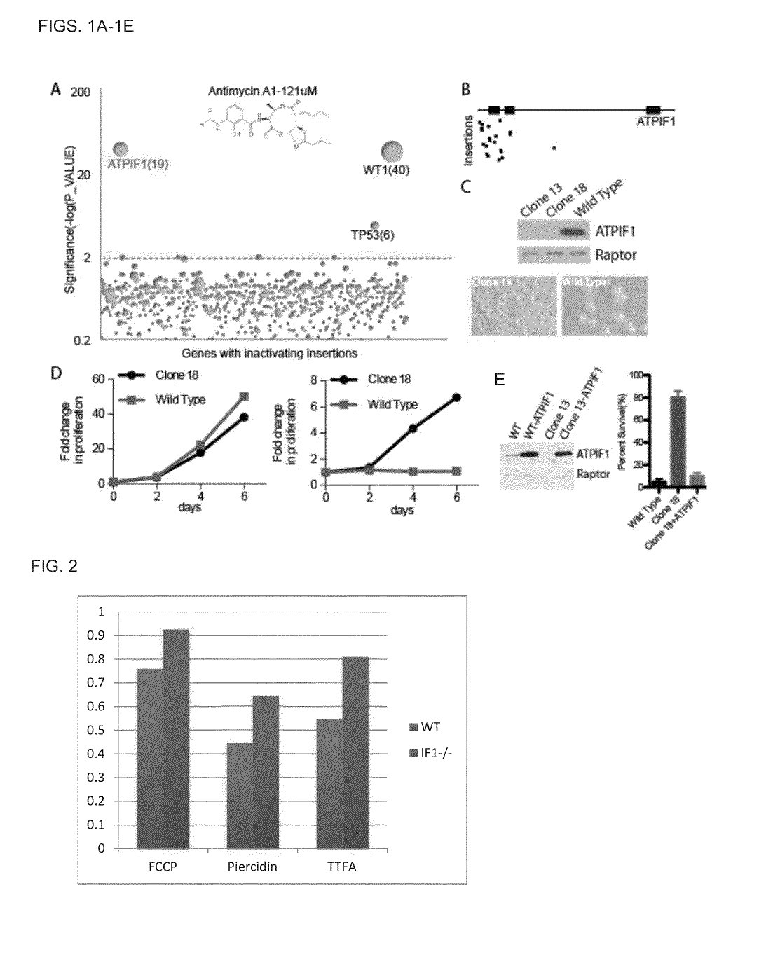

Gene trap vectors of a variety of different designs may be used in various embodiments. Various gene trap vectors are described in, e.g., PCT/US2010/041628 (WO/2011/006145) and references 5, 8, and 9. In some embodiments a gene trap vector comprises a nucleic acid construct comprising a promoterless reporter gene flanked by an upstream splice acceptor (SA) site and a downstream polyadenylation sequence. In other words, the promoterless reporter gene is positioned downstream from a splice acceptor site and upstream from a polyA sequence (also referred to as a “polyA site” or “polyA signal”). FIG. 1 shows an example of a promoterless gene trap construct in schematic form, wherein the reporter gene encodes green fluorescent protein (GFP). When inserted into an intron of an expressed gene, the gene trap construct is transcribed from the endogenous promoter of that gene in the form of a fusion transcript in which the exon(s) upstream of the insertion site is spliced in frame to the reporter gene. Such a gene trap vector may be referred to as a “promoter trap” gene trap vector. Transcription terminates prematurely at the inserted polyA site, so that the resulting fusion transcript encodes a truncated and non-functional version of the cellular protein fused to the reporter. The reporter allows identification of cells in which the gene trap vector has inserted into an actively transcribed locus. Such gene trap vectors both inactivate and report the expression of the trapped gene at the insertion site and provide a nucleic acid tag that permits rapid identification of the disrupted gene. In some embodiments a gene trap vector does not encode a reporter but instead encodes a different protein. In some embodiments a gene trap vector does not encode a protein but simply causes premature termination at a polyA site inserted into an endogenous gene.

A variety of splice acceptor sites can be used in a gene trap vector in various embodiments. In some embodiments a SA site is an adenoviral SA site. In some embodiments a SA from the long fiber gene of adenovirus type 40 is used (Carette et al. 2005 The Journal of Gene Medicine 7(8) 1053-1062). Other strong adenoviral SA sites are those derived from the fiber or hexon gene of different adenoviral serotypes. A variety of polyA sequences can be used in the gene trap vector. In some embodiments a polyA sequence is a bovine growth hormone polyA sequence.

In some embodiments a gene trap vector is a polyA trap vector. A polyA trap vector comprises a nucleic acid construct comprising (i) a reporter gene comprising a nucleic acid sequence that encodes a reporter, operably linked to a promoter; and (ii) a splice donor (SD) site located downstream of the reporter gene. The gene trap vector lacks a polyA sequence, so that efficient synthesis of the reporter can only occur if the vector inserts in an intron and a polyA site is provided by splicing to downstream exons. When inserted into an intron of an endogenous gene, the transcript expressed from the gene trap promoter is spliced to the downstream exons of the endogenous gene, the most 3′ of which comprises a polyA sequence, resulting in a fusion transcript that terminates with the polyA sequence of the endogenous gene. Since the fusion transcript is expressed from the inserted promoter, polyA trap vectors trap genes independently of whether the endogenous gene is expressed. The reporter allows identification of cells in which the gene trap vector has inserted into an intron, and the inserted DNA can be used to identify genomic sequences close to the insertion site. In some embodiments of the invention the SD site in an adenoviral SD site. In some embodiments, a polyA trap vector further comprises an IRES sequence downstream of the termination codon of the reporter gene and upstream of the splice donor site. This approach can be useful to overcome nonsense-mediated decay that might otherwise occur, e.g., if the termination codon of the reporter gene is e.g., more than about 55 nucleotides upstream of the final splice junction site.

A variety of different promoters can be used, e.g., in a gene trap vector that comprises a promoter. A promoter capable of directing expression in a near-haploid mammalian cell in which the gene trap vector is used can be selected by one of ordinary skill in the art. In some embodiments a promoter is an RNA polymerase II promoter (i.e., a promoter that directs transcription by RNA polymerase II). In some embodiments a promoter is a constitutive promoter. In some embodiments a promoter is a strong promoter active in a wide range of mammalian cell types, such as the CMV immediate-early promoter or major intermediate-early promoter, other mammalian viral promoters such as the herpes simplex virus (HSV) promoter, SV40 or other polyoma virus promoters, or adenovirus promoters. In some embodiments a promoter is a mammalian promoter, such as the elongation factor-1alpha (EF1alpha), phosphoglycerate kinase-1 (PGK), histone, or hTERT promoter. In some embodiments a promoter is active in one or more cell types or cell lineages of interest and is not active, or is substantially less active, in many or most other cell types or lineages. For example, if a near-haploid mammalian cell is a hematopoietic cell, a promoter active in hematopoietic lineage cells may be used. In some embodiments a promoter is regulatable, e.g., inducible or repressible. Examples of regulatable promoters include heat shock promoters, metallothionein promoter, and promoters that comprise an element responsive to a small molecule such as tetracycline or a related compound (e.g., doxycycline), or a hormone. For example, in some embodiments an inducible promoter comprises a hormone response element that renders the promoter responsive to a ligand for a hormone receptor. Hormone receptors include, e.g., the estrogen, progesterone, and glucocorticoid receptors. Ligands include physiological ligands, e.g., estrogen, progesterone, or cortisol, and non-physiological ligands, e.g., tamoxifen, dexamethasone. It will be understood that in various embodiments the cell expresses or is modified to express or contain appropriate trans-acting proteins typically comprising a DNA binding domain, activation or repression domain, and ligand-binding domain, that render the promoter responsive to a ligand.

In some embodiments a gene trap vector comprises first and second nucleic acid constructs that contain first and second reporter genes, respectively. The reporter genes are typically different. The first nucleic acid construct comprises a reporter gene operably linked to a promoter active in a near-haploid mammalian cell of interest. The other nucleic acid construct comprises a promoterless gene trap construct or a polyA trap construct such as those described above. A reporter encoded by the first reporter gene is used to identify cells in which the gene trap vector has integrated into the genome. A reporter encoded by the second reporter gene is used to identify cells in which such integration occurs in an endogenous gene. In some embodiments a first reporter gene encodes a selectable marker and a second reporter gene encodes a detectable marker.

In some embodiments a gene trap vector comprising a transposon is used to perform insertional mutagenesis in near-haploid mammalian cells. In some embodiments a transposon is a piggyBac transposon. A piggyBac transposon system is described in Wang, W., et al., Genome Res. 2009 19: 667-673. It contains splice acceptor (SA) Beta-geo or SA-T2A-Beta-gal-T2A-Neo gene-trap cassette flanked by the 59 and 39 PB terminal DNA repeats (59 PBTR and 39 PBTR).

In some embodiments insertion of a gene-trap vector is reversible, i.e., the inserted nucleic acid construct can be readily excised and the insertion site repaired. For example, in some embodiments recognition sites for a site-specific recombinase such as LoxP or FRT are inserted at the both ends of the gene-trap cassette, so that the integrated vectors can excised with the corresponding recombinase (e.g., Cre or Flp). In embodiments in which a transposon is used, the corresponding transposase may be used to reverse the insertion. In some embodiments a precise excision of the inserted gene trap construct occurs while in some embodiments a small amount of heterologous DNA remains after excision (e.g., a LoxP site). In some embodiments, reversal of the phenotype of a mutant cell upon excision of the gene trap construct confirms that the insertion was responsible for the phenotype. Thus in some embodiments excision of the gene trap construct is used to confirm that the gene is one whose modulation affects the mitochondrial phenotype, e.g., that the gene is one whose inhibition results in the particular mitochondrial phenotype observed.

Gene trap constructs may be made using standard methods of recombinant DNA technology and genetic engineering and can be introduced into cells using various types of vectors. In certain embodiments a gene trap vector is a viral vector, e.g., a retroviral (e.g., lentiviral), adenoviral, or herpes viral vector that comprises the gene trap construct, e.g., as part of its genome. The viral vector can be a virus (viral particle), which is used to infect cells, thereby introducing the gene trap construct. Following infection, at least a portion of the viral genome or a copy thereof integrates into the cellular genome, typically at random sites within the cell's DNA. In certain embodiments a retroviral vector is employed to deliver the gene trap construct to a near-haploid mammalian cell. Retroviral vectors and methods of using retroviruses to introduce exogenous DNA into mammalian cells are well known in the art. A retroviral vector typically comprises LTRs, which can be derived from various types of retroviruses. LTR(s) may be genetically modified to provide desired properties, and the viral genome can be modified, e.g., to lack promoter activities and/or to comprise regulatory elements suitable for propagation and selection in bacteria, such as an origin of replication and an antibiotic resistance marker. The gene trap construct is positioned between the LTRs. Infectious, replication-competent retroviral gene-trap particles can be produced by transfecting a retroviral plasmid comprising the gene trap construct into a retrovirus packaging cell line using standard methods. The packaging cells are cultured, and viral particles released into the media are collected (e.g., as supernatants) for subsequent use, e.g., to infect mammalian hear-haploid cells. In some embodiments a gene trap vector is a plasmid. In some embodiments, a plasmid gene trap vector is linearized prior to introducing it into cells.

In some embodiments, near-haploid mammalian cells are contacted with a gene trap vector under conditions suitable for uptake of the vector and insertion of the construct into the genome. A wide variety of methods can be used to introduce a gene trap vector into near-haploid mammalian cells. Examples include viral infection (e.g., retroviral infection), transfection (e.g., using calcium-phosphate or lipid-based transfection reagents), electroporation, microinjection, etc. One of skill in the art can select an appropriate method based, e.g., on the nature of the vector and cell. It will be appreciated that, typically, not all cells contacted with a gene trap vector will take up the vector, and stable insertion of the construct into the genome may not occur in all cells that take up the vector. In some embodiments insertional mutagenesis is performed such that the average number of insertions per cell is between 0.1 and 2, e.g., between 0.5 and 1. The average number of insertions can be controlled, for example, by using an appropriate ratio of cells to vectors.

The number of cells used can vary. In some embodiments of any aspect or embodiment herein referring to a plurality of cells, population of cells, cell sample, or similar terms, the number of cells is between 10⁴ and 10¹³ cells. In some embodiments the number of cells may be at least about 10⁵, 10⁶, 10⁷, 10⁸, 10⁹, 10¹⁰, 10¹¹, 10¹² cells, or more. In some embodiments, the number of cells mutagenized or screened is between 10⁵ and 10¹² cells, e.g., at least 10⁶, 10⁷, 10⁸, 10⁹, 10¹⁰, 10¹¹, up to about 10¹². In some embodiments a mutagenesis or screen is performed using multiple populations of cells and/or is repeated multiple times. In some embodiments, the number of cells examined or assessed is between 10⁵ and 10¹² cells, e.g., at least 10⁶, 10⁷, 10⁸, 10⁹, 10¹⁰, 10¹¹, up to about 10¹². In some embodiments smaller numbers of cells are of use, e.g., between 1-10⁴ cells. In some embodiments a population of cells is contained in an individual vessel, e.g., a culture vessel such as a culture plate, flask, or well. In some embodiments a population of cells is contained in multiple vessels. In some embodiments two or more cell populations are pooled to form a larger population. In some embodiments a population of near-haploid mammalian cells comprises cells that collectively have insertions in at least 50%, at least 75%, at least 90%, at least 95%, at least 96%, at least 97%, at least 98%, at least 99%, or 100% of the genes present in cells of that species.

In some embodiments, near-haploid mammalian cells that have been contacted with a gene trap vector under conditions suitable for uptake and insertion of the construct are maintained in culture for a period of time prior to identifying, isolating, or assessing cells that have incorporated the construct into their genome and/or that exhibit a mitochondrial phenotype of interest. For example, cells may be cultured for between 1 and about 20 days prior to identification, isolation, or characterization. In some embodiments, cells that have taken up the construct and, in some embodiments, have the construct inserted into their genome, are identified or isolated. In some embodiments, cells are identified or isolated based at least in part on a reporter, e.g., a reporter encoded by the gene trap vector and inserted into the genome. For example, cells can be subjected to sorting or cultured under selective conditions so as to eliminate at least, e.g., 95%, 98%, 99%, 99.9%, or more of the cells that do not express a reporter. Remaining cells are then assessed to determine whether they have a mitochondrial phenotype of interest. In some embodiments a reporter-based selection step is not performed. Instead, a mutagenized cell pool is characterized with regard to a mitochondrial phenotype without such selection. In some embodiments, e.g., in the case of a promoterless gene trap vector, omitting a reporter-based selection may broaden the mutagenized cell population to include types of gene-trap insertions that may otherwise be less amenable to identification, e.g., poorly expressed genes.

In some embodiments a selection approach based on cell survival is used, wherein a mitochondrial phenotype of interest comprises cell survival in the presence of a particular selection agent (or other selective conditions) that affect, e.g., impair, at least one mitochondrial function, or wherein cell survival serves as an indicator of a mitochondrial phenotype of interest. For example, as discussed further below, in some embodiments a mitochondrial phenotype comprises resistance of a cell to a mitochondrial poison. Cells that survive in the presence of the mitochondrial poison are isolated, and one or more genes mutated in one or more of the cells are identified. In some embodiments cells that have been contacted with a gene trap vector are maintained in culture under non-selective conditions (e.g., in the absence of a selection agent) for a period of time prior to being contacted with a selection agent, e.g., in order to allow time for changes in gene expression and gene product level resulting from a mutation to occur and potentially give rise to a resistant phenotype.

In some embodiments cells that have a mitochondrial phenotype of interest are expanded in culture after being isolated or identified, e.g., after having survived a survival-based selection. In some embodiments cells are expanded for between 2 and 30 days.

A variety of methods can be used to identify genes that have a gene trap vector or portion thereof inserted therein. In some embodiments inverse PCR is used to identify genomic sequences flanking the insertion. In some embodiments splinkerette PCR is used (Horn, C., et al., Nat. Genet., 39: 807-8, 2007). In some embodiments 5′-RACE (rapid amplification of cDNA ends) is used to amplify cellular sequences contained in a gene-trap fusion transcript (see, e.g., Nature Methods, 2(8), 2005). See also Stanford, W., et al. Methods in Enzymology, Vol. 420, 2006). Examples of identifying genes mutagenized using a gene trap vector are described in PCT/US2010/041628 (WO/2011/006145) and references 5, 8, and 9. In some embodiments, sequences flanking the insertion are recovered and sequenced from large populations of cells simultaneously using “high throughput”, “next-generation”, or “massively parallel” sequencing. Such sequencing techniques can comprise sequencing by synthesis (e.g., using Solcxa technology), sequencing by ligation (e.g., using SOLiD technology from Applied Biosystems), 454 technology, or pyrosequencing. In some embodiments thousands, tens of thousands or more sequencing reactions are performed in parallel, generating millions or even billions of bases of DNA sequence per “run”. See, e.g., Shendure J & Ji H. Nat. Biotechnol., 26(10):1135-45, 2008, Mardis E R (2008) Next-generation DNA sequencing methods. Annu Rev Genomics Hum Genet. 9:387-402; and/or Metzger, M., Nat Rev Genet. 2010; 11(1):31-46, for non-limiting discussion of some of these technologies. It will be appreciated that sequencing technologies are evolving and improving rapidly. In some embodiments massively parallel sequencing by synthesis is used. In some embodiments Linear Amplification Mediated-PCR (LAM-PCR), followed by ssDNA linker ligation and massively parallel sequencing is used. The pools or populations of cells are selected for a mitochondrial phenotype of interest, and genomic regions that are enriched for insertions are identified. Such regions contain candidate genetic elements, e.g., genes, involved in the phenotype of interest. In some embodiments 10,000 or more, e.g., between 10,000 and 100,000; 10,000 and 500,000; or between 10,000 and 1 million, 5 million, 10 million, 20 million, 50 million, 100 million, insertions, or more, are analyzed. Once the DNA is isolated and, in some embodiments amplified, it can be cloned into a vector and/or sequenced. The DNA can be used as a probe to identify further sequences located nearby in the genome, e.g., by probing a cDNA or genomic library. The sequence can be used to search sequence databases, e.g., publicly available databases such as those available through Entrez at the National Center for Biotechnology Information website (http://www.ncbi.nlm.nih.gov/), e.g., GenBank, RefSeq, Protein, and Nucleotide. Since the human genome is completely sequenced it will generally be possible to readily identify most genes based on a relatively small amount of partial sequence data. Sequences may be aligned with the human genome using appropriate software. The University of California, Santa Cruz (UCSC) Genome Browser website (http://genome.ucsc.edu/) provides a large database of publicly available sequence and annotation data along with an integrated tool set useful for, e.g., examining the genomes of organisms, aligning sequence to genomes, and performing various other analyses (Rhead B, et al. The UCSC Genome Browser database: update 2010. Nucleic Acids Research. 2010; 38:D613-D619). In some embodiments Bowtie alignment software is used (Langmead B, et al. Genome Biology. 2009; 10). Insertion sites can be identified as located in genomic regions annotated to contain genes.

In some embodiments a method of analysis focuses on insertions obtained in a given screen that are present in genes and compares them to insertions present in an unselected mutagenized population of near-haploid cells (e.g., a population mutagenized at the same time as the cells subjected to screening). Enrichment of a particular gene in a particular screen can be calculated by comparing how often that gene is mutated in the screen compared to how often the gene carries an insertion in a control dataset obtained by analyzing insertion sites in an unselected population. A p-value (optionally corrected for false discovery rate) can be calculated using a suitable statistical test, such as the one-sided Fisher exact test. In some embodiments, analysis comprises obtaining a proximity index for a given insertion as the inverse value of the average distances with its neighboring insertion sites. The inverse value is calculated from the average distance (in base pairs) between the given insertion and the two neighboring upstream insertions and the two next downstream insertion sites. This method of analysis identifies insertion-rich regions and includes sense and antisense insertions and facilitates identification of non-annotated elements.

Cells can be cultured under varying culture conditions prior to, during, or after a screen. Conditions that may be varied include, e.g., culture medium, pH, osmotic pressure, temperature, gas mixture, cell density, culture surface or culture matrix (e.g., in the case of 3 dimensional cultures). Typical culture medium components include, e.g., sugars, amino acids, minerals, vitamins, hormones, growth factors, and lipids. In some embodiments a mitochondrial phenotype occurs or is detectable only under certain conditions or becomes more readily detectable under certain conditions. In some embodiments culture conditions are selected to mimic a state that may exist in vivo in a subject who has a mitochondrial disorder.

Any suitable method can be used to identify mutant cells having a mitochondrial phenotype of interest. Various methods of use are described herein (see, e.g., Section V). In some embodiments a mitochondrial phenotype is detected in individual mitochondria or cells. In some embodiments a mitochondrial phenotype is detected by making a measurement or observation on a cell or population of cells. In some embodiments the percentage of mitochondria in a cell or cell population that exhibit a mitochondrial phenotype may be determined examining the cell, cell population, or a portion thereof, or by making a measurement on a cell, cell population, or a portion thereof. In some embodiments a mitochondrial phenotype is detectable as a statistically significant change in at least one quantifiable property. In some embodiments a mitochondrial phenotype is detectable as a change of at least 5%, 10%, 20%, 30%, 40%, 50%, 60%, 70%, 80%, 90%, 95% or more as compared with a control value. In some embodiments a mitochondrial phenotype is detectable as a change (increase or decrease) of at least 1.1, 1.2, 1.5, 2, 3, 4, 5, 10, 20, 50, or 100-fold as compared with a control value. In some embodiments a mitochondrial phenotype is detectable in at least 5%, 10%, 20%, 30%, 40%, 50%, 60%, 70%, 80%, 90%, 95%, 96%, 97%, 98%, 99% or more of a cell's mitochondria. In some embodiments a mitochondrial phenotype is detectable in at least 5%, 10%, 20%, 30%, 40%, 50%, 60%, 70%, 80%, 90%, 95%, 96%, 97%, 98%, 99% or more of the cells in a cell sample.

### IV. Mitochondrial Poisons as Models for Mammalian Mitochondrial Disorders

In some aspects, the invention relates to use of mitochondrial poisons to model mitochondrial disorders in mammalian cells, e.g., for purposes of identifying therapeutic targets for mitochondrial disorders. Cells that are contacted with a mitochondrial poison may undergo alteration in, e.g., loss or reduction, of one or more mitochondrial functions. For example, a mitochondrial poison may inhibit oxidative phosphorylation. Such changes can have deleterious effects and may, in at least some instances, cause cell death or substantially decrease cell proliferation. The disclosure provides the recognition that mammalian cell death or reduced cell proliferation resulting from a mitochondrial poison can be used as the basis for screens to identify mammalian genes whose modulation inhibits the deleterious effects of the mitochondrial poison. The disclosure further provides the insight that mammalian genes whose modulation inhibits deleterious effects of mitochondrial poisons are candidate targets for modulation for purposes of treating mitochondrial disorders. In certain aspects, methods for identifying such genes are provided herein.

In some embodiments mutagenized mammalian cells are contacted with a mitochondrial poison at a concentration and for a time sufficient to kill or substantially inhibit proliferation of a majority of cells. Cells that survive such a selection process (resistant cells) are analyzed to identify one or more mutated genes. In some embodiments such a gene is a candidate target for modulation, e.g., inhibition, to (i) improve or preserve mitochondrial function; (ii) inhibit apoptosis or necrosis; (iii) protect against mitochondrial dysfunction; and/or (iv) treat a mitochondrial disorder. In some embodiments, the mutagenized mammalian cells are near-haploid mammalian cells.

In some embodiments, genes whose mutation or inhibition renders cells resistant to a mitochondrial poison are identified as targets for development of therapeutic agents for treatment of mitochondrial disorders. It will be understood that resistance to a mitochondrial poison or other agent may refer to resistance at a specified concentration or over a specified concentration range, e.g., a concentration or concentration range that would be lethal to control cells that are not resistant. In some embodiments resistance to a mitochondrial poison refers to ability of a cell to survive and, in some embodiments proliferate, in the presence of or following exposure to a mitochondrial poison under conditions (e.g., concentration and time period) that would typically be lethal to control cells that are not resistant. In some embodiments resistance to a mitochondrial poison refers to ability of a cell to survive and, in some embodiments proliferate, in the presence of or following exposure to a mitochondrial poison under conditions (e.g., concentration and time period) that would cause control cells that are not resistant to die or essentially cease proliferating. In some embodiments a cell that is resistant under such conditions may exhibit one or more sub-lethal effects, such as a somewhat reduced proliferation rate. A first cell, cell population, or cell line that exhibits less severe effects than a second cell, cell population, or cell line may be said to have increased resistance as compared to the second cell, cell population, or cell line. “Resistant”, “resistance”, “increased resistance” and like terms are used interchangeably with “not sensitive”, “sensitivity”, “decreased sensitivity” and like terms, respectively. “Sensitive”, “sensitivity”, “increased sensitivity” and like terms are used interchangeably with “not resistant”, “lack of resistance”, “decreased resistance” and like terms.

In some embodiments a mammalian cell is contacted with a modulator of the gene, and at least one phenotype or function of the cell's mitochondria is assessed. In some embodiments the cell has mitochondrial dysfunction, e.g., deficient mitochondrial function. In some embodiments, if the cell exhibits improved mitochondrial phenotype or function, the modulator is identified as a candidate agent useful for (i) improving or preserving mitochondrial function; (ii) inhibiting apoptosis or necrosis; and/or (iii) protecting against mitochondrial dysfunction; and/or (iv) treating a mitochondrial disorder. In some embodiments the modulator is identified as a candidate therapeutic agent for treatment of a mitochondrial disorder if the cell is protected against mitochondrial dysfunction that would otherwise kill the cell or reduce its capacity to carry out one or more of its normal functions.

The term “protect against mitochondrial dysfunction” refers to reducing, preventing, or limiting the extent of mitochondrial dysfunction or one or more adverse effects associated with mitochondrial dysfunction. In some embodiments protecting against mitochondrial dysfunction comprises (a) improving or preserving at least one mitochondrial function in a cell that has an abnormality, e.g., a deficiency, in one or more mitochondrial functions; (b) at least in part counteracting, inhibiting, or compensating for mitochondrial dysfunction or at least one effect of mitochondrial dysfunction. In some embodiments protection against mitochondrial dysfunction comprises reducing the likelihood that a cell at risk of or having mitochondrial dysfunction will die as a consequence mitochondrial dysfunction. In some embodiments protection against mitochondrial dysfunction comprises reducing the severity of structural and/or functional damage that occurs as a consequence of mitochondrial dysfunction. In some embodiments the likelihood of cell death or percentage of cells that die over a given time period is reduced by at least 10%, 20%, 30%, 40%, 50%, 60%, 80%, 90%, 95%, or more.)

In some embodiments mitochondrial function (or dysfunction) may be assessed by detecting or measuring an appropriate indicator. Examples of indicators of mitochondrial function are described in Section V and include membrane potential, oxygen consumption, respiration, ATP production, matrix pH, and membrane integrity, to name a few. In some embodiments, improvement or preservation of a mitochondrial function results in at least in part restoring to a normal level, or preserving, an indicator of a mitochondrial function in a cell having deficient mitochondrial function. In some embodiments improvement of a mitochondrial function results in increasing to a level above normal (where such increased level is beneficial) or decreasing to a level below normal (where such decreased level is beneficial) at least one indicator of mitochondrial function in a cell having abnormal or normal mitochondrial function. In some embodiments protection against mitochondrial dysfunction or improvement in mitochondrial function results in improvement in a clinically relevant parameter such as a clinical score, prognosis, or outcome and/or an improvement in a biomarker that correlates with or is predictive of clinical benefit. Protection against mitochondrial dysfunction and/or improved mitochondrial function may result from changes in molecules, structures, processes, or events outside mitochondria, from changes in mitochondrial molecules, structures, processes, or events, from changes in direct interactions between mitochondrial and extra-mitochondrial genes and/or gene products, and/or from changes in the level, localization, activity, or interactions of molecules that may be produced, used, or metabolized by mitochondria, e.g., metabolites, substrates, precursors, intermediates, cofactors, products, etc.

In general, any mitochondrial poison may be used in various embodiments. In general, mammalian cells can be contacted with a mitochondrial poison by adding the mitochondrial poison to cell culture medium either before or while the culture medium is used to culture cells. In some embodiments a mitochondrial poison is used at a concentration sufficient to kill at least 80%, 85%, 90%, 95%, 96%, 97%, 98%, 99%, 99.5%, 99.9%, 99.95%, 99.99%, 99.999%, 99.9995%, 99.9999% or more of unselected, unmutagenized mammalian cells when contacted with such cells for a given time period, e.g., a predetermined time period. In some embodiments a mitochondrial poison is used at a concentration sufficient to cause at least 80%, 85%, 90%, 95%, 96%, 97%, 98%, 99%, 99.5%, 99.9%, 99.95%, 99.99%, 99.999%, 99.9995%, 99.9999% or more of unselected, unmutagenized mammalian cells to cease proliferating when contacted with such cells for a given time period. In some embodiments a mitochondrial poison is used at a concentration sufficient to kill at least 80%, 85%, 90%, 95%, 96%, 97%, 98%, 99%, 99.5%, 99.9%, 99.95%, 99.99%, 99.999%, 99.9995%, 99.9999% or more of unselected, mutagenized mammalian cells when contacted with such cells for a given time period. In some embodiments a mitochondrial poison is used at a concentration sufficient to cause at least 80%, 85%, 90%, 95%, 96%, 97%, 98%, 99%, 99.5%, 99.9%, 99.95%, 99.99%, 99.999%, 99.9995%, 99.9999% or more of unselected, mutagenized mammalian cells to cease proliferating when contacted with such cells for a given time period. In some embodiments a mitochondrial poison is used at a concentration that reduces the number of viable mutagenized cells by at least 80%, 85%, 90%, 95%, 96%, 97%, 98%, 99%, 99.5%, 99.9%, 99.95%, 99.99%, 99.995%, 99.999%, 99.9995%, 99.9999% or more when contacted with such cells for a given time period. In some embodiments a given time period, e.g., a predetermined time period, is between 24 hours and 4 weeks, e.g., between 24 and 72 hours, between 72 and 168 hours, between 1 and 3 weeks, e.g., about 2 weeks. It will be understood that the culture medium may be changed and/or supplemented with a mitochondrial poison one or more times during the culture period. In the context of screening for cells that exhibit resistance to a mitochondrial poison, “unselected mammalian cells” refers to mammalian cells not known to be resistant to the mitochondrial poison, not having been previously selected to be resistant to the mitochondrial poison, and not descended from such cells. In some embodiments an appropriate concentration is one at which the mitochondrial poison has a relatively or highly specific effect on mitochondria (e.g., on a particular mitochondrial protein, complex, or process) as compared with its effects on non-mitochondrial cellular components or processes. In some embodiments approximately the minimum concentration effective to kill a predetermined percentage of cells within a given time is used. In some embodiments between about 1 and 2 or between about 2 and 5 times the minimum concentration effective to kill a predetermined percentage of cells within a given time is used.

In some embodiments a suitable concentration is selected by testing the effect of a range of concentrations of the selected mitochondrial poison to kill non-mutagenized cells, e.g., non-mutagenized cells of the cell line on which a screen is to be performed. In some embodiments a suitable concentration is selected at least in part by testing the effect of a range of concentrations of the selected mitochondrial poison to kill mutagenized cells, e.g., mutagenized cells of the cell line on which a screen is performed. Cells are contacted with the mitochondrial poison and cell survival or proliferation is assessed at various time points. The testing may be performed as part of an initial screen. In some embodiments an appropriate concentration is between 1 nM and 1 mm, e.g., between 1 nM and 100 nm, between 100 nm and 1 μm, between 1 μm and 10 μm, between 10 μm and 100 μm, between 100 μm and 500 um, or between 500 μm and 1 mm. Once a suitable concentration or concentration range is determined, the same or a similar concentration or concentration range may be used in subsequent screens.

In some embodiments a mitochondrial poison is a complex I inhibitor. Complex I inhibitors include, e.g., rotenone, amytal, pieridicin A, MPP+ (1-methyl-4-phenylpyridinium), bullatacin, and mycothiazole. In some embodiments a mitochondrial poison is a complex II inhibitor. Complex II inhibitors include, e.g., atpenin A5 (AA5; Axxora LLC, San Diego Calif.), malonate, diazoxide (DZX), 3-nitropropionic acid, and nitroxyl. In some embodiments a mitochondrial poison is a complex III inhibitor. Complex III inhibitors include, e.g., antimycin A, myxothiazol, and stigmatellin. In some embodiments a mitochondrial poison is a complex IV inhibitor. Complex IV inhibitors include, e.g., cyanides (compounds that comprise a—C≡N functional group, also termed nitriles) such as hydrogen cyanide, sodium cyanide, potassium cyanide, trimethylsilyl cyanide (CH₃)₃SiCN or other compounds that readily release HCN or the cyanide ion; azides, sulfides (e.g., hydrogen sulfide), and carbon monoxide. In some embodiments a complex IV inhibitor binds to heme or a component thereof. In some embodiments a mitochondrial poison is a complex V inhibitor. Complex V inhibitors include, e.g., oligomycin B, DCCD (dicyclohexylcarbodiimide), and venturicidin. In some embodiments a mitochondrial poison is an uncoupling agent. An “uncoupling agent” is a chemical agent that uncouples oxidation from phosphorylation in the metabolic cycle so that ATP synthesis does not occur. Uncoupling agents include ionophores that disrupt electron transfer by short-circuiting the proton gradient across mitochondrial membranes. Uncoupling agents include, e.g., dinitrophenol, valinomycin, nigericin, and FCCP (carbonylcyanide p-trifluoromethoxyphenylhydrazone). In some embodiments a mitochondrial poison is an inhibitor of a mitochondrial transporter, such as the adenine nucleotide translocator (ANT, sometimes called ANT1 or solute carrier family 25 member 4, encoded by SLC25A4 (103220)). ANT inhibitors include, e.g., atractyloside, carboxyatractyloside, and bongkrekic acid. Analogs of any of the afore-mentioned mitochondrial poisons may be used. In some embodiments a mitochondrial poison may be relatively or highly specific for a particular mitochondrial protein, complex, or process. In some embodiments a mitochondrial poison inhibits multiple mitochondrial proteins, complexes, or processes. For example, a complex I inhibitor may be capable of inhibiting one or more other OXPHOS complexes in addition to inhibiting complex I.

In some embodiments a mitochondrial poison is a nucleoside analog, e.g., a nucleoside analog reverse transcriptase inhibitor (NTRI), a nucleotide analog reverse-transcriptase inhibitors (NtRTI), or a non-nucleoside reverse transcriptase inhibitor (NNRTI). In some embodiments a NRTI is Zidovudine (also called AZT, ZDV, and azidothymidine), Didanosine (also called ddI), Zalcitabine (also called ddC and dideoxycytidine), Stavudine (also called d4T), Lamivudine (also called 3TC), Abacavir, also called ABC), Emtricitabine (also called FTC), Entecavir (also called ETV), Apricitabine (also called ATC). In some embodiment a NtRTI is Tenofovir (also called TDF), Adefovir, also known as bis-POM PMPA). In some embodiments a NNRTI is Efavirenz, Nevirapine, Delavirdine, Etravirine, or Rilpivirine. In some embodiments a NRTI, NtRTI, or NNRTI is approved for treatment of HIV and/or Hepatitis B infection. In some embodiments a NTRI, NtRTI, or NNRTI may cause mitochondrial toxicity at least in part by: inhibiting DNA polymerase gamma (the polymerase responsible for synthesis of mitochondrial DNA, also referred to as mitochondrial DNA polymerase), causing mitochondrial DNA depletion (reduction in mitochondrial DNA content), mitochondrial RNA depletion (reduction in mitochondrial RNA content), or having a direct effect on mitochondrial adenylate kinase and adenosine nucleotide translocator (ANT) or on an OXPHOS component or subunit thereof. Mitochondrial toxicity due to NRTIs, NtRTIs, and NNRTIs is discussed in further detail in Apostolova, N., et al., Trends in Pharmacological Sciences (2011), 32(12): 715-725.

In some embodiments, a mitochondrial poison is used as a selection agent in a haploid genetic screen, as described herein (e.g., in Section III and in the Examples). Mutagenized near-haploid mammalian cells, e.g., near-haploid mammalian cells that have been mutagenized with a gene trap vector, are contacted with a mitochondrial poison. Cells that survive the selection (i.e., cells that are resistant to the mitochondrial poison) are obtained, and insertion sites are identified. One or more genes in which insertions occur among the surviving cells at a frequency greater than the frequency at which they occur in an unselected mutagenized population are identified. In some embodiments such a gene is identified as a candidate target whose modulation, e.g., inhibition, has potential to protect a cell against the mitochondrial poison. In some embodiments such a gene is identified as a candidate target for modulation, e.g., inhibition, to (i) improve or preserve mitochondrial function; (ii) inhibit apoptosis or necrosis; and/or (iii) protect against mitochondrial dysfunction; and/or (iv) treat a mitochondrial disorder. In some embodiments a gene identified in a haploid genetic screen is confirmed as being one whose modulation protects against mitochondrial dysfunction. In some embodiments, confirmation comprises introducing a functional copy of the gene into a mutant cell lacking a functional copy of the gene and assessing the cell for resistance to the mitochondrial poison. If the cell is no longer resistant, the gene is confirmed as one whose modulation, e.g., inhibition, protects the cell against the mitochondrial poison. In some embodiments confirmation comprises functionally inactivating the gene in a cell that has a functional copy of the gene and is sensitive to the mitochondrial poison, e.g., a wild type cell. If functionally inactivating the gene in the wild type cell results in resistance to the mitochondrial poison, the gene is confirmed as one whose modulation, e.g., inhibition, protects the cell against the mitochondrial poison. In some embodiments the gene is functionally inactivated by inhibiting its expression using RNAi. In some embodiments, if functional inactivation of a gene results in resistance to a mitochondrial poison, an inhibitor of the gene is a candidate agent for treatment of mitochondrial disorders.

In some embodiments, a mitochondrial poison is used as a selection agent in a screen that involves genetically modifying mammalian cells, so as to result in increased or decreased functional expression of a gene. In some embodiments, a gene that is normally expressed by the cell is inhibited. In some embodiments, the level of a gene product is increased. In some embodiments mammalian cells are modified by introducing into the cells a library of nucleic acids that correspond in sequence to a plurality of genes. In some embodiments the library is introduced into cells as a pool. The nucleic acids may comprise a barcode, which may be used to facilitate subsequent identification of the nucleic acid. In some embodiments members of the library are introduced individually into cells in different vessels (e.g., different wells in a multiwell plate), e.g., as an array. In some embodiments the nucleic acids contain a sequence to be expressed, operably linked to appropriate expression control elements (e.g., a promoter) capable of directing expression in the cells. In some embodiments, expression of the sequence in a cell causes the cell to have increased or decreased functional expression of a gene e.g., as compared to control cells that have not been modified to contain the exogenous nucleic acid. Cells into which the library has been introduced are contacted with a mitochondrial poison, e.g., as described above. After a suitable period of time surviving cells are identified or isolated. The exogenous nucleic acid sequences previously introduced into such cells are identified. In some embodiments, e.g., if the nucleic acids were introduced individually, the identity of a nucleic acid that conferred resistance may be evident from the location or number of a vessel containing resistant cells. In some embodiments, genes to which such sequences correspond are identified as candidate genes whose modulation, e.g., inhibition, has potential to protect a cell against the mitochondrial poison. In some embodiments such a gene is identified as one whose modulation, e.g., inhibition, improves mitochondrial function. In some embodiments such a gene is identified as one whose modulation, e.g., inhibition, has potential to inhibit apoptosis or necrosis. In some embodiments such a gene is identified as one whose modulation, e.g., inhibition, has potential to protect a cell against mitochondrial dysfunction. In some embodiments such a gene is identified as a candidate target for development of therapeutic agents treat mitochondrial disorders. For example, in some embodiments, if inhibition of a particular gene results in resistance to a mitochondrial poison, an inhibitor of the gene is a candidate agent for treatment of mitochondrial disorders. In some embodiments, if increasing the level of a gene product results in resistance to a mitochondrial poison, the gene product, or an activator or inducer of the gene product, is a candidate agent for treatment of mitochondrial disorders.

In some embodiments, a nucleic acid “corresponds” to a gene if the nucleic acid comprises a sequence that is at least 80%, 90%, 95%, 96%, 97%, 98%, 99%, or 100% identical to or at least 80%, 90%, 95%, 96%, 97%, 98%, 99%, or 100% complementary to at least a portion of the gene or an expression product of the gene over at least 10, 12, 15, 20, 50, 100, 500 nucleotides, or more. In some embodiments a library comprises members that correspond to at least about 50%, 60%, 70%, 80%, 90%, 95%, 96%, 97%, 98%, 99%, or more of the known or predicted genes present in a genome of a mammalian species of interest. In some embodiments a library comprises members that correspond to at least 10,000, at least 12,000, at least 15,000, or at least 20,000 genes. In some embodiments the genes are human genes. In some embodiments the genes are mouse genes.

In some embodiments, a library is an open reading frame (ORF) or RNAi library. In some embodiments an RNAi library is a short hairpin RNA (shRNA) library or siRNA library or microRNA library. An RNAi library may be used to perform a loss-of-function screen, e.g., to identify genes and/or gene products, whose loss of function results in resistance to a mitochondrial poison. In some embodiments an ORF library is a cDNA library. An ORF library may be used to perform a gain-of-function screen, e.g., to identify genes and/or gene products, whose expression or activation results in resistance to a mitochondrial poison. In some embodiments nucleic acids in a library are contained in a suitable vector for expression in mammalian cells. In some embodiments a retroviral, e.g., a lentiviral vector, is used. In some embodiments nucleic acids are introduced into cells such that each cell contains on average not more than 1 distinct nucleic acid construct. Methods of generating nucleic acid libraries, e.g., ORF, siRNA, shRNA libraries are known in the art, and a number of such libraries are commercially available. Examples of shRNA libraries are described in Root, D., et al., Nat Methods (2006); 3, 715-719; and/or Luo et al. Proc. Natl. Acad. Sci. USA. (2008); 105:20380-20385. In some embodiments an shRNA library comprises a shRNA library from The RNAi Consortium (http://www.broadinstitute.org/rnai/public/), which may be obtained from Sigma-Aldrich or Open Biosystems. Examples of ORF libraries are described in Yang, X, et al., Nature Methods (2011); 8, 659-661 and references therein. In some embodiments an ORF library comprises the hORFeome V8.1 collection (Yang, supra).

In some embodiments a library comprises multiple nucleic acids that correspond to at least some of the genes. For example, an RNAi library may multiple different RNAi agents that target the same gene. In some embodiments different portions of a transcript are targeted. An ORF library may comprise multiple ORFs transcribed from the same gene. The gene products may, for example, differ as a result of alternative splicing, RNA editing, etc. In some embodiments a library comprises a focused collection of nucleic acids that correspond to genes that share a property of interest. For example, in some embodiments the genes encode proteins that have a particular enzymatic activity (e.g., kinase activity), or play a role in a particular biological process. In some embodiments a library comprises nucleic acids that correspond to genes that encode mitochondrial proteins.

The process of contacting mammalian cells that have been modified to have increased or decreased functional expression of genes with a mitochondrial poison can be performed as described for haploid genetic screens. For example, time periods, concentrations, cell number, etc., may be as described for haploid genetic screens.

A wide variety of cell types can be used in various aspects and embodiments described herein. A cell may originate from any organism of interest, e.g., a vertebrate, e.g., a mammal. In some embodiments, a cell is a primate cell, e.g., a monkey cell or a human cell. In some embodiments a cell is a primary cell, immortalized cell, non-cancer cell, or cancer cell. In some embodiments a cell is a member of a cell line. In some embodiments a cell line is descended from a single cell. In some embodiments a cell line is descended from multiple cells isolated from a single individual. In some embodiments, a cell is an epithelial cell. In some embodiments, a cell is fibroblast. In some embodiments, a cell is a hematopoietic cell. In some embodiments a cell originates from breast, bladder, bone, brain, bronchus, cervix, colon, endometrium, esophagus, larynx, liver, lung, nerve, muscle, ovary, pancreas, prostate, stomach, kidney, skin, testis, or thyroid gland. In some embodiments, a cell is a tumor cell. In some embodiments a cell is an immortalized, nontumorigenic cell. Numerous cell lines are known in the art, many of which can be obtained from repositories such as the American Type Culture Collection, Coriell Cell Repositories, European Collection of Cell Cultures, Japanese Collection of Research Bioresources, or from a variety of commercial suppliers. Examples of cell lines include HeLa, Vero, RD, CHO, HEK-293, COS, HMEC, MDCK, NIH-3T3, HEp-2, A549, and BEAS-2B. In some embodiments, a cell is a near-haploid cell. In some embodiments, a cell is a KBM7 cell. In some embodiments a cell is generated from a stem cell. For example, a cell may be a differentiated cell type derived from a stem cell. In some embodiments a stem cell is an induced pluripotent stem cell, embryonic stem cell, neural stem cell, hepatic stem cell, mesenchymal stem cell, or hematopoietic stem cell. Protocols suitable for deriving numerous different cell types from pluripotent cells or adult stem cells in vitro are known to those of ordinary skill in the art. In some embodiments, for example, a hepatocyte, neuron, skeletal muscle cell, or cardiac muscle cell is derived from a stem cell in vitro.

In some embodiments a cell is a rho0 (ρ⁰) cell (also referred to as ρ0 cell), e.g., a mammalian ρ⁰ cell, e.g., a human ρ⁰ cell. ρ⁰ cells are cells that lack mtDNA, e.g., because the) endogenous mtDNA has been lost or depleted). ρ⁰ cells may be generated, for example, by exposing cells to various agents that deplete mtDNA, such as ethidium bromide.

In some embodiments a cell is a cytoplasmic hybrid cell (“cybrid cell” or “cybrid”), e.g., a mammalian cybrid cell, e.g., a human cybrid cell. Cybrid cells are cells that combine the nuclear genome from one source with the mitochondrial genome from another source. Cybrids can be constructed by fusing enucleated cells harboring mitochondria comprising wild type or altered mtDNA of interest with ρ⁰ cells (see, e.g., Vithayanil, S A, et al, Meth. Mol. Biol., 837:219-230, 2012). In some aspects, cybrid near-haploid mammalian cells are provided. In some embodiments cybrid, near-haploid mammalian cells are generated by depleting endogenous mtDNA from near-haploid mammalian cells (e.g., KBM7 cells) and fusing the resulting cells to enucleated cells harboring mitochondria comprising wild type or altered mtDNA of interest. In some embodiments a screen to identify genes that affect mitochondrial phenotype is performed using cybrid near-haploid mammalian cells. In some embodiments a screen to identify genes that confer resistance to a mitochondrial poison is performed using cybrid mammalian cells. In some embodiments the cybrid cells harbor mutant mtDNA, e.g., mtDNA having a mutation that results in a mitochondrial disorder. Such cells may be useful, e.g., to identify genes that affect a mitochondrial phenotype associated with the disorder and/or to test a candidate agent of potential use to treat the disorder.

In some embodiments a cell is an induced pluripotent stem (iPS) cell derived from a subject suffering from a mitochondrial disorder or a cell derived by differentiation of such an iPS cell. For example, in some embodiments the cell is a hepatocyte, neuron, or muscle cell derived from such an iPS cell.

In some embodiments a cell, e.g., a stem cell, is genetically engineered to have one or more mutations associated with a mitochondrial disorder. In some embodiments the mutation is one that causes a mitochondrial disorder with a dominant or recessive inheritance pattern. Examples of such mutations and disorders are described further herein.

In some embodiments, an agent that modulates a gene that is a candidate target for treatment of mitochondrial disorders is identified or obtained. In some embodiments, an agent that modulates a gene that is a candidate target for treatment of mitochondrial disorders is tested in a system that serves as a model of a mitochondrial disorder. For example, in some embodiments an inhibitor of a gene, mutation of which results in resistance to a mitochondrial poison, is tested in a model of a mitochondrial disorder. In some embodiments the agent is tested in a system in which a mitochondrial poison is used to model a mitochondrial disorder. In some embodiments the agent is tested in a system that does not comprises using a mitochondrial poison to model a mitochondrial disorder. In some embodiments, if the agent shows evidence of a protective effect in the model system, the agent is confirmed as a useful agent to treat a mitochondrial disorder.

In some embodiments isolated mitochondria serve as a model of a mitochondrial disorder. In some embodiments, mitochondria that exhibit mitochondrial dysfunction are used. In some embodiments the mitochondria are obtained from cells that harbor a mutation in a gene, wherein the mutation is associated with a mitochondrial disorder.

In some embodiments an agent is tested in a cell-based system that serves as a model of a mitochondrial disorder. In some embodiments, cells that exhibit mitochondrial dysfunction are used. In some embodiments the cells harbor a mutation in a gene, wherein the mutation is associated with a mitochondrial disorder. In some embodiments the cell is a cybrid cell, a ρ⁰ cell, a cell that has been or is exposed to a mitochondrial poison, a cell derived from a subject suffering from a mitochondrial disorder (e.g., derived from an iPS cell derived from such a subject), or a cell that has been genetically engineered to have one or more mutations or genetic variations associated with a mitochondrial disorder. In some embodiments the cells are human cells.

In some embodiments, a cell may be genetically modified using an endonuclease that is targeted to selected DNA sequences. For example, cells with mutations in genes that affect mitochondrial function may be generated. Mutations corresponding to those found in human mitochondrial disorders may be engineered. Examples of site-specific nucleases include zinc-finger nucleases (ZFNs), meganucleases, and TALENs as well as RNA directed nucleases such as CRISPR/Cas systems. ZFNs comprise DBDs derived from or designed based on DBDs of zinc finger (ZF) proteins. TALENs comprise DBDs derived from or designed based on DBDs of transcription activator-like (TAL) effectors of various Xanthomas species, e.g., plant pathogenic Xanthomonas spp. The nuclease portion of a ZFN or TALEN is typically a FokI endonuclease or variant thereof. A nuclease introduced into a cell or expressed from a nucleic acid construct in the cell cleaves genomic DNA at one or more targeted sites, followed by repair by non-homologous end joining or homology-directed repair. In some embodiments precise alterations in the genome of a cell may be generated by introducing a donor nucleic acid containing the desired alteration in addition to expressing or introducing the nuclease. See, e.g., WO2011097036; Urnov, F D, et al., Nature Reviews Genetics (2010), 11: 636-646; Miller J C, et al., Nat. Biotechnol. (2011) 29(2):143-8; Cermak, T., et al. Nucleic Acids Research, 2011, Vol. 39, No. 12 e82; Gaj, T., et al., Trends Biotechnol. 2013 July; 31(7):397-405 and references in any of the foregoing, for further description of nuclease-based genetic modification systems.

In some embodiments an agent is tested in a model of an isolated organ or tissue. For example, in some embodiments cells are cultured in or on a three-dimensional scaffold. In some embodiments the scaffold comprises a hydrogel. In some embodiments the scaffold comprises a polymer. In some embodiments a polymer is a synthetic polymer, e.g., PEG. In some embodiments a polymer is a naturally occurring or synthetic polypeptide or polysaccharide. In some embodiments cells of interest comprise hepatocytes, myocytes (e.g., cardiomyocytes), or neurons. In some embodiments cells comprise fibroblasts. For example, hepatocytes and fibroblasts may be co-cultured. In some embodiments a scaffold comprises substances that may provide a supportive microenvironment for cells associated therewith. Such substances may include, e.g., growth factors, extracellular matrix (ECM) components such as ECM proteins or portions thereof (e.g., RGD-containing peptides). In some embodiments an engineered in vitro model of parenchymal tissue (e.g., human liver) that remains functional for at least several weeks, e.g., at least 3 weeks, is used. In some embodiments microfabrication techniques are used to create 2-D and 3-D cultures that comprise parenchymal cells (e.g., primary human hepatocytes) spatially arranged in a bounded geometry by non-parenchymal cells in a micropatterned coculture. See, e.g., PCT/US2006/020019 (WO2006127768) or Khetani S R, Bhatia S N. Nat. Biotechnol. 2008; 26:120-126, for examples.

In some embodiments an agent is tested in a system comprising an isolated organ or tissue section or slice. In some embodiments a brain section or slice is used. In some embodiments an isolated organ or organoid comprises an isolated liver or liver organoid. Organoid refers to a three-dimensional cellular structure that resembles an organ or tissue of the body. In general, organoids comprise multiple differentiated cell types that are found in the relevant organ or tissue in vivo and reproduce the spatial morphology and cell-cell interactions as found in that organ or tissue. In some embodiments an organoid is an epithelial organoid. Methods for preparing organoids are known to those of ordinary skill in the art.

In some embodiments a non-human animal serves as an animal model for a mitochondrial disorder that affects humans. In some embodiments the animal is a transgenic animal. As used herein, a “transgenic animal” is any animal at least some of whose cells comprise an engineered alteration in the nuclear or mitochondrial genome of at least some of its cells or comprise a heritable extrachromsomal element in at least some of its cells. A genetic alteration may comprise one or more insertions, deletions, substitutions, rearrangements, or additions. In some embodiments a transgenic animal comprises an exogenous nucleic acid sequence present as an extrachromosomal element or stably integrated in all or a portion of its cells. Unless otherwise indicated, it will be assumed that a transgenic animal comprises stable changes to the germline sequence. Methods of generating transgenic animals are known in the art. During the initial construction of the animal, animals in which only some cells have the altered genome may be generated. Such mosaic animals may be used as models or for breeding purposes in order to generate the desired transgenic animal. Animals having a heterozygous alteration can be generated by breeding of mosaic animals. Male and female heterozygotes are typically bred to generate homozygous animals in some embodiments tetraploid complementation is used. In some embodiments exogenous genetic material (e.g., DNA) is from a different species than the animal host, or is altered in sequence, e.g., in a coding or non-coding sequence. The introduced DNA may comprise a wild-type gene, naturally occurring polymorphism, or a genetically manipulated sequence, for example having deletions, substitutions or insertions in the coding or non-coding regions. In some embodiments an introduced gene or portion thereof comprises a coding sequence, which may be operably linked to expression control elements, e.g., a promoter, and optionally other regulatory sequences. Engineered cells include “knock-out”, “knock-down”, and “knock-in” cells. Transgenic animals include “knock-out”, “knock-down”, and “knock-in” animals. Knock-down or knock-out cells or animals have a partial or complete loss of function in one or both alleles of an endogenous gene of interest. For example, a gene may be at least partly deleted or functionally inactivated, e.g., by an insertion. Also encompassed are cells and animals engineered to express RNAi agents (e.g., short hairpin RNA) or antisense agents to inhibit expression of a gene. Knockouts have a partial or complete loss of function in one or both alleles of an endogenous gene of interest. In a knockout, in some embodiments the target gene expression is undetectable or insignificant. For example, in some embodiments the function of an endogenous gene is substantially decreased so that expression is not detectable or only present at insignificant levels. This may be achieved by a variety of mechanisms, including introduction of a disruption of the coding sequence, e.g., insertion of one or more stop codons, insertion of a DNA fragment, deletion of coding or non-coding sequence (e.g., promoter region, 3′ regulatory sequences, enhancers), substitution of stop codons for coding sequence, etc. In some embodiments the exogenous DNA sequences are ultimately deleted from the genome, leaving a net change to the native sequence. Transgenic animals include conditional knock-outs, for example, where alteration of a target gene occurs upon exposure of the animal to a substance that promotes target gene alteration, introduction or induction of expression of an enzyme that promotes recombination at the target gene site (e.g., Cre in the Cre-lox system), or other method for directing the target gene alteration postnatally. Knockins have an introduced gene or portion thereof with altered genetic sequence and/or function from the endogenous gene, wherein the introduced gene or portion thereof replaces or alters the endogenous gene. Expression control sequences (e.g., promoter, enhancer) may be constitutive or inducible in various embodiments. In some embodiments expression control sequences (e.g., promoter, enhancer) are tissue-specific. For example, an expression control sequence originating from a gene that is selectively or specifically expressed in a hepatocyte, neuron, or other cell type of interest may be used.

In some embodiments a knockout in an animal is regulatable, e.g., inducible, and/or tissue or cell type specific in vivo. For conditional mutagenesis, a target gene may be modified by the insertion of two loxP sites that allow the excision of the flanked (floxed) gene segment through Cre-mediated recombination. Expression of Cre may be under control of a regulatable promoter or Cre activity may be regulated by a small molecule. For example, Cre may be fused to a steroid hormone ligand binding domain so that its activity is regulated by receptor ligands. Cre-ER(T) or Cre-ER(T2) recombinases may be used, which comprise a fusion protein between a mutated ligand binding domain of the human estrogen receptor (ER) and the Cre recombinase, the activity of which can be induced by 4-hydroxy-tamoxifen. Conditional mutant mice are obtained by crossing the floxed strain with a Cre transgenic line such that the target gene becomes inactivated in vivo in the cells in which Cre is expressed. By using a Cre transgenic line in which Cre expression is under control of a tissue or cell type specific regulatory region, e.g., a cell type specific promoter, excision is limited to those tissues in which Cre is expressed. Other methods of generating inducible and/or tissue or cell type specific knockouts known in the art may be used. For example, different recombinases (e.g., Dre or the Flp/Frt system) may be used, and/or other means of rendering recombinase activity regulatable may be used. Examples of methods and reagents useful for generating transgenic and knockout animals, including inducible and conditional, are described in Hofker, M H & van Deursen, J M (eds.) Methods Mol. Biol. Vol. 693, 2011, Humana Press; Anegon, I. (ed.) Methods Mol. Biol. Vol. 597, 2010, Humana Press.

In some embodiments non-human animals, e.g., mice, harboring precise genetic alterations, e.g., in genes that affect mitochondrial function, e.g., genes that are mutated in a mitochondrial disorder, may be generated using site-specific nucleases to target DNA sequences of interest in ES cells or zygotes.

In some embodiments the animal is a chimeric animal, wherein the chimeric animal comprises transplanted human cells or tissue. For purposes hereof, introduction of one or more individual cells or a tissue sample into a subject (e.g., a non-human mammal or a human) may be referred to as “grafting”, and the introduced Cell(s) or tissue may be referred to as a “graft”. A non-human subject to whom an agent is administered at least in part for testing purposes or on which a procedure is performed at least in part for testing purposes may be referred to as a “test animal”. In some embodiments a test animal is a rodent, e.g., a rabbit, rat, or mouse. In some embodiments, the introduced cells are of a different species than the test animal, i.e., the graft is a “xenograft”. For example, the cells can be human cells. In some embodiments, the test animal is immunocompromised. Immunocompromised animals are known in the art. For example, the test animal may be selected to have a functionally deficient immune system or may be treated (e.g., with radiation or an immunosuppressive agent or surgery such as removal of the thymus) so as to reduce immune system function. In some embodiments, the test animal has a naturally occurring or engineered mutation that renders it immunodeficient. In some embodiments, the test animal is a SCID mouse, NOD mouse, NOD/SCID mouse, nude mouse, and/or Rag1 and/or Rag2 knockout mouse, or a rat having similar properties with respect to its immune system (e.g., a nude rat). In some embodiments, the immunocompromised test animal substantially lacks T cells and/or B cells. In some embodiments, the test animal is not immunocompromised. In some embodiments, the test animal is transgenic. In some embodiments, the cells are of the same species as the test animal. In some embodiments the cells are substantially isogenic to the test animal, e.g., the cells originate from an animal of the same inbred strain as the test animal. In some embodiments the transplanted human cells or tissue originate from a subject suffering from a mitochondrial disorder. In some embodiments the transplanted human cells or tissue are genetically modified to comprise a mutation associated with a mitochondrial disorder. In some embodiments the transplanted human cells or tissue are genetically modified to have a mutation in a gene encoding a mitochondrial protein or accessory factor. In some embodiments a human ectopic artificial liver (HEAL) or humanized rodent (e.g., mouse or rat) with ectopic artificial liver tissues comprising human hepatocytes is used (see, e.g., Chen, A, et al., Proc Natl Acad Sci USA. 2011; 108(29): 11842-11847, or Katoh, M., Toxicology. 2008; 246(1):9-17; Hasegawa, M., et al., Biochem Biophys Res Commun. (2011) 405(3):405-10 and references in any of the foregoing for description). In some embodiments, human hepatocytes with a mutation associated with a mitochondrial disorder are used, e.g., to produce a HEAL or chimeric rodent comprising a HEAL. In some embodiments a HEAL or chimeric rodent comprising a HEAL is exposed to a mitochondrial poison or liver toxin that acts on mitochondria. In some embodiments human hepatocytes are derived from iPS cells derived from a subject suffering from a mitochondrial disorder and harbor the mutations and/or genetic variations, if any, that contribute to causing the disorder. In some embodiments liver buds created in vitro from human pluripotent stem cells, e.g., iPS cells, are used. In some embodiments vascularized and functional human liver is generated by transplantation of such liver buds into a suitable animal host such as an immunocompromised rodent. See Takebe, T., et al., Nature. 2013 Jul. 3. doi: 10.1038/nature 12271. [Epub ahead of print] for description of generating human liver buds in vitro from human iPS cells and using them to generate livers in mice.

In some embodiments a non-human animal serves as a model for PD. In some embodiments an animal model for PD utilizes a dopaminergic neurotoxin such as 6-hydroxydopamine (6OHDA) or 1-methyl 4-phenyl 1,2,3,6-tetrahydropyridine (MPTP). In some embodiments an animal model is a transgenic animal. In some embodiments an animal model for PD is used in which expression or activity of one or more PD-linked genes (e.g. alpha-synuclein, DJ-1, LRRK2, Parkin, UCH-L1, PINK1) is manipulated to elicit a PD phenotype. For example, in some embodiments an animal has a disruption or deletion in the gene encoding DJ-1. In some embodiments an animal model for PD is used in which manipulation (e.g., knockout or knockdown or other means of reducing function) of one or more mitochondrial respiratory genes elicits a PD phenotype. Knockout of mitochondrial transcription factor A (TFAM) elicits a PD phenotype. Transgenic mice (MitoPark mice) have TFAM selectively knocked out in dopaminergic neurons. The nigral dopamine neurons of MitoPark mice show respiratory chain dysfunction, accompanied by the development of intraneuronal inclusions and eventual cell death. In early adulthood, the MitoPark mice show a slowly progressing loss of motor function that accompanies these cellular changes. Various animal models for PD are described in Harvey B K, Transgenic rodent models of Parkinson's disease. Acta Neurochir Suppl. 2008; 101:89-92. MitoPark mice are further described in Ekstrand M I, et al., Proc Natl Acad Sci USA. 2007; 104(4):1325-30 and Ekstrand M I, et al., Parkinsonism Relat Disord. 2009; 15 Suppl 3:S185-8.

In some embodiments an animal model for GRACILE syndrome is used, e.g., mice generated by introducing the Bcs1l 232A>G mutation (21). Homozygous mutant mice after 3 weeks of age develop striking similarities to the human disease: growth failure, hepatic glycogen depletion, steatosis, fibrosis, and cirrhosis, as well as tubulopathy, complex III deficiency, lactacidosis, and short lifespan.

In some embodiments an animal model expresses a dominant negative form of a mitochondrial protein. In some embodiments the dominant negative form is a variant associated with a mitochondrial disorder. For example, expression of a mutant human ND4 subunit of complex I in the mouse visual system using a recombinant adeno-associated viral vector induces optic neuropathy (Qi, X et al., Invest Ophthalmol Vis Sci. 2007; 48(1):1-10). The mutant form contained an arginine-to-histidine substitution at residue 340 and was equipped with a mitochondrial targeting sequence from ATPc. A recombinant adeno-associated viral vector was used to deliver intraocular injection. The animal may serve as an animal model for LHON or LHON+.

In some embodiments an animal model has a functionally inactivated OPA1 gene. The animal may serve as a model for mitochondrial disorder DOA or DOA+. For example, the animal may have a mutation in the gene encoding OPA1 or may express a short hairpin RNA targeted to OPA1 In some embodiments a mutant mouse carries a Q285X mutation in the Opal gene, resulting in a truncated protein, e.g., as described (Davies, et al, Hum. Molec. Genet. 16: 1307-1318, 2007). The homozygous mutation was embryonic lethal by 13.5 days postcoitum. Fibroblasts from adult heterozygotes showed an increase in mitochondrial fission and fragmentation. In addition, electron microscopy revealed the slow onset of optic nerve degeneration; reduced visual function in heterozygotes was demonstrated by optokinetic drum testing and the circadian running wheel.

In some embodiments an animal model has a mutation in mtDNA of at least some of its mitochondria. In some embodiments an animal model is a transmitochondrial animal, e.g., a transmitochondrial rodent, e.g., a transmitochondrial mouse. In some embodiments a transmitochondrial non-human mammal is generated by the introduction of mitochondria carrying pathogenic mutant mtDNAs into zygotes and/or embryonic stem (ES) cells or iPS cells of the respective species. Transmitochondrial mice useful as models for mitochondrial DNA-based diseases and methods of making thereof have been described (e.g., Nakada K, Hayashi J. Exp Anim. 2011; 60(5):42′-31).

In some embodiments an animal model is depleted of mtDNA in one or more tissues or cell types or throughout the animal. mtDNA depletion may be achieved using various approaches. In some embodiments mtDNA depletion is accomplished by expression of mitochondrially-targeted restriction endonuclease Pst I in one or more tissues or cell types.

Mitochondrial transcription factor A (TFAM) is encoded by the nuclear gene TFAM. The TFAM protein is imported into mitochondria where it binds mtDNA promoters and activates transcription, which is necessary for gene expression and provides the RNA primers necessary for initiation of mtDNA replication by mitochondrial DNA polymerase. TFAM also nonspecifically coats mtDNA and plays an important role in mtDNA maintenance in mammals. TFAM deficiency leads to mtDNA depletion, reduction in levels of mitochondrial transcripts, and severe respiratory chain deficiency. Animals with cell type or tissue-specific TFAM knockouts exhibit such phenotypes in the cells or tissues that have reduced TFAM activity. In some embodiments an animal with a cell type or tissue-specific TFAM knockout is used as a model for a mitochondrial disease, e.g., a mtDNA disorder or disorder characterized by mitochondrial dysfunction in the relevant tissue or cell type. A number of mouse models involving tissue or cell type specific knockout of TFAM are known in the art. As noted above, transgenic mice (MitoPark mice) have TFAM selectively knocked out in dopaminergic neurons. A mouse model engineered to have TFAM deficiency specifically in pancreatic beta-cells faithfully mimics features of mitochondrial diabetes in humans. The mice developed diabetes from the age of 5 weeks and displayed severe mtDNA depletion, deficient oxidative phosphorylation and abnormal appearing mitochondria in islets at the ages of 7-9 weeks. These and other mouse models of mitochondrial dysfunction, e.g., Surfl deficient mice, thymidine kinase 2 (TK2) deficient mice, Deletor mice, may be used in certain embodiments. These and other models are reviewed in Tyynismaal& Suomalainen, EMBO reports (2009) 10, 137-143 and/or Dogan and Trifunovic, Physiol. Res. 60 (Suppl. 1): S61-S70, 2011 and described in further detail in references cited in either of these. Exemplary methods of breeding and genotyping of Tfam conditional knockout mice are described in Ekstrand M, Larsson N G. Methods Mol. Biol. 2002; 197:391-400.

### V. Assessing Mitochondrial Phenotype or Function

Any of a wide variety of methods known in the art may be used to assess mitochondrial phenotype or function in order to, e.g., identify mutant cells that have a mitochondrial phenotype of interest, assess the effect of modulating a gene, assess the effect of an agent, perform a screen, investigate the mechanism of action of an agent, diagnose a mitochondrial disorder, or monitor the effect of therapy. Mitochondrial phenotypes can be assessed, for example, by assaying reporter or sensor molecules, performing assays of amount or activity of mitochondrial enzymes, staining with dyes, measuring electrical changes, among others. Methods such light microscopy, fluorescence microscopy, confocal microscopy, light scattering, light absorbance, spectrophotometry, time resolved fluorescence, scintillation counting, etc., can be used. In some embodiments, detecting a mitochondrial phenotype (or change in cell phenotype) comprises detecting a signal (e.g., produced by a label) indicative of a mitochondrial phenotype (or change in mitochondrial phenotype). Various methods useful for assessing mitochondrial phenotype are described in Leister, Dario; Herrmann, Johannes (Eds.) Mitochondria—Practical Protocols. Methods in Molecular Biology, Vol. 372. Humana Press, 2007; Palmeira, C M and Moreno, A J, et al (eds.) Mitochondrial Bioenergetics: Methods and Protocols. Methods in Molecular Biology. Vol. 810. Humana Press, 2012; Wong, L J C. Mitochondrial Disorders Biochemical and Molecular Analysis. Methods in Molecular Biology. Vol. 837. Humana Press, 2012; and/or Rodenburg, R J, J Inherit Metab Dis. 2011; 34(2):283-92, and references therein. Rodenburg reviews methods that may be used, among other things, in diagnosis of mitochondrial disorders. The method of detection will generally depend on the particular entity or phenomenon being detected. For example, fluorescent or luminescent substances, e.g., fluorescent dyes or proteins may be optically detected by, e.g., fluorescence microscopy, confocal or multiphoton microscopy, flow cytometry, or fluorescent plate reader. In some embodiments a charge coupled device (CCD) camera is used. Data may be acquired at a single time point or multiple time points. Appropriate software may be used for data collection, analysis, display, and/or storage. In some embodiments a mitochondrial phenotype is assessed in living cells. In some embodiments a mitochondrial phenotype is assessed in isolated mitochondria. For example, once a gene that affects mitochondrial phenotype has been identified in a screen using living cells, the effect of modulating the gene may be assessed in mitochondria isolated from cells in which the gene has been mutated or modulated. In some embodiments isolated mitochondria are obtained and contacted. with an agent, and the effect of the agent on at least one mitochondrial phenotype or function is assessed.

Many mitochondrial phenotypes and functions can be assessed using various fluorescent small molecules, sometimes termed “dyes”. A range of mitochondrion-selective dyes ale available that can be used to monitor mitochondrial morphology and/or function. Such dyes are often lipophilic cationic compounds that equilibrate across membranes and accumulate in the mitochondrial membrane matrix space in approximately inverse proportion to Δψₘ, such that a more negative Δψₘ will accumulate more dye, and vice versa. A number of such dyes are described in The Molecular Probes® Handbook—A Guide to Fluorescent Probes and Labeling Technologies, 11^{th} ed (Life Technologies Corp., Carlsbad, Calif.). Such dyes can be used to assess mitochondrial activity, localization, or number or to monitor the effects of agents or conditions. In some embodiments an assay based at least in part on fluorescence resonance energy transfer (FRET) or bioluminescence resonance energy transfer (BRET) is used. In some embodiments cells are contacted with a first dye or protein that labels mitochondria and a second dye or protein that is sensitive to a particular analyte (e.g., protons, oxygen, calcium ions, ATP, ROS). Mitochondria are detected or imaged by detecting by detecting the first dye, and the analyte is detected, quantified, or imaged by detecting the second dye. In some embodiments this information is used to specifically determine the level or distribution of the analyte in mitochondria.

MitoTracker® probes (Life Technologies) are cell-permeant mitochondrion-selective fluorescent dyes that contain a mildly thiol-reactive chloromethyl moiety that can be used to label mitochondria. The probe passively diffuses across the plasma membrane and accumulates in active mitochondria. In some embodiments, once mitochondria are labeled, the cells can be treated with aldehyde-based fixatives to allow further processing of the sample. Endogenously biotinylated proteins in mammalian cells are present almost exclusively in mitochondria, where biotin synthesis occurs. In some embodiments an avidin or streptavidin or derivative thereof is used to label mitochondria. For example, a fluorophore- or enzyme-labeled avidin or streptavidin derivative may be used. In some embodiments mitochondria are labeled using a reporter protein comprising a mitochondrial targeting sequence. In some embodiments the protein is expressed intracellularly and localizes to mitochondria. In some embodiments the reporter protein is introduced into cells from the exterior and localizes to mitochondria. In some embodiments the reporter protein is pH-sensitive or sensitive to an analyte such as Ca²⁺ or a small organic molecule.

Reactive oxygen species (ROS) can be detected, e.g., in live cells, using ROS-sensitive dyes such as 2′,7′-dichlorodihydrofluorescein diacetate (DCF-DA) or MitoSOX Red mitochondrial superoxide indicator (Life Technologies) or using electron spin resonance. MitoSOX Red is a cationic derivative of dihydroethidum that can be used to detect superoxide.

Inner mitochondrial membrane potential can be assessed using fluorescent dyes. For example, JC-1 and JC-9 are dual-emission potential-sensitive probes (Life Technologies). JC-1 probe (5,5′,6,6′-tetrachloro-1,1′,3,3′-tetraethylbenzimidazolylcarbocyanine iodide) exists as a monomer at low concentrations or at low membrane potential. However, at higher concentrations or higher potentials, JC-1 forms red-fluorescent “J-aggregates” that exhibit a broad excitation spectrum and an emission maximum at ˜590 nm. The emission of this dye can be used as a sensitive measure of mitochondrial membrane potential. In some embodiments the ratio of red-to-green JC-1 fluorescence is used as a measure of membrane potential.

Mitochondrial permeability transition pore opening can be assessed using a Image-iT LIVE Mitochondrial Transition Pore Assay or MitoProbe Transition Pore Assay Kit (Life Technologies) or an assay based on the same principle is used. This assay employs the acetoxymethyl (AM) ester of calcein, a colorless and nonfluorescent esterase substrate, and CoCl₂, a quencher of calcein fluorescence, to selectively label mitochondria. Cells are loaded with calcein AM, which passively diffuses into the cells and accumulates in cytosolic compartments, including the mitochondria. Once inside cells, calcein AM is cleaved by intracellular esterases to liberate the polar fluorescent dye calcein, which does not cross the mitochondrial or plasma membranes in appreciable amounts over relatively short periods of time. The fluorescence from cytosolic calcein is quenched by the addition of CoCl₂, while the fluorescence from the mitochondrial calcein is maintained in the absence of mitochondrial transition pore opening. As a control, cells that have been loaded with calcein AM and CoCl₂ can also be treated with a Ca2+ ionophore such as ionomycin to allow entry of excess Ca2+ into the cells, which triggers mitochondrial pore opening and subsequent loss of mitochondrial calcein fluorescence.

Intramitochondrial pH can assessed using a pH-sensitive reporter protein or pH-sensitive dye such as a seminaphtharhodafluor (SNARF) dye, e.g., SNARF-1 or genetically encoded pH-sensitive proteins such as pHluorins, deGFPs, pHlameleons (see, e.g., Esposito, A, et al., Biochemistry (2008); 47 (49): 13115-13126 and references therein. Such agents, for example, exhibit a spectral shift upon changes in pH.

Other useful mitochondrial-selective dyes include various rhodamines and rosamines (e.g, such as rhodamine 123, rhodamine 6G, tetramethylrhodamine methyl and ethyl esters, and tetramethylrosamin), styryl dyes DASPMI, and DASPEI, and nonyl acridine orange. For example, in some embodiments tetramethyl rhodamine methyl ester (TMRM) or tetramethylrhodamine ethyl ester (TMRE) is used to assess inner mitochondrial membrane potential.

Individual mitochondrial enzymes can be evaluated by spectrophotometric and/or radiochemical assays or by immunologically-based methods such as Western blot, immunohistochemistry, ELISA assays, etc. Such studies may, for example, assess OXPHOS activity or the amount, activity, or substrate affinity of one or more of complexes I, II, III, IV, or V or component(s) thereof. Assays that determine the amount of one or more OXPHOS complexes may be performed, for example, using Blue Native gel electrophoresis followed by Western blot analysis. In some embodiments an assay to quantify OXPHOS enzyme activit(ies) is based on spectrophotometry. Coenzyme Q can also be measured directly by HPLC in samples of cultured cells, muscle, blood, or other sample types, e.g., for diagnostic purposes. Activities of pyruvate dehydrogenase or TCA cycle enzymes can be assessed. Functional activities or processes such as ATP synthesis, mitochondrial oxygen consumption, and substrate oxidation rates can be measured in cells. Representative examples of assays of mitochondrial phenotype or function, including assays for activity or amount of complexes I-V are described in Rodenburg, R J, cited above, and references therein. In some embodiments fibroblasts or muscle biopsy samples may be used, e.g., when such assays are conducted for diagnostic purposes

In some embodiments cells are permeabilized, e.g., to allow substrates, ADP, candidate agents, and/or other assay ingredients to reach the mitochondria and enter the matrix via mitochondrial transporters. In some embodiments cells are permeabilized using a detergent (e.g., digitonon, saponin) or electrically.

Several techniques are available to measure oxygen consumption, including polarography with oxygen electrodes or fluorescent/luminescent probes. In some embodiments mitochondrial oxygen consumption is measured using a Clark-type electrode. In some embodiments mitochondrial respiration is tested using a respirometry platform such as the XF24 Extracellular Flux Analyzer (Seahorse Bioscience, Billerica, Mass.). The XF24 system can be used to measure in real time the uptake and excretion of metabolic end products. For example, it may be used to measure the extracellular flux changes of oxygen and protons in the media immediately surrounding adherent cells cultured in a XF24-well microplate format. The XF24 system is described in, e.g., Wu M, et al. Am J Physiol Cell Physiol, 2007; 292: C125-136. The extent to which mitochondrial respiration contributes to total cellular oxygen consumption can be determined by using an inhibitor of the mitochondrial respiratory chain, such as rotenone. In some embodiments the rotenone-sensitive oxygen consumption rate can be used to specifically identify respiration in the mitochondria. In some embodiments the rotenone-resistant rate reflects the nonmitochondrial respiration rate. XF measurements are nondestructive; thus the metabolic rate of the same cell population can be measured repeatedly over time, while up to four different testing agents can be injected sequentially or simultaneously into each well. Upon completion of an XF assay, other types of biological assays such as cell viability can be performed on the same cell population. In some embodiments an XF24 system is modified to facilitate its use with non-adherent cells. For example, nonadherent cells may be placed in a depression in the middle of the well that keeps them in close proximity to the probe head and a screen may be used to protect them from turbulence (see Wikstrom, J D, et al., PLoS One. 2012; 7(5):e33023).

The mitochondrial energy generating system can be analyzed in a variety of ways. In some embodiments, by using ¹⁴C labeled pyruvate, malate, or succinate, the conversion rates of these substrates can be determined by measuring the amounts of released ¹⁴CO₂ as parameters for the overall capacity of the mitochondrial energy generating system. The oxygen consumption rate can be assessed in the presence of different substrates, e.g., pyruvate+malate, and can be measured by respirometry or using fluorescent probes, e.g., as described above. The rate of synthesis of ATP in the presence of different mitochondrial substrates is also representative for the capacity of the mitochondrial energy-generating system. In some embodiments cellular ATP level is measured using the CellTiter-Glo Luminescent Cell Viability Assay (Promega, Madison, Wis.).

In some embodiments cell viability is assessed. Cell viability may be assessed on the same population of cells or on a population of cells subjected to the same conditions or agents. In some embodiments cell viability is measured using an assay that does not rely on ATP measurement. For example, a calcein AM assay or trypan blue exclusion assay may be used.

In some embodiments apoptosis is assessed, e.g., using TUNEL or detecting a marker of apoptosis such as annexin V or phosphatidylserine on the cell surface.

In some embodiments mtDNA is analyzed, e.g., to evaluate a subject suspected of having a mitochondrial disorder or to assess the effect of an agent on a cell. In some embodiments mtDNA is analyzed by sequencing. Complete mitochondrial DNA of humans and a variety of other mammals (and other organisms) has been sequenced, and sequences are available in public databases. In some embodiments, a human mitochondrial DNA sequence is the Revised Cambridge Reference Sequence (rCRS) of the Human Mitochondrial DNA. The rCRS sequence has been assigned number NC_{—}012920 gi:251831106 in GenBank. MITOMAP (www.mitomap.org) is a human mitochondrial genome database that provides, among other things, a compendium of polymorphisms and mutations of the human mitochondrial DNA. In some embodiments a partial or complete mtDNA sequence is compared with the rCRS sequence and/or with sequences available in MITOMAP. In some embodiments comparative genomic hybridization (CGH), e.g., array comparative genomic hybridization (aCGH) is used.

### VI. Mitochondrial Disorders

In some embodiments, the present disclosure provides methods of treating mitochondrial disorders. In some embodiments the methods comprise administering a modulator of a target gene identified as described herein to a subject in need of treatment for a mitochondrial disorder. In some embodiments the modulator is an inhibitor of a gene whose mutation results in resistance to a mitochondrial poison.

Mitochondrial dysfunction is involved in a wide range of disorders. In some embodiments a mitochondrial disorder arises at least in part from a mutation in a gene encoding a mitochondrial protein. In some embodiments a mitochondrial disorder arises at least in part from a mutation in nuclear DNA. In some embodiments a mitochondrial disorder arises at least in part from a mutation in a nuclear gene or from a deficiency or dysfunction of a gene product of a nuclear gene. In some embodiments the nuclear gene encodes a mitochondrial protein. In some embodiments the nuclear gene encodes a protein that regulates expression, localization, post-translational modification, activity, or assembly of a mitochondrial protein or complex. Certain mitochondrial disorders and genes, mutation in which is associated with a mitochondrial disorder, are discussed in this section. A compendium of numerous human genes and genetic phenotypes that occur in humans, including many associated with mitochondrial disorders, is provided in McKusick V. A. (1998) Mendelian Inheritance in Man. A Catalog of Human Genes and Genetic Disorders, 12th Edn. The Johns Hopkins University Press, Baltimore, Md. and its online updated version Online Mendelian inheritance in Man (OMIM), available at the National Center for Biotechnology Information (NCBI) website at http://www.ncbi.nlm.nih.gov/omim. Numbers assigned to various disorders and gene names in OMIM are sometimes provided herein in parentheses. In some embodiments a mitochondrial disorder is characterized by mitochondrial dysfunction that is not known to be attributable to mutation of a particular gene or genes.

In some embodiments a mitochondrial disorder is caused at least in part by a mutation in mtDNA. The mutation may affect some or all of the subject's mitochondria. Homoplasmy refers to a situation in which all mtDNAs in the system under discussion (e.g. a cell, tissue or organism) are genetically identical, e.g., all having the same normal sequence or all having a sequence containing the same one or more mutations. Heteroplasmy refers to a situation in which two or more mtDNA genotypes coexist in the system under discussion (e.g. a cell, tissue or organism), e.g., These terms are typically used in the context of mitochondrial disorders to distinguish between disorders in which all mtDNA are mutated (homoplasmic disorders) or in which some mtDNA are mutated while others are not (heteroplasmy). Mutations that arise in mtDNA include mtDNA rearrangements such as deletions, inversions or duplications, point mutations, or copy number depletion. They may be sporadic, maternally inherited, or Mendelian in character. Primary mtDNA mutations occur sporadically or exhibit maternal inheritance. In some embodiments mtDNA copy number or degree of heteroplasmy is assessed.

In some embodiments a mitochondrial disorder, e.g., a mitochondrial disorder arising at least in part from a mutation in mtDNA is maternally inherited. In some embodiments a mitochondrial disorders is inherited in a Mendelian pattern. In some embodiments a mitochondrial disorder arises sporadically, i.e., it is not inherited from a parent. A mutation may be in the germ line or somatic.

In some embodiments a mitochondrial disorder is caused at least in part by a mutation in a nuclear or mitochondrial gene that encodes a component of complex I, II, III, IV, or V. In some embodiments a mitochondrial disorder is caused at least in part by a mutation in a nuclear or mitochondrial gene that encodes an assembly factor for complex I, II, IV, or V. In some embodiments an assembly factor (typically a protein) is involved in transcription and/or translation of a subunit of complex I-V (e.g., a mitochondrion-encoded subunit), processing of a preprotein, membrane insertion, or cofactor biosynthesis or transport or incorporation.

In general, a mutation that causes mitochondrial disorder may comprise any type of alteration in DNA sequence, relative to a normal sequence, in various embodiments. In general, certain mutations may result in abnormal expression level and/or activity of a gene product. In some embodiments a mutation results in abnormal expression level and/or activity of a gene product that is a component of a metabolic pathway as compared with a level of expression or activity. In general, a mutation may affect any region of a gene. In some embodiments a mutation is in a region of a gene that is transcribed. In some embodiments a mutation results in an alteration in an encoded polypeptide sequence, as compared to a normal polypeptide sequence. In some embodiments a mutation is a nonsense mutation, missense mutation, frameshift mutation, or a mutation that impairs proper splicing (e.g., a splice site mutation). In some embodiments a mutation is in a regulatory region of a gene. In some embodiments a mutation results in abnormal expression of the gene containing the mutation. For example, a mutation may result in increased or decreased level of a gene product in at least some cells, as compared with a normal level of the gene product. In some embodiments, a mutation results in a deficiency of functional gene product. For example, a mutation may result in an alteration in an encoded gene product that causes the gene product to have reduced activity relative to a normal gene product or to interfere with activity of a normal gene product encoded by another allele of the gene in a diploid organism. A mutation in a regulatory region of a gene may result in a decreased synthesis of a gene product encoded by the gene. A normal nucleic acid (DNA, RNA) or polypeptide sequence may be, e.g., (i) a nucleic acid or polypeptide sequence in which the nucleotide or amino acid, respectively, present at each position in the sequence has a prevalence of at least 1% in a population or (ii) a nucleic acid or polypeptide sequence whose expression and activity do not differ detectably from that of the nucleic acid or polypeptide sequence of (i). A normal sequence may be, e.g., the most common sequence present in a population, a reference sequence (e.g., an NCBI RefSeq sequence, or a UniProt reference sequence), or a sequence in which the nucleotide or amino acid present at each position of the sequence is the most common nucleotide or amino acid present at that position in a population. In some embodiments a mutation has a prevalence of less than 0.5%, less than 0.1%, less than 0.05%, or less than 0.001% in a population. In some embodiments a mutation may result in an expression level or activity that lies outside a normal range for expression level or activity of a gene product, i.e., below the lower limit of normal or above the upper limit of normal. A normal range may be, e.g., a range that is accepted in the art as normal. In some embodiments a normal range may be defined as a range that would encompass at least 95% of values measured in a population. In some embodiments, a “population” may be the general population, e.g., of a city, state, country or other region. In some embodiments a population may consist of individuals without any known condition that directly affects the range being established. A normal range or normal sequence may be obtained by evaluating a representative sample of a population.

In some embodiments a mitochondrial disorder is associated with a mutation in a gene that does not (or is not known to) encode a mitochondrial protein. The mutation may result in biochemical abnormalities in a cell, which in turn result in mitochondrial dysfunction.

In some embodiments a mitochondrial disorder is associated with an abnormally increased or decreased level of a product, metabolite, cofactor, or intermediate normally found in or produced at least in part by mitochondria.

In some embodiments a mitochondrial disorder is characterized by cell or tissue loss (cell death). In some embodiments, cell or tissue loss occurs at least in part due to apoptosis. Apoptosis is a process of programmed cell death that may occur in multicellular organisms, in which cells activate an intracellular death program and kill themselves in a controlled manner. Biochemical events occurring in apoptosis lead to characteristic changes in cell morphology and result in cell death. Changes typically occurring in apoptosis include blebbing, cell shrinkage, nuclear fragmentation, chromatin condensation, and chromosomal DNA fragmentation. Cells undergoing apoptosis may also display characteristic markers on their cell surface, such as phosphatidylserine (PS). Apoptotic cells can be detected in a variety of ways, such as by detecting new DNA ends (e.g., using TUNEL) or by detecting markers such as PS (which can be detected using labeled Annexin V). Apoptosis can be triggered by extracellular signals (the extrinsic pathway of apoptosis) via activation of cell surface “death receptors” such as Fas or from inside the cell (the intrinsic pathway of apoptosis). The intrinsic pathway is initiated by various stimuli that cause changes in the inner mitochondrial membrane, resulting in opening of the mitochondrial permeability transition (MPT) pore, loss of the mitochondrial transmembrane potential, and release into the cytosol of various mitochondrial proteins that normally are present in the intermembrane space. For example, release of cytochrome c from mitochondria can trigger apoptosis via the intrinsic pathway. The control and regulation of these apoptotic mitochondrial events occurs at least in part through members of the Bcl-2 family of proteins. In some instances, the extrinsic pathway relies on recruitment of the intrinsic pathway to cause apoptosis. Both the extrinsic and intrinsic pathways lead to activation of a proteolytic cascade involving caspase proteins. The pathway is initiated by cleavage of one or more “initiator” caspases, which cleave and activate downstream “executioner’ caspases. Executioner caspases activate other executioner caspases as well as target proteins in the cell, leading to the biochemical and morphological changes outlined above. Further information regarding apoptosis and the role of mitochondria in apoptosis may be found in, e.g., Wang, C. and Youle, R J, et al., Annu. Rev. Genet. 2009. 43:95-118, Tait and Green, Nat Rev Mol Cell Biol 11:621-632, 2010, and/or references cited in either of these.

In some embodiments a mitochondrial disorder characterized by cell or tissue loss is Parkinson's disease (PD), which results from dysfunction and/or death of dopaminergic neurons having cell bodies in the substantia nigra, often accompanied by accompanied by intracellular aggregates positive for α-synuclein (α-syn). PD is a progressive motor disease typically characterized by tremor, rigidity (stiffness), and bradykinesia (slowness of movement). Without wishing to be bound by any theory, many of the major molecular events associated with PD, such as α-syn buildup and decreased activity of Parkin (mutations in which are known to cause a form of Parkinson's disease), may exert their effects at least in part through mitochondrial dysfunction. Indeed, studies have found that high levels of α-syn inhibit the activity of complex I (Devi, L., et al. Mitochondrial Import and Accumulation of α-Synuclein Impair Complex I in Human Dopaminergic Neuronal Cultures and Parkinson Disease Brain. Journal of Biological Chemistry 283, 9089-9100 (2008) and increase ROS levels in DA neurons (Parihar, M., et al., Mitochondrial association of alpha-synuclein causes oxidative stress. Cellular and Molecular Life Sciences 65, 1272-1284 (2008) while loss of function of Parkin in both mice (Palacino, J. et al., Mitochondrial Dysfunction and Oxidative Damage in parkin-deficient Mice. Journal of Biological Chemistry 279, 18614-18622 (2004) and humans (Müftüoglu, M., et al., Mitochondrial complex I and IV activities in leukocytes from patients with parkin mutations. Movement Disorders 19, 544-548 (2004) leads to a decrease in the respiratory capacity of mitochondria. Treating mice with mitochondrial complex-I inhibitors, such as rotenone or MPTP, results in a Parkinsonian phenotype, with loss of dopaminergic neurons in the substantia nigra accompanied by intracellular aggregates positive for α-synuclein (α-syn) and ubiquitin (Betarbet, R., et al., Chronic systemic pesticide exposure reproduces features of Parkinson's disease. Nat Neurosci 3, 1301-1306 (2000); Fomai, F., et al., Parkinson-like syndrome induced by continuous MPTP infusion: Convergent roles of the ubiquitin-proteasome system and α-synuclein. PNAS US 102, 3413-3418 (2005), providing further evidence of the connection between PD and mitochondrial dysfunction. Other mitochondrial disorders characterized by cell or tissue loss (e.g., due to apoptosis) include Huntington's disease, mitochondrial disorders involving optic atrophy (e.g., retinal ganglion cell death) such as Leber's hereditary optic neuropathy, dominant autosomal-dominant optic atrophy, Charcot-Marie-Tooth disease type 2 (CMT2A), and glaucoma, among others.

In some embodiments a mitochondrial disorder is any disorder characterized by hypoxia, ischemia, and/or ischemia-reperfusion injury. Ischemia-reperfusion injury refers to tissue damage caused when blood supply returns to a tissue after a period of ischemia or lack of oxygen. The absence of oxygen and nutrients from blood during the ischemic period creates a condition in which the restoration of circulation results in tissue damage as a result of factors such as oxidative stress and inflammation. For example, ischemia-reperfusion can result in increased production of free radicals and reactive oxygen species that damage cells, in some cases resulting in cell or tissue loss. In some embodiments a disorder characterized by hypoxia, ischemia, and/or ischemia-reperfusion injury is stroke (e.g., ischemic stroke), myocardial infarction, or trauma (e.g., brain trauma). In some embodiments hypoxia, ischemia, and/or ischemia-reperfusion injury occurs as a result of surgery, e.g., cardiopulmonary bypass surgery, coronary artery bypass graft surgery (CABG), organ transplant, aneurysm repair, plastic surgery, or flap surgery. In some embodiments ischemia/reperfusion injury damages the heart, lung, kidney, and/or brain.

In some embodiments a mitochondrial disorder is an orphan disease. In some embodiments the disorder affects about 1 in 1,500 people, about 1 in 2,000 people, about 1 in 2,500 people, or less. In some embodiments a mitochondrial disorder is a life-threatening or chronically debilitating disease in at least some subjects, e.g., at least 10%, 25%, 50%, 75%, 90%, or more of subjects diagnosed with the disease.

In some embodiments a mitochondrial disorder is a neurodegenerative disorder. In some embodiments a neurodegenerative disorder affects one or more components of the central nervous system (brain, spinal cord, optic nerve, and/or retina). Neurodegenerative disorders that affect the CNS include, e.g., Alzheimer's disease, Parkinson's disease, Huntington's disease, Friedrich's ataxia, and amyotrophic lateral sclerosis (ALS). In some embodiments a neurodegenerative disorder is a movement disorder. In some embodiments a neurodegenerative disorder affects one or more basal ganglia. In some embodiments a neurodegenerative disorder affects the peripheral nervous system. In some embodiments a neurodegenerative disorder affects motor neurons.

In some embodiments a mitochondrial disorder is a neurodevelopmental disorder such as autism spectrum disorder (ASD) or Rett syndrome. Several lines of evidence implicate a role for mitochondrial dysfunction in ASD (reviewed in Dhillon, S., et al. Curr Genomics.; 12(5): 322-332; Haas, R H., Dev Disabil Res Rev. 2010 June; 16(2):144-53.).

Rett syndrome is caused mainly by mutations in the gene encoding methyl-CpG binding protein 2 (MeCP2), a transcriptional repressor involved in chromatin remodeling and the modulation of RNA splicing (Chahrour M and Zoghbi H Y. Neuron. 2007; 56(3):422-37). Morphological abnormalities of mitochondria, functional defects of the mitochondrial respiratory chain, and evidence of increased oxidative stress have been observed in Rett patients. MeCP2 deficiency has been associated with defects in mitochondrial enzyme expression and activity (Gibson, J H, et al. BMC Neuroscience 2010, 11:53 and references therein).

In some embodiments a mitochondrial disorder is a psychiatric disorder such as schizophrenia or bipolar disorder. Several lines of evidence implicate a role for mitochondrial dysfunction in these disorders (Clay, H B, et al., Int J Dev Neurosci. 2011; 29(3):311-24).

In some embodiments a mitochondrial disorder is a hepatic disorder, which term is used herein to refer to any disorder that affects the structure or function of the liver, e.g., any disorder in which death or dyfunction of liver cells (e.g., hepatocytes) occurs. Mitochondrial dysfunction is a major mechanism of liver injury and plays a role several acute and chronic hepatic disorders such as alcoholic and non-alcoholic fatty liver disease (NAFLD), drug-induced steatohepatitis, viral hepatitis, biliary cirrhosis, ischemia/reperfusion injury, and transplant rejection. Hepatic disorders include those induced by drugs (e.g., acetaminophen), toxins (e.g., alcohol), and hepatotropic viruses (e.g, hepatitis B or C virus, among others). Hepatic inflammation and fibrosis are a prominent component of many chronic liver disorders. Apoptosis is implicated as playing a significant role in promoting inflammation and/or fibrosis, potentially resulting in permanent liver damage (Malhi, H, et al., Physiol Rev 90: 1165-1194, 2010).

In some embodiments a mitochondrial disorder is a muscular disorder, also termed a myopathy, e.g., a myodegenerative disorder, i.e., a disorder affecting development, structure, or function of one or more muscles or muscle types (e.g., cardiac, smooth, striated muscle). In some embodiments a muscular disorder is chronic progressive external ophthalmoplegia (CPEO), a disorder that causes extraocular muscle weakness. CPEO may occur as part of a syndrome involving more than one part of the body, such as Kearns-Sayre syndrome (KSS), also known as oculocraniosomatic disease or oculocraniosomatic neuromuscular disease with ragged red fibers. KSS involves a triad of CPEO, bilateral pigmentary retinopathy, and cardiac conduction abnormalities. Other areas of involvement can include cerebellar ataxia, proximal muscle weakness, deafness, diabetes mellitus, growth hormone deficiency, hypoparathyroidism, or other endocrinopathies.

In some embodiments a mitochondrial disorder is an ocular disorder, i.e., a disorder affecting eye development or one or more functions or structures of the eye. In some embodiments a mitochondrial disorder affects a sensory structure such as the optic nerve or retina or a portion thereof. In some embodiments an ocular disorder is characterized by death of retinal ganglion cells (RGCs). In some embodiments an ocular disorder is Leber optic atrophy, also called Leber's hereditary optic neuropathy (LHON; OMIM #535000). LHON is usually due to one of three pathogenic mitochondrial DNA (mtDNA) point mutations. These mutations are at nucleotide positions 11778 G to A, 3460 G to A and 14484 T to C, respectively in the ND4, ND1, and ND6 subunit genes of complex I. Mutations in genes encoding components of complex III or IV have also been implicated. LHON can also be associated with minor neurological abnormalities, in which case it may be referred to as Leber's “plus”. Clinical manifestations may include postural tremor, motor disorder, Parkinsonism with dystonia, peripheral neuropathy, multiple sclerosis-like syndrome, cerebellar ataxia, anarthria, dystonia, spasticity, or mild encephalopathy. The 11778 mitochondrial DNA mutation has been associated with familial multisystem degeneration with parkinsonism (Simon, D. K., et al. Neurology 53, 1787-1793 (1999). In some embodiments an ocular disorder is autosomal-dominant optic atrophy (DOA; OMIM #165500). LHON and DOA share pathological similarities, marked by the selective loss of retinal ganglion cells (RGCs) and the early involvement of the papillomacular bundle. In DOA, the majority of affected families harbour mutations in the OPA1 gene. The OPA1 gene (605290) encodes a protein that localizes to the inner mitochondrial membrane and regulates several important cellular processes including stability of the mitochondrial network, mitochondrial bioenergetic output, and sequestration of proapoptotic cytochrome c oxidase molecules within the mitochondrial cristae spaces (Yu-Wai-Man et al., 2010). Optic atrophy 1 (OMIM #165500) and syndromic optic atrophy, also known as DOA+ syndrome (OMIM #125250), also caused by heterozygous mutations in the OPA1 gene, is a neurologic disorder characterized most commonly by an insidious onset of visual loss and sensorineural hearing loss in childhood with variable presentation of other clinical manifestations. A predominantly complex I respiratory chain defect has been identified confirming that optic nerve degeneration in LHON and DOA is due at least in part to disturbed mitochondrial function. In some embodiments an ocular disorder is diabetic retinopathy, glaucoma, or age-related macular degeneration. As noted above, various mitochondrial myopathies affect the eye (e.g., extraocular muscles) as well.

In some embodiments a mitochondrial disorder is a metabolic disorder. In some embodiments a metabolic disorder is diabetes (e.g., type I or type II diabetes mellitus), impaired glucose tolerance, impaired fasting glucose, insulin resistance, or obesity.

In some embodiments a mitochondrial disorder is MELAS syndrome (“mitochondrial encephalopathy, lactic acidosis, and stroke”; OMIM #540000); MERFF syndrome (“myoclonic epilepsy ragged red fiber syndrome”); NARP (neuropathy, ataxia, retinitis pigmentosa); MNGIE (myopathy and external ophthalmoplegia, neuropathy, gastrointestinal encephalopathy); Kearns-Sayre disease; Pearson's syndrome; diabetes mellitus and deafness (DAD) or maternally inherited diabetes and deafness (MIDD),

In some embodiments a mitochondrial disorder is caused by a mutation in the BCS1L gene. For example, GRACILE syndrome (growth retardation, aminoaciduria, cholestasis, iron overload, lactic acidosis, and early death; also referred to as Finnish lethal neonatal metabolic syndrome (FLNMS); lactic acidosis Finnish, with hepatic hemosiderosis; and Fellman syndrome; OMIM #603358) is caused by a homozygous mutation in the BCS gene (232A>G), resulting in a Ser78 to Gly mutation (Visapaa, I, et al., GRACILE syndrome, a lethal metabolic disorder with iron overload, is caused by a point mutation in BCS1L. Am J Hum Genet. 71, 863-876 (2002)). Autosomal recessive mitochondrial complex III deficiency (OMIM #124000), which can also be caused by mutations in the BCS1L gene, is a severe multisystem disorder with onset at birth of lactic acidosis, hypotonia, hypoglycemia, failure to thrive, encephalopathy, and delayed psychomotor development. Bjornstad syndrome (OMIM #262000) is an autosomal recessive disorder characterized by sensorineural hearing loss and pili torti that is caused by mutations mutations in the BCS1L gene. Mitochondrial complex III deficiency may also arise due to mutations in the UQCRQ, UQCRB, or TTC19 genes. An out-of-frame cytochrome b gene deletion in a patient with parkinsonism was found to be associated with impaired complex III assembly and an increase in free radical production (Rana, M., et al., Annals of Neurology 48, 774-781 (2000).

NARP (OMIM #551500) is caused by mutations in the mitochondrial gene MT-ATP6 gene, which encodes the MT-ATP6 protein, a subunit of ATP synthase. Neuropathy, ataxia, and retinitis pigmentosa (NARP) is a condition that causes a variety of signs and symptoms chiefly affecting the nervous system. Beginning in childhood or early adulthood, most people with NARP experience numbness, tingling, or pain in the arms and legs (sensory neuropathy); muscle weakness; and problems with balance and coordination (ataxia). Many affected individuals also have vision loss caused by changes in the light-sensitive tissue that lines the back of the eye (the retina). In some cases, the vision loss results from a condition called retinitis pigmentosa. This eye disease causes the light-sensing cells of the retina gradually to deteriorate. Learning disabilities and developmental delays are often seen in children with NARP, and older individuals with this condition may experience a loss of intellectual function (dementia). Other features of NARP include seizures, hearing loss, and abnormalities of the electrical signals that control the heartbeat (cardiac conduction defects). Most individuals with NARP have a specific MT-ATP6 mutation in 70% to 90% of their mitochondria. When this mutation is present in a higher percentage of a person's mitochondria, e.g., greater than 90% to 95%, it can cause maternally inherited Leigh syndrome.

Leigh syndrome (OMIM #256000) refers to a group of frequently lethal early-onset progressive neurodegenerative disorders with a characteristic neuropathology consisting of focal, bilateral lesions in one or more areas of the central nervous system, including the brainstem, thalamus, basal ganglia, cerebellum, and spinal cord. The lesions are areas of demyelination, gliosis, necrosis, spongiosis, or capillary proliferation. Clinical symptoms depend on which areas of the central nervous system are involved. The most common underlying cause is a defect in oxidative phosphorylation. Mutations have been identified in both nuclear- and mitochondrial-encoded genes involved in energy metabolism, including genes encoding subunits of mitochondrial respiratory chain complexes I, II, III, IV, and V, assembly factors, and components of the pyruvate dehydrogenase complex. Mutations associated with Leigh syndrome have been identified in various complex I genes and assembly) factors including mitochondrial-encoded MTND2 (516001), MTND3 (516002), MTND5 (516005), and MTND6 (516006), the nuclear-encoded NDUFS1 (157655), NDUFS3 (603846), NDUFS4 (602694), NDUFS7 (601825), NDUFS8 (602141), NDUFA2 (602137), NDUFA9 (603834), NDUFA10 (603835), NDUFA12 (614530), C8ORF38 (612392), and C20ORF7 (612360), the complex I assembly factor NDUFAF2 (609653); a complex II gene: the flavoprotein subunit A (SDHA; 600857); the BCS1L gene (603647), which is involved in the assembly of complex III; complex IV genes and assembly factors including MTCO3 (516050) and nuclear-encoded COX10 (602125), COX15 (603646), SCO2 (604272), and in SURF1 (185620), which is involved in the assembly of complex IV, and TACO1 (612958); a complex V gene: the mitochondrial-encoded MTATP6; mitochondrial tRNA proteins MTTV (590105), MTTK (590060), MTTW (590095), and MTTL1 (590050); components of the pyruvate dehydrogenase complex (e.g., DLD and PDHA1; also called X-linked Leigh syndrome, OMIM #308930). The French Canadian (or Saguenay-Lac-Saint-Jean) type of Leigh syndrome with COX deficiency (LSFC; OMIM #220111) is caused by mutation in the LRPPRC gene (607544). Deficiency of coenzyme Q10 (caused by homozygous or compound heterozygous mutation in the COQ2 gene; OMIM #607426) can present as Leigh syndrome.

Mohr Tranebjaerg syndrome (OMIM #304700) is caused by mutations in the TIMM8A (DDP) gene. Mutation of the same gene has been found as the cause of Jensen syndrome, also called opticoacoustic nerve atrophy with dementia (OMIM #311150). The demonstrated involvement of DDP in the import of mitochondrial proteins implies that the underlying defect of the Mohr-Tranebjaerg syndrome is a defect in mitochondrial oxidative phosphorylation (OXPHOS), specifically due to deficiencies in carrier proteins. This is consistent with the fact that phenotypes associated with the systemic OXPHOS defects resulting from mutations in mitochondrial DNA produce a variety of clinical symptoms that overlap with those of the Mohr-Tranebjaerg syndrome.

In some embodiments a mitochondrial disorder is associated with acute or chronic exposure to a pesticide (e.g., a herbicide, insecticide, or fungicide) or other agent that acts as a mitochondrial poison. In some embodiments pesticide exposure causes damage to and/or death of dopaminergic neurons, leading to Parkinson's disease. In some embodiments a pesticide is maneb (MB), a Mn-containing ethylene-bis-dithiocarbamate (EBDC) fungicide or mancozeb (MZ), a EBDC fungicide that is structurally similar to MB but contains both Zn and Mn. Both of these agents have been associated with neurotoxicity and mitochondrial dysfunction (Domico L M et al. Neurotoxicology. 27(5):816-25). In some embodiments a pesticide is fenpyroximate, fenazaquin, or tebunfenpyrad. In some embodiments the pesticide is paraquat. In some embodiments a pesticide is a dithiocarbamate fungicide such as ziram. In some embodiments a pesticide inhibits one or more OXPHOS components. In some embodiments a pesticide inhibits complex I. In some embodiments a mitochondrial disorder is associated with treatment with a therapeutic agent that may act as a mitochondrial poison such as an NRTI, NtRTI, or NNRTI.

In some embodiments a mitochondrial disorder arises at least in part from a mutation in mitochondrial DNA (mtDNA) or from a deficiency or dysfunction of a gene product of a mitochondrial gene or from damage to mtDNA arising from any cause. In some embodiments a mutation is a point mutation. In some embodiments a point mutation alters the sequence of an encoded protein. In some embodiments a mutation is a deletion. A deletion may remove at least a portion of a coding region or promoter of one or more genes. In some embodiments a mutation is a rearrangement or duplication. In some embodiments multiple mutations are present. Diseases caused by mutation in mtDNA include Kearns-Sayre syndrome, MELAS syndrome, MERRF, Leber's hereditary optic neuropathy, Pearson's syndrome, and progressive external ophthalmoplegia. Some diseases, such as Kearns-Sayre syndrome, Pearson's syndrome, and progressive external ophthalmoplegia are thought to be due to large-scale mtDNA rearrangements, multiple mtDNA deletions, and/or mtDNA depletion, whereas other diseases such as MELAS syndrome, Leber's hereditary optic neuropathy, MERRF (OMIM #545000), and others are due at least in part to point mutations in mtDNA. MERRF syndrome represents a phenotype that can be produced by mutation in more than one mitochondrial gene, e.g., MTTK, MTTL1, MTTH, MTTS1, MTTS2, or MTTF.

In some embodiment mtDNA depletion and/or mtDNA deletions may be caused at least in part by a mutation in a gene that encodes a gene product that is involved in maintenance, repair, and/or replication of mtDNA or in nucleotide metabolism. In some embodiments the gene product is a helicase, polymerase, ssDNA binding protein, or a protein or nucleic acid that associates with or activates a mtDNA helicase or mtDNA polymerase. In some embodiments a mitochondrial disorder is a mitochondrial DNA depletion syndrome (MDS) such as mitochondrial DNA depletion syndrome-1 (MNGIE TYPE; OMIM #603041), caused by homozygous or compound heterozygous mutations in the nuclear-encoded thymidine phosphorylase gene (TYMP; 131222); MTDPS2 (609560), caused by mutation in the TK2 gene (188250); MTDPS3 (OMIM #251880), caused by mutations in the DGUOK gene (601465); MTDPS4A (OMIM #203700) or MTDPS4B (OMIM #613662), both caused by mutations in the POLG gene (174763); MTDPS5 (OMIM #612073), caused by mutations in the SUCLA2 gene (603921); MTDPS6 (256810), caused by mutations in the MPV17 gene (137960); MTDPS7 (OMIM #271245), caused by mutations in the C10ORF2 gene (606075, the encoded protein is a mtDNA helicase also known as TWINKLE); MTDPS8A (OMIM #612075) or MTDPS8B (OMIM #612075), both caused by mutations in the RRM2B gene (604712); MTDPS9 (OMIM #245400), caused by mutation in the SUCLG1 gene (611224); MTDPS10 (221350), caused by mutations in the AGK gene (610345); and MTDPS11 (615084), caused by mutations in the MGME1 gene (615076). It will be understood that mutations in at least some of these genes may be associated with multiple different syndromes, which may affect different organs, and/or have different symptoms. For example, mutations in the gene known as C10ORF2 in humans are a cause of a variety of syndromes associated with mtDNA deletions, such as adult-onset progressive external ophthalmoplegia, infantile-onset spinocerebellar ataxia, and premature aging which are associated with multiple mtDNA deletions. Different mutations may result in different syndromes or may result in the same syndrome but with differing levels of severity.

In some embodiments a mitochondrial disorder comprises a complex I deficiency (OMIM #252010). Isolated deficiency of mitochondrial respiratory chain complex I can be caused by mutations in multiple different genes, both nuclear-encoded and mitochondrial-encoded. In some embodiments a complex I deficiency results from mutation in any of the subunits of complex I. In some embodiments complex I deficiency results from mutation in nuclear-encoded subunit genes, including NDUFV1 (161015), NDUFV2 (600532), NDUFS1 (157655), NDUFS2 (602985), NDUFS3 (603846), NDUFS4 (602694), NDUFS6 (603848), NDUFS7 (601825), NDUFS8 (602141), NDUFA2 (602137), NDUFA11 (612638), NDUFAF3 (612911), NDUFA10 (603835), NDUFB3 (603839), NDUFA1 (300078) or the complex I assembly genes B17.2L (609653), HRPAP20 (611776), C20ORF7 (612360), NUBPL (613621), and NDUFAF1 (606934). In some embodiments a complex I deficiency results from mutation in other nuclear-encoded genes, including FOXRED1 (613622) and ACAD9 (611103; see 611126). In some embodiments a complex I deficiency with mitochondrial inheritance is associated with mutation in MTND1 (516000), MTND2 (516001), MTND3 (516002), MTND4 (516003), MTND5 (516005), MTND6 (516006). Features of complex I deficiency may also be caused by mutation in other mitochondrial genes, including MTTS2 (590085).

In some embodiments a mitochondrial disorder comprises a complex II deficiency (OMIM #252011). In some embodiments a complex II deficiency is caused at least in part by a mutation in the SDHA (600857) or SCHAF1 (612848) gene.

In some embodiments a mitochondrial disorder comprises a complex III deficiency (discussed elsewhere herein).

In some embodiments a mitochondrial disorder comprises a complex IV deficiency (OMIM #220110). Mutations associated with complex IV deficiency have been identified in several mitochondrial COX genes, MTCO1 (516030), MTCO2 (516040), MTCO3 (516050), as well as in mitochondrial tRNA(ser) (MTTS1; 590080) and tRNA(leu) (MTTL1; 590050). Mutations in nuclear genes include those in COX10 (602125), COX6B1 (124089), SCO1 (603644), FASTKD2 (612322), C2ORF64 (613920), and C12ORF62 (614478). COX deficiency caused by mutation in SCO2 (604272) is associated with fatal infantile cardioencephalomyopathy (604377). Complex IV deficiency associated with Leigh syndrome (see 256000) may be caused by mutation in the SURF1 gene (185620), COX15 gene (603646), or TACO1 gene (612958). Complex IV deficiency associated with the French-Canadian type of Leigh syndrome (LSFC; 220111) is caused by mutation in the LRPPRC gene (607544).

In some embodiments a mitochondrial disorder comprises a mitochondrial complex V deficiency. Complex V deficiency may arise from mutations in any of a number of nuclear or mitochondrial genes.

In some embodiments a mitochondrial disorder is a combined oxidative phosphorylation deficiency, e.g., combined oxidative phosphorylation deficiency 1, 2, 3, 4, 5, 6, 7, 8, or 9.

In some embodiments a mitochondrial disorder is caused at least in part by a mutation in a gene that encodes an enzyme involved in biosynthesis or transport of a component of the electron transport chain that is not part of Complexes I-IV, such as coenzyme Q10 (ubiquinone) or cytochrome C. In some embodiments a mitochondrial disorder is coenzyme Q10 deficiency.

In some embodiments a mitochondrial disorder is caused at least in part by a defect in mitochondrial DNA replication and/or repair and/or a defect in nucleotide metabolism. Defects in mtDNA replication, mtDNA repair, or nucleotide metabolism can cause mitochondrial genetic diseases due to mtDNA deletions, point mutations, or depletion, which can ultimately cause loss of oxidative phosphorylation. Mitochondrial DNA is replicated and repaired by DNA polymerase γ (Pol γ) the only known DNA polymerase to be found in animal cell mitochondria, acting in conjunction with replication factors such as the mitochondrial single-stranded DNA binding protein and the mitochondrial DNA helicase (C10orf2 or Twinkle). The holoenzyme of pol γ in humans consists of a catalytic subunit (encoded by POLG at chromosomal locus 15q25) and a dimeric form of its accessory subunit (encoded by POLG2 at chromosomal locus 17q24.1). The catalytic subunit is a 140 kDa enzyme (p140) that has DNA polymerase, 3′-5′ exonuclease and 5′ dRP lyase activities. The accessory subunit is a 55 kDa protein (p55) required for tight DNA binding and processive DNA synthesis. The pol γ holoenzyme functions in conjunction with the mitochondrial DNA helicase, Twinkle or C10orf2, and the mtssB to form the minimal replication apparatus. Mutations in POLG, POLG2 and C10ORF2 can cause mitochondrial disorders. POLG mutation-related disorders are currently defined by at least six major phenotypes of neurodegenerative disease that include: Alpers-Huttenlocher syndrome (AHS, also called mitochondrial DNA depletion syndrome-4A; OMIM 203700), childhood myocerebrohepatopathy spectrum (MCHS), myoclonic epilepsy myopathy sensory ataxia (MEMSA), the ataxia neuropathy spectrum (ANS), autosomal recessive progressive external ophthalmoplegia (arPEO), and autosomal dominant progressive external ophthalmoplegia (adPEO). Mutations in C10orf2 are mainly associated with adPEO but have also been described as a cause of epileptic encephalopathy with mtDNA depletion or infantile-onset spinocerebellar at Defects in nucleotide metabolism are associated with mutations in TYMP, TK2, DGOUK, and RRM2B. Thymidine Phosphorylase (TP) is part of the pyrimidine salvage pathway required for the reversible reaction catalyzing thymidine and phosphate to thymine and deoxyribose-1-phosphate. Defects in TYMP, the gene that encodes TP, causes mitochondrial neurogastrointestinal encephalopathy (MNGIE) due to the accumulation of thymidine and uracil in the blood, which leads to mitochondrial DNA depletion, multiple deletions, and point mutations in affected tissues. Mutations in the mitochondrial thymidine kinase gene (TK2) are associated with multi-tissue mtDNA depletion syndrome or mtDNA depletion that mainly affects muscle and causes fatal myopathy. Deoxyguanosine kinase, encoded by the DGUOK gene is the other mitochondrial deoxyribonucleoside kinase and phosphorylates the purine nucleosides into nucleotide monophosphates. Currently, two forms of deoxyguanosine kinase deficiency associated with mutations in DGUOK have been described, the hepatocerebral mitochondrial DNA depletion syndrome which presents as a multisystem disorder and an isolated hepatic disease later in infancy or childhood. Ribonucleotide reductase is made up of two subunits, a large catalytic subunit, R1, and the smaller R2 subunit. Cells have two forms of the R2 subunit, a cell cycle regulated form that is maximally expressed in S-phase, and a p53-inducible form known as p53R2, encoded by the RRM2B gene, that is required for a basal level of DNA repair and mtDNA synthesis in non-proliferating cells. Mutations in RRM2B are associated with mtDNA depletion, resulting in a variety of syndromes and symptoms including PEO and mitochondrial neurogastrointestinal encephalopathy and mtDNA depletion in muscle. See Copeland, W C, Crit. Rev Biochem Mol. Biol. 2012; 47(1): 64-74 and references therein for further discussion of mitochondria; disorders associated with defects in mitochondrial DNA replication, DNA repair and/or nucleotide metabolism associated with mutations in genes encoding gene products that function directly in mtDNA replication (e.g., POLG, POLG2 and C10ORF2) or in metabolism of deoxynucleotide triphosphate pools used as the precursors for DNA replication. In some embodiments a mitochondrial depletion of deletion syndrome is caused a mutation in a gene encoding a protein that is not directly involved in either of these processes, such as OPA1, MPV17 (137960), ANT1 (103220), or SUCLA2 (603921), SUCLG1 (611224) (discussed further in Copeland W C. Inherited mitochondrial diseases of DNA replication. Annu Rev Med. 2008; 59:131-46).

In some embodiments a mitochondrial disorder is characterized by an abnormality in synthesis, metabolism, or structure of a component of the inner or outer mitochondrial membrane. In some embodiments a mitochondrial membrane component is a lipid. In some embodiments a lipid is cardiolipin (CL). For example, in some embodiments a mitochondrial disorder is Barth syndrome (OMIM #302060).

Mitochondrial dysfunction has been strongly implicated in the process of aging and age-related neurodegenerative diseases. Studies of human mitochondrial DNA (mtDNA) have found that tissue, such as the brain, exhibit significantly higher levels of mtDNA deletions with increasing age (Corral-Debrinski, M., et al. Nat Genet. 2, 324-329 (1992). In addition, there are numerous signs of decreased mitochondrial respiratory function in brain (Lin, M. T., et al. Human Molecular Genetics 11, 133-145 (2002), skeletal muscle (Trounce, I., et al, The Lancet 333, 637-639 (1989).) and liver tissue (Yen, T.-C., et al. Biochemical and Biophysical Research Communications 165, 994-1003 (1989) taken from elderly patients. Without wishing to be bound by any theory, mitochondrial dysfunction may lead to aging and age-related pathologies at least in part as a result of deficiencies in oxidative phosphorylation and/or increases in reactive oxygen species (ROS) generation (Lin, M. T. & Beal, M. F. Nature 443, 787-795 (2006). In some embodiments an agent that protects against mitochondrial dysfunction is useful to inhibit one or more effects of aging in a subject. In some embodiments an agent that protects against mitochondrial dysfunction is useful to prolong lifespan of a subject. In some embodiments the subject has not been diagnosed and is not suspected of having a mitochondrial disorder.

Methods for diagnosing mitochondrial disorders are known in the art (see, e.g., Harrison's Principles of Internal Medicine, 18th Edition; McGraw-Hill Professional, 2011). In some embodiments a mitochondrial disorder is diagnosed at least in part based on clinical symptoms and/or signs. In some embodiments a mitochondrial disorder is diagnosed at least in part based on detecting a mutation in nuclear or mitochondrial DNA or an alteration in mitochondrial copy number or morphology in a sample obtained from a subject. A mutation may be detected using any method known in the art. In some embodiments the mitochondrial genome of a subject is at least in part sequenced. In some embodiments the nuclear genome is at least in part sequenced. In some embodiments one or more genes whose mutation is associated with a mitochondrial disorder is sequenced or otherwise assessed for presence of a mutation. In some embodiments the sample is obtained from a tissue or organ affected by the disorder. In some embodiments a mitochondrial disorder is diagnosed at least in part based on a muscle biopsy. Muscle fibers may be stained with a suitable stain, e.g., Gömöri trichrome stain. A muscle biopsy may reveal an accumulation of enlarged mitochondria, which produces a dark red staining of the muscle fibers sometimes termed “ragged red fibers”. Excessive amounts of ragged red fibers constitute evidence of a mitochondrial myopathy.

In some embodiments a mitochondrial disorder may be diagnosed at least in part based on analysis of a sample obtained from a subject. In some embodiments the method comprises detecting the level of a metabolite or product normally produced at least in part in mitochondria. Defects in the mitochondrial energy-generating system may lead to altered metabolite levels in tissues or body fluids such as blood, urine, and/or CSF. For example, high lactate levels may occur due to reduced pyruvate utilization by the mitochondria. Detection of such altered metabolite levels may be used in diagnosis or in monitoring effects of therapy. In some embodiments a mitochondrial disorder may be diagnosed at least in part based on biochemical studies of living cells obtained from a subject (e.g., fibroblasts, lymphocytes) or mitochondria isolated from such cells. Examples of methods for biochemical analysis of cells are described elsewhere herein.

In some embodiments a mitochondrial disorder may be diagnosed at least in part based on analysis of a DNA or RNA sample obtained from the subject. As described above, many mitochondrial disorders are associated with mutations in particular nuclear or mitochondrial genes. Such genes or portions thereof can be sequenced, or mutations can be detected using methods known in the art. For example, PCR or other nucleic acid amplification methods can be used to amplify DNA or RNA, which can be detected in a variety of ways such as hybridization-based methods. Multiplexed PCR or other amplification methods are useful. Signal amplification assays include branched chain DNA assays and hybrid capture assays. Transcription based amplification and nucleic acid sequence based amplification (NASBA) may be used. In some embodiments allele-specific primer extension or allele-specific hybridization is used. Microarrays, e.g., oligonucleotide micorarrays, can be used, having probes for different alleles attached thereto. A microarray can be a solid phase or suspension array (e.g., a microsphere-based approach such as the Luminex platform). Mutations in a number of different genes are associated with Parkinson's disease. Such Parkinson's disease-linked genes include, e.g. α-synuclein and genes at the PARK1-PARK9 loci, e.g., encoding DJ-1, LRRK2, Parkin, UCH-L1, or PINKK1. The AD&FTD and PD Mutation Databases make available curated information of sequence variations in genes causing Mendelian forms of Alzheimer disease, frontotemporal lobar degeneration, and Parkinson's disease. These publicly available databases can be accessed at www.molgen.ua.ac.be/ADMutations and www.molgen.ua.ac.be/FTDMutations for the AD&FTD Mutation Database, and www.molgen.ua.ac.be/PDmutDB for the PD Mutations Database.

In some embodiments a subject at risk of a mitochondrial disorder may be identified at least in part based on analysis of a nucleic acid sample obtained from the subject. In some embodiments treatment may be started prior to onset of symptoms.

In some embodiments prenatal diagnosis may be performed. In some embodiments prenatal diagnosis can be performed at least in part by measuring respiratory chain or enzyme activities in chorionic villi or amniocytes. In some embodiments treatment may be started prior to or at birth or shortly thereafter (e.g., up to 1 day or up to 1 week after birth).

### VII. ATPIF1 as a Target for Treatment of Mitochondrial Disorders in Mammals

In some aspects, the invention relates to the identification of ATPase inhibitory factor 1 (ATPIF1) as a target for treatment of mitochondrial disorders. As described herein, Applicants discovered that loss of function of ATPIF confers protection against a variety of mitochondrial poisons. Using a gene trap mutagenesis strategy in a near-haploid mammalian cell line (KBM7), Applicants showed that insertions into the ATPIF1 gene rendered the cells resistant to exposure to the mitochondrial poison antimycin A, a complex III inhibitor. Cells in which the ATPIF1 gene was functionally disabled by insertional mutagenesis remained viable and able to proliferate when exposed to antimycin A at a concentration and for a time that was lethal to the great majority (more than 99.99%) of control (unmutagenized) KBM7 cells. Restoring ATPIF1 function by expressing ATPIF1 in ATPIF1⁻ KBM cells restored sensitivity to antimycin A. Furthermore, knockdown of endogenous ATPIF1 expression in unmutagenized cells using short hairpin RNA (shRNA) rendered these cells resistant to antimycin A. Cells in which the ATPIF1 gene was functionally disabled by insertional mutagenesis also showed decreased sensitivity to a variety of other mitochondrial poisons, e.g., FCCP, piercidin, and TTFA. Furthermore, the ATPIF1 gene was also identified in an independent haploid screen designed to identify genes conferring resistance to the mitochondrial poison FCCP. In some aspects, the disclosure provides the insight that inhibiting ATPIF1 protects against (confers protection against) mitochondrial dysfunction. In some aspects, the disclosure provides the insight that inhibiting ATPIF1 provides a means to treat a variety of mitochondrial disorders.)

ATPIF1 is a ˜10 kilodalton protein that is highly conserved across a wide range of eukaryotic species. One of skill in the art will readily be able to obtain ATPIF1 genomic, cDNA and mRNA sequences and ATPIF1 protein sequences from publicly available databases such as those available through Entrez. The human gene encoding ATPIF1 has been assigned GeneID: 93974 in the Gene database of the National Center for Biotechnology Information (NCBI; www.ncbi.nlm.nih.gov) and is located on chromosome 1 (28562602.28564616). Genes encoding ATPIF1 from mouse, rat, and cow (bovine) have been assigned the following Gene IDs: Gene ID: 11983 (Mus musculus); Gene ID: 25392 (Rattus norvegicus); Gene ID: 327699 (Bos taurus). Accession numbers for the human ATPIF1 mRNA and protein Reference Sequences from the NCBI are listed in Table 1. Official symbol and official name refer to those assigned by the Human Gene Nomenclature Committee (HUGO). ATPIF1 is sometimes abbreviated IF1 herein.

TABLE 1
Human Gene Symbols, Names, and NCBI RefSeq Accession Numbers

| Gene Official | Gene Official | | |
|---|---|---|---|
| Symbol | Name | mRNA | Protein |
| ATPIF1 | ATPase | NM_016311 | NP_057395 |
| | inhibitory | (transcript 1) | (isoform 1 precursor) |
| | factor 1 | NM_178190 | NP_835497 |
| | | (transcript 2) | (isoform 2 precursor) |
| | | NM_178191 | NP_835498 |
| | | (transcript 3) | (isoform 3 precursor) |

ATPIF1 is naturally produced as a precursor protein comprising a mitochondrial targeting sequence (MTS). The precursor protein is cleaved in cells to produce a mature ATPIF1 protein, i.e., the MTS (amino acids 1-˜25) is removed when the protein is imported into mitochondria. Sequences of representative naturally occurring human ATPIF1 polypeptides and nucleic acids (e.g., mRNA and/or cDNA) encoding them, can be found under the accession numbers mentioned above. It will be understood by those of ordinary skill in the art that polymorphisms of the ATPIF1 gene naturally exist among the human population (and among other mammals). Examples of polymorphisms in ATPIF1 may be found in databases such as dbSNP. The term “ATPIF1” is intended to encompass genes and gene products comprising naturally occurring polymorphisms. Three ATPIF1 isoforms have been identified in humans, of which isoform 1 is the longest. Isoforms 2 and 3 have distinct C-termini as compared with isoform 1. The term “ATPIF1” is intended to encompass ATPIF1 isoform 1, ATPIF1 isoform 2, and ATPIF1 isoform 3. Certain embodiments of any aspect herein may be directed towards all three isoforms. For example, in some embodiments an ATPIF1 inhibitor inhibits all three isoforms. Certain embodiments of any aspect herein may be directed towards any one or more particular isoforms. For example, in some embodiments an ATPIF1 inhibitor inhibits one or two ATPIF1 isoforms. ATPIF1 isoform 1 is the major isoform and is expressed in KBM7 cells. Certain embodiments of any aspect herein may be directed at least to isoform 1. In some embodiments an ATPIF1 inhibitor inhibits at least isoform 1.

Information regarding ATPIF1, including alignments of human ATPIF1 with ATPIF1 of various other species is available. ATPIF1 polypeptides have been purified from naturally occurring sources and have been produced recombinantly (see, e.g., Ichikawa, M. and Ogura, C., Journal of Bioenergetics and Biomembranes (2003); 35(5), 339-407), and references therein). Structures (e.g., crystal structure) of mature bovine ATPIF1 containing the mutation H49K are available (Cabezón E, et al., The EMBO Journal (2001) 20, 6990-6996). The atomic coordinates and structure factors were deposited in the Protein Data Bank (accession numbers 1gmj and r1gmjsf, respectively).

ATPIF1 can bind to and inhibit the F0-F1 ATP synthase (thus inhibiting its reversal to become an ATPase) under conditions of matrix acidification (low matrix pH), such as may occur in a variety of mitochondrial disorders or other states in which mitochondrial respiration is impaired (11, 12). ATPIF1 acts as a homodimer, simultaneously inhibiting two F1-ATP ATPase units. Binding of ATPIF1 to ATP synthase depends at least in part on pH. Bovine ATPIF1 has been shown to have two oligomeric states, tetramer (inactive) and dimer (active), favored by pH values above and below about 6.5-7.0, respectively (Cabezón E, et al., J. Biol. Chem. (2000); 275, 25460-25464) and it is reasonable to expect ATPIF1 to exhibit similar behavior in other mammalian species. The H49K mutation has been described as shifting the equilibrium between active and inactive conformations of ATPIF1 toward the active state in which it binds to F1-ATP synthase (Schnizer, R, et al., Biochim Biophys Acta. (1996); 1292(2):241-8). A crystal structure of bovine ATPIF1-inhibited F1-ATP synthase (crystals were generated in the presence of ATP) is also available (Cabezon, E. et al. Nat. Struct. Biol., (2003); 10, 744-750). Coordinates and structure factors were deposited in the Protein Data Bank (accession code 1OHH). If desired, a structure for human ATPIF1 or ATPIF1-inhibited F1-ATP synthase can be obtained or generated by modeling based on the bovine structure.

Upon inhibition of the electron transport chain (ETC), mitochondrial membrane potential (ΔΨm) decreases and the F1-F0 ATP synthase reverses, consuming ATP to pump protons into the intermembrane space (Campanella et al., 2008; Campanella et al., 2009; Lu et al., 2001). Normally an inactive tetramer, ATPIF1 dissociates into active dimers upon a large decrease in ΔΨm and subsequently inhibits reversal of the F1-F0 ATP synthase, an adaptive mechanism to prevent ATP consumption during periods of nutrient and oxygen deprivation (Cabezon et al., 2001; Campanella et al., 2008; Campanella et al., 2009; Fujikawa et al., 2012; Lu et al., 2001). While inhibiting the ATPase activity of F0-F1 ATPase under conditions of matrix acidification (such as may occur in a variety of mitochondrial disorders or other states in which mitochondrial respiration is impaired) may help maintain cellular ATP, it results in abnormal membrane potential. Without wishing to be bound by any theory, ATPIF1 activity may thus contribute to depolarization of the mitochondrial membrane. Inhibiting ATPIF1 may reduce or prevent depolarization of the mitochondrial membrane that would otherwise be caused by the binding of ATPIF1 to the F0-F1 ATPase and resulting inhibition of its ATPase activity. Reducing or preventing depolarization of the inner mitochondrial membrane may reduce or prevent release of mitochondrial substances, e.g., proteins, ions, metabolites, etc., that may otherwise occur as a result of depolarization. For example, pro-apoptotic proteins and/or substances that induce or contribute to necrosis may be released. As described herein, maintenance of ΔΨm through inhibiting APTIF1 enhances survival under ETC dysfunction despite its effect on ATP conservation. Thus, maintenance of ΔΨₘ is the more important process for survival under ETC dysfunction than conservation of ATP.

In some aspects, the disclosure provides methods of increasing resistance of a cell to a mitochondrial poison, the methods comprising inhibiting ATPIF1 in the cell. In some aspects, the disclosure provides methods of increasing resistance of a subject to a mitochondrial poison, the method comprising inhibiting ATPIF1 in at least some cells of the subject. In some embodiments such methods reduce the likelihood of a cell or subject to experience deleterious effects (e.g., cell death or damage) due to a mitochondrial poison or reduce the severity of such effects. In some embodiments inhibiting ATPIF1 comprises contacting a cell with an ATPIF1 inhibitor. In some embodiments inhibiting ATPIF1 comprises administering an ATPIF1 inhibitor to a subject.

In some embodiments methods of inhibiting apoptosis of a cell are provided, the methods comprising contacting a cell at risk of undergoing apoptosis with an ATPIF1 inhibitor. In some embodiments methods of inhibiting necrosis are provided, the methods comprising contacting a cell at risk of undergoing necrosis with an ATPIF1 inhibitor. In some embodiments a cell at risk of undergoing apoptosis or necrosis has a defect or deficiency in the electron transport chain, e.g., in complex I, II, III, IV, or in an electron carrier (cytochrome c or coenzyme Q).

In some embodiments methods of preserving cell viability under conditions of ATP depletion are provided, the methods comprising contacting a cell at risk of ATP depletion or experiencing ATP depletion with an ATPIF1 inhibitor.

In some embodiments methods of preserving mitochondrial inner membrane potential are provided, the methods comprising contacting a cell at risk of loss of inner mitochondrial membrane potential or experiencing loss of inner mitochondrial membrane potential with an ATPIF1 inhibitor.

In some embodiments a cell at risk of or experiencing ATP depletion or loss of inner mitochondrial membrane potential has been exposed to conditions associated with ATP depletion or loss of inner mitochondrial membrane potential. In some embodiments a cell at risk of or experiencing ATP depletion or loss of inner mitochondrial membrane potential has been exposed to a mitochondrial poison that inhibits or depletes a component of the electron transport chain, e.g., a mitochondrial poison that inhibits or depletes a component of complex I, II, III, or IV or that inhibits or depletes an electron carrier.

In some embodiments methods of protecting a cell against mitochondrial dysfunction are provided, the methods comprising contacting a cell at risk of mitochondrial dysfunction or experiencing mitochondrial dysfunction with an ATPIF1 inhibitor. In some embodiments methods of protecting a subject against mitochondrial dysfunction are provided, the methods comprising administering an ATPIF1 inhibitor to a subject at risk of mitochondrial dysfunction or suffering from mitochondrial dysfunction.

In some aspects, methods of selecting a therapeutic agent for a subject are provided. In some embodiments a method comprises (a) providing a subject in need of treatment for a mitochondrial disorder; and (b) selecting an ATPIF1 inhibitor as a therapeutic agent for the subject. In some embodiments the method comprises determining that the subject suffers from a mitochondrial disorder. In some embodiments, the method further comprises administering an ATPIF1 inhibitor to the subject. In some embodiments the method further comprises monitoring the effect of an ATPIF1 inhibitor on the subject following administration of the ATPIF1 inhibitor. For example, at least one indicator of mitochondrial function or at least one clinical parameter associated with a mitochondrial disorder may be assessed one or more times after administration.

In some aspects, methods of determining whether a subject is a suitable candidate for treatment with an ATPIF1 inhibitor are provided. In some embodiments, a method comprises determining that a subject suffers from or is at risk of a mitochondrial disorder; wherein if the subject suffers from or is at risk of a mitochondrial disorder, the subject is a suitable candidate for treatment with an ATPIF1 inhibitor. In some embodiments, the method further comprises administering an ATPIF1 inhibitor to the subject.

In some aspects, methods of treating a subject in need of treatment for a mitochondrial disorder are provided. In some embodiments, a method comprises administering an ATPIF1 inhibitor to the subject. In some embodiments, a method of treatment comprises providing a subject in need of treatment for a mitochondrial disorder. In some embodiments, a method of treatment comprises diagnosing a subject as having a mitochondrial disorder. The subject may have one or more symptoms or signs of a mitochondrial disorder. In some embodiments, a method of treatment comprises diagnosing a subject as being at risk or having a mitochondrial disorder. In some embodiments the subject harbors a mutation that causes a mitochondrial disorder in at least some individuals having the mutation. In some embodiments the subject has been exposed to a toxic agent known to cause or contribute to a mitochondrial disorder in at least some individuals exposed to it. In some embodiments the toxic agent comprises a mitochondrial poison. In some embodiments, the method comprises administering an ATPIF1 inhibitor to the subject. In some embodiments an ATPIF inhibitor is used to treat any of the mitochondrial disorders discussed in Section VI hereof. In certain embodiments the mitochondrial disorder is characterized by cell or tissue loss (e.g., due to apoptosis). In some embodiments the mitochondrial disorder is Parkinson's disease; Huntington's disease; GRACILE syndrome; a mitochondrial disorder involving optic atrophy (e.g., retinal ganglion cell death) such as Leber's hereditary optic neuropathy, dominant autosomal-dominant optic atrophy, Charcot-Marie-Tooth disease type 2 (CMT2A), or glaucoma; or a disorders associated with hypoxia, ischemia, or ischemia-reperfusion injury. In some embodiments a mitochondrial disorder may be associated with abnormal expression or activity of ATPIF1. For example, in some embodiments a mitochondrial disorder may be associated with inappropriately elevated expression or activity of ATPIF1. In some embodiments inappropriately elevated expression or activity of APTIF1 may result in inappropriately inhibition of the F0-F1 ATPase by ATPIF1. In some embodiments an ATPIF1 inhibitor is used to inhibit appropriately elevated expression or activity of APTIF1.

In some aspects, compositions and methods useful to modulate, e.g., inhibit, ATPIF1 are provided. The term “ATPIF1 inhibitor” refers to an agent that inhibits ATPIF1 expression and/or inhibits one or more activities of ATPIF1. In some embodiments an agent is an “ATPIF1 inhibitor” if one or more ATPIF1 activities is reduced in the presence of the agent as compared with its absence and/or if the level or amount of ATPIF1 protein or gene product is reduced in the presence of the agent as compared with its absence. In some embodiments, an ATPIF1 modulator, e.g., an ATPIF1 inhibitor act directly on ATPIF1, i.e., the agent physically interacts with ATPIF1, e.g., binds to ATPIF1. In some embodiments, an ATPIF1 inhibitor acts indirectly on ATPIF1. An ATPIF1inhibitor can be, e.g., a small molecule, nucleic acid, oligonucleotide, polypeptide, peptide, lipid, phospholipid, etc. In some embodiments, an ATPIF1 inhibitor is an RNAi agent, antisense oligonucleotide, aptamer, or antibody. In some embodiments, an ATPIF1 inhibitor is a small molecule.

In some embodiments an ATPIF1 activity comprises binding to F1-ATP synthase. In some embodiments an ATPIF1 activity comprises ATPIF1 dimer formation. In some embodiments an ATPIF1 activity comprises physically associating with a protein other than F1-ATP synthase or ATPIF1 itself. As described in Example 3, Applicants identified a number of proteins that physically interact with ATPIF1. In some embodiments an ATPIF1 activity comprises physically interacting with a protein listed in Table 2.

Methods of inhibiting ATPIF1 encompass methods that result in a decreased amount of ATPIF1 polypeptide and methods that interfere with at least one ATPIF1 activity. In some embodiments, ATPIF1 is inhibited by inhibiting or interfering with ATPIF1 expression, so that a decreased amount of ATPIF1 is produced. A variety of methods useful for inhibiting or interfering with expression can be used in various embodiments. In general, such methods result in decreased synthesis of ATPIF1 polypeptide and as a result, a reduction in the total level of ATPIF1 activity present.

In some embodiments, ATPIF1 expression is inhibited using RNA interference (RNAi). Examples of sequences for RNAi agents (e.g., shRNAs) that inhibit ATPIF1 expression are provided in the Examples. Additional sequences can be selected using various approaches known in the art. If desired, such sequences can be selected to minimize “off-target” effects. In some embodiments, position-specific chemical modification is used to reduce potential off-target effects. In some embodiments, at least two different siRNAs targeted to the ATPIF1 gene are used (e.g., in combination). In some embodiments an RNAi agent is used as an ATPIF1 inhibitor, e.g., for therapeutic or research purposes. In some embodiments an RNAi agent that inhibits ATPIF1 is used to confirm that the effect of a second compound, e.g., a small molecule, is due to an effect on ATPIF1 (rather than on another protein). For example, a small molecule that is a putative specific inhibitor of ATPIF1 may be expected to have less effect or an effect in a cell in which ATPIF1 expression is inhibited by RNAi, as compared with the effect of such compound in a control cell in which ATPIF1 expression is not inhibited by RNAi.

In some embodiments expression of a gene encoding ATPIF1 is inhibited using an antisense approach. Antisense approaches encompass methods in which one or more single-stranded oligonucleotides complementary to RNA (e.g., mRNA) that encodes a protein whose inhibition is desired (e.g., ATPIF1) is contacted with cells, e.g., in a culture medium or by administration to a subject. The single-stranded oligonucleotide enters cells and hybridizes to a RNA target. Such hybridization may result in, e.g., degradation of mRNA by RNase H or blockage of translation. The oligonucleotide may comprise a sequence at least about 80%, 85%, 90%, 95%, 99%, or 100% complementary to a RNA target over at least 10, 12, 14, 16, 18, 20, 22, 24, 26, 28, or 30 nt. The oligonucleotide sequence may be selected to minimize off-target effects. For example, a sequence that has less than about 70%, 75%, 80%, 85%, 90%, 95%, 99%, or 100% complementarity to known or predicted mRNAs (other than the target) of a species to which the antisense agent is to be administered over at least 10, 12, 14, 16, 18, 20, 22, 24, 26, 28, or 30 nt may be selected.

In some embodiments, an ATPIF1 inhibitor comprises an antibody or portion thereof. In some embodiments, the antibody is a single-chain antibody, diabody, triabody, or minibody. Standard methods of antibody production known in the art can be used to produce an antibody, e.g., a monoclonal antibody, that binds to ATPIF1. In some embodiments, an animal, e.g., a mouse, rabbit, etc., is immunized with ATPIF1 or a portion thereof, antibody producing cells are isolated, and a monoclonal antibody is identified using hybridoma technology. In some embodiments, the mouse is a transgenic mouse comprising at least some unrearranged human immunoglobulin gene sequences and in some embodiments having targeted disruption of endogenous heavy and light chain murine sequences. In some embodiments, an antibody is identified or produced at least in part using recombinant nucleic acid technology (e.g., phage or yeast display). See, e.g., Lonberg N. Fully human antibodies from transgenic mouse and phage display platforms. Curr Opin Immunol. 20(4):450-9, 2008. In some embodiments an antibody comprises a single polypeptide chain that can be expressed intracellularly.

In some embodiments, an ATPIF1 inhibitor comprises a peptide. In some embodiments a peptide is identified using a display technique, such as phage display, ribosome display, or yeast display. In some embodiments, a peptide comprises one or more non-standard amino acids. In some embodiments, a peptide is cyclic. For example, the peptide can be cyclized via a disulfide bond or covalent linkage, e.g., between the N- and C-terminal amino acids, between the N- or C-terminal amino acid an internal amino acid, or between two internal amino acids.

In some embodiments an ATPIF1 inhibitor comprises an engineered protein designed and/or selected to bind to ATPIF1. In some embodiments the protein is an engineered binding protein that is distinct from antibodies, such as an affibody, anticalin, adnectin, or darpin.

In some embodiments an ATPIF1 inhibitor (e.g., a polypeptide) comprises a MTS.

In some embodiments an ATPIF1 inhibitor inhibits mitochondrial localization of ATPIF1 protein.

In some embodiments an ATPIF1 inhibitor induces degradation of ATPIF1 protein.

In some embodiments, an ATPIF1 inhibitor comprises a small molecule. The small molecule may, for example, bind to ATPIF1 and inhibit its dimerization or inhibit its ability to bind to F1-ATP synthase. In some embodiments, an ATPIF1 inhibitor, e.g., a small molecule, comprises a reactive functional group that reacts with a functional group of ATPIF1, resulting in covalent attachment of the ATPIF1 inhibitor to ATPIF1. In some embodiments a reactive functional group is an aldehyde, haloalkane, alkene, fluorophosphonate (e.g., alkyl fluorophosphonate), Michael acceptor, phenyl sulfonate, methylketone, e.g., a halogenated methylketone or diazomethylketone, fluorophosphonate, vinyl ester, vinyl sulfone, or vinyl sulfonamide. In some embodiments, an ATPIF1 inhibitor comprises an electrophilic group that reacts with an amino acid side chain of ATPIF1. For example, the electrophilic group may react with an amino acid side chain containing a nucleophile such as a hydroxyl or sulfhydryl group. For example, the amino acid may be cysteine, serine, or threonine. Moieties sometimes referred to in the art as “cysteine traps” may be used in various embodiments. In some embodiments a cysteine-reactive moiety is a maleimide, isothiazolinone, tetrazole, lactam, or carbamate.

In some embodiments an agent indirectly inhibits ATPIF1. “Indirect inhibition” refers to inhibition of a target (e.g., ATPIF1) by a mechanism that does not require physical interaction between the agent and the target. For example, in some embodiments the agent inhibits expression or activity of a polypeptide that is involved in localization or post-translational modification of ATPIF1, wherein such localization or post-translational modification is important for ATPIF1 molecular function.

In some aspects, methods of identifying agents useful for modulating, e.g., inhibiting, ATPIF1 are provided. In some embodiments reagents, compositions, and systems useful for performing one or more of the methods are provided. In some aspects, the invention provides a method of determining whether a test agent is a candidate agent, the method comprising the step determining whether the test agent inhibits an ATPIF1 polypeptide, wherein if the test agent inhibits an ATPIF1 polypeptide the test agent is a candidate agent for (i) improving mitochondrial function; (ii) inhibiting apoptosis or necrosis; (iii) preserving cell viability under conditions of ATP depletion; (iv) preserving mitochondrial inner membrane potential, e.g., under conditions associated with loss of inner membrane potential; (v) protecting a cell against mitochondrial dysfunction; and/or (vi) treatment of mitochondrial disorders.

In some embodiments, a method comprises determining whether a test agent inhibits expression of ATPIF1, wherein if the test agent inhibits ATPIF1 expression the test agent is a candidate agent. In some embodiments, the method comprises determining whether the test agent inhibits an activity of an ATPIF1 polypeptide, wherein if the compound inhibits an ATPIF1 activity of the polypeptide, the test agent is a candidate agent. In some embodiments an ATPIF1 activity comprises binding to F1-ATP synthase or a subunit thereof, ATPIF1 dimer formation, or physically associating with a protein other than F1-ATP synthase or a subunit thereof (e.g., ATP synthase subunit alpha or beta) or ATPIF1 itself (e.g., a protein listed in Table 2 other than an ATP synthase subunit or ATPIF1 itself.

The term “ATPIF1 polypeptide” refers to a polypeptide whose sequence comprises or consists of the sequence of a naturally occurring ATPIF1 polypeptide of a multicellular organism (e.g., a vertebrate, e.g., a mammal, such as a primate (e.g., a human), a rodent, a bovine, etc.) or a variant thereof. For purposes of the present disclosure the terms “ATPIF1” or “native ATPIF1” or “naturally occurring ATPIF1” are used interchangeably and encompass any polypeptide identical in sequence to ATPIF1 (precursor or mature form) found in nature, e.g., in a mammalian cell, e.g., in a human cell. In some embodiments, an ATPIF1 polypeptide is a native ATPIF1. In some embodiments, an ATPIF1 polypeptide is a variant of ATPIF1 (“ATPIF1 variant”). ATPIF1 variants include polypeptides that differ by one or more amino acid substitutions, additions, or deletions, relative to ATPIF1. An addition can be an insertion within the polypeptide or an addition at the N- or C-terminus. In some embodiments, the number of amino acids substituted, deleted, or added can be for example, about 1 to 20, e.g., about 1 to 10, e.g., about 1 to 5, e.g., 1, 2, 3, 4, or 5. In some embodiments, a ATPIF1 variant comprises a polypeptide whose sequence is at least 70%, 75%, 80%, 85%, 90%, 95%, 96%, 97%, 98%, 99%, or more identical to ATPIF1 (e.g., from human) over at least 50%, 60%, 70%, 80%, 85%, 90%, 95%, 96%, 97%, 98%, 99%, or 100% of a mature ATPIF1. In some embodiments, an ATPIF1 variant comprises a polypeptide whose sequence is at least 70%, 75%, 80%, 85%, 90%, 95%, 96%, 97%, 98%, 99%, or more identical to ATPIF1 (e.g., from human) over at least 50%, 60%, 70%, 80%, 85%, 90%, 95%, 96%, 97%, 98%, 99%, or 100% of an ATPIF1 precursor protein. In some embodiments an ATPIF1 polypeptide comprises a mature ATPIF1 that has a methionine at the N-terminus.

In some embodiments, an ATPIF1 polypeptide comprises or consists of an ATPIF1 fragment. An ATPIF1 fragment is a polypeptide that is shorter than ATPIF1 and is identical to ATPIF1 over the length of the shorter polypeptide. In some embodiments, an ATPIF1 fragment is at least 50%, 60%, 70%, 80%, 85%, 90%, 95%, 96%, 97%, 98%, or 99% as long as native ATPIF1. In some embodiments, a fragment is a protein in which one or more amino acids at the N-terminus of ATPIF1 are deleted. In some embodiments, a fragment is a protein in which one or more amino acids at the C-terminus of ATPIF1 are deleted.

In some embodiments, an ATPIF1 polypeptide comprises a heterologous polypeptide portion. In some embodiments a heterologous portion comprises a sequence that is not present in or homologous to native ATPIF1. A heterologous portion may be, e.g., between 5 and about 5,000 amino acids long. In some embodiments a heterologous portion is at least 5 and no more than 10; 15; 20; 30; 50; 100; or 500 amino acids long. In some embodiments, a heterologous portion comprises a sequence that is found in a different polypeptide, e.g., a functional domain of a different polypeptide. In some embodiments, a heterologous portion comprises a sequence useful for purifying, expressing, solubilizing, and/or detecting the polypeptide. In some embodiments, a heterologous portion comprises a polypeptide “tag”, e.g., an affinity tag or epitope tag. For example, a tag can be an affinity tag (e.g., HA, TAP, Myc, 6XHis, Flag, GST), fluorescent or luminescent protein (e.g., EGFP, ECFP, EYFP, Cerulean, DsRed, mCherry), solubility-enhancing tag (e.g., a SUMO tag, NUS A tag, SNUT tag, or a monomeric mutant of the Ocr protein of bacteriophage T7). See, e.g., Esposito D and Chatterjee DK. Curr Opin Biotechnol.; 17(4):353-8 (2006). In some embodiments, a tag can serve multiple functions. A tag is often relatively small, e.g., ranging from a few amino acids up to about 100 amino acids long. In some embodiments a tag is more than 100 amino acids long, e.g., up to about 500 amino acids long, or more. In some embodiments, a ATPIF1 polypeptide has a tag located at the N- or C-terminus, e.g., as an N- or C-terminal fusion. In some embodiments an ATPIF1 polypeptide comprises multiple tags. In some embodiments, a tag is cleavable, so that it can be removed from the polypeptide, e.g., by a protease. In some embodiments, this is achieved by including a sequence encoding a protease cleavage site between the sequence encoding the portion homologous to ATPIF1 and the tag. Examples of proteases include, e.g., thrombin, TEV protease, Factor Xa, PreScission protease, etc. In some embodiments, a tag is a “self-cleaving” tag. See, e.g., PCT/US05/05763. Sequences encoding a tag can be located 5′ or 3′ with respect to a polynucleotide encoding the polypeptide (or both). In some embodiments a tag or other heterologous sequence is separated from the rest of a polypeptide by a polypeptide linker. For example, a linker can be a short polypeptide (e.g., 15-25 amino acids). In some embodiments a linker is composed of small amino acid residues such as serine, glycine, and/or alanine. In some embodiments a heterologous domain comprises a mitochondria (targeting sequence from a protein other than ATPIF1.

In some embodiments, an ATPIF1 variant is a functional variant, i.e., the variant at least in part retains at least one biological activity of ATPIF1. In some embodiments, a functional variant retains sufficient activity to be distinguishable from a non-homologous protein or biologically inactive ATPIF1 polypeptide when used in an assay for binding to F0-F1 ATPase. In some embodiments, a functional ATPIF1 variant retains at least 10%, 20%, 30%, 40%, 50%, 60%, 70%, 80%, 90%, 95%, 96%, 97%, 98%, 99%, or more of at least one activity of ATPIF1, e.g., about equal activity.

One of ordinary skill in the art can generate functional ATPIF1 variants. In some embodiments, an ATPIF1 variant comprises one or more conservative amino acid substitutions relative to ATPIF1. Conservative substitutions may be made on the basis of similarity in side chain size, polarity, charge, solubility, hydrophobicity, hydrophilicity and/or the amphipathic nature of the residues involved. As known in the art, such substitutions are, in general, more likely to result in a variant that retains activity as compared with non-conservative substitutions. Of course non-conservative substitutions are often compatible with retaining function as well. In some embodiments, a substitution or deletion does not alter or delete an amino acid that is highly conserved across different species. In some embodiments, an alteration is at an amino acid that is not well conserved among ATPIF1 polypeptides of different species. In some embodiments an ATPIF1 variant is tested in one or more cell-free and/or cell-based assays to assess activity. In some embodiments a functional ATPIF1 variant comprises at least residues 20-40 of a mature mammalian ATPIF1, e.g., mature human ATPIF1. In some embodiments a functional ATPIF1 variant comprises at least residues 15-45 or at least residues 14-47 of a mature mammalian ATPIF1, e.g., mature human ATPIF1.

In some embodiments, a variant of ATPIF1 that has substantially reduced activity as compared with the activity of native ATPIF1 (e.g., less than 10% of the activity of native ATPIF1) is useful as an ATPIF1 inhibitor. In some embodiments such a polypeptide interferes with a function of native ATPIF1 in mitochondria. For example, in some embodiments the variant dimerizes with endogenous ATPIF1 and results in an inactive dimer. In some embodiments the variant includes a portion of ATPIF1 that participates in dimerization but lacks at least one residue that participates in binding to ATPIF1. In some embodiments the variant stabilizes a tetrameric form of ATPIF1. In some embodiments the variant binds to F0-F1 ATPase but fails to inhibit it. In some embodiments a variant of ATPIF1 that has substantially reduced activity as compared with the activity of native ATPIF1 is useful a control or as an immunogen or for crystallization or binding studies.

An ATPIF1 polypeptide, e.g., a native ATPIF1 polypeptide or an ATPIF1 variant can be produced using standard recombinant DNA techniques. Nucleic acids encoding ATPIF1 readily be obtained, e.g., from cells that express ATPIF1 (e.g., by PCR or other amplification methods or by cloning) or by synthesis based on a known ATPIF1 cDNA or polypeptide sequence or ordered from commercial suppliers. One of skill in the art would know that due to the degeneracy of the genetic code, numerous different nucleic acid sequences would encode a desired polypeptide. Optionally, a sequence is codon-optimized for expression in a host cell of choice. A nucleic acid that encodes an ATPIF1 variant can readily be generated, e.g., by modifying a sequence that encodes native ATPIF1 using, e.g., site-directed mutagenesis, or by other standard methods.

In some embodiments a nucleic acid encoding an ATPIF1 polypeptide (or other desired polypeptide), operably linked to appropriate expression control elements is introduced into prokaryotic or eukaryotic cells, usually using a vector such as a plasmid or virus. In some embodiments an ATPIF1 polypeptide is produced using in vitro translation. Examples of cells include, e.g., bacterial cells (e.g., E. coli), insect cells, mammalian cells, plant cells, fungal cells (e.g., yeast). One of ordinary skill in the art will be aware of suitable expression control elements (e.g., promoters). Promoters may be constitutive or regulatable, e.g., inducible or repressible. Examples of promoters suitable for use in bacterial cells include, e.g., Lac, Trp, Tac, araBAD (e.g., in a pBAD vectors), phage promoters such as T7 or T3. Examples of expression control sequences useful for directing expression in mammalian cells include, e.g., the early and late promoters of SV40, adenovirus or cytomegalovirus immediate early promoter, or viral promoter/enhancer sequences, retroviral LTRs, promoters or promoter/enhancers from mammalian genes, e.g., actin, EF-1 alpha, metallothionein, etc. The polyhedrin promoter of the baculovirus system is of use to express proteins in insect cells. One of skill in the art will be aware of numerous expression vectors that contain appropriate expression control element(s), selectable markers, cloning sites, etc., and can be conveniently used to express a polypeptide of interest. Optionally, such vectors include sequences encoding a tag, to allow convenient production of a polypeptide comprising a tag. Suitable methods for introducing vectors into bacteria, yeast, plant, or animal cells (e.g., transformation, transfection, infection, electroporation, etc.), and, if desired, selecting cells that have taken up the vector and deriving stable cell lines, are known to those of ordinary skill in the art. Transgenic animals or plants that express the polypeptide can be produced using methods known in the art.

In some embodiments cells expressing an ATPIF1 polypeptide are maintained in culture for a suitable time period, and the polypeptide is isolated and optionally further purified. In some embodiments an ATPIF1 polypeptide is isolated from cells or tissues obtained from an organism that naturally expresses the polypeptide. Standard protein isolation/purification techniques can be used. In some embodiments, affinity-based methods are used. For example, an antibody that binds to ATPIF1 can be employed. In the case of a tagged ATPIF1 polypeptide, appropriate isolation methods can be selected depending on the particular tag used. For example, an antibody to a tag can be used.

In some embodiments, a method is performed using an ATPIF1 polypeptide identical in sequence to a native ATPIF1. In some embodiments, a method is performed using a functional ATPIF1 variant. In some embodiments, the functional variant used in an inventive assay retains at least 20%, 30%, 40%, 50%, 60%. 70%, 80%, 90%, 95%, 96%, 97%, 98%, 99%, or more of the F0-F1 ATPase binding activity of native ATPIF1. In some embodiments an agent identified as an inhibitor using a ATPIF1 variant can be further tested using native ATPIF1 to confirm its ability to inhibit the native polypeptide.

Any of a wide variety of agents may be used as test agents may be used in various embodiments. For example, a test agent may be a small molecule, polypeptide, peptide, nucleic acid, oligonucleotide, lipid, carbohydrate, or hybrid molecule. Agents can be obtained from natural sources or produced synthetically. Agents may be at least partially pure or may be present in extracts or other types of mixtures. Extracts or fractions thereof can be produced from, e.g., plants, animals, microorganisms, marine organisms, fermentation broths (e.g., soil, bacterial or fungal fermentation broths), etc. In some embodiments, a compound collection (“library”) is tested. The library may comprise, e.g., between 100 and 500,000 compounds, or more. Compounds are often arrayed in multiwell plates. They may be dissolved in a solvent (e.g., DMSO) or provided in dry form, e.g., as a powder or solid. Collections of synthetic, semi-synthetic, and/or naturally occurring compounds may be tested. Compound libraries can comprise structurally related, structurally diverse, or structurally unrelated compounds. Compounds may be artificial (having a structure invented by man and not known to be found in nature) or naturally occurring. In some embodiments a library comprises at least some compounds that have been identified as “hits” or “leads” in a drug discovery program and/or analogs thereof. A compound library may comprise natural products and/or compounds generated using non-directed or directed synthetic organic chemistry. A compound library may be a small molecule library. Other libraries of interest include peptide or peptoid libraries, ORF libraries, cDNA libraries, and oligonucleotide libraries.

A library may be focused (e.g., composed primarily of compounds having the same core structure, derived from the same precursor, or having at least one biochemical activity in common). Compound libraries are available from a number of commercial vendors such as Tocris BioScience, Nanosyn, BioFocus, and from government entities. For example, the Molecular Libraries Small Molecule Repository (MLSMR), a component of the U.S. National Institutes of Health (NIH) Molecular Libraries Program distributes a collection of >300,000 chemically diverse compounds with known and unknown biological activities for use, e.g., in high-throughput screening assays (see https://mli.nih.gov/mli/). The NIH Clinical Collection (NCC) is a plated array of approximately 450 small molecules that have a history of use in human clinical trials. These compounds are considered highly “drug-like” and have known safety profiles. In some embodiments, a collection of compounds comprising “approved human drugs” may be tested. An “approved human drug” is an agent that has been approved for use in treating humans by a government regulatory agency such as the US Food and Drug Administration, European Medicines Evaluation Agency, or a similar agency responsible for evaluating at least the safety of therapeutic agents prior to allowing them to be marketed. A test agent may be, e.g., an antineoplastic, antibacterial, antiviral, antifungal, antiprotozoal, antiparasitic, antidepressant, antipsychotic, anesthetic, antianginal, antihypertensive, antiarrhythmic, antiinflammatory, analgesic, antithrombotic, antiemetic, immunomodulator, antidiabetic, lipid- or cholesterol-lowering (e.g., statin), anticonvulsant, anticoagulant, antianxiety, hypnotic (sleep-inducing), hormonal, or anti-hormonal drug, etc. In some embodiments an agent has undergone at least some preclinical or clinical development or has been determined or predicted to have “drug-like” properties, such as being compliant with Lipinski's Rule of Five (Lipinski; F., et al. (2001). “Experimental and computational approaches to estimate solubility and permeability in drug discovery and development settings”. Adv Drug Del Rev). For example, an agent may have completed a Phase I trial or at least a preclinical study in non-human animals and shown evidence of safety and tolerability. In some embodiments an agent is not an agent that is found in a cell culture medium known or used in the art, e.g., for culturing vertebrate, e.g., mammalian cells, e.g., an agent provided for purposes of culturing the cells, or, if the agent is found in a cell culture medium known or used in the art, the agent may be used at a different, e.g., higher, concentration when used in a method or composition described herein. In some embodiments, an agent identified as described herein, e.g., an ATPIF1 inhibitor identified as described herein, may have an unknown structure and/or may be part of a mixture comprising multiple potentially active agents. A variety of techniques useful for determining the structures of agents are known and may be used to determine the structure, if desired, such as NMR, infrared (IR) spectroscopy, ultraviolet-visible (UV-Vis) spectroscopy, mass spectrometry, X-ray crystallography, etc. A variety of techniques useful for separating agents are known and may be used to separate agents present in a mixture. In some embodiments at least 10; 100; 500; 1,000; 5,000; 10,000; 25,000; 50,000; 100,000; 200,000; 500,000 agents are tested. In some embodiments such testing is performed within 1-7 days, 1-4 weeks, 4-12 weeks, 12-26 weeks, 26-52 weeks, or more.

Determining whether or to what extent an agent inhibits ATPIF1 expression can be carried out in a variety ways. Agents that inhibit ATPIF1 expression can be identified by contacting cells with a test agent, maintaining the cells in culture for a suitable period of time (e.g., sufficient time to allow degradation of existing ATPIF1 gene product), and then measuring the level of ATPIF1 gene product (e.g., mRNA or protein). Methods known in the art can be used for measuring RNA or protein. A variety of different hybridization-based or amplification-based methods are available to measure RNA. Examples include Northern blots, microarray (e.g., oligonucleotide or cDNA microarray), reverse transcription (RT)-PCR (e.g., quantitative RT-PCR), or reverse transcription followed by sequencing. The TaqMan® assay and the SYBR® Green PCR assay are commonly used real-time PCR techniques. Other assays include the Standardized (Sta) RT-PCRT™ (Gene Express, Inc., Toledo, Ohio) and QuantiGene® (Panomics, Inc., Fremont, Calif.). In some embodiments the level of ATPIF1 mRNA is measured. In other embodiments, a reporter-based system is used. In some embodiments, a reporter-based system comprises a nucleic acid in which expression control elements of the ATPIF1 gene are operably linked to a sequence that encodes a reporter. Examples of reporters are discussed above.

Methods for assessing the efficacy of an RNAi agent to silence expression of a target gene can involve use of a sequence in which the mRNA target of an shRNA or siRNA (or a portion of the target) is cloned downstream of a sequence that encodes a reporter, so that a bicistronic mRNA transcript encoding both the target sequence and the reporter is produced. Target gene knockdown results in the degradation (or translational inhibition) of the mRNA transcript, which causes a proportional decrease in the expression of the reporter protein.

Agents that modulate, e.g., inhibit, ATPIF1 activity can be identified using a variety of different cell-free or cell-based assays. A cell-free assay typically involves an isolated target molecule or complex. For example, the target molecule or complex could be present in a cell or tissue lysate or fraction thereof (e.g., a lysate made from cells that express the target molecule) or could be an at least partially purified or synthesized (e.g., recombinantly produced) target. A tissue lysate may be made from any tissue containing cells that express ATPIF1. In some embodiments, an isolated polypeptide has been synthesized using recombinant nucleic acid techniques or in vitro translation. In some embodiment a test agent is contacted with the target, e.g., by preparing a composition comprising the test agent and the target. One or more parameters are measured, e.g., binding of the test agent to the target, activity of the target, etc. The composition can comprise other component(s) necessary or helpful for identifying an agent of interest. In some embodiments, a composition for use in a binding assay or activity assay comprises ATP. In some embodiments, a composition for use in a binding assay or activity assay comprises lipid-containing membrane or component thereof. Such membranes or components may be naturally occurring (e.g., components present in cell or mitochondrial membranes), artificial, or combination thereof in various embodiments. For example, the composition can contain a lipid membrane bilayer, lipid vesicles, etc. Optionally, a lipid bilayer is immobilized on a surface. In some embodiments the lipids comprise phospholipids.

A variety of cell-free assays may be performed to identify agents that modulate, e.g., inhibit, an ATPIF1 polypeptide. In some embodiments, an assay detects whether a test compound binds to an ATPIF1 polypeptide and/or quantifies one or more characteristics of such binding. Numerous binding assay formats are known in the art. In some embodiments, a label-free assay is used. In some embodiments the ATPIF1 polypeptide, test compound, or both is/are detectably labeled. In some embodiments an ATPIF1 polypeptide or a compound to be tested for ability to bind to and/or inhibit activity of an ATPIF1 polypeptide is noncovalently or covalently attached to a solid support. In some embodiments, a solid support is an article having a rigid or semi-rigid surface. In some embodiments, at least one surface of the support is substantially flat. In other embodiments, a support is approximately spherical. A support can be composed of an inorganic or organic material or combination thereof. In some embodiments, a support is composed at least in part of a metal, ceramic, glass, plastic, gel, or other matrix. Such articles may, for example, take the form of plates (e.g., multiwell plates), slides, particles (e.g., “beads”, e.g., magnetic beads), pellets, bars, rods, pins, disks, chips, filters, or other suitable forms. In some embodiments, a support comprises a sensor, e.g., a sensor capable of detecting changes in binding. For example, the sensor may detect a change in weight or a signal such as fluorescence. In some embodiments, the support comprises an electrode. In some embodiments, compounds are arranged as a small molecule microarray. Compounds could be present in multiple locations on a surface, in individual wells or vessels, etc. See, e.g., Vegas A J, et al., Chem Soc Rev. 37(7):1385-94, 2008. Noncovalent attachment could be, e.g., by adsorption of the polypeptide or compound to the surface (which may be coated with a substance to facilitate such adsorption), via an affinity-based mechanism, or other means of immobilizing the ATPIF1 polypeptide or test agent so that it remains physically associated with the support. In some embodiments, an antibody is used to attach an ATPIF1 polypeptide or test agent to a support. In some embodiments, an ATPIF1 polypeptide or test agent is attached to a support via a biotin-avidin interaction or other strong binding interaction, wherein one of two binding partners is attached directly or indirectly to the support and the other binding partner is attached to the entity to be immobilized.

In some embodiments, multiple test agents are immobilized in different locations (e.g., in an array format). ATPIF1 polypeptide is added and the composition is maintained for a suitable time period to allow binding to occur. In some embodiments, unbound material is removed by washing, and ATPIF1 polypeptide is detected using a suitable binding agent (e.g., an antibody) or, if the polypeptide is detectably labeled, by detecting a signal. In some embodiments, a washing step is not necessary. For example, binding may be detected by measuring a change in fluorescence polarization (FP), FRET, or electrochemiluminecence. A variety of in-solution fluorescence-based strategies of use for high-throughput quantification of protein interactions are described in Hieb, A R, et al., Nucleic Acids Res. 2012; 40(5):e33. In some embodiments such an assay is adapted for use in screening to identify test agents that disrupt or enhance a specific interaction, e.g., an ATPIF1 PPI. FP assays are reviewed in Lea W A & Simeonov A. Expert Opin Drug Discov. 2011; 6(1):17-32.

In some embodiments, ATPIF1 polypeptide is immobilized, test agents are added, and binding is measured using similar approaches. In some embodiments a transition-metal-based fluorescence polarization assay is used. For example, an ATPIF1 polypeptide-biotin conjugate is immobilized in a streptavidin-coated well plate, to which is added an ATPIF1 polypeptide labeled with a luminescent transition-metal complex such as Ru(bpy)₃. An increase in the fluorescence polarization (FP) signal is observed in the wells coated with ATPIF1 polypeptide-biotin compared to wells to which the ATPIF1 polypeptide-biotin conjugate has not been added. Agents are screened for their ability to inhibit ATPIF1 polypeptide dimerization. In some embodiments an agent that inhibits ATPIF1 polypeptide dimerization may induce dissociation of already existing ATPIF1 polypeptide dimers using the HTFP assay. In some embodiments test agents are added to wells containing the immobilized ATPIF1 polypeptide-biotin conjugate. Labeled ATPIF1 polypeptide is then added. The FP signal from the wells is assessed. If the signal from a particular well is less than a signal from a control well (e.g., a well to which a test agent was not added), the test agent that was added to that particular well is identified as am inhibitor of ATPIF1 dimerization. In some embodiments labeled ATPIF1 polypeptide is added to wells containing the immobilized ATPIF1 polypeptide-biotin conjugate and dimerization is allowed to occur. Test agents are added to the wells. Labeled ATPIF1 polypeptide is then added. The FP signal from the wells is assessed. If the signal from a particular well is less than a signal from a control well (e.g., a well to which a test agent was not added), the test agent from that particular well is identified as an inhibitor of ATPIF1 dimerization.

In some embodiments two or more assays are performed with a test agent or library, e.g., a FRET assay and an FP assay. In some embodiments two or more assays are performed in the same well. Test agents that are “hits” in both assays are identified,

In some embodiments peptides that bind to an ATPIF1 polypeptide are identified using a display technique, e.g., phage display, and then screened for ability to inhibit an ATPIF1 polypeptide PPI, e.g., ATPIF1 polypeptide dimerization.

Any of the methods may include appropriate controls to, e.g., reduce the number of false positives.

In some embodiments, surface plasmon resonance (SPR) is used to measure kinetics (on and/or off rates) and/or binding strength (affinity) between a test agent and an ATPIF1 polypeptide. For example, using SPR technology (e.g., systems such as those available from Biacore, Life Sciences, GE Healthcare) the binding and dissociation of a test compound to a protein immobilized on a chip can be measured, and the measured values compared with those obtained when a solution not containing the test compound is loaded on the chip. A test agent capable of binding to the protein can be selected on the basis of the binding and dissociation rate and/or binding level. Other useful methods for detecting and/or quantifying binding include use of a quartz crystal microbalance, optical cantilever, microchannel resonator, dual polarisation interferometer, coupled waveguide plasmon resonance, immunoprecipitation or other antibody-based detection methods, isothermal titration and differential scanning calorimetry, differential scanning fluorimetry, capillary electrophoresis, resonance energy transfer, electrochemiluninesce, photonic crystals, and fluorescent correlation analysis.

In some embodiments, an aptamer, peptide, polypeptide, or small molecule that is known to bind to a ATPIF1 polypeptide is labeled and used as a tool for screening test agents (e.g., small molecules) for ability to bind to and/or inhibit activity of the ATPIF1 polypeptide. The label can comprise, e.g., a radioactive, fluorescent, luminescent, or other readily detectable moiety. The ability of a test compound to compete with the labeled aptamer, peptide or small molecule can be detected and serves as an indicator of the binding of the test compound to the ATPIF1 polypeptide. For example, a scintillation proximity assay (SPA) can be used. In some embodiments of an SPA for identifying agents that bind to an ATPIF1 polypeptide, the ATPIF1 polypeptide is attached to beads containing a scintillant material. The beads are typically located in wells or other vessels. In another embodiment, an ATPIF1 polypeptide is attached to scintillant material is embedded directly into wells. A radiolabelled compound capable of binding to the ATPIF1 polypeptide and a test compound are added to the well. Binding of the radiolabelled compound to the ATPIF1 polypeptide results in a signal. The signal is reduced in the presence of a test compound that competes with the radiolabelled compound for binding. See, e.g., J. Fraser Glickman, et al., Scintillation Proximity Assays in High-Throughput Screening. Assay and Drug Development Technologies. 6(3): 433-455, 2008, for review of SPA. Similar assays can be performed using filters.

In some embodiments, an agent is identified that binds to an ATPIF1 polypeptide with a Kd equal to or less than approximately 1 mM, 500 μM, 100 μM, 50 μM, 10 μM, 5 μM., or 1 μM. In some embodiments, an agent is identified that binds to an ATPIF1 polypeptide with a Kd equal to or less than approximately 500 nM, 100 nM, 50 nM, or 10 nM. In some embodiments, is identified that binds to an ATPIF1 polypeptide with a Kd between 0.1-10 nM. Agents that bind to an ATPIF1 polypeptide may be further tested, e.g., in cell-free or cell-based assays, to determine the extent to which they inhibit ATPIF1 activity.

In some embodiments an assay is performed at a pH at which ATPIF1 would normally bind to and inhibit F0-F1 ATPase.

A variety of different assays can be employed to identify and/or characterize agents that modulate, e.g., inhibit, at least one ATPIF1 activity. In embodiments an ATPIF1 activity comprises an ATPIF1 protein-protein interaction (PPI). In some embodiments an ATPIF1 PPI comprises dimerization of ATPIF1. In some embodiments an ATPIF1 PPI comprises binding of ATPIF1 or an ATPIF1 dimer to a different polypeptide or complex (e.g., F0-F1 ATP synthase or a subunit thereof). Assays of use to detect protein-protein interactions can be adapted for use with an ATPIF1 polypeptide to characterize PPIs and/or to assess agents for their potential to inhibit an ATPIF1 PPI. In some embodiments a proximity-dependent assay (proximity assay) is used. Proximity-dependent assays include a variety of different assays in which a signal is generated or altered when two or more entities whose interaction is of interest or is to be detected are in close proximity to one another. In some embodiments a proximity-dependent assay uses Alpha (Amplified Luminescent Proximity Homogeneous Assay) Screen® technology (PerkinElmer). This technology makes use of two types of beads: donor beads and acceptor beads. Donor beads contain a photosensitizer, phthalocyanine, which converts ambient oxygen to an excited and reactive form of O2, singlet oxygen, upon illumination at 680 nm. Singlet oxygen can diffuse approximately 200 nm in solution. If an acceptor bead is within that distance, energy is transferred from the singlet oxygen to thioxene derivatives within the acceptor bead, resulting in light production. If the donor bead is not in proximity of an acceptor bead, the singlet oxygen falls to ground state and no signal is produced. In an Alpha PPI assay, one protein is captured on donor beads, and the other protein is captured on acceptor beads. The beads are maintained in solution. When the two proteins interact, the donor bead is brought into proximity of the acceptor bead, and excitation of the donor bead will result in signal generation dependent on the presence of a PPI. Two proteins of interest can comprise a different tag to facilitate their capture to donor and acceptor bead. If dimerization is to be assessed, two aliquots of the protein can be prepared with different tags for capture to donor and acceptor beads. Agents that alter a PPI can be identified by detecting a difference in signal generated in the presence of the test agent as compared with a control well lacking the test agent.

In some embodiments a proximity assay is a “protein fragment complementation assay” (PCA), in which a reporter molecule (typically a protein) capable of generating a detectable signal is reconstituted as a result of interaction between proteins of interest, each of which comprises a fragment of the reporter molecule, often at the N- or C-terminus. Reconstitution of the reporter molecule results, e.g., in a protein that can be directly or indirectly detected. Fragments of the reporter molecule are selected that produce no or low signal by themselves and have low affinity for each other but have the capacity to reassemble to form a detectable reporter molecule when brought into proximity. The sequence of a fragment of a reporter molecule can be altered to, e.g., reduce spontaneous assembly of the fragments. For purposes hereof, a polypeptide useful for reconstitution as a reporter molecule in a PCA assay may be referred to as a “PCA fragment”. Examples of PCAs include enzyme complementation assays, fluorescence complementation assays, luciferase complementation assays, and protease complementation assays. Exemplary reporter proteins of use in PCAs include enzymes such as dihydrofolate reductase and β-lactamase; fluorescent proteins such as green fluorescent protein (GFP) and variants thereof; and luciferases such as firefly luciferase, Gaussia luciferase, and Renilla luciferase. The split ubiquitin assay and the split tobacco etch virus (TEV) protease assay are exemplary protease complementation assay. In some embodiments of a protease complementation assay, reconstitution of the fragments results in a proteolytically active protein that activates a reporter by proteolytic cleavage. Cleavage of the reporter results directly or indirectly in a detectable signal. Two-hybrid screens may be considered a type of PCA assay, in which the PCA fragments comprise the binding domain (BD) and activating domain (AD) of a transcription factor. Agents that alter a PPI can be identified by detecting a difference in signal generated in the presence of the test agent as compared with a control well lacking the test agent. Various proximity-based assays, reporter molecules, and PCA fragments are discussed in Kerppola, T., Chem Soc Rev. 2009; 38(10): 2876-2886.

In some embodiments, a readout for a proximity assay is based on resonance energy transfer (RET), e.g., fluorescence resonance energy transfer (FRET), luminescence resonance energy transfer (LRET), or bioluminescence resonance energy transfer (BRET). A wide variety of RET-based assays can be implemented. In general, such assays make use of a distance-dependent interaction involving energy transfer between two moieties (sometimes termed a donor and acceptor). FRET is a distance-dependent interaction between the electronic excited states of two moieties in which excitation is transferred from a donor moiety to an acceptor moiety without emission of a photon, resulting in emission from the FRET acceptor. LRET has similarities to FRET but uses a luminescent moiety, e.g., a lanthanide as the energy-transfer donor. BRET is analogous to FRET but uses a luminescent or luminescence-generating biomolecule such as luciferase, aequorin, or a derivative thereof as an energy donor and a fluorescent moiety, e.g., a biomolecule such as green fluorescent protein (GFP) as the acceptor, thus eliminating the need for an excitation light source (reviewed in Pfleger, K. an Eidne, K., Nature Methods, 3(3), 165-174, 2006). To implement a RET-based PPI assay, a first protein of interest is labeled with a RET donor, and a second protein of interest is labeled with an appropriate RET acceptor. If dimerization is to be assessed, first and second aliquots of the protein of interest are labeled with a RET donor and RET acceptor, respectively. Assays may detect acceptor emission, donor quenching (decreased emission from the RET donor), and/or an alteration in the fluorescence lifetime of the donor. Assays can make use of increases in acceptor emission, decreases in acceptor emission, donor quenching, reduction in donor quenching, and/or increase or decrease in fluorescence lifetime of the donor to detect increased or decreased proximity of a RET donor and RET acceptor. Nonfluorescent acceptors, also referred to as quenchers are of use and include dabcyl and QSY dyes. Such molecules are capable of absorbing the energy of an excited fluorescent label when located in close proximity and of dissipating that energy without the emission of visible light. Numerous suitable donor/acceptor pairs are known in the art. A wide variety of different RET donors and acceptors are of use. RET donors and acceptors include molecules in various classes including: organic materials (including “traditional” dye fluorophores, quenchers, and polymers; inorganic materials such as metal chelates, metal and semiconductor nanocrystals (e.g., “quantum dots”, and fluorophores of biological origin such as fluorescent proteins and amino acids; and biological compounds that exhibit bioluminescensce upon enzymatic catalsysis. Specific examples of RET donors and acceptors include acridine dyes; Alexa dyes; BODIPY, cyanine dyes; fluorescein and derivatives thereof; rhodamine derivatives thereof; GFP and derivatives thereof; blue, sapphire, yellow, red, orange, and cyan fluorescent proteins and derivatives thereof; monomeric red fluorescent protein (mRFPI) and derivatives such as those known as “mFruits”, e.g., mCherry, mStrawberry, etc., quantum dots, etc. Organic UV dyes are typically pyrene, naphthalene, and coumarin-based structures. Visible/near IR dyes include a number of fluorescein, rhodamine, and cyanine-based derivatives. One of ordinary skill in the art will readily be able to select appropriate RET donor and acceptor pairs. There are numerous resources in the literature to assist with such selection. See, e.g., The Molecular Probes Handbook—A Guide to Fluorescent Probes and Labeling Technologies, 11^{th} edition (Life Technologies), which describes numerous fluorescent and otherwise detectable molecules and methods for their use and modification. See also Trinquet, E. and Mathis, G., MoI. Biosyst., 2: 380-387, 2006; Sapsford, K, et al, Angewandte Chemie Int. Ed, 45: 4562-4588, 2006 for further information on RET. A nonlimiting list of exemplary FRET donor/acceptor pairs includes: coumarin/fluorescein; fluorescein/rhodamine; Cy3.5/Cy5; Alexa fluors/GFP; YFP/GFP; CYPet/YPet, etc. Dye/quencher combinations include rhodamine/Dabcyl and Cy3/QSY9. RET donor and acceptor moieties are commercially available from a number of suppliers including Life Technologies, Amersham Biosciences, Pierce, Biosearch Technologies, etc. Certain RET donors and acceptors may be suitable for cell-based assays; some may be suitable for assays employing isolated or purified polypeptides; and some may be suitable for both types of assays. For example, if the assay is to be conducted using whole cells, it may be advantageous to select RET donors and acceptors that comprise polypeptides (referred to herein as “RET polypeptides”), allowing them to be incorporated as part of a fusion protein, e.g., a fusion protein comprising a first portion comprising an ATPIF polypeptide and a second portion comprising a RET polypeptide.

A polypeptide comprising an ATPIF1 polypeptide and a RET polypeptide or PCA fragment can be encoded by a nucleic acid construct that comprises an open reading frame (ORF) encoding the RET polypeptide in frame with an ORF encoding the ATPIF1 polypeptide. The two ORFs may be separated by a polynucleotide sequence that encodes a linker region. The linker region may be a short polypeptide chain (e.g., 1-50 amino acids, e.g., 5-25 or 5-15 amino acids). The precise length and sequence are typically not critical. Small amino acid residues such as serine, glycine, and alanine are of use. Examples include (Gly-Ser)n, (Thr-Ser-Pro)n, (Gly-Gly-Gly)n, (GIy-Ala)n, and (Glu-Lys)n, wherein n is 1 to 15, and variants in which any of the amino acid residues is repeated with the proviso that the total number of amino acids is within one of the aforementioned ranges. The resulting ORF may be translated in vitro or in cells to produce the fusion protein. In some embodiments the ORF encoding the RET polypeptide or PCA fragment is appended at the 5′ or 3′ end of the ORF that encodes the ATPIF1 polypeptide. It may be desirable to append the RET donor or acceptor or PCA fragment at the terminus opposite the region of the ATPIF1 polypeptide expected to participate in a physical interaction. In some embodiments a RET donor or acceptor or PCA fragment is appended at the C-terminus. In some embodiments aRET donor or acceptor or PCA fragment is appended at the N-terminus. The nucleic acid construct may be inserted into a vector in operable association with expression control elements such as a promoter, promoter/enhancer, etc. Appropriate polyadenylation and termination signals, etc., may be included. The vector is introduced into an appropriate host cell using art-accepted methods appropriate for the host cell. The ORF is transcribed and translated in the cell to produce the fusion protein. The fusion protein may be purified and used in a cell-free assay or cells may be used in a cell-based assay.

A “cell-based assay” is an assay in which viable cells that express or contain an ATPFI1 polypeptide are contacted with a test agent (e.g., by adding the test agent to cell culture medium), and a parameter of interest such as ATPIF1 level or activity is assessed. In some embodiments the effect of the test agent on an ATPIF1 PPI is assessed. For example, in some embodiments the effect of a test agent on ATPIF1 dimerization is assessed, e.g., as described above. In some embodiments, the effect of the test agent on the ability of the ATPIF1 polypeptide to inhibit ATPase activity of the F0-F1 ATPase is assessed. For example, ATP synthesis can be measured in the presence or absence of the test agent under conditions in which ATPIF1 would normally inhibit ATP activity of F0-F1 ATPase. In some embodiments the ability of a test agent to increase or decrease the level of F0-F1 ATPase inhibition is assessed.

In some embodiments an agent capable of causing a decrease in level or activity of ATPIF1, of at least 50% when used in a cell-free or cell-based assay at a concentration equal to or less than approximately 1 mM, 500 μM, 100 μM, 50 μM, 10 μM, 5 μM, or 1 μM, may be screened for, identified, selected, designed, provided, or used. In some embodiments an agent capable of causing a decrease in level or activity of ATPIF1 of at least 50% (i.e., a decrease to 50% or less of the activity that would be expected in the absence of the agent) when used in a cell-free or cell-based assay at lower concentrations, e.g., equal to or less than approximately 500 nM, 100 nM, 50 nM, or 10 nM or less, may be identified, selected, designed, or used. In some embodiments an agent capable of causing a decrease in level or activity of ATPIF1 of at least 50% when used at a concentration between 0.1-10 nM, may be screened for, identified, selected, designed, provided, or used.

An agent identified as a modulator, e.g., an inhibitor, of an ATPIF1 polypeptide can be tested in cell culture or in animal models (“in vivo”). In some embodiments, such testing is used to assess the effect of the agent on mitochondrial phenotype or function. In some embodiments, such testing is performed to assess the ability of the agent to treat a mitochondrial disorder. In some embodiments, cells are contacted with an ATPIF1 inhibitor and a mitochondrial poison. In some embodiments the mitochondrial poison is used at a concentration and for a time at which it would result in detectable mitochondrial dysfunction in the absence of an ATPIF1 inhibitor. The ability of the agent to inhibit such mitochondrial dysfunction is assessed. If the agent detectably reduces mitochondrial dysfunction, the agent is identified as a candidate agent for treatment of mitochondrial disorders. In some embodiments the mitochondrial poison is used at a concentration and for a time at which it would kill at least some of the cells in the absence of an ATPIF1 inhibitor. The ability of the agent to inhibit cell) death is assessed. If the agent detectably reduces or delays cell death, the agent is identified as a candidate agent for treatment of mitochondrial disorders. In some embodiments, an agent that modulates ATPIF1, e.g., an ATPIF1 inhibitor, is tested in a system that serves as a model of a mitochondrial disorder, wherein the system does not comprise use of a mitochondrial poison. Examples of such systems are described in Section IV. In some embodiments, if the agent shows evidence of a protective effect in the model, the agent is confirmed as a useful agent for treatment of a mitochondrial disorder.

In some embodiments, a cell naturally expresses ATPIF1. In some embodiments a cell is modified so that it expresses ATPIF1 polypeptide at a higher or lower level than would be the case in the absence of the modification. In some embodiments expression level is normalized, e.g., based on expression of a “housekeeping” gene. Commonly used housekeeping genes include, e.g., beta-actin, tubulin, (EF1alpha), etc.

In some embodiments, a cell is of a type that is susceptible to a mitochondrial poison. In some embodiments a cell type is of a type that is affected by a mitochondrial disorder. In some embodiments an agent is tested in two or more different cell types.

In some embodiments, a cell used in a method described herein is genetically modified or selected to have a property that facilitates its use to test agents. For example, in some embodiments a cell is genetically modified or selected to have reduced or absent expression of one or more molecular pumps that may otherwise transport a test agent out of the cell. In some embodiments, the cell is modified to facilitate detection of a mitochondrial phenotype or function.

Cells can be contacted with test agents(s) and/or mitochondrial poisons for various periods of time. In certain embodiments cells are contacted with test agent(s) and/or mitochondrial poisons for between 1 hour and 20 days, e.g., for between 12 and 48 hours, between 48 hours and 5 days, e.g., about 3 days, between 5 days and 10 days, or any intervening range or particular value. Cells can be contacted with a test agent during all or part of a culture period. In some embodiments, cells are contacted with a mitochondrial poison prior to contacting the cells with a test agent. In some embodiments, cells are contacted with a test agent prior to contacting them with a mitochondrial poison. A range of concentrations and/or time periods can be tested to identify an appropriate concentration, time period, or combination thereof.

If desired, cytotoxicity can be assessed e.g., by detecting cell lysis (which may be evident as clear areas or “plaques” in a cell monolayer) or using any of a variety of assays for cell viability and/or proliferation such as a cell membrane integrity assay, a cellular ATP-based viability assay, a mitochondrial reductase activity assay, a BrdU, EdU, or H3-Thymidine incorporation assay, a DNA content assay using a nucleic acid dye, such as Hoechst Dye, DAPI, Actinomycin D, 7-aminoactinomycin D or propidium iodide, a cellular metabolism assay such as AlamarBlue, MTT, XTT, and CellTitre Glo, etc.

The effect of an agent may be expressed as the 50% inhibitory concentration (IC₅₀), defined as the lowest concentration of agent that results in a 50% decrease in the parameter being assessed, as compared with a control well that does not contain the agent. If desired, an IC₉₀ can be assessed in a similar manner. In some embodiments, one or more compound(s) with a desired IC₉₀ or IC₉₀ is identified. In some embodiments, an IC₅₀ and/or IC₉₀ is no greater than 100 mg/ml, e.g., no greater than 10 mg/ml, e.g., no greater than 1.0 mg/ml, e.g., no greater than 100 μg/ml, e.g., no greater than 10 μg/ml, e.g., no greater than 5 μg/ml or no greater than 1 μg/ml. In some embodiments, an IC50 and/or IC90 is less than or equal to 500 μM. In some embodiments, an IC50 and/or IC90 less than or equal to 10 μM. In some embodiments, an IC50 and/or IC90 less than or equal to 10 M. In some embodiments, an IC50 and/or IC90 is in the nanomolar range, i.e., less than or equal to 1 μM.

In some embodiments, a high throughput screen (HTS) is performed. A high throughput screen can utilize cell-free or cell-based assays. High throughput screens often involve testing large numbers of compounds with high efficiency, e.g., in parallel. For example, tens or hundreds of thousands of compounds can be routinely screened in short periods of time, e.g., hours to days. Often such screening is performed in multiwell plates containing, e.g., e.g., 96, 384, 1536, 3456, or more wells (sometimes referred to as microwell or microtiter plates or dishes) or other vessels in which multiple physically separated cavities or depressions or areas are present in or on a substrate. High throughput screens can involve use of automation, e.g., for liquid handling, imaging, data acquisition and processing, etc. Certain general principles and techniques that may be applied in embodiments of a HTS of the present invention are described in Macarron R & Hertzberg R P. Design and implementation of high-throughput screening assays. Methods Mal Biol., 565:1-32, 2009 and/or An W F & Tolliday N J., Introduction: cell-based assays for high-throughput screening. Methods Mol. Biol. 486:1-12, 2009, and/or references in either of these. Useful methods are also disclosed in High Throughput Screening: Methods and Protocols (Methods in Molecular Biology) by William P. Janzen (2002) and High-Throughput Screening in Drug Discovery (Methods and Principles in Medicinal Chemistry) (2006) by Jorg Hüser.

In some embodiments, one or more screens is/are performed to identify agents that bind to and/or inhibit ATPIF1 polypeptide, and the ability of one or more agents identified in such screen(s) (or analogs or derivatives thereof) to improve mitochondrial function and/or to protect a cell against mitochondrial dysfunction is then assessed.

In some embodiments a method comprises (a) producing an ATPIF1 inhibitor; and (b) assessing the ability of the ATPF1 inhibitor to improve mitochondrial function and/or to protect a cell against mitochondrial dysfunction

In some aspects, compositions comprising components appropriate to perform any of the methods described herein are provided. In some embodiments, an assay system comprises components suitable for identifying an ATPIF1 inhibitor.

In some embodiments, the invention provides a cell culture or cell line comprising near-haploid cells that have an insertion into the ATPIF1 locus or otherwise lack expression of ATPIF1. The near-haploid cells are of a species, e.g., a mammal, e.g., a human, whose somatic cells are normally diploid. In some embodiments, a near-haploid cell or cell line expresses a variant ATPIF1 polypeptide. In some embodiments the variant is a tagged functional ATPIF1 polypeptide. In some embodiments expression of the variant is inducible or repressible. Optionally the near-haploid cell or cell line has an insertion (e.g., a gene trap vector insertion) in the endogenous ATPIF1 gene. In some embodiments the near-haploid cell line or subclones thereof may gain chromosomes over time and become non-near haploid. In some embodiments cells having insertions in the ATPIF1 gene or otherwise having reduced or absent ATPIF1 expression are useful for assessing candidate ATPIF1 modulators. In some embodiments cells having insertions in the ATPIF1 gene or otherwise having reduced or absent ATPIF1 expression are useful for exploring the role of ATPIF1 on mitochondrial or cell physiology.

In some embodiments, information derived from sequence analysis, mutational analysis, and/or structural analysis may be used in the identification or analysis of ATPIF1 modulators, e.g., ATPIF1 inhibitors. For example, in some embodiments a structure (e.g., a two-dimensional or three-dimensional structure) of a target, e.g., a ATPIF1 protein, generated at least in part using, e.g., nuclear magnetic resonance, homology modeling, and/or X-ray crystallography is used. In some embodiments a structure of ATPIF1 bound to F0-F1 ATPase may be used. Residues in the ATPIF1 and F1-ATPase protein complexes form numerous interactions. In some embodiments a compound is designed to interfere with one or more such interactions.

In some embodiments a computer-aided computational approach sometimes referred to as “virtual screening” is used in the identification of candidate ATPIF1 modulators, e.g., candidate ATPIF1 inhibitors. Structures of compounds may be screened for ability to bind to a region (e.g., a “pocket”) accessible to the compound. The region may be a known or potential active site or any region accessible to the compound, e.g., a concave region on the surface or a cleft. A variety of docking and pharmacophore-based algorithms are known in the art, and computer programs implementing such algorithms are available. Commonly used programs include Gold, Dock, Glide, FlexX, Fred, and LigandFit (including the most recent releases thereof). See, e.g., Ghosh, S., et al., Current Opinion in Chemical Biology, 10(3): 194-2-2, 2006; McInnes C., Current Opinion in Chemical Biology; 11(5): 494-502, 2007, and references in either of the foregoing articles, which are incorporated herein by reference. In some embodiments a virtual screening algorithm may involve two major phases: searching (also called “docking”) and scoring. During the first phase, the program automatically generates a set of candidate complexes of two molecules (test compound and target molecule) and determines the energy of interaction of the candidate complexes. The scoring phase assigns scores to the candidate complexes and selects a structure that displays favorable interactions based at least in part on the energy. To perform virtual screening, this process may be repeated with a large number of test compounds to identify those that, for example, display the most favorable interactions with the target. In some embodiments, low-energy binding modes of a small molecule within an active site or possible active site are identified. Variations may include the use of rigid or flexible docking algorithms and/or including the potential binding of water molecules.

Numerous small molecule structures are available and can be used for virtual screening. A collection of compound structures may sometimes referred to as a “virtual library”. For example, ZINC is a publicly available database containing structures of millions of commercially available compounds that can be used for virtual screening (http://zinc.docking.org/; Shoichet, J. Chem. Inf. Model., 45(1):177-82, 2005). A database containing about 250,000 small molecule structures is available on the National Cancer Institute (U.S.) website (at http://129.43.27.140/ncidb2/). In some embodiments multiple small molecules may be screened, e.g., up to 50,000; 100,000; 250,000; 500,000, or up to 1 million, 2 million, 5 million, 10 million, or more. Compounds can be scored and, optionally, ranked by their potential to bind to a target. Compounds identified in virtual screens can be tested in cell-free or cell-based assays or in animal models to confirm their ability to inhibit activity of ATPIF1 and/or to assess their effect on mitochondrial phenotype, function, or dysfunction, to assess their effect on or survival or proliferation or cells having mitochondrial dysfunction.

Computational approaches can be used to predict one or more physico-chemical, pharmacokinetic and/or pharmacodynamic properties of compounds identified in physical or virtual screens. For example, absorption, distribution, metabolism, and excretion (ADME) parameters can be predicted. Such information can be used, e.g., to select hits for further testing or modification. For example, small molecules having characteristics typical of “drug-like” molecules can be selected and/or small molecules having one or more undesired characteristics can be avoided.

Additional compounds that inhibit, ATPIF1 can be identified or designed based on initial compounds (“hits”) identified in a physical or virtual screen such as those described above. In some embodiments, structures of hit compounds are examined to identify a pharmacophore, which can be used to design additional compounds (“derivatives”).

An additional compound may, for example, have one or more improved (i.e., more desirable) pharmacokinetic and/or pharmacodynamic properties as compared with an initial hit or may simply have a different structure. For example, a compound may have higher affinity for the molecular target of interest (e.g., ATPIF1), lower affinity for a non-target molecule, greater solubility (e.g., increased aqueous solubility), increased stability, increased bioavailability, and/or reduced side effect(s), etc. Optimization can be accomplished through empirical modification of the hit structure (e.g., synthesizing compounds with related structures and testing them in cell-free or cell-based assays or in non-human animals) and/or using computational approaches. Such modification can make use of established principles of medicinal chemistry to predictably alter one or more properties.

In some embodiments, an agent that causes a decrease in ATPIF1 level or activity of at least 50% (i.e., a decrease to 50% or less of the activity that would be expected in the absence of the agent) when used in a cell-free or cell-based assay at a concentration equal to or less than approximately 1 mM, 500 μM, 100 μM, 50 μM, 10 μM, 5 μM, or 1 μM is identified, tested, produced, or used. In some embodiments, an agent that causes a decrease in ATPIF1 level or activity of at least 50% (i.e., a decrease to 50% or less of the activity that would be expected in the absence of the agent) when used in a cell-free or cell-based assay at lower concentrations, e.g., equal to or less than approximately 500 nM, 100 nM, 50 nM, or 10 nM or less is identified, tested, produced, or used. In some embodiments, an agent that causes a decrease in ATPIF1 activity of at least 50% when used at a concentration between 0.1-10 nM is identified, tested, produced, or used.

In some aspects, gene therapy is contemplated in order to inhibit ATPIF1. Gene therapy encompasses methods that comprise use of nucleic acids as therapeutic agents to treat disease by supplementing or altering the genome within an individual's cells in vivo. Also encompassed are methods that comprise use of nucleic acids as therapeutic agents to treat disease by supplementing or altering the genome of cells ex vivo and administering the cells to a subject. The cells may be derived from the subject or from a suitable donor. Gene therapy typically comprises introducing a nucleic acid that encodes a therapeutic protein or therapeutic RNA into a cell, thereby supplementing the cell's own genetic information. The nucleic acid or a copy thereof is expressed by the cell, resulting in the production of a therapeutic protein or RNA by the cell. In general, the therapeutic protein or RNA may be any protein or RNA that is useful to treat a disease. In some embodiments the nucleic acid that encodes a therapeutic protein or RNA is contained in a vector, which is used to introduce the nucleic acid into cells. The vector may be referred to as a “gene therapy vector”. It will be understood that a gene therapy vector may be used for therapeutic purposes, for non-therapeutic purposes in which gene transfer is desired, or both.

In some aspects, the present disclosure provides a gene therapy vector that encodes a protein or RNA that inhibits expression or activity of ATPIF1. In some embodiments a gene therapy vector is an RNAi vector, e.g., the vector is used to introduce a nucleic acid encoding a shRNA, or miRNA (e.g., an artificial miRNA) that inhibits ATPIF1 into cells. In some embodiments a gene therapy is a viral vector. In some embodiments a gene therapy vector is non-viral. A variety of vectors suitable for use as gene therapy vectors are known in the art and may be used in various embodiments. Examples of viruses useful for gene therapy include, e.g., retrovirus (e.g., lentivirus), adenovirus, adeno-associated virus, herpes simplex virus, poxvirus, or baculovirus. In some embodiments viral particles (virions) are used as gene therapy vectors. In some embodiments plasmids containing appropriate components of the viral genome are used. In some embodiments, a nucleic acid encoding the therapeutic polypeptide or RNA may be stably maintained by integration into the cell's nuclear genome or by episomal persistence. In some embodiments a gene therapy vector is generally not pathogenic to individuals of the species to which it is administered. In some embodiments a gene therapy vector capable of infecting or entering non-dividing cells is used.

To produce a viral gene therapy vector a nucleic acid comprising a sequence encoding a protein or RNA to be produced in the cell is inserted at an appropriate position in the viral genome. Typically, a modified viral genome is used in which at least some of the viral genes and/or regulatory regions have been disabled or removed from the genome to render the virus replication-incompetent and/or to provide room for inserting a nucleic acid to be delivered to a cell. The resulting viral genome typically retains at least those elements required in cis for integration and/or maintenance in a cell. The viral genome, which may be incorporated into a plasmid, may be introduced into a cell to be used to produce viral particles Viral gene products required for producing viral particles may be provided in trans in a production system, e.g., by helper virus, plasmids, or transgenic producer cells.

In some embodiments a retroviral vector may be used. Retrovirus vectors may be based on gammaretroviruses such as murine leukemia virus (MLV) which may be pseudotyped with envelope proteins from other viruses such as the gibbon ape leukemia virus envelope protein (GALV) or vesicular stomatitis virus G protein (VSV-G) to permit transduction of human cells. A typical retrovirus genome comprises long terminal repeats (LTRs) flanking a primer binding site (PBS), packaging signal (ψ), and polypurine tract (PPT) regulatory regions and the viral genes gag, pro, pol, and env. In some embodiments lentiviral vectors based on, e.g., human immunodeficiency virus type-1 (HIV-1) are used. Lentiviruses typically contain the retrovial genes mentioned above as well as additional genes (e.g., tat) and regulatory regions, at least some of which are deleted in typical lentiviral vectors. In some embodiments a self-inactivating (SIN) vector may be used. Such vectors may be created by deletion of at least part of the U3 portion of the 3′ LTR. In general, the nucleic acid encoding a desired protein or RNA, e.g., a polypeptide or RNA that inhibits ATPIF1 expression or activity, is inserted between the LTRs. Typically an expression cassette comprising a nucleic acid sequence encoding a desired protein or RNA operably linked to a promoter is inserted, although expression may in some embodiments be achieved using the 5′ LTR as a promoter. Exemplary retroviral, and lentiviral vectors are described in US Pat. Pub. No. 20050251872 and US Pat. Pub. No. 20040259208. In some embodiments a second or third generation lentiviral vector may be used. In some embodiments a packaging system in which the Tat protein has been eliminated from the packaging and the Rev protein is expressed on an independent plasmid may be used.

In some embodiments an adenoviral vector may be used. The human adenovirus (Ad) family consists of seven species designated A-G. At least 57 different serotypes have been identified to date. In some embodiments a vector from species C, e.g., serotype 2 (Ad2) or serotype 5 (Ad5) is used. First, second, and third generation Ad vectors are known in the art, distinguished by removal of increasing amounts of the native viral genome (see, e.g., Dormond, E., et al., Biotechnol Adv. 2009 March-April; 27(2):133-44). The third generation AdV genome comprises cis-acting elements, i.e., the packaging signal (ψ) and the inverted terminal repeats (ITR), and is devoid of viral genes. A vector comprising an expression cassette containing a nucleic acid encoding the desired gene product, e.g., encoding a polypeptide or RNA that inhibits ATPIF1 expression or activity, operably linked to a promoter and inserted between the adenoviral ITRs, may be generated using any of a variety of methods.

Adeno-associated virus is a small (20 nm) replication-defective, nonenveloped virus. The AAV genome a single-stranded DNA (ssDNA) about 4.7 kilobase long. The genome comprises inverted terminal repeats (ITRs) at both ends of the DNA strand, and two open reading frames (ORFs): rep and cap. The AAV genome integrates most frequently into a particular site on chromosome 19. Random incorporations into the genome take place with a negligible frequency. The integrative capacity may be eliminated by removing at least part of the rep ORF from the vector resulting in vectors that remain episomal and provide sustained expression at least in non-dividing cells. To use AAV as a gene transfer vector, a nucleic acid comprising a nucleic acid sequence encoding a desired protein or RNA, e.g., encoding a polypeptide or RNA that inhibits ATPIF1, operably linked to a promoter, is inserted between the inverted terminal repeats (ITR) of the AAV genome.

Further information regarding these and other gene therapy vectors of interest and various methods of making and using such vectorsis described in Friedmann, T. (ed.), Gene Transfer Vectors for Clinical Application. Methods in Enzymology. Volume 507, Elsevier, Inc., 2012. For example, chapter 12 discusses adeno-associated virus vectorology and manufacturing; chapter 13 discusses gene delivery to the retina; chapter 14 discusses generation of hairpin-based RNAi vectors. Adeno-associated viruses (AAV) and their use as vectors, e.g., for gene therapy, are also discussed in Snyder, R O and Moullier, P., Adeno-Associated Virus Methods and Protocols, Methods in Molecular Biology, Vol. 807. Humana Press, 2011. One of ordinary skill in the art will appreciate that many different vectors and/or production methods are available and may be used in various embodiments.

A wide variety of vectors that are of particular use for expression of RNAi agents, e.g., shRNA or miRNA are available. In some embodiments a vector for expression of an shRNA comprises an RNA pol III promoter and RNA pol III terminator sequence. Examples of vectors useful for expression of shRNA include, e.g., pMKO.1 (a retroviral vector based on MuLV), pLKO.1 (a lentiviral vector), pAAV-U6-puro (Cell Biolabs, Inc., San Diego, Calif. Cat. No. VPK-412), to name but a few. In some embodiments an H1, 7SK, or tRNA promoter may be used instead of U6, or an RNA pol II promoter may be used. In some embodiments a vector for expression of a miRNA comprises an RNA pol II promoter. In some embodiments a vector for expression of a miRNA comprises flanking sequences found adjacent to a naturally occurring miRNA, e.g., a human miRNA. An artificial miRNA sequence complementary to ATPIF1 mRNA (e.g., complementary to the ATPIF1 3′UTR may be inserted between the flanking sequences (in the position that would be occupied by a native miRNA). In some embodiments an expression cassette encoding a miRNA may be inserted into an intron, which may be a synthetic intron. In some embodiments multiple shRNAs or miRNAs, e.g., 2, 3, 4, or more, targeted to ATPIF1 mRNA may be expressed from a single vector.

In some embodiments a virus vector may be modified to alter its tropism as compared to a naturally occurring virus or a virus that naturally has a desired tropism may be selected. “Tropism” as used herein refers to affinity of a virus or other vector for a particular tissue, cell type, and/or species, e.g., the capacity of a viral particle or other vector to infect or transduce (i.e., introduce a nucleic acid into, whether with or without other components) specific species, or specific tissues or cell types within those species. In some embodiments it may be desirable to alter the natural tropism of a virus or other vector, e.g., in order to facilitate efficient infection or transduction of target cells or reduce infection or transduction of non-target cells. For example, it may be desirable to: (i) broaden the range of cell types or species that a vector can transduce or infect to include one or more desired target cell types or species; (ii) narrow the range of cell types or species that a vector can transduce or infect to exclude one or more non-target cell types or species; (iii) increase the percentage of cells of one or more desired cell types that will be infected or transduced by a given number of viral particles or vector molecules; (iv) increase selectivity for certain cell types as compared with other cell types, or any combination of the foregoing. Desired target cells may be, for example, cells that typically exhibit evidence of dysfunction or are at risk of cell death due to a mitochondrial disorder. Non-target cells may, for example, be cells whose function is typically not severely or not significantly impaired by a mitochondrial disorder and/or that may be adversely affected by the vector or by the agent to be delivered. In some embodiments a modification may increase tropism for human cells. In some embodiments a modification may increase tropism for cells of a particular cell type to be targeted, e.g., a cell type that is particularly prone to be affected adversely by a mitochondrial disorder. In some embodiments a modification may increase tropism for hepatocytes, neurons or particular neuron subtypes, or skeletal or cardiac muscle cells, neurons.

A variety of methods known in the art may be used to modify tropism. In some embodiments such methods comprise modifying a virus so that it displays a suitable ligand to bind to a cell surface protein or structure that serves as a receptor for the virus. For example, viruses may be pseudotyped with naturally occurring viral attachment proteins or portions thereof from other viral strains or viral families that naturally have the capacity to infect the target cell type(s) or with artificial viral attachment proteins (e.g., chimeric proteins that comprise a portion of the native viral protein and a portion of an attachment protein of a different viral strain or species). Viral attachment proteins are proteins that viruses use to attach to cells. They are typically at least in part displayed on the surface of a virion envelope or capsid, allowing them to physically interact with, e.g., bind to, a receptor for the virus. The term “receptor” in this context refers to any cellular molecule or complex, typically a protein or proteoglycan, exposed at least in part at the surface of a cell and that facilitates viral attachment, fusion, or entry of a viral particle or viral genome, including those referred to in the art as receptors or co-receptors for particular viruses. The In some embodiments, one or more of a virus's native attachment protein is disabled or deleted from the viral genome or modified so as to decrease tropism for cells of one or more cell type or species that it would otherwise infect. In some embodiments a targeting moiety is inserted into a native viral protein that is at least in part exposed at the surface of the virus by, e.g., modifying the viral sequence encoding the protein to incorporate a sequence encoding the targeting moiety. In some embodiments the protein is a capsid protein, envelope protein, or fiber protein, The targeting moiety may be, e.g., a single chain antibody, non-antibody polypeptide, or peptide. In some embodiments the targeting moiety comprises a ligand for a cell surface protein of a target cell, e.g., a cell surface protein that naturally serves as a receptor for a virus. In some embodiments a viral particle may be covalently or noncovalently modified after its production by conjugating a targeting moiety to the capsid or envelope. In general, the targeting moiety may comprise an antibody, polypeptide, aptamer, small molecule, or any other agent that can be covalently attached to a virion surface, e.g., to a capsid protein or envelope protein. Covalent conjugation may be achieved, e.g., by coupling to exposed thiol groups or other suitably reactive functional groups of a protein exposed at the surface of a capsid or envelope. Such groups may be naturally occurring or introduced by genetic engineering. In some embodiments a targeting moiety is noncovalently attached to a viral particle using a specific binding pair such as avidin-biotin. For example, biotin is coupled to the vector and then bound to an avidin which is fused or attached to a targeting moiety. In some embodiments a bispecific antibody that has specificity for the vector and specificity for a target cell (e.g., for a target cell surface protein) may be used. Similar approaches may be used to target non-viral delivery vehicles such as liposomes, lipid-based particles, nanoparticles, microparticles, polymeric particles, or other delivery vehicles.

In some embodiments targeting one or more cell types of interest is accomplished at least in part by use of a particular serotype or strain from within a virus family that has the desired tropism. In some embodiments AAV8 or AAV9 may be selected for targeting hepatocytes or neurons. In some embodiments AAV5 may be selected for targeting neurons.

In some embodiments a tissue specific or cell type specific regulatory region may be used to direct expression of a nucleic acid encoding an ATPIF1 inhibitor in desired target tissues or cells. The tissue specific or cell type specific regulatory region may be operably linked to a sequence encoding the ATPIF1 inhibitor and incorporated into a vector, e.g., a viral vector. In some embodiments the regulatory region is hepatocyte-specific, neuron-specific, or muscle-specific. Numerous hepatocyte-specific regulatory regions are known in the art. In general, hepatocyte-specific regulatory regions may be derived from a wide variety of genes that are specifically expressed in hepatocytes. Examples of hepatocyte-specific promoters include the alpha1 anti-trypsin promoter, albumin promoter, transthyretin promoter, and alpha fetoprotein promoter. In some embodiments a liver-specific regulatory region containing a thyroid hormone-binding globulin promoter sequence, two copies of an alpha1-microglobulin/bikunin enhancer sequence, and a leader sequence may be used (Franco, L M, et al., Molecular Therapy (2005) 12, 876-884). In some embodiments a liver specific promoter described in US Pat. Pub. No. 20080153156 may be used. In some embodiments the vector comprises regulatory regions that are minimized in size. In some embodiments a liver-specific regulatory region comprises one or more copies of a liver-specific enhancer element, such as an enhancer element from apolipoprotein E gene, albumin gene, or alpha1-microglobulin gene, operably linked to the promoter, e.g., inserted upstream of the promoter. In some embodiments a liver specific transcriptional enhancer described in US Pat. Pub. No. 20040259208 may be used. In some embodiments the expression cassette includes a liver-specific hepatic control region (HCR) enhancer in combination with a liver-specific promoter. For example, in some embodiments the expression cassette comprises the human A 1-antitrypsin (HAAT) promoter in combination with the hepatic control region (HCR) of the human apolipoprotein E gene (Nathwani, A. C., et al., (2006) Blood. 107, 2653-2661). In some embodiments the promoter comprises the hepatic nuclear factor 1 binding element. In some embodiments human muscle creatine kinase (MCK) promoter, optionally together with the human muscle creatine kinase enhancer, may be used to direct muscle-specific expression (Hauschka, S. D. (1996) Mol. Cell. Biol. 16, 5058-5068). In some embodiments a human synapsin promoter (Thiel, G., et al., Proc. Natl. Acad. Sci. U.S.A. 88, 3431-3435) or human enolase promoter (Nielsen T T, et al., (2009) J Gene Med 11:559-569) may be used to direct neuron-specific expression.

In general, selective targeting of gene therapy to a particular tissue, organ, or cell type may be achieved using (i) physical targeting such as may result due to viral tropism or use of a targeting moiety; (ii) transcriptional targeting through use of tissue or cell type specific regulatory regions; (iii) local administration to a desired target tissue or organ; (iv) any combination of (i)-(iii).

In some embodiments a gene therapy vector may be prepared according to standards rendering it suitable for administration to human subjects. In some embodiments a gene therapy vector preparation suitable for administration to human subjects is free of detectable levels of replication-competent virus. In some embodiments a gene therapy vector preparation suitable for administration to human subjects is prepared without use of products isolated from humans or animals, such as serum. In some aspects, the present disclosure provides such preparations. In some aspects, the present disclosure provides producer cell lines capable of producing viral particles comprising a nucleic acid encoding an ATPIF1 inhibitor.

Assays compositions, systems, and components thereof (e.g., nucleic acid constructs, vectors, cell lines) useful for performing any of the screening methods (e.g., methods for identification of candidate agents) are provided. Where the disclosure describes an assay, assay composition(s) and assay system(s), of use for performing an assay and each component of the assay composition, all combinations thereof, as well as methods and reagents for preparing the composition and its components are provided. Kits comprising one or more components useful for performing any of the methods are provided. Kits may comprise instructions for performing any of the methods.

In some embodiments one or more agents, e.g., one or more known modulators of ATPIF1 is selected for further testing, development, or use. A selected hit may be referred to as a “lead” or “lead agent”. For example, a lead may be an agent that is determined or predicted to have higher potency, greater selectivity for a target, one or more drug-like properties, potential for useful modification, or any other propert(ies) of interest, e.g., as compared with one or more other hits, e.g., as compared with the majority of other hits. Further testing may comprise, e.g., resynthesis of a hit, retesting of a hit in the same or a different assay, etc. Development of an agent may comprise producing an altered agent, e.g., an altered lead agent. In some embodiments structures of hit compounds may be examined to identify a pharmacophore, which may be used to design additional compounds (e.g., structural analogs). In some embodiments any of the methods may comprise producing an altered agent, e.g., an altered lead agent. In some embodiments a method comprises modifying an agent to achieve or seek to achieve an alteration in one or more properties, e.g., (1) increased affinity for a target of interest; (2) decreased affinity for a non-target molecule, (3) increased solubility (e.g., increased aqueous solubility); (4) increased stability (e.g., in vivo); (5) increased potency; (6) increased selectivity, e.g., for ATPIF1; (7) a decrease in one or more side effects (e.g., decreased adverse side effects, e.g., decreased toxicity); (8) increased therapeutic index; (9) one or modified pharmacokinetic properties (e.g., absorption, distribution, metabolism and/or excretion); (10) modified onset of therapeutic action or duration of effect; (11) modified, e.g., increased, oral bioavailability; (12) modified, e.g., increased, tissue penetration; (13) modified, e.g., increased, cell permeability; (14) modified, e.g., increased, delivery to mitochondria; (15) modified, e.g., increased, increased ability to cross the blood-brain barrier (increased ability to cross the blood-brain barrier may be desirable in some embodiments if the agent is to be used to treat disorders of central nervous system; decreased ability to cross the blood-brain barrier may be desirable in some embodiments if the agent has adverse effects on the CNS); (16) altered immunogenicity; (17) altered plasma protein binding.

In some embodiments any of the methods further comprises determining an in vitro activity or in vivo activity or toxicology profile of an altered agent, e.g., an altered lead agent. One or more additional alterations may be performed, e.g., based at least in part on such analysis. Multiple cycles of alteration and testing may be performed, thereby generating additional altered agents. In some embodiments any of the methods may further comprise performing a quantitative structure activity relationship analysis of multiple hit, lead, or altered agents. Alteration may be accomplished through at least partly random or non-predetermined modification, predetermined modification, and/or using computational approaches in various embodiments. In some embodiments alteration may make use of established principles or techniques of medicinal chemistry, e.g., to predictably alter one or more properties. In some embodiments, a first library of test agents is screened using any of the methods described herein, one or more test agents that are “hits” or “leads” is identified, and at least one such hit or lead is subjected to systematic structural alteration to create a second library of compounds structurally related to the hit or lead. The second library is then screened using methods described herein or other methods.

In some embodiments any of the methods may comprise producing an altered agent, e.g., an altered lead agent, by modifying an agent to incorporate or be attached to a label, which may optionally be used to detect or measure the agent or a metabolite of the agent, e.g., in a pharmacokinetic study. In some embodiments any of the methods may comprise producing an altered agent, e.g., an altered lead agent, by modifying an agent to incorporate or be attached to a second moiety (or more than two moieties). In some embodiments a second (or additional) moiety comprises a linker, tag, or targeting moiety. In some embodiments a second (or additional) moiety may modify one or more properties (1)-(17) listed above. In some embodiments a modification may increase delivery of the agent to, or accumulation of the agent at, a site of desired activity in the body of a subject. A site may be, e.g., an organ, tissue, cellular compartment (e.g., cytoplasm, organelle), etc.

In some embodiments a moiety that enhances cell permeability may comprise a protein transduction domain (PTD). “Cell permeability” is used interchangeably with “cell uptake” herein and is not intended to imply any particular mechanism. Uptake may comprise traversal of the plasma membrane into the cytoplasm. A PTD is a peptide or peptoid that can enhance uptake by cells, e.g., mammalian cells, of an entity that comprises it or to which it is attached. Many PTDs are known in the art. Examples of PTDs include various sequences rich in amino acids having positively charged side chains (e.g., guanidino-, amidino- and amino-containing side chains (e.g., U.S. Pat. No. 6,593,292) such as arginine-rich peptides, sequences from HIV Tat protein (e.g., U.S. Pat. No. 6,316,003); penetratin (sequence derived from the homeodomain of Antennapedia); sequences from a phage display library (e.g., U.S. 20030104622); MTS peptide (sequence derived from the Kaposi fibroblast growth factor signal peptide), etc. Organelle-specific PTDs provide a means to target specific subcellular sites. See, e.g., Jain M, et al. Cancer Res. 65:7840-7846, 2005; Torchilin V P. Adv Drug Deliv Rev.58:1532-1555, 2006; Juliano R L, et al. Wiley Interdiscip Rev Nanomed Nanobiotechnol. 1:324-335, 2009; Stewart K M, et al. Org Biomol Chem. 6(13):2242-55, 2008; Fonseca S B, et al., Adv Drug Deliv Rev., 61(11):953-64, 2009; Heitz F, et al., Br J. Pharmacol., 157(2):195-206, 2009, and references in any of the foregoing, which are incorporated herein by reference. In some embodiments, a PTD may be used to enhance cell uptake of a small molecule, RNAi agent, aptamer, polypeptide, microparticle, or nanoparticle that comprises a test agent or ATPIF1 modulator.

In some embodiments an ATPIF1 inhibitor comprises or is physically associated with a moiety that increases mitochondrial localization of the agent, e.g., that increases entry of the agent into the mitochondria. In some embodiments an ATPIF1 inhibitor is modified to comprise or be physically associated with such a moiety. In some embodiments the moiety is lipophilic and enhances passage across cell and/or mitochondrial membranes. In some embodiments the moiety targets the agent to mitochondria. Mitochondrial targeting moieties in certain embodiments can include a variety of peptides, peptide mimetics, and non-peptide species. In some embodiments such a moiety is conjugated to an ATPIF1 inhibitor or expressed as a fusion protein with an ATPIF1 inhibitor in order to target the agent to mitochondria. In some embodiments a mitochondrial targeting moiety comprises a naturally occurring mitochondrial targeting signal (MTS). In some embodiments an MTS comprises the MTS of E1α pyruvate dehydrogenase. In some embodiments a mitochondrial targeting moiety comprises a functional variant of a naturally occurring MTS. MTSs are often N-terminal, or less frequently C-terminal, cleavable amino acid sequences of e.g., about 15-40 residues in length, which are often positively charged with relatively few negatively charged residues. A MTS may, e.g., comprise multiple basic (e.g., arginine), hydrophobic (e.g., alanine, leucine), and polar residues (e.g., serine). The targeting signal is generally proteolytically removed by mitochondria processing peptidase during import or inside the mitochondrial matrix. Some mitochondrial proteins are targeted to mitochondria by similar internal sequences that do not undergo cleavage. MTSs are believed to form amphipathic α-helices, which may be important for their recognition by the translocation machineries in the mitochondrial outer (TOM complex) and inner (TIM complex) membranes. In general a sequence having amphiphilicity in combination with localized positive charges from basic residues may direct successful mitochondrial import. Exemplary peptides that may be used to enhance mitochondrial import, include, e.g., SS peptides or XJB peptide mimetics or a series of cationic, lipophilic cell-permeable mitochondrial penetrating peptides at least 4-8 amino acids in length comprising lysine (K) and arginine (R) or d-arginine (r) (selected to provide positive charge), and phenylalanine (F) and cyclohexylalanine (FX) residues (to impart lipophilicity) (Horton, K H, et al., Chemistry & Biology 15: 375-382 (2008). SS tetrapeptides feature a common structural motif of alternating aromatic and basic residues. XJB peptides are derived from the sequence of gramicidin S antibiotics. Non-peptide mitochondrial targeting species include various lipophilic cationic compounds such as triphenylphosphonium (TPP) or a derivative thereof, e.g., a lower alkyl derivative thereof (e.g., a C1-6 alkyl, e.g., methyl derivative), (2-oxo-ethyl)-triphenyl-phosphonium, or stearyltriphenyl phosphonium. See, e.g., Hoye, A T, et al., Accounts of Chemical Research, Vol. 41(1): 87-97 (2008) and/or Mossalam, M., et al., Ther Deliv. 1(1): 169-193 (2010) or references in either of the foregoing for additional discussion of mitochondrial targeting. In some embodiments an endosomolytic agent may be used to increase intracellular release of an agent that is taken up by endosomes.

In some embodiments an ATPIF1 inhibitor comprises or is modified to comprise or be physically associated with a moiety that increases passage across the blood brain barrier (BBB). In some embodiments passage across the BBB is useful for an agent to be used to treat a mitochondrial disorder affecting the CNS, e.g., Parkinson's disease.

In some embodiments a ATPIF1 inhibitor is modified to increase its lipophilicity by, e.g., conjugating a lipophilic moiety thereto.

In some embodiments an agent, e.g., an ATPIF1 inhibitor, may be conjugated to a moiety such as polyethylene glycol (PEG) or a derivative thereof, or another biocompatible organic polymer (either naturally occurring or artificial), resulting in an agent of increased size that has an increased circulation time in the body (e.g., after intravenous administration). The moiety may have a molecular weight, or average molecular weight, of, e.g., between 10 kD and 200 kD in various embodiments. PEGylation (the process of covalent attachment of a polyethylene glycol polymer chain to another molecule) may be achieved by incubation of a reactive derivative of PEG with the target molecule. In some embodiments the covalent attachment of PEG to an agent may “mask” the agent from the immune system (reducing immunogenicity and antigenicity), and increase the hydrodynamic size of the agent, which prolongs its circulatory time by reducing renal clearance. PEGylation may provide enhanced water solubility to hydrophobic agents. In some embodiments an ATPIF1 inhibitor may be released (e.g., in vivo) from a moiety to which it is conjugated, such as PEG. In some embodiments the ATPIF1 inhibitor is conjugated to a moiety via a cleavable linker. The linker may, for example, be acid-labile or may be cleaved by an intracellular or extracellular enzyme. In some embodiments an acid-labile linker comprises a vinyl ether, ortho ester, or hydrazine. In some embodiments the linker is cleaved at a pH typically found in endosomes.

In some embodiments an ATPIF1 inhibitor comprises or is physically associated with, e.g., covalently or noncovalently linked to, a targeting moiety that targets the agent to a particular cell type or tissue, e.g., cells or tissues comprising dysfunctional mitochondria or at risk of mitochondrial dysfunction. In some embodiments, a targeting moiety comprises an agent, e.g., antibody, aptamer, small molecule, or polypeptide, that binds to a cell surface marker. In some embodiments the antibody is a single chain antibody or single domain antibody. In some embodiments the polypeptide is an engineered binding protein that is distinct from antibodies, such as an affibody, anticalin, adnectin, or darpin. In some embodiments, a targeting moiety comprises a ligand that binds to a cell surface marker. In some embodiments the cell surface marker is a receptor. In some embodiments the targeting moiety comprises a compound that is a natural ligand of the receptor or a variant of such a compound.

In some embodiments a targeting moiety targets an ATPIF1 inhibitor to liver cells, e.g., hepatocytes. In some embodiments a moiety suitable for targeting to liver cells, e.g., hepatocytes, comprises cholesterol. In some embodiments a moiety suitable for targeting to hepatocytes comprises a ligand for an asialoglycoprotein receptor. In some embodiments the ligand is N-acetylgalactosamine. In some embodiments a moiety suitable for targeting to hepatocytes comprises a ligand for an LDL receptor family protein. In some embodiments the ligand for an LDL receptor family protein is low density lipoprotein receptor-related protein associated protein 1 (Gene ID 4043 (human gene); Gene ID 16976 (mouse gene) or a variant thereof that binds to an LDL receptor.

In some embodiments an altered ATPIF1 inhibitor may be produced as a fusion protein. In some embodiments an altered ATPIF1 inhibitor may be produced at least in part by covalently attaching a second moiety to the agent. In some embodiments an ATPIF1 inhibitor and a second moiety are linked using a linker. A wide variety of linkers, reactive functional groups useful for covalent attachment, and methods of linking various molecules or other entities are known in the art and may be used in various embodiments. Various examples are described in Hermanson, G., Bioconjugate Techniques, 2^{nd} ed., Academic Press (2008). One of ordinary skill in the art will be able to select appropriate linkers and methods. Any suitable linker and/or method can be used to link an ATPIF1 inhibitor to a targeting moiety in order to generate a targeted ATPIF1 inhibitor. For example, a bifunctional linker may be used. In some embodiments, a linker comprises a cleavage site for an intracellular enzyme, so that the ATPIF1 inhibitor may be released from the targeting moiety inside cells that contain the enzyme.

In some embodiments any of the methods may comprise producing a composition by formulating an agent, e.g., a lead agent or altered agent, e.g., an altered lead agent, with a pharmaceutically acceptable carrier. In some embodiments any of the methods may comprise testing a lead or altered agent in vivo, by administering one or more doses of the composition to a subject, optionally a subject having a mitochondrial disorder, and evaluating one or more pharmacokinetic parameters, evaluating the effect of the agent on the subject, e.g., monitoring for beneficial or adverse effects. In some embodiments any of the methods may comprise testing a lead or altered agent in an animal model in vivo, by administering one or more doses of the composition to a non-human animal that serves as a model for a mitochondrial disorder and evaluating the effect of the agent on one or more symptoms or signs of the disorder. In some embodiments samples or data may be acquired at multiple time points, e.g., during or after a dose or series of doses. In some embodiments a suitable computer program may be used for data analysis, e.g., to calculate one or more pharmacokinetic parameters. In certain embodiments, the subject is a mouse, rat, rabbit, dog, cat, sheep, pig, non-human primate, or human. It will be understood that an altered agent, e.g., an altered lead agent, may be produced using any suitable method. In some embodiments an agent or an intermediate obtained in the course of synthesis of the agent may be used as a starting material for alteration. In some embodiments an altered agent may be synthesized using any suitable materials and/or synthesis route.

In some embodiments, at least some information regarding a screen to identify agents that modulate, e.g., inhibit, ATPIF1 is stored on a computer-readable medium. In some embodiments the screen is a physical screen (e.g., using one or more cell-free or cell-based assays). In some embodiments the screen is a virtual screen. The information may include, e.g., screening protocols, results obtained from the screen or from additional screens (e.g., raw data collected during the screen, identity of hit compounds, predicted properties of hits, leads, or altered leads, or results of additional testing of hits, leads, or altered leads), and/or protocols of or results obtained from tests performed on agents identified in the screen (e.g., tests in cell-based or animal models of mitochondrial disorders). Test agents may be ranked e.g., according to their effect on ATPIF1 expression or activity.

In some embodiments screens or assays described herein may identify agents that activate or enhance ATPIF1 expression or activity. Such agents may be of use, e.g., to investigate the role of ATPIF1 in mitochondrial physiology, as controls in assays that test the effect of candidate ATPIF1 inhibitors, etc. ATPIF1 activators/enhancers may be useful for purposes of inhibiting the F0-F1 ATPase. Modifications may be made to such activators/enhancers as described herein for inhibitors.

Methods and compositions described herein with regard to ATPIF1 may be applied to other targets identified as described herein. Modifications to the compositions and/or methods can be made as appropriate depending, e.g., on the identity of the target and its activities. For example, in some aspects, such methods and compositions in which the target is TP53 are provided. The gene encoding human TP53 (Gene ID: 7157) was identified in the same near-haploid screen in which ATPIF1 was identified (see Example 1).

### VIII. Pharmaceutical Compositions, Gene Therapy, Methods of Treatment

An agent identified, selected, or designed according to a method described herein can be used for any of a variety of purposes in various embodiments. In some embodiments, an agent is useful for therapeutic purposes, e.g., as a therapeutic agent for a subject in need of treatment for a mitochondrial disorder.

In some embodiments, a subject in need of treatment for a mitochondrial disorder exhibits at least one symptom or sign of the disorder. In some embodiments, a subject in need of treatment for a mitochondrial disorder is at risk of a mitochondrial disorder and/or at risk of developing symptoms and/or signs of the disorder as compared with an average member of the general population, optionally matched with regard to age, gender, and/or other demographic variables. A subject may be “at risk of a mitochondrial disorder” in any of a variety of circumstances. “At risk of” implies at increased risk of, relative to the risk the subject would have in the absence of one or more circumstances, conditions, or attributes of the subject, and/or relative to the risk that an average, healthy member of the population would have. Examples of conditions that place a subject “at risk” include, but are not limited to, exposure to agents that damage mitochondria, mutations in genes associated with mitochondrial disorders, family history of mitochondrial disorder, or any other condition that within the judgment and skill of a health care provider place the subject at sufficient risk of a mitochondrial disorder as to merit treatment.

In some embodiments, a recipient of an organ, tissue, or cell transplant is treated with an ATPIF1 inhibitor, e.g., to reduce the likelihood or severity of mitochondrial dysfunction in the transplanted cells, tissues, or organ(s). Such treatment could commence prior to, during, or after the transplant procedure in various embodiments.

In some aspects, ex vivo uses are contemplated. For example, organs, tissues, or cells intended for use in transplantation (e.g., xenotransplantation or transplantation into an individual of the same species) can be contacted ex vivo with an ATPIF1 inhibitor, e.g., to reduce mitochondrial dysfunction that might otherwise occur in such organ, tissue, or cell due, e.g., to physiologic stress occurring during harvest, storage, transport, transplantation, or post-transplant. In some embodiments organs, tissues, or cells intended for use in transplantation are contacted ex vivo with an ATPIF1 inhibitor to reduce cell death due at least in part to mitochondrial dysfunction.

In some embodiments a method of treatment includes a step of identifying a subject suffering from or at risk of a mitochondrial disorder. In some embodiments a method of treatment includes a step of diagnosing a subject as suffering from or at risk of a mitochondrial disorder.

Agents and compositions disclosed herein and/or identified or validated using a method described herein may be administered by any suitable means such as orally, intranasally, subcutaneously, intramuscularly, intravenously, intra-arterially, parenterally, intraperitoneally, intrathecally, intratracheally, ocularly, sublingually, vaginally, rectally, dermally, or by inhalation, e.g., as an aerosol. Depending upon the type of condition (e.g., mitochondrial disorder) to be treated, agents may, for example, be inhaled, ingested or administered by systemic routes. Thus, a variety of administration modes, or routes, are available. The particular mode selected will depend, of course, upon the particular agent selected, the particular condition being treated and the dosage required for therapeutic efficacy. The methods, generally speaking, may be practiced using any mode of administration that is medically or veterinarily acceptable, meaning any mode that produces acceptable levels of efficacy without causing clinically unacceptable (e.g., medically or veterinarily unacceptable) adverse effects. The term “parenteral” includes intravenous, intramuscular, intraperitoneal, subcutaneous, intraosseus, and intrasternal administration, e.g., by injection or infusion techniques. In some embodiments, a route of administration is parenteral or oral. Optionally, a route or location of administration is selected based at least in part on the location where cells having dysfunctional mitochondria are located. For example, an agent such as a small molecule, RNAi agent, or gene therapy vector may be administered locally to a target tissue or organ, e.g., a tissue or organ that exhibits evidence or symptoms of mitochondrial dysfunction or that typically exhibits evidence of dysfunction in individuals who have a particular mitochondrial disorder. “Local administration” encompasses (1) administration directly into or near a target tissue or organ, (2) into or near a blood vessel that directly supplies a target tissue or organ, or (3) into a fluid-filled extravascular compartment in fluid communication with the target tissue or organ (including inhalational administration where the target tissue or organ is a component of respiratory system such as the lung, intrathecal or intraventricular administration where the target organ or tissue is a component of the central nervous system such as the brain). “Near” in this context refers to locations up to 1 cm, 5 cm, or 10 cm from an edge or border of the target tissue, organ, or blood vessel. In some embodiments an agent, e.g., a small molecule, RNAi agent, or gene therapy vector, is locally administered to the liver, e.g., by injection or infusion injection into the portal vein or hepatic artery or directly into the liver parenchyma, e.g., to treat a subject with a mitochondrial disorder that affects the liver. In some embodiments, inhaled medications are of use. Such administration allows direct delivery to the lung, although it could also be used to achieve systemic delivery, e.g., to treat a disease affecting the liver, nervous system, muscles, etc. In some embodiments, intrathecal or intraventricular administration may be of use, e.g., in a subject with a mitochondrial disorder affecting the central nervous system. In some embodiments local administration to the brain is performed by stereotactic injection into the parenchyma of the brain or by intrathecal or intraventricular injection, infusion, or implantation. In some embodiments convection-enhanced delivery or step cannulae may be used to enhance delivery to the brain. In some embodiments nasal administration is used to deliver an agent to the brain. Other appropriate routes and devices for administering therapeutic agents will be apparent to one of ordinary skill in the art.

Suitable preparations, e.g., substantially pure preparations, of an active agent (e.g., an ATPIF1 inhibitor) may be combined with one or more pharmaceutically acceptable carriers or excipients, etc., to produce an appropriate pharmaceutical composition. In some embodiments, a pharmaceutically acceptable compositions for administration to a subject comprises (i) an ATPIF1 inhibitor; and (ii) a pharmaceutically acceptable carrier or excipient. The term “pharmaceutically acceptable carrier or excipient” refers to a carrier (which term encompasses carriers, media, diluents, solvents, vehicles, etc.) or excipient which does not significantly interfere with the biological activity or effectiveness of the active ingredient(s) of a composition and which is not excessively toxic to the host at the concentrations at which it is used or administered. Other pharmaceutically acceptable ingredients can be present in the composition as well. Suitable substances and their use for the formulation of pharmaceutically active compounds is well-known in the art (see, for example, “Remington's Pharmaceutical Sciences”, E. W. Martin, 19th Ed., 1995, Mack Publishing Co.: Easton, Pa., and more recent editions or versions thereof, such as Remington: The Science and Practice of Pharmacy. 21st Edition. Philadelphia, Pa. Lippincott Williams & Wilkins, 2005, for additional discussion of pharmaceutically acceptable substances and methods of preparing pharmaceutical compositions of various types).

A pharmaceutical composition is typically formulated to be compatible with its intended route of administration. For example, preparations for parenteral administration include sterile aqueous or non-aqueous solutions, suspensions, and emulsions. Aqueous carriers include water, alcoholic/aqueous solutions, emulsions or suspensions, including saline and buffered media, e.g., sodium chloride solution, Ringer's dextrose, dextrose and sodium chloride, lactated Ringer's. Examples of non-aqueous solvents are propylene glycol, polyethylene glycol, vegetable oils such as olive oil, and injectable organic esters such as ethyl oleate. fixed oils, polyethylene glycols, glycerine, propylene glycol or other synthetic solvents; preservatives, e.g., antibacterial agents such as benzyl alcohol or methyl parabens; antioxidants such as ascorbic acid or sodium bisulfate; chelating agents such as ethylenediaminetetraacetic acid; buffers such as acetates, citrates or phosphates, and agents for the adjustment of tonicity such as sodium chloride or dextrose. pH can be adjusted with acids or bases, such as hydrochloric acid or sodium hydroxide. Such parenteral preparations can be enclosed in ampoules, disposable syringes or multiple dose vials made of glass or plastic. Pharmaceutical compositions and agents for use in such compositions may be manufactured under conditions that meet standards or criteria prescribed by a regulatory agency such as the US FDA (or similar agency in another jurisdiction) having authority over the manufacturing, sale, and/or use of therapeutic agents. For example, such compositions and agents may be manufactured according to Good Manufacturing Practices (GMP) and/or subjected to quality control procedures appropriate for pharmaceutical agents to be administered to humans.

For oral administration, agents can be formulated by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Suitable excipients for oral dosage forms are, e.g., fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl cellulose, sodium carboxymethylcellulose, and/or polyvinylpyrrolidone (PVP). If desired, disintegrating agents may be added, such as the cross linked polyvinyl pyrrolidone, agar, or alginic acid or a salt thereof such as sodium alginate. Optionally the oral formulations may also be formulated in saline or buffers for neutralizing internal acid conditions or may be administered without any carriers. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art.

Formulations for oral delivery may incorporate agents to improve stability in the gastrointestinal tract and/or to enhance absorption.

For administration by inhalation, pharmaceutical compositions may be delivered in the form of an aerosol spray from a pressured container or dispenser which contains a suitable propellant, e.g., a gas such as carbon dioxide, a fluorocarbon, or a nebulizer. Liquid or dry aerosol (e.g., dry powders, large porous particles, etc.) can be used. The disclosure contemplates delivery of compositions using a nasal spray or other forms of nasal administration. Several types of metered dose inhalers are regularly used for administration by inhalation. These types of devices include metered dose inhalers (MDI), breath-actuated MDI, dry powder inhaler (DPI), spacer/holding chambers in combination with MDI, and nebulizers.

For topical applications, pharmaceutical compositions may be formulated in a suitable ointment, lotion, gel, or cream containing the active components suspended or dissolved in one or more pharmaceutically acceptable carriers suitable for use in such comporisition.

For local administration to the eye, pharmaceutical compositions may be formulated as solutions or micronized suspensions in isotonic, pH adjusted sterile saline, e.g., for use in eye drops, or in an ointment. In some embodiments intraocular administration is used. Routes of intraocular administration include, e.g., intravitreal injection, retrobulbar injection, peribulbar injection, subretinal, sub-Tenon injection, and subconjunctival injection. In some embodiments an intraocular implant (sometimes termed an “insert”) is used to deliver an agent to the eye. In some embodiments a gene therapy vector is administered by subretinal injection.

Pharmaceutical compositions may be formulated for transmucosal or transdermal delivery. For transmucosal or transdermal administration, penetrants appropriate to the barrier to be permeated may be used in the formulation. Such penetrants are generally known in the art. Pharmaceutical compositions may be formulated as suppositories (e.g., with conventional suppository bases such as cocoa butter and other glycerides) or as retention enemas for rectal delivery.

In some embodiments, a pharmaceutical composition includes one or more agents intended to protect the active agent(s) against rapid elimination from the body, such as a controlled release formulation, implant, microencapsulated delivery system, etc. Compounds may be encapsulated or incorporated into particles, e.g., microparticles or nanoparticles. Biodegradable, biocompatible polymers can be used, such as ethylene vinyl acetate, polyanhydrides, polyglycolic acid, PLGA, collagen, polyorthoesters, polyethers, and polylactic acid. Methods for preparation of such formulations will be apparent to those skilled in the art. For example, and without limitation, a number of particle-based delivery systems are known in the art for delivery of siRNA. Use of such compositions is contemplated. In some embodiments lipidoid particles are used. In some embodiments non-lipid particles are used. Liposomes or other lipid-based particles can also be used as pharmaceutically acceptable carriers. In some embodiments a macroscopic implant is used to deliver an agent systemically or locally. In some embodiments the implant is implanted in the CNS, e.g., in the brain.

In some embodiments, a pharmaceutically acceptable derivative of a ATPIF1 inhibitor, e.g., a ATPIF1 inhibitor described herein or identified or validated as described herein, is provided. As used herein, a pharmaceutically acceptable derivative of a particular agent includes, but is not limited to, pharmaceutically acceptable salts, esters, salts of such esters, or any other adduct or derivative which upon administration to a subject in need thereof is capable of providing the compound, directly or indirectly. Thus, pharmaceutically acceptable derivatives can include salts, prodrugs, and/or active metabolites. The term “pharmaceutically acceptable salt” refers to those salts which are, within the scope of sound medical judgment, suitable for use in contact with the tissues of humans and/or lower animals without undue toxicity, irritation, allergic response and the like, and which are commensurate with a reasonable benefit/risk ratio. A wide variety of appropriate pharmaceutically acceptable salts are well known in the art. Pharmaceutically acceptable salts include, but are not limited to, those derived from suitable inorganic and organic acids and bases. A pharmaceutically acceptable derivative of an APTIF1 inhibitor may be formulated and, in general, used for the same purpose(s).

Pharmaceutical compositions, when administered to a subject in need of treatment for a disorder are, in at least some embodiments, administered for a time and in an amount sufficient to treat the disease or condition for which they are administered. Therapeutic efficacy and toxicity of active agents can be assessed by standard pharmaceutical procedures in cell cultures or experimental animals. The data obtained from cell culture assays and animal studies can be used in formulating a range of dosages suitable for use in humans or other subjects. Different doses for human administration can be further tested in clinical trials in humans as known in the art. The dose used may be the maximum tolerated dose or a lower dose. A therapeutically effective dose of an active agent in a pharmaceutical composition may be within a range of about 0.001 to about 100 mg/kg body weight, about 0.01 to about 25 mg/kg body weight, about 0.1 to about 20 mg/kg body weight, about 1 to about 10 mg/kg. Other doses include, for example, about 1 μg/kg to about 500 mg/kg, about 100 μg/kg to about 5 mg/kg). In some embodiments a single dose is administered while in other embodiments multiple doses are administered. Those of ordinary skill in the art will appreciate that appropriate doses in any particular circumstance depend upon the potency of the agent(s) utilized, and may optionally be tailored to the particular recipient. The specific dose level for a subject may depend upon a variety of factors including the activity of the specific agent(s) employed, severity of the disease or disorder, the age, body weight, general health of the subject, etc.

It may be desirable to formulate pharmaceutical compositions, particularly those for oral or parenteral compositions, in unit dosage form for ease of administration and uniformity of dosage. Unit dosage form, as that term is used herein, refers to physically discrete units suited as unitary dosages for the subject to be treated; each unit containing a predetermined quantity of active agent(s) calculated to produce the desired therapeutic effect in association with an appropriate pharmaceutically acceptable carrier. In some embodiments a pharmaceutically acceptable unit dosage form contains a predetermined amount of an ATPIF1 inhibitor, such amount being appropriate to treat a subject in need of treatment for a mitochondrial disorder.

It will be understood that a therapeutic regimen may include administration of multiple unit dosage forms over a period of time. In some embodiments, a subject is treated for between 1-7 days. In some embodiments a subject is treated for between 7-14 days. In some embodiments a subject is treated for between14-28 days. In other embodiments, a longer course of therapy is administered, e.g., over between about 4 and about 10 weeks, 10-26 weeks, 26-52 weeks, or longer. In some embodiments, treatment is continued for 1-5 years, or more. In some embodiments, treatment may be continued indefinitely. For example, a subject at risk of a mitochondrial disorder may be treated for any period during which such risk exists or the subject desires to avoid developing or to control the severity of symptoms or signs of a mitochondrial disorder. A subject may receive one or more doses a day, or may receive doses every other day or less frequently, within a treatment period. Treatment courses may be intermittent. For example, a subject may be treated when symptoms recur or may be monitored and treated when an indicator of impending symptoms or worsening of a disorder is detected.

In some embodiments, two or more different ATPIF1 inhibitors are administered. In some embodiments, an ATPIF1 inhibitor is administered in combination with a second compound useful for treating a mitochondrial disorder. In some embodiments “in combination” refers to administration of two or more agents with the knowledge that the two agents are useful for treating a particular disorder or each agent is administered for the purpose of treating or contributing to treatment of the disorder. In some embodiments of combined administration (i) a dose of the second compound is administered before more than 90% of the most recently administered dose of the first agent has been metabolized to an inactive form or excreted from the body; or (ii) doses of the first and second compound are administered at least once within 24 hours to 2 weeks of each other, or (iii) the agents are administered during overlapping time periods (e.g., by continuous or intermittent infusion); or (iv) any combination of the foregoing. The agent may be, but need not be, administered together as components of a single composition. In some embodiments, they may be administered individually at substantially the same time (by which is meant within less than 10 minutes of one another). In some embodiments they may be administered individually within a short time of one another (by which is meant less than 3 hours, sometimes less than 1 hour, apart). The agents may be, but need not, be administered by the same route of administration. When administered in combination with a second agent, the effective amount of a first agent needed to elicit a particular biological response may be less or more than the effective amount of the first agent when administered in the absence of the second compound (or vice versa), thereby allowing an adjustment of the amount dose of the either or both agent(s) relative to the amount that would be needed if one agent were administered in the absence of the other. For example, when agents are administered in combination (e.g., an ATPIF1 inhibitor and a second agent), a sub-therapeutic dosage of either of the agents, or a sub-therapeutic dosage of both, may be used in certain embodiments. A “sub-therapeutic amount” as used herein refers to an amount which is less than that amount which would be expected to produce a therapeutic result in the subject if administered in the absence of the other agent, e.g., less than a recommended amount. The effects of multiple agents may, but need not be, additive or synergistic. One or more of the compounds may be administered multiple times.

In some embodiments, e.g., embodiments in which a disorder is associated with a mutation, e.g., loss-of-function mutation or deletion, a treatment approach comprises at least in part correcting the underlying genetic defect by, e.g., repairing or at least in part replacing a mutated or otherwise dysfunctional gene. In some embodiments an ATPIF1 inhibitor is used in combination with such therapy. Administration of the ATPIF1 inhibitor may maintain viability or function of at least some cells that would otherwise be lost prior to administering the corrective treatment or prior to the corrective treatment becoming effective. In some embodiments an ATPIF1 inhibitor is administered in a composition together with a gene therapy vector designed to repair or at least in part replace a mutated or dysfunctional gene responsible for a mitochondrial disorder.

In some embodiments an additional agent comprises an anti-oxidant. In some embodiments a mitochondria-targeted antioxidant is used, e.g., MitoQ, which contains the antioxidant quinone moiety covalently attached to a lipophilic triphenylphosphonium cation. In some embodiments an anti-oxidant is coenzyme Q10 or α-tocopherol.

In some embodiments, a composition comprising an ATPIF1 inhibitor and a second agent useful for treating a mitochondrial disorder is provided. In some embodiments, a unit dosage form comprising the two (or more) agents is provided.

In some embodiments, pharmaceutical packs or kits comprising one or more containers (e.g., vials, ampoules, bottles) containing a pharmaceutically acceptable ATPIF1 inhibitor and, optionally, one or more other pharmaceutically acceptable ingredients, are provided. Optionally associated with such container(s) can be a notice in the form prescribed by a governmental agency regulating the manufacture, use or sale of pharmaceutical products, which notice reflects approval by the agency of manufacture, use or sale for human administration. The notice may describe, e.g., doses, routes and/or methods of administration, approved indications (e.g., mitochondrial disorders that the agent or pharmaceutical composition has been approved for use in treating), mechanism of action, or other information of use to a medical practitioner and/or patient. Different ingredients may be supplied in solid (e.g., lyophilized) or liquid form. Each ingredient will generally be suitable as aliquoted in its respective container or provided in a concentrated form. Kits may also include media for the reconstitution of lyophilized ingredients. The individual containers of the kit are preferably maintained in close confinement for commercial sale.

In some embodiments, an agent identified using a method described herein is useful for research purposes, e.g., to further study the role of ATPIF in normal physiologic processes or pathologic processes. For example, an agent can be used to further study the role of ATPIF1 in mitochondrial physiology.

One skilled in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the invention where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. For example, any one or more agents, disorders, subjects, or combinations thereof, can be excluded.

Where the claims or description relate to a composition of matter, e.g., a compound it is to be understood that methods of making or using the composition of matter according to any of the methods disclosed herein, and methods of using the composition of matter for any of the purposes disclosed herein are aspects of the invention, unless otherwise indicated or unless it would be evident to one of ordinary skill in the art that a contradiction or inconsistency would arise. Where the claims or description relate to a method, e.g., a method of identifying a compound, it is to be understood that methods of using the compound, or formulating a composition comprising the compound, as described herein, are aspects of the invention, unless otherwise indicated or unless it would be evident to one of ordinary skill in the art that a contradiction or inconsistency would arise.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”. “Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. It should also be understood that unless otherwise indicated or evident from the context, any product or composition described herein may be considered “isolated”.

### EXAMPLES

### Example 1

### Haploid Genetic Screen Using Antimycin Identifies “ATPIF1 Loss” as Conferring Resistance Against Complex III Inhibition

In order to identify potential drug targets for treatment of mitochondrial disorders, we decided to use inhibitors of oxidative phosphorylation (OXPHOS) to model mitochondrial diseases and search for genes, loss-of-function of which can confer resistance to mitochondrial dysfunction caused by the poison. To that end, we explored use of a screening platform based on gene inactivation in human cells using insertional mutagenesis that has recently been developed using the KBM7 CML cell line, which is haploid for all chromosomes except chromosome 8 (5). In this system, insertional mutagenesis is accomplished using a gene trap vector. Our screening approach entailed treating mutagenized KBM7 cells with inhibitors of oxidative phosphorylation, isolating cells able to survive such treatment, and identifying genes enriched for mutations in the surviving cell population.

We used antimycin (see FIG. 1A), a well-characterized complex III inhibitor, which binds to Qi site of cytochrome c reductase and inhibits oxidation of ubiquinol in the electron transport chain of oxidative phosphorylation (10), as our initial OXPHOS inhibitor. Mutated KBM7 haploid cells were treated with antimycin for 2 weeks. Antimycin-resistant cells were selected, pooled and genomic DNA was isolated. Sequences flanking the insertion sites were then amplified using an inverse-PCR protocol, followed by massively parallel sequencing. These sequences were subsequently mapped to the human genome and enrichment for mutations in genes was calculated by comparing a gene's mutation frequency in resistant cells to that in the unselected control data set. We identified a set of genes enriched for mutations in the antimycin resistant cell population and our screen identified ATPIF1 as the top hit, having the highest score and the lowest p value (FIG. 1A). We next subcloned the resistant cell population to obtain ATPIF1 null cells (FIG. 1B). These clonally derived cell lines that contain gene-trap insertions in ATPIF1 show complete loss of expression of ATPIF1 protein and displayed marked resistance to antimycin treatment (FIG. 1C-D). The susceptibility of KBM7 clones to antimycin was restored by expression of the ATPIF1 cDNA (encoding ATPIF1 isoform 1), validating the specificity of the phenotype.

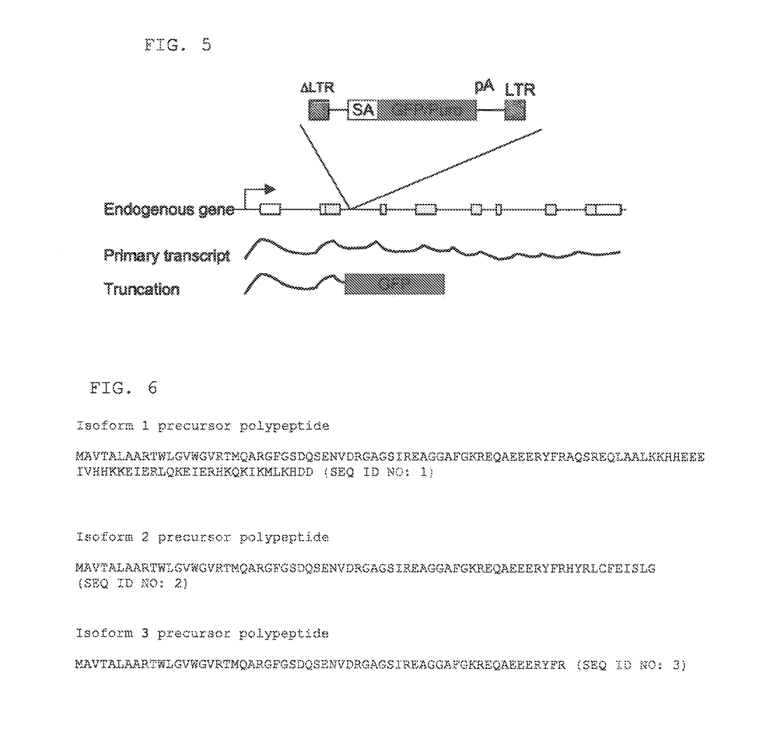

To further confirm the ability of ATPIF1 loss-of-function to confer resistance to antimycin A, we used three different short hairpin RNAs (shRNA) from the The RNAi Consortium TRC3 library (Broad Institute, Cambridge, Mass.) to inhibit APTIF1 expression in wild type KBM7 cells (FIG. 3C, upper panel) and tested the cells for antimcyin A resistance. shRNA-mediated inhibition of KBM7 expression conferred decreased sensitivity to antimycin A across a range of antimycin A concentrations, thus further validating that the resistance of the initially isolated antimycin A-resistant mutant KBM7 cells arose from loss-of-function of ATPIF1 (FIG. 3C, lower panel).

Below are the TRC codes and sequence information for each hairpin used in the assay. The shRNAs are in the PLKO1 vector.

shATPIF1-2: TRCN0000146646

Sequence Information

Target Sequence:
(SEQ ID NO: 4)
CCATGAAGAAGAAATCGTTCA
Hairpin Sequence:
(SEQ ID NO: 5)
5′-GCCGG-CCATGAAGAAGAAATCGTTCA-CTCGAG-TGAACGATTTCT
TCTTCATGG-TTTTTTG-3′

shATPIF1-5: TRCN0000150110

Sequence Information

Target Sequence:
(SEQ ID NO: 6)
CACCATGAAGAAGAAATCGTT
Hairpin Sequence:
(SEQ ID NO: 7)
5′-GCCGG-CACCATGAAGAAGAAATCGTT-CTCGAG-AACGATTTCTTC
TTCATGGTG-TTTTTTG-3′

shATPIF1-7: TRCN0000149949

Sequence Information

Target Sequence:
(SEQ ID NO: 8)
GAGCGTCTGCAGAAAGAAATT
Hairpin Sequence:
(SEQ ID NO: 9)
5′-CCCGG-GAGCGTCTGCAGAAAGAAATT-CTCGAG-AATTTCTTTCTG
CAGACGCTC-TTTTTTG-3′

For the viability assays described in this Example and Example 2, 200,000 cells were used for each sample. Cells were treated with the desired concentration of drug and viability was assessed four days later using 7-AAD and FACS analysis.

### Example 2

### ATPIF1 Loss Confers Resistance to Multiple Mitochondrial Poisons

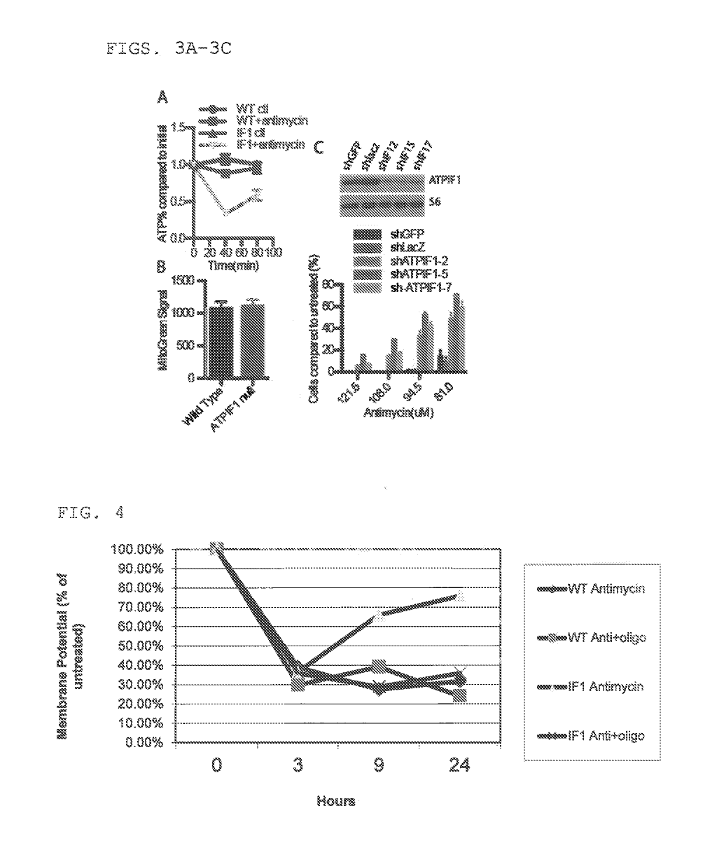

We investigated whether loss of ATPIF1 function would confer resistance to other OXPHOS inhibitors in addition to antimycin. Indeed, ATPIF1 null cells were resistant to complex I inhibitor (piercidin A), FCCP (uncoupler) and complex II inhibitor (TTFA) (FIG. 2), although the degree to which ATPIF1 loss conferred resistance was greater in the case of antimycin (a complex III inhibitor). Thus, ATPIF1 loss is thus able to confer resistance to inhibitors of at least three of the five protein complexes of the respiratory chain. These results further support the potential of ATPIF1 as a therapeutic target for treatment of mitochondrial disorders. In FIG. 2, the Y-axis represent fraction of surviving cells relative to control cells not treated with the agent.

### Example 3

### Testing Potential Mechanisms of Resistance to Antimycin in Cells Lacking ATPIF1 Function

(1) Alterations of Cellular ATP Levels:

ATPIF1 expression has previously been shown to be essential for survival following ischemia, by inhibiting ATP synthase hydrolytic activity and preserving cellular ATP levels (13). Because increased cellular ATP level is associated with greater viability, we investigated whether ATPIF1 null cells had greater ATP levels following antimycin treatment. Our initial experiments treating cells with antimycin demonstrated that ATPIF1 null cells actually have significantly lower levels of initial ATP compared to WT cells (FIG. 3A), which suggests that a change in ATP levels cannot explain the mechanism of resistance.

(2) Alterations in Number and/or Structure of Mitochondria:

There is evidence that ATPIF1 can modulate mitochondrial ultrastructure and thus cellular respiratory capacity (12). We therefore considered the possibility that cells resistant to antimycin treatment possessed altered mitochondrial number and structure. Our initial experiments suggest that mitochondria number in ATPIF1 null cells are not significantly different compared to controls as assessed by Mitotracker Green staining (FIG. 3B).

(3) Change in Membrane Potential:

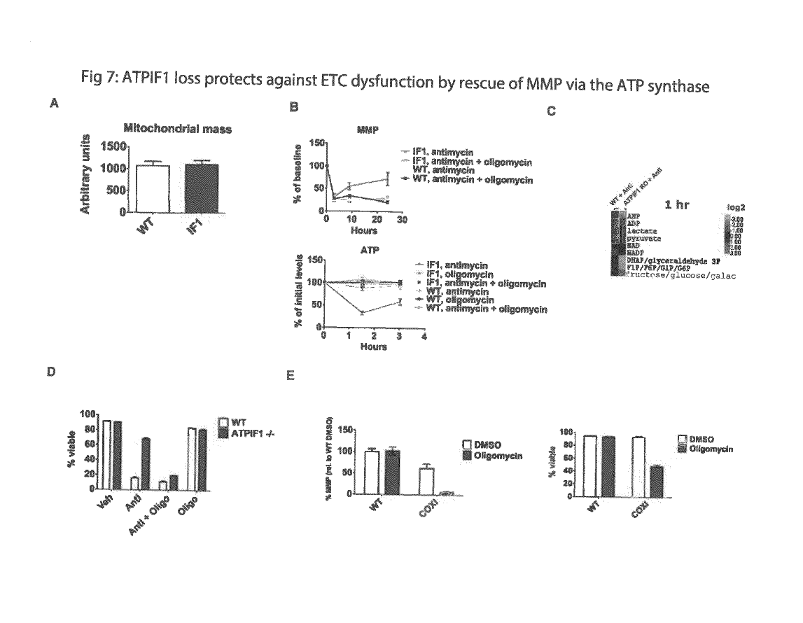

When cellular respiration is impaired, the electron transport chain stalls and protons can accumulate in the matrix, leading to depolarization of the inner mitochondrial membrane and reversal of the ATP synthase pump. As mentioned earlier, ATPIF1 binds to and inhibits the ATP synthase under conditions of matrix acidification, which maintains cellular ATP at the expense of abnormal membrane potential. However, it is not immediately clear whether it may be more beneficial to allow mitochondria to depolarize, as this may promote the release of proapoptotic inducers and thereby lead to cell death. Thus, it is possible that ATPIF1 null cells survive antimycin treatment by utilizing the ATP synthase in reverse mode (as an ATPase) to preserve membrane potential upon antimycin treatment. Many of the OXPHOS deficiencies in human disease also show similar defects in membrane potential. It thus is an attractive possibility that loss of ATPIF1 expression can ameliorate membrane potential (14). We tested this possibility and found that membrane potential is rescued by loss of ATPIF1 (FIG. 4).

Membrane potential was assessed using tetramethyl rhodamine methyl ester (TMRM). For the TMRM time course: 200,000 cells were used for each time point. Cells were treated with 121 uM antimycin and incubated in 25 nM TMRM (final concentration) in IMDM 20 minutes from the end of their time point at 37° C. and then analyzed by FACS analysis. Dead cells were excluded using 7-AAD.

(4) Change in Metabolism:

Because antimycin potently inhibits oxidative phosphorylation in cells, it is possible that ATPIF1 null cells may be resistant to antimycin treatment at least partly as a consequence of using alternative metabolic pathways and decreasing their dependency on OXPHOS. ATPIF1 cells have higher respiratory capacity compared to wild type cells.

(5) Alternative Binding Partners:

Acidification of the mitochondrial matrix leads to ATPIF1 binding to the F1-F0 ATP synthase. We reasoned that it is thus possible that under conditions of antimycin treatment, there are also induced interactions between ATPIF1 and other proteins. As such, one potential mechanism of resistance to antimycin treatment in ATPIF1 null cells could be abrogation of deleterious IF1-mediated protein-protein interactions that are induced upon complex III inhibition. In order to test this possibility, we are using Immunoprecipitation-Mass Spec (IP-MS) using Flag tagged ATPIF1. This approach has previously been used successfully in our laboratory to identify mTORC1 and mTORC2 components (17, 18). We have performed preliminary IP-MS experiments and identified the F1-F0 ATP synthase components and various other mitochondrial proteins as interactors of ATPIF1, even in the absence of antimycin treatment (Table 2).

TABLE 2
Proteins Identified as Interacting with ATPIF1

| | FLAG- | FLAG- | MW |
|---|---|---|---|
| Protein: | ATPIF1 | OMP25TM | (Kda) |
| ATP synthase subunit alpha | 381 | 11 | 60 |
| ATP synthase subunit beta | 375 | 7 | 57 |
| ATPase inhibitor, | 175 | 3 | 12 |
| mitochondria; isoform 1 | | | |
| ATP synthase subunit | 59 | 2 | 33 |
| gamma, isoform L | | | |
| ATP synthase subunit O, | 47 | 3 | 23 |
| mitochondrial precursor | | | |
| ATP synthase subunit b, | 44 | 3 | 29 |
| mitochondrial precursor | | | |
| ATP synthase subunit d, | 26 | 1 | 18 |
| mitochondrial isoform a | | | |
| ATP synthase subunit g, | 14 | 0 | 11 |
| mitochondrial | | | |
| 60 kDa heat shock protein, | 28 | 4 | 61 |
| mitochondrial | | | |
| mitochondrial-processing | 14 | 0 | 58 |
| peptidase subunit alpha | | | |
| mitochondrial-processing | 17 | 0 | 54 |
| peptidase subunit beta | | | |
| UDP-glucose: glycoprotein | 15 | 5 | 107 |
| glucosyltransferase 1 | | | |
| protein zyg-11 homolog B | 19 | 0 | 84 |
| ornithine aminotransferase | 16 | 1 | 49 |

These data suggest that even in the absence of mitochondrial dysfunction, ATPIF1 is bound to a fraction of the F1-F0 ATP synthase complexes; raising the possibility that ATPIF1 indeed has at least one previously unidentified role, the loss of which confers antimycin resistance. We will perform similar IP-MS experiments in the presence and absence of antimycin. We expect that our analysis of proteins that differentially interact with ATPIF1 in the presence of antimycin will further elucidate mechanisms of resistance to complex III inhibition and uncover new roles for ATPIF1 in mitochondrial physiology.

### Example 4

### ATPIF1 Loss Protects Against Electron Transport Chain Dysfunction by Rescue of Mitochondrial Membrane Potential Via the ATP Synthase

Materials and Methods—Examples 4, 5 and 6

Drug Concentrations:

Drugs, unless otherwise indicated, were used at the following concentrations. Antimycin at 120 uM; oligomycin at 1 uM; 2′,3′-dideoxyinosine (ddI) at 118 uM. All drugs were acquired from Sigma-Aldrich except piericidin was from Enzo Life Sciences and blasticidin was from Invivogen.

Mitochondrial Mass Measurements:

200,000 cells were stained with 50 nM MitoTracker FM Green (Invitrogen) in RPMI media for 30 minutes at 37° C. Samples were kept on ice and then spun at 4000 rpm for 5 minutes at 4° C. in a table-top microcentrifuge, supernatant aspirated, cells washed once with ice-cold PBS, resuspended in ice-cold PBS with 2 ug/mL 7-AAD viability dye (Invitrogen), incubated for 5 minutes on ice, and analyzed by FACS. Only 7-AAD negative live cells were analyzed.

MMP Measurements:

200,000 cells were stained with 25 nM TMRM dye (Invitrogen) in parent media of cells. In the case of adherent cells, 10 uM verapamil (Sigma-Aldrich) was added to help retention of the dye. For suspension cells, the incubation of the dye was done for 30 minutes at 37° C., whereas for adherent cells, the incubation was done for 1 hour at 37° C. Suspension cells were kept on ice, then spun at 4000 rpm for 5 minutes at 4° C. in a table-top microcentrifuge, supernatant aspirated, cells washed once with ice-cold PBS, resuspended in ice-cold PBS with 2 ug/mL 7-AAD viability dye, incubated for 5 minutes on ice, and analyzed by FACS. Only 7-AAD negative live cells were analyzed. Adherent cells were first trypsinized, kept on ice, spun at 4000 rpm for 5 minutes at 4° C. in a table-top microcentrifuge, supernatant aspirated, cells washed once with ice-cold PBS, resuspended in ice-cold PBS with 2 ug/mL 7-AAD viability dye, incubated for 5 minutes on ice, and analyzed by FACS. Only 7-AAD negative live cells were analyzed.

ATP Measurements:

Equal numbers of cells were plated and ATP was determined using a CellTiterGlo assay (Promega) according to the manufacturer's instructions.

Metabolite Profiling:

4 million KBM7 cells were centrifuged at 1500 rpm for 4 minutes at 4° C. in a swinging bucket centrifuge after the desired drug treatments. The supernatant was decanted and the cell pellet kept on dry ice. Each cell pellet was washed twice with ice-cold PBS. 3 mL of 80% ice-cold MeOH was then added to each pellet and the pellet vigorously resuspended. Samples were then submitted to the Broad Institute Metabolomics Platform for further processing and analysis.

Viability Assays:

Suspension cells were kept on ice, then spun at 4000 rpm for 5 minutes at 4° C. in a table-top microcentrifuge, supernatant aspirated, cells washed once with ice-cold PBS, resuspended in ice-cold PBS with 2 ug/mL 7-AAD viability dye, incubated for 5 minutes on ice, and analyzed by FACS. For adherent cells, the overlying media and PBS used for pre-trypsinization washes was collected and combined with the suspension of trypsinized cells to gather any dead cells that may have detached into the media. The cell suspensions were kept on ice, spun at 1500 rpm for 5 minutes at 4° C. in a swinging bucket centrifuge, supernatant aspirated, cells washed once with ice-cold PBS, resuspended in ice-cold PBS with 2 ug/mL 7-AAD viability dye, incubated for 5 minutes on ice, and analyzed by FACS. Viability was assessed by 7-AAD exclusion. For HeLa, SH-SY5Y, and Malme-3M cells, 500-2,000 cells were seeded per well of white, clear-bottom 96-well plates (Greiner Bio-One), treated with drugs, and then analyzed using CellTiter-Glo according to the manufacturer's instructions (Promega). For primary hepatocytes, 100,000 cells were seeded per well of a 24-well TPP plate (Light Labs), treated with antimycin, and then analyzed using CellTiter-Glo.

Q-RT-PCR:

RNA was extracted from cells using the Qiagen RNeasy Mini Kit (Qiagen) according to the manufacturer's protocol. Primers specific to ATPIF1 mRNA were used in conjunction with a SyBr Green Kit (AB) to quantify the levels of ATPIF1 mRNA according to the manufacturer's instructions. Primers targeting ATPIF1 and ACTB mRNA were used to assess ATPIF1 mRNA levels by normalizing et values of ATPIF1 to those of ACTB: ATPIF1_mRNA_F, CTT CGG CTC GGA TCA GTC (SEQ ID NO. 10); ATPIF1_mRNA_R, CTG CCA GTT GTT CTC TAC TCT G (SEQ ID NO. 11); ACTB_mRNA_F, CCC TGG CAC CCA GCA C (SEQ ID NO. 12); ACTB_mRNA_R, GCC GAT CCA CAC GGA GTA C (SEQ ID NO. 13). Relative differences between samples were determined using the comparative Ct method

mtDNA Copy Number Assay:

mtDNA was extracted using a QiaAmp DNA Mini Kit (Qiagen) and primers specific to the mitochondrial gene ND1 and the nuclear gene Actb were used in conjunction with a SyBr Green Kit (AB) according to the manufacturer's instructions. The ratio of ND1 copies to Actb copies was used to assess the relative mtDNA/nuclear DNA copy numbers between different samples. In some experiments primers targeting the mitochondrial gene ND1 and the nuclear gene RUNX2 were used to assess mtDNA copy number by normalizing ct values of ND1 to those of RUNX2: ND1_F, CCC TAA AAC CCG CCA CAT CT (SEQ ID NO. 14); ND1_R, GAG CGA TGG TGA GAG CTA AGG T (SEQ ID NO. 15); RUNX2_F, CGC ATT CCT CAT CCC AGT ATG (SEQ ID NO. 16); RUNX2_R, AAA GGA CTT GGT GCA GAG TTC AG (SEQ ID NO. 17). Relative differences between samples were determined using the comparative Ct method.

Cell Lines and Media:

COX1 mutant cells and 143B ρ⁰ cells are described in Kwong, J Q, et al., J. Cell Biol. 2007; 179(6):1163-77. The lines are human osteosarcoma cybrid cell lines derived by fusion of 143B ρ⁰ cells with enucleated cells from a subject with normal mtDNA or a patient with a COX1 mtDNA mutation. The COX1 mutant cells have a stop-codon mutation at mtDNA nt 6930 in subunit I of complex IV (Bruno et al., Am J Hum Genet. (1999) 65(3):611-20). SH-SY5Y, and Malme-3M cells were from the ATCC. DMEM, RPMI-1640 media from Sigma; IMDM from US Biologicals; Medium 199 and GlutaMAX from Invitrogen.

Seahorse Analysis.

Oxygen consumption of intact cells was measured using an XF24 Extracellular Flux Analyzer (Seahorse Bioscience). For suspension cells, Seahorse plates were coated with Cell TAK (BD, 0.02 mg/ml in 0.1 μM NaHO3) for 20 minutes to increase adherence of suspension cells. 300,000 cells were then attached to the plate by centrifugation at 2200 rpm without brakes for 5 min. IMDM was used as the assay media for all experiments.

Electron Microscopy.

KBM7 cells were fixed in 2% glutaraldehyde in 0.1 M sodium cacodylate buffer pH 7.4 at room temperature. After post-fixation in 2% OsO4, blocks were processed for embedding in Epon 812. Thin sections were obtained, stained with uranyl acetate and lead citrate, and examined by transmission electron microscopy in a JEOL EX 1200 electron microscope. For each cell type, we analyzed 10 representative electron micrographs corresponding to sections (whole cell profiles) that went through the nucleus. For each of the 10 cells, we took a low magnification (5000×) electron micrograph from which we counted the number of mitochondria and measured the area of the cell using Image J from the NIH. We then marked each mitochondrion and took individual high magnification (40,000×) electron micrographs of all the mitochondria in the cell profile. In these 40,000× micrographs, we measured the area and cristae number of each mitochondrion.

FACS assays. For measurements of ΔΨm, 100,000 cells were incubated with TMRM (25 nM) and the indicated amounts of drugs for the indicated amounts of time before collection. For measurements of mitochondrial mass, 100,000 cells were incubated with MitoTracker Green FM (50 nM) for one hour. For primary hepatocytes, cells were assayed in suspension immediately after harvest from the liver and incubated with verapamil (20 μM) to facilitate retention of TMRM and MitoTracker Green FM signals. Afterwards, cells were collected by centrifugation, washed once with PBS, and resuspended in PBS with 7-AAD (2 μg/mL) for analysis. KBM7 cells and primary hepatocytes were centrifuged at 2000 rpm for 5 minutes and 500 rpm for 5 minutes at 4° C., respectively.

Cell Culture and Virus Transduction.

KBM7 cells were cultured in IMDM supplemented with 10% IFS and penicillin/streptomycin, except for studies using tigecycline in which they were cultured in RPMI-1640. SH-SY5Y, HeLa, Malme-3M cells were cultured in DMEM supplemented with 10% FBS and penicillin/streptomycin. HeLa WT, HeLa ρ⁰, and KBM7 cells used for ddI experiments were grown in DMEM supplemented with 10% FBS, penicillin/streptomycin, and 100 μg/mL uridine. Primary hepatocytes were cultured in Medium 199 supplemented with 10% FBS, penicillin/streptomycin, and GlutaMAX. KBM7, HeLa WT, and HeLa ρ⁰ cells stably overexpressing ATPIF1 (WT or E55A) were generated via infection with retroviruses and selected with blasticidin (10 μg/mL) for three days. Malme-3M cells stably overexpressing RAP2A or ATPIF1 were generated by infection with lentiviruses expressing the corresponding cDNAs, followed by selection with puromycin (2 μg/mL) for three days. KBM7, SH-SY5Y, and HeLa cells expressing shLuc or shATPIF1 were generated by infection with lentiviruses expressing the corresponding shRNAs. All cells were infected with spin-infection using a 30 minute spin at 2,250 rpm in media containing polybrene (4 μg/mL), followed by selection one day later.

DNA Constructs.

The ORF of ATPIF1 isoform 1 was cloned into the lentiviral vector, PLJM1-puro, and the retroviral vector, pMXs-IRES-blasticidin, using the following primers:

PLJM1_ATPIF1_F,
(SEQ ID NO. 18)
ATT ACC GGT ATG GCA GTG ACG GCG TTG;
PLJM1_ATPIF1_R,
(SEQ ID NO. 19)
ATT GAA TTC TTA ATC ATC ATG TTT TAG CAT TTT GAT
CTT CTG C;
pMXs_ATPIF1_F,
(SEQ ID NO. 20)
ATG GAT CCG CCA CCA TGG CAG TGA CGG CGT TG;
pMXs_ATPIF1_R,
(SEQ ID NO. 21)
ATG CGG CCG CTT AAT CAT CAT GTT TTA GCA TTT TGA
TCT TCT GCT TAT GG.

pljm1-Flag-RAP2A was obtained from Addgene. The E55A mutation was generated using site-directed mutagenesis with a QuikChange II XL kit (Stratagene) and the following primers:

E55A_F,
(SEQ ID NO. 22)
GAG AGA GCA GGC TGA AGC GGA ACG ATA TTT CCG AG;
E55A_R,
(SEQ ID NO. 23)
CTC GGA AAT ATC GTT CCG CTT CAG CCT GCT CTC TC.

Immunoblots.

Cells were washed twice with ice-cold PBS and harvested in standard RIPA buffer containing Complete EDTA-free protease inhibitor (Roche). Proteins from total lysates were resolved by 12-16% SDS-PAGE and analyzed by immunoblotting using the indicated antibodies (1:1000).

ATPIF1−/− Mice.

All animal studies and procedures were approved by the MIT Institutional Animal Care and Use Committee. ATPIF1−/− C57BL/6N mice were obtained from the International Knockout Mouse Consortium (Brown and Moore, 2012) and maintained on a standard light-dark cycle with food and water ad libitum. Genotyping primers were designed to distinguish between the native WT allele, the gene-trap allele, and the lacZ cassette: ATPIF1_geno_F, CGG AAA AAC AGC AGG GAA AT (SEQ ID NO. 24); ATPIF1_geno_R, GGC ATT GGA CTG GGG TTT AC (SEQ ID NO. 25); lacZ_geno_F, ATT AGG GCC GCA AGA AAA CT (SEQ ID NO. 26); lacZ_geno_R, CTG TAG CGG CTG ATG TTG AA (SEQ ID NO. 27). PCR with ATPIF1_geno_F, ATPIF1_geno_R gives a 221 bp product for the WT allele and a 163 bp product for the gene-trap allele. PCR with lacZ_geno_F and lacZ_geno_R gives a 192 bp product for the lacZ cassette.

Primary Hepatocyte Cultures.

Primary hepatocytes were isolated from 8-18 week old mice with the indicated genotypes by collagenase perfusion. Prior to plating, cells were incubated with ACK (Ammonium-Chloride-Potassium) lysing buffer for two minutes to eliminate red blood cells. Cells were maintained in Medium 199 supplemented with 10% FBS, penicillin/streptomycin, and GlutaMAX. Media was changed daily and cells were treated as indicated.

Results

Several lines of evidence strongly suggest that ATPIF1 loss protects against electron transport chain dysfunction by rescue of mitochondrial membrane potential via the ATP synthase.

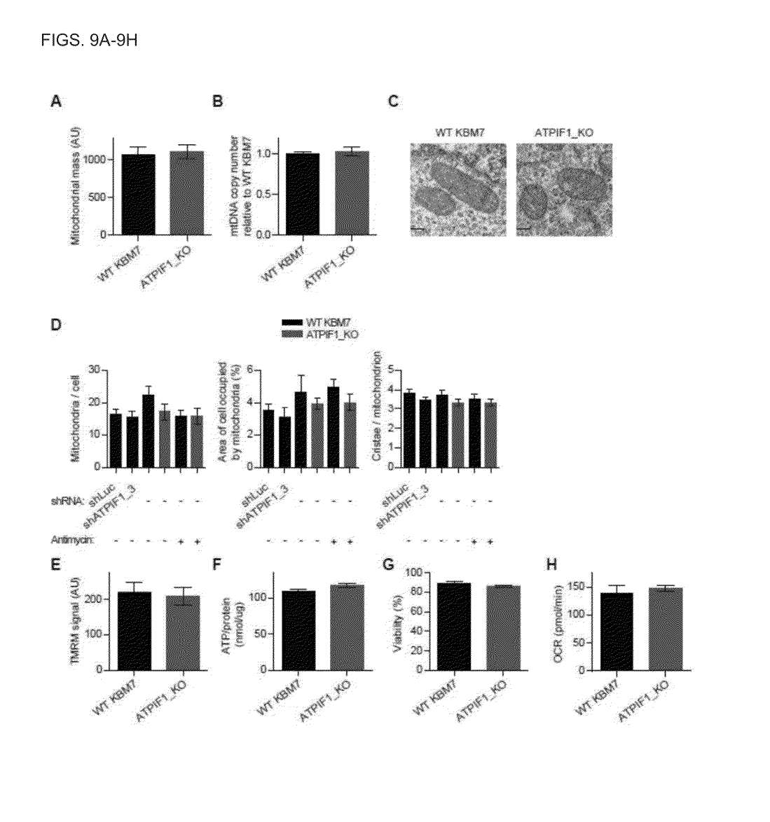

Consistent with results described in Example 2, measurement of mitochondrial mass of WT and IF1 KO KBM7 cells as determined by Mitotracker Green staining and FACS revealed no significant difference in mitochondrial mass, indicating that ATPIF1 loss does not appear to have an effect on mitochondrial number or size, nor did it alter a number of general aspects of cellular and mitochondrial physiology (FIGS. 3B and 7A and 9).

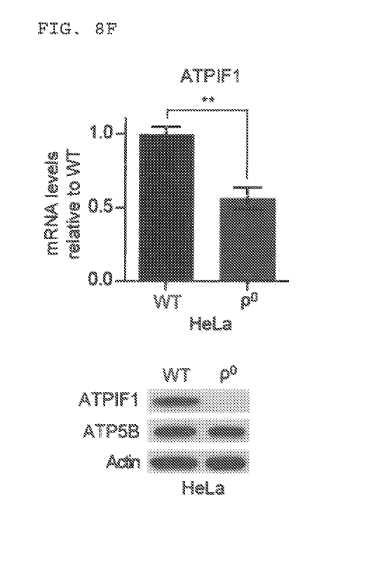

Mitochondrial membrane potential (MMP) and ATP levels were determined in WT and IF1 KO KBM7 cells in response to different mitochondrial toxins by TMRM staining with FACS and CellTiterGlo assays, respectively. Antimycin is a complex III inhibitor, oligomycin is an inhibitor of the ATP synthase. As shown in FIG. 7B (upper panel) IF1 KO cells exhibited significant recovery in MMP after loss of MMP caused by exposure to antimycin, whereas WT cells did not. ATP levels decreased significantly in IF1 KO cells exposed to antimycin but not in WT cells exposed to antimycin. These results indicate that IF1 KO cells can use the ATP synthase to maintain MMP by pumping protons out of the mitochondrial matrix at the expense of ATP consumption (ATP synthase operating “in reverse”), whereas WT cells cannot. In addition, oligomycin, a blocker of the ATP synthase, completely prevents IF1 KO cells from rescuing their MMP and using ATP, confirming that the recovery of MMP in IF KO cells is due to activity of the ATP synthase.

Metabolite profiling of WT and IF1 KO KBM7 cells after 1 hour of antimycin treatment was performed. The results (FIG. 7D) showed that IF1 KO KBM7 cells have more depletion of glycolytic metabolites (i.e. DHAP, glyceraldehyde 3P, and F6P) than WT cells, which is consistent with IF KO cells having to increase glycolysis to maintain ATP to allow ATP synthase to operate in reverse.

Viability of WT and IF1 KO KBM7 cells in response to different mitochondrial toxins (Anti=antimycin, Oligo=oligomycin) was assessed by 7-AAD staining and FACS (FIG. 7D). Consistent with the data in FIGS. 7B and 7C, blocking ATP synthase with oligomycin eliminates the beneficial effect seen with IF1 KO cells under antimycin treatment. The addition of oligomycin to antimycin-treated IF1 KO cells decreased ΔΨm, increased ATP levels, but led to decreased survival, suggesting that maintenance of ΔΨm is more important than preservation of ATP in ameliorating complex III blockade in KBM7 cells. We also examined the mitochondrial mass, mitochondrial DNA (mtDNA) copy number, mitochondrial ultrastructure, and resting ΔΨm, ATP, viability, and oxygen consumption of WT and ATPIF1_KO KBM7 cells, but found no significant differences, thus showing ATPIF1 loss does not have general effects on mitochondrial metabolism and cellular physiology. Collectively, these data demonstrate that ATPIF1 loss confers resistance to electron transport chain dysfunction (such as that caused by complex III blockade) through maintenance of ΔΨm via reversal of the F1-F0 ATP synthase.

Consistent with the results in KBM7 cells, SH-SY5Y and HeLa cells expressing an shRNA targeting ATPIF1 were more resistant to antimycin than cells expressing a control hairpin (FIG. 7F). In addition, we found that overexpression of ATPIF1 in Malme-3M, a cell line with low endogenous levels of ATPIF1, increased their sensitivity to antimycin (FIG. 7G).

MMP and viability of cybrid cells having mtDNA derived from a normal (WT) subject or from a patient with a deficiency in COX1, a mitochondrially encoded gene important for proper complex IV activity and thus important for ETC activity, were measured. As shown in FIG. 7E, oligomycin causes a dramatic decrease in MMP and kills the cells having patient-derived mtDNA but not cells having mtDNA derived from healthy subjects (WT). These observations suggest that the ATP synthase is used in reverse in cells derived from patients with mitochondrial deficiencies, as adding oligomycin reduces MMP and kills the patient-derived cells but not cells derived from healthy subjects. This result indicates that maintenance of MMP can be critical for cells derived from patients with mitochondrial deficiencies. All experiments: n=3 and error bars are SEM.

### Example 5

### Inhibition of ATPIF1 is Beneficial in Several Models of ETC Dysfunction

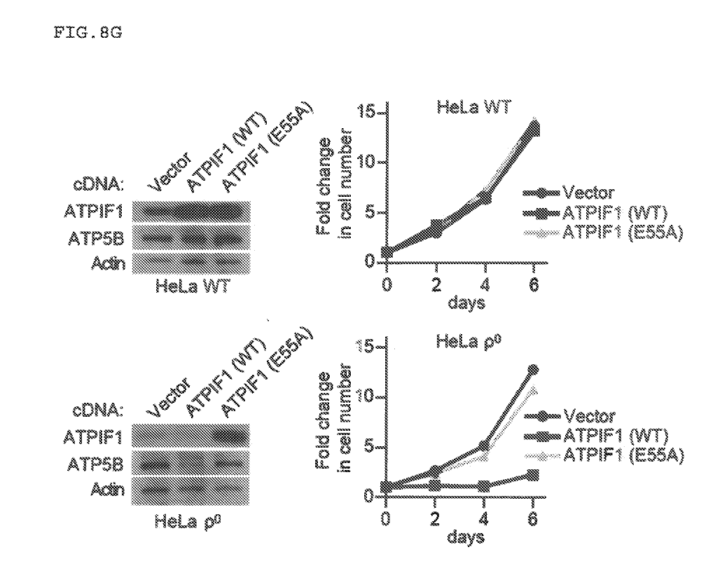

Viability of WT vs ATPIF1 KO KBM7 cells in response to different inhibitors of the ETC was assessed using the cell viability dye 7AAD. Piericidin A and MPP+ are both complex I inhibitors (Darrouzet et al., 1998), whereas tigecycline is an inhibitor of mitochondrial translation (\check{S}krtié et al., 2011). As shown in FIG. 8A, cells lacking ATPIF1 have increased viability as compared to wild type cells. Thus, loss of ATPIF1 protects against these various different insults against the mitochondrial ETC, not just antimycin (complex III inhibition) (confirming results presented in Example 3).

ATPIF1 mRNA and protein levels in WT and ρ0 (mtDNA-depleted) cells (derived from 143B cells) were measured using Q-RT-PCR and immunoblot analysis, respectively. As shown in FIG. 8B, ATPIF1 expression is reduced to begin within ρ⁰ cells, which suggests that there is a selection to lose ATPIF1 activity in cells with no mtDNA (i.e. very severe ETC dysfunction), which is consistent with the findings of our screen.

MMP and proliferation of WT vs ρ⁰ cells overexpressing a control protein (RAB) or ATPIF1 were measured (FIG. 8C). Adding back ATPIF1 in WT cells had little effect on MMP and proliferation but, in ρ⁰ cells, strongly reduced MMP and cell proliferation. These results show that losing ATPIF1 activity is necessary in ρ⁰ cells as its forced expression in ρ⁰ cells dramatically reduced MMP and cell viability but had no significant effect on WT cells.

We also found that HeLa ρ⁰ cells intrinsically possess low mRNA and protein levels of ATPIF1, when compared to their WT counterparts (FIG. 8F). ρ⁰ cells are devoid of mtDNA and consequently have defects in complexes I, III, and IV, resulting in undetectable ETC activity (Jazayeri et al., 2003). Previous work has shown that ρ⁰ cells maintain ΔΨm by using the electrogenic exchange of ATP and ADP, coupled to ATP hydrolysis by an F1-F0 ATP synthase defective in pumping protons, and that this activity is important for cellular health (Appleby et al., 1999; Buchet and Godinot, 1998). We therefore hypothesized that there could be a strong selective pressure to decrease ATPIF1 levels under severe ETC dysfunction in order to facilitate reversal of the F1-F0 ATP synthase. To address this, we overexpressed WT ATPIF1 or a mutant ATPIF1 harboring an E55A substitution that renders the protein unable to interact with the F1-F0 ATP synthase (Ichikawa et al., 2001). Overexpression of WT ATPIF1, but not E55A ATPIF1, strongly impaired proliferation in HeLa ρ⁰ cells but not in HeLa WT cells (FIG. 8G). The differences observed between WT and E55A ATPIF1 were not simply a result of E55A ATPIF1 protein instability because both variants of ATPIF1 were overexpressed to a similar degree, as seen in the immunoblots of HeLa WT cells (FIG. 8G). Intriguingly, at the time of collection, we found that the surviving HeLa ρ⁰ cells infected with virus expressing WT ATPIF1 had lower amounts of ATPIF1 than those infected with virus expressing E55A ATPIF1, which is consistent with a selection against ATPIF1 activity on the F1-F0 ATP synthase in the ρ0 state (FIG. 8G). Collectively, these data demonstrate that reduced ATPIF1 activity is essential for the viability of human ρ⁰ cells lacking a mitochondrial genome.

Failure to maintain proper amounts of the mitochondrial genome is a distinctive feature of a class of severe respiratory chain disorders known as mtDNA depletion syndromes (Lee and Sokol, 2007). Because of our interest in ATPIF1 inhibition as a potential strategy for ameliorating severe ETC dysfunction, we asked if loss of ATPIF1 alone was sufficient to improve cell viability during progressive mtDNA depletion. To do this, we exposed WT and IF1 KO KBM7 cells for 50 days to the drug 2′,3′-dideoxyinosine (ddI), an inhibitor of mtDNA replication, which depletes cells of their mtDNA (Lewis et al., 2003; Walker et al., 2002). During the time period of days 0-18 (“Early”), days 19-34 (“Middle”), or days 35-50 (“Late”), we measured the proliferative ability, mtDNA copy number, and viability of cells to essentially get a snapshot of the cellular behavior at defined timepoints of ddI exposure. ddI led to an immediate decrease in mtDNA copy number in the initial days of treatment, concomitant with a decrease in cell proliferation that was roughly equivalent between WT and ATPIF1_KO KBM7 cells (FIG. 8D, upper panel (Early)). It has been observed from previous studies that this amount of mtDNA depletion still allows for residual ETC function (Jazayeri et al., 2003) and so it is unlikely that ATPIF1 was maximally activated in the WT KBM7 cells at this point. mtDNA was progressively depleted with each successive week of ddI treatment and, by about day 25, both WT and ATPIF1_KO KBM7 cells had trace amounts of mtDNA (FIG. 8D, middle panel (Middle)). While both WT and ATPIF1_KO KBM7 cells proliferated slower than their untreated counterparts, ATPIF1_KO cells demonstrated a significantly faster rate of proliferation than WT KBM7 cells, consistent with loss of ATPIF1 improving cell viability under conditions of severe ETC dysfunction (FIG. 8D, Middle). As shown in FIG. 8D, losing ATPIF1 activity is sufficient to protect KBM7 cells from the early (days 0-day 18) and intermediate effects (days 19-day 34) of ddI-induced mtDNA depletion, which suggests that inhibiting ATPIF1 is a promising potential therapeutic strategy for mtDNA depletion syndromes.

ATPIF1 protein levels, proliferation, mtDNA copy number, and viability of WT and IF KO KBM7 cells under long-term exposure to ddI were determined (FIG. 8E). It was found that long-term exposure to ddI selects for WT cells that have greatly reduced ATPIF1 protein levels and activity (FIG. 8E, Western blot shown in left panel). These ATPIF1-low WT cells behave similarly in terms of viability (FIG. 8E, second panel from left), mtDNA copy number (FIG. 8E, second panel from right) and proliferation (FIG. 8E, right panel) when compared to the IF1 KO KBM7 cells. To reach this level of selection, the WT cells had to undergo numerous rounds of proliferation and death, consequences which one would want to avoid in a patient with a mtDNA depletion syndrome or other mitochondrial disorder as this would likely have pathologic manifestations. Treatment with an ATPIF1 inhibitor may reduce or remove this selective pressure.

### Example 6

### ATPIF1 Loss in Primary Hepatocytes Ameliorates the Effects of Complex III Blockade

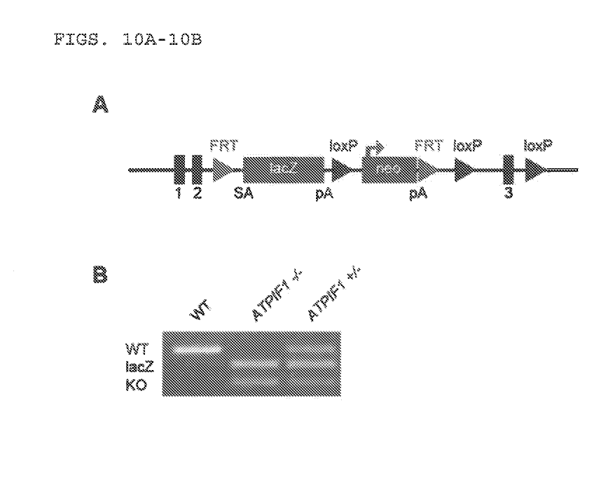

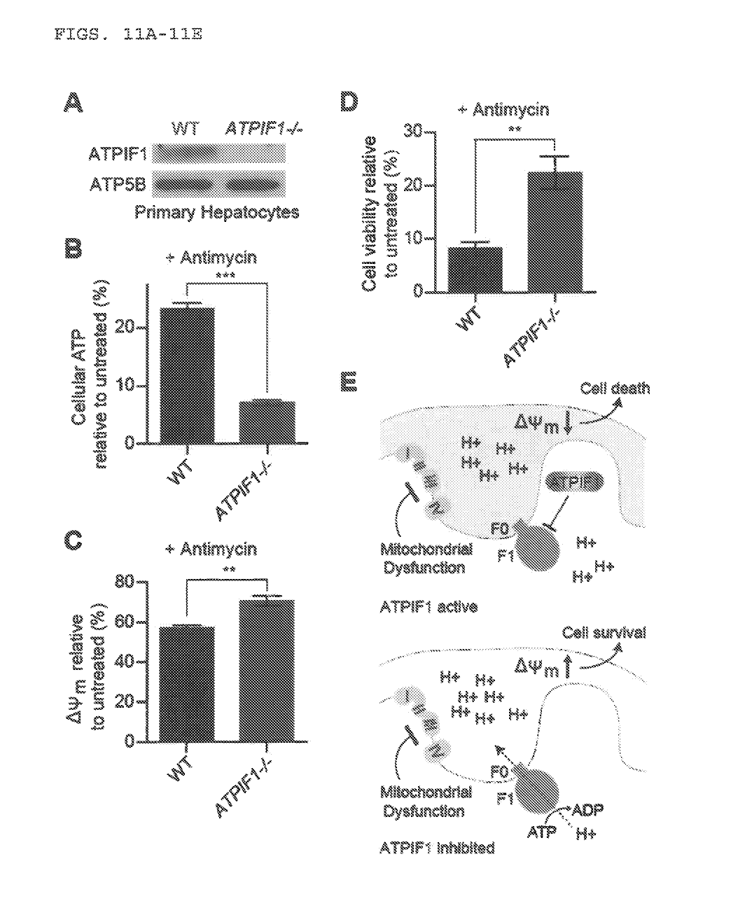

Because severe forms of mitochondrial respiratory chain disorders can lead to cell death and loss of tissue parenchyma in organs such as the liver (Lee and Sokol, 2007; Morris, 1999), we transitioned to a more physiological context and asked if loss of ATPIF1 in hepatocytes could ameliorate ETC dysfunction and improve cell viability. WT and ATPIF1^{-/-} mice were obtained from the International Knockout Mouse Consortium (Brown and Moore, 2012) (FIG. 10) and primary hepatocytes isolated from ATPIF1^{-/-} mice had undetectable amounts of ATPIF1 (FIG. 11A). Consistent with the results seen in cell lines, antimycin treatment led to a greater decrease in cellular ATP (FIG. 11B) and a greater increase in ΔΨₘ (FIG. 11C) in ATPIF1^{-/-} hepatocytes than in WT hepatocytes, indicating that there was greater reversal of the F1-F0 ATP synthase in ATPIF1^{-/-} hepatocytes. Importantly, ATPIF1^{-/-} hepatocytes had increased cell viability relative to WT hepatocytes following treatment with antimycin (FIG. 11D), which demonstrates that the beneficial effects of ATPIF1 loss under severe ETC dysfunction are not limited to rapidly proliferating cancer cell lines, but can also occur in post-mitotic, differentiated cells that better recapitulate the metabolism of tissues affected in severe mitochondrial respiratory chain disorders (Vander Heiden et al., 2009). The smaller effects of ATPIF1 loss on hepatocyte viability during antimycin treatment, as compared to KBM7 cells, are partially due to the frailty of primary mouse hepatocytes when cultured ex vivo (Edwards et al., 2013; Klaunig et al., 1981). Taken together, these data demonstrate that ATPIF1 loss in primary hepatocytes can ameliorate the effects of complex III blockade.

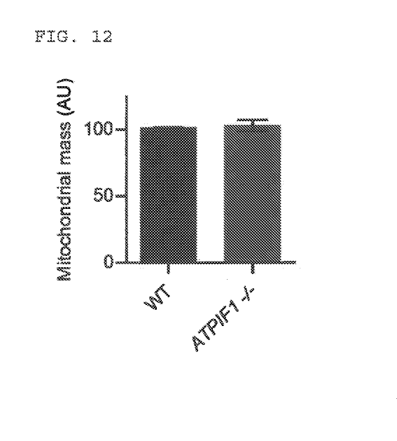

Our findings in primary hepatocytes suggest that hepatic delivery of RNAi constructs targeting ATPIF1 either via adeno-associated virus or lipid nanoparticles, both of which delivery approaches have seen clinical efficacy in gene therapy of the liver, will have therapeutic value (Fitzgerald et al.; Nathwani et al., 2011). Notably, ATPIF1^{-/-} mice appear phenotypically normal and their hepatocytes exhibit no significant alterations in ATP synthase activity or mitochondrial structure (Nakamura et al., 2013). In agreement with these findings, we did not observe any significant differences in the mitochondrial mass of WT and ATPIF1^{-/-} primary hepatocytes (FIG. 12). Taken together, these data suggest that ATPIF1 inhibition is relatively well-tolerated.

### Example 7

### Identification of Additional Targets for Treatment of Mitochondrial Disorders

The results from the antimycin screen described above validate our haploid screen approach as a method for identifying genes with potential as targets to treat mitochondrial deficiencies. Other compounds such as oligomycin (complex V inhibitor), TTFA (complex II inhibitor), rotenone (complex I inhibitor) and FCCP (uncoupling agent) can be screened using this approach. Such screening is expected to open new perspectives for treatment of mitochondrial disorders by identifying potential gene targets for therapy as well as improve understanding of mitochondrial physiology by elucidating functions of these genes and their gene products.

### Example 8

### Testing ATPIF1 Inhibitor in Animal Model of GRACILE Syndrome

Mice harboring a homozygous Bcs1I 232A>G mutation (21) are treated starting at birth or starting at 2 or 4 weeks after birth with an siRNA that inhibits ATPIF1. The siRNA is administered intravenously using standard methods (e.g., daily injection into the tail vein). A range of doses providing different levels of ATPIF1 inhibition is tested. In other experiments, mice are treated with a vector that directs expression of an shRNA that inhibits ATPIF1. In some experiments an AAV, Ad, or retroviral vector is used. In some experiments expression of the shRNA is driven by an RNA pol II promoter. In some experiments expression of the shRNA is driven by an RNA pol III promoter. The vector is administered by intravenous injection to the tail vein, intraperitoneally, into the portal vein, or by direct administration to the hepatic parenchyma. Mice are monitored for symptoms similar to those found in the human disease GRACILE syndrome, i.e., growth failure, hepatic glycogen depletion, steatosis, fibrosis, and cirrhosis, as well as tubulopathy, complex III deficiency, lactacidosis. The development and severity of such symptoms in treated mice is compared with that of control mice not treated with the siRNA or vector. Average lifespan is compared between the groups. A reduction in incidence and/or severity of symptoms and/or an increased lifespan in any of the treated groups as compared with untreated controls confirms the usefulness of ATPIF1 inhibition as a therapeutic strategy.

### Example 9

### Testing Effect of ATPIF1 Loss of Function in Neurotoxin-Induced Parkinson's Disease Animal Model

ATPIF1 is ablated in adult mouse dopaminergic (DA) neurons using the Tamoxifen-inducible CreERT2/loxP system (Indra A. K., et al. (1999) Temporally-controlled site-specific mutagenesis in the basal layer of the epidermis: comparison of the recombinase activity of the tamoxifen-inducible Cre-ER(T) and Cre-ER(T2) recombinases. Nucleic Acids Res. 27, 4324-4327), in which the expression of Cre recombinase fused to a modified ligand-binding domain of the estrogen receptor (CreER(T2) is controlled by the regulatory elements of the dopamine active transporter (DAT; SLC6A3) gene. Mice in which both copies of the ATPIF1 gene are flanked by loxP sites are obtained (ATPIF1^{f1/f1} mice). An ATPIF1^{f1/f1}/DATCreERT2 mouse line is generated by mating ATPIF1^{f1/f1} mice with DATCreERT2 mice. To induce ATPIF1 deletion in adult DA neurons, 8- to 10-wk-old ATPIF1-knockout mice are injected with Tamoxifen. At various time points, mice are either intraperitoneally injected once daily for 3 days with 20 mg/kg body weight 1-methyl-4-phenyl-1,2,3,6 tetrahydropyridine hydrochloride (MPTP; Sigma, St, Louis, Mo., USA) or administered 6OHDA by unilateral stereotactic intrastriatal injection. 6OHDA (Sigma) is diluted in 0.9% NaCl to 5.0 mg/ml final concentration, and a total dose of 15.0 mg (3 μl) is injected using a glass capillary into the striatum. Sham-operated control mice receive 3 μl 0.9% NaCl intrastriatal injection to the same coordinates. Littermates harboring floxed ATPIF1 alleles but not treated with Tamoxifen are used as controls.

Exemplary methods for analyzing the mice are found in Ekstrand, Mich., et al., PNAS 104 (4): 1325-1330 (2007) and/or Domanskyi, A., et al., FASEB J. 25, 2898-2910 (2011).

Mice in each group are sacrificed at various time points following administration of MPTP or 6OHDA for analysis of relevant regions of the brain (e.g., substantia nigra, striatum). Neuronal numbers and morphology are analyzed on micrographs from stained brain sections using histology and immunohistochemistry, and electron microscopy is performed. Striatal tyrosine hydroxylase (TH) and DAT immunoreactivity are assessed. Striatal dopamine content measurements are performed using an HPLC-electrochemical detection method. Increased striatal dopamine levels, a reduction in nigral dopamine neuron intraneuronal inclusions, and/or a reduction in nigral dopamine neuron cell death in any of the treated groups as compared with controls confirms the usefulness of ATPIF1 inhibition as a therapeutic strategy.

A rotarod assay (to measure forelimb and hindlimb motor coordination and balance), footprint analysis, open field testing, and grip strength measurement are performed. Mice are monitored for symptoms similar to those found in Parkinson's disease in humans, e.g., loss of motor function. The development and severity of such symptoms in treated mice is compared with that of control mice. Average lifespan is compared between the groups. A reduction in incidence and/or severity of symptoms and/or an increased lifespan in any of the ATPIF1-ablated groups as compared with controls confirms the usefulness of ATPIF1 inhibition as a therapeutic strategy.

### Example 10

### Testing ATPIF1 Inhibitor in Animal Model of Parkinson's Disease

MitoPark mice are treated starting at birth or starting at 3, 6, 12, or 15 weeks after birth with an siRNA that inhibits ATPIF1. The siRNA is administered intravenously using standard methods (e.g., daily injection into the tail vein). A range of doses providing different levels of ATPIF1 inhibition is tested. In other experiments, mice are treated with a vector that directs expression of an shRNA that inhibits ATPIF1. In some experiments an AAV, Ad, or retroviral vector is used. In some experiments expression of the shRNA is driven by an RNA pol. II promoter. In some experiments expression of the shRNA is driven by an RNA pol III promoter. The vector is administered by intravenous injection to the tail vein, intraperitoneally, into the portal vein, or by direct administration to the hepatic parenchyma.

Mice in each group are sacrificed at various time points for analysis of relevant regions of the brain as described in Example 9. Increased striatal dopamine levels, a reduction in nigral dopamine neuron intraneuronal inclusion, s and/or reduction in nigral dopamine neuron cell death in any of the treated groups as compared with untreated controls confirms the usefulness of ATPIF1 inhibition as a therapeutic strategy.

A rotarod assay (to measure forelimb and hindlimb motor coordination and balance), footprint analysis, open field testing, and grip strength measurement are performed. Mice are monitored for symptoms similar to those found in Parkinson's disease in humans, e.g., loss of motor function. The development and severity of such symptoms in treated mice is compared with that of control mice not treated with the siRNA. Average lifespan is compared between the groups. A reduction in incidence and/or severity of symptoms and/or an increased lifespan in any of the treated groups as compared with untreated controls confirms the usefulness of ATPIF1 inhibition as a therapeutic strategy.

### Example 11

### Testing ATPIF1 Inhibition in Animal Models of mtDNA Depletion

mtDNA depletion syndromes often cause liver failure. The ability of inhibition of ATPIF1 to protect against depletion of hepatic mtDNA is tested in mice in which hepatic mtDNA depletion is induced either pharmacologically by drugs such as ddI (didanosine (2′,3′-dideoxyinosine) or genetically by ablation of TFAM, the gene encoding the master mitochondrial transcription factor (mitochondrial transcription factor A; Gene ID 7019 (human gene); Gene ID 21780 (mouse gene). It is known that loss of TFAM activity leads to mtDNA depletion.

Mice engineered to have a liver-specific inducible knockout of TFAM are generated, termed TFAM knockout (KO) mice hereafter. Liver-specific knockout of TFAM is confirmed by DNA, RNA, and protein analysis.

In a set of experiments, a group of TFAM knockout mice are treated with a vector containing that directs expression of an shRNA targeted to ATPIF1. The vector is administered intravenously by injection into the tail vein, intraperitoneally, into the portal vein, or by direct administration to the hepatic parenchyma. In some experiments an AAV, Ad, or retroviral vector is used. In some experiments expression of the shRNA is driven by an RNA pol II promoter. In some experiments the promoter is hepatocyte-specific. In some experiments expression of the shRNA is driven by an RNA pol III promoter. In some experiments the vector is administered prior to inducing loss of ATPIF1. In some experiments the vector is administered after loss of ATPIF1 is induced.

In a set of experiments, a group of TFAM knockout mice are treated with an siRNA that inhibits ATPIF1. The siRNA is administered intravenously using standard methods (e.g., daily injection into the tail vein). A range of doses providing different levels of ATPIF1 inhibition is tested. In some experiments the siRNA is administered starting prior to inducing loss of ATPIF1. In some experiments the siRNA is administered at the same time as loss of ATPIF1 is induced after loss of ATPIF1 is induced.

In a set of experiments, ddI is administered to group of mice to induce hepatic mtDNA depletion. The mice are treated with a vector that directs expression of an shRNA targeted to ATPIF1. The vector is administered intravenously by injection into the tail vein, intraperitoneally, into the portal vein, or by direct administration to the hepatic parenchyma. In some experiments an AAV, Ad, or retroviral vector is used. In some experiments expression of the shRNA is driven by an RNA pol II promoter. In some experiments the promoter is hepatocyte-specific. In some experiments expression of the shRNA is driven by an RNA pol III promoter. In some experiments the vector is administered prior to administering ddI. In some experiments the vector is administered at the same time as or after administering ddI.

In a set of experiments, ddI is administered to group of mice to induce hepatic mtDNA depletion. The mice are treated with an siRNA that inhibits ATPIF1. The siRNA is administered intravenously using standard methods (e.g., daily injection into the tail vein). A range of doses providing different levels of ATPIF1 inhibition is tested. In some experiments the siRNA is administered starting prior to administration of ddI. In some experiments the siRNA is administered starting at the same time as or after administering ddI.

In each set of experiments, biomarkers of liver damage are assessed in blood samples obtained at various time points. Such biomarkers may include markers of liver injury and/or markers of liver function. Parameters that may be measured to assess liver function include prothrombin time (PT/INR), aPTT, albumin, and/or bilirubin (direct and indirect). Parameters that may be measured to assess liver injury include liver transaminases aspartate transaminase (AST), alanine aminotransferase (ALT), the AST/ALT ratio, and/or alkaline phosphatase.

Mice in each group are sacrificed and liver histology and immunohistochemistry are performed to assess markers of liver function and/or liver injury. Liver tissue is assessed for evidence of necrosis, apoptosis, altered mitochondrial number, function, and/or morphology (e.g., by electron microscopy). A reduced level of liver damage in mice treated with the vector or siRNA as compared to control mice that are not treated confirms the usefulness of ATPIF1 inhibition as a therapeutic strategy.

Average lifespan is compared between the groups. An increased lifespan in any of the treated groups as compared with untreated controls confirms the usefulness of ATPIF1 inhibition as a therapeutic strategy.

### REFERENCES

1. S. DiMauro, E. A. Schon, N Engl J Med 348, 2656 (Jun. 26, 2003).
2. J. Zheng et al., Metabolism, (Dec. 28, 2011).
3. M. Lagouge et al., Cell 127, 1109 (Dec. 15, 2006).
4. M. Kitada, S. Kume, N. Imaizumi, D. Koya, Diabetes 60, 634 (February, 2011).
5. J. E. Carette et al., Science 326, 1231 (Nov. 27, 2009).
6. S. Grimm, Nat Rev Genet. 5, 179 (March, 2004).
7. M. P. Bayona-Bafaluy, F. Sanchez-Cabo, P. Fernandez-Silva, A. Perez-Martos, J. A. Enriquez, Mitochondrion 11, 467 (May, 2011).
8. J. E. Carette et al., Nat Biotechnol 29, 542 (June, 2011).
9. J. E. Carette et al., Nature 477, 340 (Sep. 15, 2011).
10. B. Lai et al., Exp Neurol 194, 484 (August, 2005).
11. M. Galkin, R. Venard, J. Vaillier, J. Velours, F. Haraux, Eur J Biochem 271, 1963 (May, 2004).
12. M. Campanella et al., Cell Metab 8, 13 (July, 2008).
13. W. Rouslin, C. W. Broge, J Biol Chem 271, 23638 (Sep. 27, 1996).
14. M. McKenzie et al., J Biol Chem 282, 36845 (Dec. 21, 2007).
15. K. V. Tormos et al., Cell Metab 14, 537 (Oct. 5, 2011).
16. N. S. Chandel et al., J Biol Chem 275, 25130 (Aug. 18, 2000).
17. D. H. Kim et al., Cell 110, 163 (Jul. 26, 2002).
18. D. H. Kim et al., Mol Cell 11, 895 (April, 2003).
19. P. Benit, S. Lebon, P. Rustin, Biochim Biophys Acta 1793, 181 (January, 2009).
20. I. Visapaa et al., Am J Hum Genet. 71, 863 (October, 2002).
21. P. Leveen et al., Hepatology, (2011; 53(2):437-47; epub Dec. 28, 2010).
22. Brown, S. M., and Moore, M. (2012.). Mamm Genome 23, 632-640.
23. Darrouzet, E., et al., FEBS Letters 431, 34-38.
24. Edwards, M., et al. (2013). Isolation of mouse hepatocytes. In Cytochrome P450 Protocols. I. R. Phillips, E. A. Shephard, and P. R. Ortiz de Montellano, eds. (Humana Press), pp. 283-293.
25. Klaunig, J., Goldblatt, P., Hinton, D., Lipsky, M., and Trump, B. (1981). Mouse liver cell culture. In Vitro 17, 926-934.
26. Lee, W. S., and Sokol, R. J. (2007). Liver disease in mitochondrial disorders. Semin Liver Dis 27, 259-273.
27. \check{S}krtić, M., Sriskanthadevan, S., Jhas, B., Gebbia, M., Wang, X., Wang, Z., Hurren, R., Jitkova, Y., Gronda, M., Maclean, N., et al. (2011). Cancer Cell 20, 674-688.
28. Jazayeri, M., Andreyev, A., Will, Y., Ward, M., Anderson, C. M., and Clevenger, W. (2003). Journal of Biological Chemistry 278, 9823-9830.
29. Campanella, M., Parker, N., Tan, C. H., Hall, A. M., and Duchen, M. R. (2009). IF1: setting the pace of the FIFO-ATP synthase. Trends in biochemical sciences 34, 343-350.
30. Lu, Y.-M., Miyazawa, K., Yamaguchi, K., Nowaki, K., Iwatsuki, H., Wakamatsu, Y., Ichikawa, N., and Hashimoto, T. (2001). Journal of Biochemistry 130, 873-878
31. Fujikawa, M., Imamura, H., Nakamura, J., and Yoshida, M. (2012). Journal of Biological Chemistry 287, 18781-18787.
32. Appleby, R. D., Porteous, W. K., Hughes, G., James, A. M., Shannon, D., Wei, Y.-H., and Murphy, M. P. (1999). European Journal of Biochemistry 262, 108-116.
33. Buchet, K., and Godinot, C. (1998). Journal of Biological Chemistry 273, 22983-22989.
34. Ichikawa, N., Karaki, A., Kawabata, M., Ushida, S., Mizushima, M., and Hashimoto, T. (2001). Journal of Biochemistry 130, 687-693.
35. Lewis, W., Day, B. J., and Copeland, W. C. (2003). Nat Rev Drug Discov 2, 812-822.
36. Walker, U. A., Setzer, B., and Venhoff, N. (2002). AIDS 16, 2165-2173.
37. Morris, A. A. M. (1999). Liver 19, 357-368.
38. Fitzgerald, K., Frank-Kamenetsky, M., Shulga-Morskaya, S., Liebow, A., Bettencourt, B. R., Sutherland, J. E., Hutabarat, R. M., Clausen, V. A., Karsten, V., Cehelsky, J., et al. Effect of an RNA interference drug on the synthesis of proprotein convertase subtilisin/kexin type 9 (PCSK9) and the concentration of serum LDL cholesterol in healthy volunteers: a randomised, single-blind, placebo-controlled, phase 1 trial. The Lancet.
39. Vander Heiden, M. G., Cantley, L. C., and Thompson, C. B. (2009). Science 324, 1029-1033.
40. Nathwani, A. C., Tuddenham, E. G. D., Rangarajan, S., Rosales, C., McIntosh, J., Linch, D. C., Chowdary, P., Riddell, A., Pie, A. J., Harrington, C., et al. (2011). New England Journal of Medicine 365, 2357-2365.
41. Nakamura, J., Fujikawa, M., and Yoshida, M. (2013). Bioscience Reports 33.

1.-202. (canceled)

203. A method of identifying a candidate drug for a mitochondrial disorder, the method comprising identifying an ATPIF1 modulator.

204. The method of claim 203 comprising identifying an ATPIF1 inhibitor.

205. The method of claim 203 comprising: (a) contacting a test agent with a polypeptide comprising an ATPIF1 polypeptide; (b) determining whether the test agent binds to ATPIF1; and (c) identifying the test agent as a candidate drug for a mitochondrial disorder if the test agent binds to the ATPIF1 polypeptide.

206.-207. (canceled)

208. The method of claim 203 comprising: (a) contacting a test agent with a cell; (b) determining whether the test agent inhibits expression or activity of ATPIF1 in the cell; and (c) identifying the test agent as a candidate drug for a mitochondrial disorder if the test agent inhibits expression or activity of ATPIF1 in the cell.

209.-249. (canceled)

250. A method of inhibiting death or degeneration of a mammalian cell that has mitochondrial dysfunction, the method comprising contacting the cell with an ATPIF1 inhibitor.

251. (canceled)

252. The method of claim 250, wherein the mammalian cell has a defect in oxidative phosphorylation.

253. (canceled)

254. The method of claim 250, wherein the mammalian cell originates from a subject suffering from a mitochondrial disorder characterized by loss or degeneration of cells having mitochondrial dysfunction.

255.-259. (canceled)

260. The method of claim 250, wherein the mammalian cell is a hepatocyte.

261. The method of claim 250, wherein the mammalian cell is a neuron.

262. (canceled)

263. The method of claim 250, wherein the mammalian cell has been exposed to a mitochondrial poison.

264.-265. (canceled)

266. A method of treating a mammalian subject in need of treatment for a mitochondrial disorder, the method comprising administering an ATPIF1 inhibitor to the subject.

267. The method of claim 266, wherein the mitochondrial disorder is characterized by liver dysfunction.

268. The method of claim 266, wherein the mitochondrial disorder is a neurodegenerative disorder.

269. The method of claim 266, wherein the mitochondrial disorder is Parkinson's disease, an optic atrophy, or GRACILE syndrome.

270.-273. (canceled)

274. The method of claim 266, wherein the ATPIF1 inhibitor inhibits expression of ATPIF1.

275.-303. (canceled)

304. The method of claim 266 comprising administering a vector comprising a nucleic acid construct comprising a sequence that encodes a polynucleotide that inhibits ATPIF1 expression or activity when expressed in a mammalian cell, wherein the sequence is operably linked to a promoter capable of directing transcription of the sequence in a mammalian cell to the subject.

305. The method of claim 304, wherein the vector is administered locally to an organ affected by the mitochondrial disorder.

306. (canceled)

307. The method of claim 304, wherein the vector comprises a viral vector capable of transducing human hepatocytes or neurons.

308. The method of claim 304, wherein the polynucleotide comprises an shRNA, siRNA, or miRNA.

309. The method of claim 250, wherein the ATPIF1 inhibitor comprises an RNAi agent or antisense agent.
